SEQUENCE LISTING (227 sequences)

SEQ ID NO: 1 - protein, 11 aa, synthetic construct
GYSITSGYFWN

SEQ ID NO: 2 - protein, 9 aa, synthetic construct
YISYDGSNN

SEQ ID NO: 3 - protein, 11 aa, synthetic construct
PSPGTGYAVDY

SEQ ID NO: 4 - protein, 6 aa, synthetic construct
SGYFWN

SEQ ID NO: 5 - protein, 16 aa, synthetic construct
YISYDGSNNYNPSLKS

SEQ ID NO: 6 - protein, 11 aa, synthetic construct
PSPGTGYAVDY

SEQ ID NO: 7 - protein, 8 aa, synthetic construct
GYSITSGY

SEQ ID NO: 8 - protein, 5 aa, synthetic construct
SYDGS

SEQ ID NO: 9 - protein, 11 aa, synthetic construct
PSPGTGYAVDY

SEQ ID NO: 10 - protein, 9 aa, synthetic construct
GYSITSGYF

SEQ ID NO: 11 - protein, 7 aa, synthetic construct
ISYDGSN

SEQ ID NO: 12 - protein, 13 aa, synthetic construct
ASPSPGTGYAVDY

SEQ ID NO: 13 - protein, 7 aa, synthetic construct
TSGYFWN

SEQ ID NO: 14 - protein, 12 aa, synthetic construct
WIGYISYDGSNN

SEQ ID NO: 15 - protein, 12 aa, synthetic construct
ASPSPGTGYAVD

SEQ ID NO: 16 - protein, 16 aa, synthetic construct
RSSQSLVHSSGNTYLH

SEQ ID NO: 17 - protein, 7 aa, synthetic construct
KVSNRFS

SEQ ID NO: 18 - protein, 9 aa, synthetic construct
SQSTHVPFT

SEQ ID NO: 19 - protein, 16 aa, synthetic construct
RSSQSLVHSSGNTYLH

SEQ ID NO: 20 - protein, 7 aa, synthetic construct
KVSNRFS

SEQ ID NO: 21 - protein, 9 aa, synthetic construct
SQSTHVPFT

SEQ ID NO: 22 - protein, 16 aa, synthetic construct
RSSQSLVHSSGNTYLH

SEQ ID NO: 23 - protein, 7 aa, synthetic construct
KVSNRFS

SEQ ID NO: 24 - protein, 9 aa, synthetic construct
SQSTHVPFT

SEQ ID NO: 25 - protein, 11 aa, synthetic construct
QSLVHSSGNTY

SEQ ID NO: 26 - skipped

SEQ ID NO: 27 - protein, 9 aa, synthetic construct
SQSTHVPFT

SEQ ID NO: 28 - protein, 12 aa, synthetic construct
VHSSGNTYLHWY

SEQ ID NO: 29 - protein, 10 aa, synthetic construct
FLIYKVSNRF

SEQ ID NO: 30 - protein, 8 aa, synthetic construct
SQSTHVPF

SEQ ID NO: 31 - protein, 120 aa, synthetic construct
QVQLQESGPGLVKPSETLSLTCTVSGYSITSGYFWNWIRQPPGKGLEWIGYISYDGSNNYNPSLKSRVTISRDTSKNQFSLKLSSVTAADTAVYYCASPSPGTGYAVDYWGQGTLVTVSS

SEQ ID NO: 32 - protein, 112 aa, synthetic construct
DIQMTQSPSSLSASVGDRVTITCRSSQSLVHSSGNTYLHWYQQKPGKAPKFLIYKVSNRFSGVPSRFSGSGSGTDFTLTISSLQPEDFATYYCSQSTHVPFTFGQGTKLEIK

SEQ ID NO: 33 - protein, 10 aa, synthetic construct
GFTFSSYWMH

SEQ ID NO: 34 - protein, 10 aa, synthetic construct
RISSHGSSTS

SEQ ID NO: 35 - protein, 12 aa, synthetic construct
GGNYYDSPYFDY

SEQ ID NO: 36 - protein, 4 aa, synthetic construct
SYWM

SEQ ID NO: 37 - protein, 17 aa, synthetic construct
RISSHGSSTSYADSVKG

SEQ ID NO: 38 - protein, 12 aa, synthetic construct
GGNYYDSPYFDY

SEQ ID NO: 39 - protein, 7 aa, synthetic construct
GFTFSSY

SEQ ID NO: 40 - protein, 6 aa, synthetic construct
SSHGSS

SEQ ID NO: 41 - protein, 12 aa, synthetic construct
GGNYYDSPYFDY

SEQ ID NO: 42 - protein, 8 aa, synthetic construct
GFTFSSYW

SEQ ID NO: 43 - protein, 8 aa, synthetic construct
ISSHGSST

SEQ ID NO: 44 - protein, 14 aa, synthetic construct
ARGGNYYDSPYFDY

SEQ ID NO: 45 - protein, 6 aa, synthetic construct
SSYWMH

SEQ ID NO: 46 - protein, 13 aa, synthetic construct
WVSRISSHGSSTS

SEQ ID NO: 47 - protein, 13 aa, synthetic construct
ARGGNYYDSPYFD

SEQ ID NO: 48 - protein, 14 aa, synthetic construct
TGTSSDVGGYNYVS

SEQ ID NO: 49 - protein, 7 aa, synthetic construct
EVSYRPS

SEQ ID NO: 50 - protein, 11 aa, synthetic construct
SSYTSSSTYVV

SEQ ID NO: 51 - protein, 14 aa, synthetic construct
TGTSSDVGGYNYVS

SEQ ID NO: 52 - protein, 7 aa, synthetic construct
EVSYRPS

SEQ ID NO: 53 - protein, 11 aa, synthetic construct
SSYTSSSTYVV

SEQ ID NO: 54 - protein, 14 aa, synthetic construct
TGTSSDVGGYNYVS

SEQ ID NO: 55 - protein, 7 aa, synthetic construct
EVSYRPS

SEQ ID NO: 56 - protein, 11 aa, synthetic construct
SSYTSSSTYVV

SEQ ID NO: 57 - protein, 9 aa, synthetic construct
SSDVGGYNY

SEQ ID NO: 58 - skipped

SEQ ID NO: 59 - protein, 11 aa, synthetic construct
SSYTSSSTYVV

SEQ ID NO: 60 - protein, 10 aa, synthetic construct
VGGYNYVSWY

SEQ ID NO: 61 - protein, 10 aa, synthetic construct
LMIYEVSYRP

SEQ ID NO: 62 - protein, 10 aa, synthetic construct
SSYTSSSTYV

SEQ ID NO: 63 - protein, 121 aa, synthetic construct
EVQLVESGGGLVQPGGSLRLSCAASGFTFSSYWMHWVRQVPGKGLVWVSRISSHGSSTSYADSVKGRFTISRDNAKNALYLQMNSLRAEDTAVYYCARGGNYYDSPYFDYWGQGTLVTVSS

SEQ ID NO: 64 - protein, 111 aa, synthetic construct
QSALTQPASVSGSPGQSITISCTGTSSDVGGYNYVSWYQQHPGKAPKLMIYEVSYRPSGISNRFSGSKSGDTASLTISGLQAEDEADYYCSSYTSSSTYVVFGGGTKVTVL

SEQ ID NO: 65 - protein, 10 aa, synthetic construct
GFTFSRYSMN

SEQ ID NO: 66 - protein, 10 aa, synthetic construct
YISSSSNTIY

SEQ ID NO: 67 - protein, 8 aa, synthetic construct
DQGTPFDY

SEQ ID NO: 68 - protein, 5 aa, synthetic construct
RYSMN

SEQ ID NO: 69 - protein, 17 aa, synthetic construct
YISSSSNTIYYADSVKG

SEQ ID NO: 70 - protein, 8 aa, synthetic construct
DQGTPFDY

SEQ ID NO: 71 - protein, 7 aa, synthetic construct
GFTFSRY

SEQ ID NO: 72 - protein, 6 aa, synthetic construct
SSSSNT

SEQ ID NO: 73 - protein, 8 aa, synthetic construct
DQGTPFDY

SEQ ID NO: 74 - protein, 8 aa, synthetic construct
GFTFSRYS

SEQ ID NO: 75 - protein, 8 aa, synthetic construct
ISSSSNTI

SEQ ID NO: 76 - protein, 10 aa, synthetic construct
ARDQGTPFDY

SEQ ID NO: 77 - protein, 6 aa, synthetic construct
SRYSMN

SEQ ID NO: 78 - protein, 13 aa, synthetic construct
WVSYISSSSNTIY

SEQ ID NO: 79 - protein, 9 aa, synthetic construct
ARDQGTPFD

SEQ ID NO: 80 - protein, 11 aa, synthetic construct
RASQDISSWLG

SEQ ID NO: 81 - protein, 7 aa, synthetic construct
AAISLQS

SEQ ID NO: 82 - protein, 9 aa, synthetic construct
HQANSFPLT

SEQ ID NO: 83 - protein, 11 aa, synthetic construct
RASQDISSWLG

SEQ ID NO: 84 - protein, 7 aa, synthetic construct
AAISLQS

SEQ ID NO: 85 - protein, 9 aa, synthetic construct
HQANSFPLT

SEQ ID NO: 86 - protein, 11 aa, synthetic construct
RASQDISSWLG

SEQ ID NO: 87 - protein, 7 aa, synthetic construct
AAISLQS

SEQ ID NO: 88 - protein, 9 aa, synthetic construct
HQANSFPLT

SEQ ID NO: 89 - protein, 6 aa, synthetic construct
QDISSW

SEQ ID NO: 90 - skipped

SEQ ID NO: 91 - protein, 9 aa, synthetic construct
HQANSFPLT

SEQ ID NO: 92 - protein, 7 aa, synthetic construct
SSWLGWY

SEQ ID NO: 93 - protein, 10 aa, synthetic construct
LLIYAAISLQ

SEQ ID NO: 94 - protein, 8 aa, synthetic construct
HQANSFPL

SEQ ID NO: 95 - protein, 117 aa, synthetic construct
EVQLVESGGGLVQPGGSLRLSCTASGFTFSRYSMNWVRQAPGEGLEWVSYISSSSNTIYYADSVKGRFTISRDNAKNSLYLQMNSLRDEDTAVYYCARDQGTPFDYWGQGTLVTVSS

SEQ ID NO: 96 - protein, 107 aa, synthetic construct
EIVLTQSPSSVSASIGDRVTITCRASQDISSWLGWYQQKPGKAPKLLIYAAISLQSGVPSRFSGSESGTDFTLTISSLQPEDFATYYCHQANSFPLTFGGGTKVEIK

SEQ ID NO: 97 - protein, 10 aa, synthetic construct
GFIFRSYSMN

SEQ ID NO: 98 - protein, 10 aa, synthetic construct
SISSSSSYIY

SEQ ID NO: 99 - protein, 9 aa, synthetic construct
REYGGPFDY

SEQ ID NO: 100 - protein, 5 aa, synthetic construct
SYSMN

SEQ ID NO: 101 - protein, 17 aa, synthetic construct
SISSSSSYIYYADSVKG

SEQ ID NO: 102 - protein, 9 aa, synthetic construct
REYGGPFDY

SEQ ID NO: 103 - protein, 7 aa, synthetic construct
GFIFRSY

SEQ ID NO: 104 - protein, 6 aa, synthetic construct
SSSSSY

SEQ ID NO: 105 - protein, 9 aa, synthetic construct
REYGGPFDY

SEQ ID NO: 106 - protein, 8 aa, synthetic construct
GFIFRSYS

SEQ ID NO: 107 - protein, 8 aa, synthetic construct
ISSSSSYI

SEQ ID NO: 108 - protein, 11 aa, synthetic construct
ARREYGGPFDY

SEQ ID NO: 109 - protein, 6 aa, synthetic construct
RSYSMN

SEQ ID NO: 110 - protein, 13 aa, synthetic construct
WVSSISSSSSYIY

SEQ ID NO: 111 - protein, 10 aa, synthetic construct
ARREYGGPFD

SEQ ID NO: 112 - protein, 13 aa, synthetic construct
SGSSSNIGNDYVS

SEQ ID NO: 113 - protein, 7 aa, synthetic construct
DNNKRPS

SEQ ID NO: 114 - protein, 12 aa, synthetic construct
GTWDSSLSAGYV

SEQ ID NO: 115 - protein, 13 aa, synthetic construct
SGSSSNIGNDYVS

SEQ ID NO: 116 - protein, 7 aa, synthetic construct
DNNKRPS

SEQ ID NO: 117 - protein, 12 aa, synthetic construct
GTWDSSLSAGYV

SEQ ID NO: 118 - protein, 13 aa, synthetic construct
SGSSSNIGNDYVS

SEQ ID NO: 119 - protein, 7 aa, synthetic construct
DNNKRPS

SEQ ID NO: 120 - protein, 12 aa, synthetic construct
GTWDSSLSAGYV

SEQ ID NO: 121 - protein, 8 aa, synthetic construct
SSNIGNDY

SEQ ID NO: 122 - skipped

SEQ ID NO: 123 - protein, 12 aa, synthetic construct
GTWDSSLSAGYV

SEQ ID NO: 124 - protein, 9 aa, synthetic construct
IGNDYVSWY

SEQ ID NO: 125 - protein, 10 aa, synthetic construct
LLIYDNNKRP

SEQ ID NO: 126 - protein, 11 aa, synthetic construct
GTWDSSLSAGY

SEQ ID NO: 127 - protein, 118 aa, synthetic construct
EVQLVQSGGGLVKPGGSLRLSCVASGFIFRSYSMNWVRQAPGKGLEWVSSISSSSSYIYYADSVKGRFTISRDNAKNSLYLQMDSLRAEDTAVYYCARREYGGPFDYWGQGTLVTVSS

SEQ ID NO: 128 - protein, 111 aa, synthetic construct
QSVLTQPPSVFAAPGEKVTISCSGSSSNIGNDYVSWYQQLPGTAPKLLIYDNNKRPSGIPDRFSGSKSGTSATLGITGLQTGDEADYYCGTWDSSLSAGYVFGTGTKVTVL

SEQ ID NO: 129 - protein, 11 aa, synthetic construct
GYSITSGYFWN

SEQ ID NO: 130 - protein, 9 aa, synthetic construct
YISYDGSNN

SEQ ID NO: 131 - protein, 11 aa, synthetic construct
PSPGTGYAVDY

SEQ ID NO: 132 - protein, 6 aa, synthetic construct
SGYFWN

SEQ ID NO: 133 - protein, 16 aa, synthetic construct
YISYDGSNNYNPSLKS

SEQ ID NO: 134 - protein, 11 aa, synthetic construct
PSPGTGYAVDY

SEQ ID NO: 135 - protein, 8 aa, synthetic construct
GYSITSGY

SEQ ID NO: 136 - protein, 5 aa, synthetic construct
SYDGS

SEQ ID NO: 137 - protein, 11 aa, synthetic construct
PSPGTGYAVDY

SEQ ID NO: 138 - protein, 9 aa, synthetic construct
GYSITSGYF

SEQ ID NO: 139 - protein, 7 aa, synthetic construct
ISYDGSN

SEQ ID NO: 140 - protein, 13 aa, synthetic construct
ASPSPGTGYAVDY

SEQ ID NO: 141 - protein, 7 aa, synthetic construct
TSGYFWN

SEQ ID NO: 142 - protein, 12 aa, synthetic construct
WMGYISYDGSNN

SEQ ID NO: 143 - protein, 12 aa, synthetic construct
ASPSPGTGYAVD

SEQ ID NO: 144 - protein, 16 aa, synthetic construct
RSSQSLVHSNRNTYLH

SEQ ID NO: 145 - protein, 7 aa, synthetic construct
KVSNRFS

SEQ ID NO: 146 - protein, 9 aa, synthetic construct
SQSTHVPFT

SEQ ID NO: 147 - protein, 16 aa, synthetic construct
RSSQSLVHSNRNTYLH

SEQ ID NO: 148 - protein, 7 aa, synthetic construct
KVSNRFS

SEQ ID NO: 149 - protein, 9 aa, synthetic construct
SQSTHVPFT

SEQ ID NO: 150 - protein, 16 aa, synthetic construct
RSSQSLVHSNRNTYLH

SEQ ID NO: 151 - protein, 7 aa, synthetic construct
KVSNRFS

SEQ ID NO: 152 - protein, 9 aa, synthetic construct
SQSTHVPFT

SEQ ID NO: 153 - protein, 11 aa, synthetic construct
QSLVHSNRNTY

SEQ ID NO: 154 - skipped

SEQ ID NO: 155 - protein, 9 aa, synthetic construct
SQSTHVPFT

SEQ ID NO: 156 - protein, 12 aa, synthetic construct
VHSNRNTYLHWY

SEQ ID NO: 157 - protein, 10 aa, synthetic construct
LLIYKVSNRF

SEQ ID NO: 158 - protein, 8 aa, synthetic construct
SQSTHVPF

SEQ ID NO: 159 - protein, 120 aa, synthetic construct
QVQLQESGPGLVKPSETLSLTCTVSGYSITSGYFWNWIRQFPGKGLEWMGYISYDGSNNYNPSLKSRITISRDTSKNQFSLKLSSVTAADTAVYYCASPSPGTGYAVDYWGCGTTVTVSS

SEQ ID NO: 160 - protein, 112 aa, synthetic construct
DIQMTQSPSSLSASVGDRVTITCRSSQSLVHSNRNTYLHWYQQKPGCAPKLLIYKVSNRFSGVPSRFSGSGSGTDFTLTISSLQPEDFATYYCSQSTHVPFTFGQGTKLEIK

SEQ ID NO: 161 - protein, 10 aa, synthetic construct
GFTFRSYWMH

SEQ ID NO: 162 - protein, 10 aa, synthetic construct
RISSGGSTTN

SEQ ID NO: 163 - protein, 12 aa, synthetic construct
GDNYYDSPYFDY

SEQ ID NO: 164 - protein, 5 aa, synthetic construct
SYWMH

SEQ ID NO: 165 - protein, 17 aa, synthetic construct
RISSGGSTTNYADSVKG

SEQ ID NO: 166 - protein, 12 aa, synthetic construct
GDNYYDSPYFDY

SEQ ID NO: 167 - protein, 7 aa, synthetic construct
GFTFRSY

SEQ ID NO: 168 - protein, 6 aa, synthetic construct
SSGGST

SEQ ID NO: 169 - protein, 12 aa, synthetic construct
GDNYYDSPYFDY

SEQ ID NO: 170 - protein, 8 aa, synthetic construct
GFTFRSYW

SEQ ID NO: 171 - protein, 8 aa, synthetic construct
ISSGGSTT

SEQ ID NO: 172 - protein, 14 aa, synthetic construct
ARGDNYYDSPYFDY

SEQ ID NO: 173 - protein, 6 aa, synthetic construct
RSYWMH

SEQ ID NO: 174 - protein, 13 aa, synthetic construct
WVSRISSGGSTTN

SEQ ID NO: 175 - protein, 13 aa, synthetic construct
ARGDNYYDSPYFD

SEQ ID NO: 176 - protein, 14 aa, synthetic construct
TGTSSDVGGYNYVS

SEQ ID NO: 177 - protein, 7 aa, synthetic construct
EVSYRPS

SEQ ID NO: 178 - protein, 11 aa, synthetic construct
SSYTSSSTYVV

SEQ ID NO: 179 - protein, 14 aa, synthetic construct
TGTSSDVGGYNYVS

SEQ ID NO: 180 - protein, 7 aa, synthetic construct
EVSYRPS

SEQ ID NO: 181 - protein, 11 aa, synthetic construct
SSYTSSSTYVV

SEQ ID NO: 182 - protein, 14 aa, synthetic construct
TGTSSDVGGYNYVS

SEQ ID NO: 183 - protein, 7 aa, synthetic construct
EVSYRPS

SEQ ID NO: 184 - protein, 11 aa, synthetic construct
SSYTSSSTYVV

SEQ ID NO: 185 - protein, 9 aa, synthetic construct
SSDVGGYNY

SEQ ID NO: 186 - skipped

SEQ ID NO: 187 - protein, 11 aa, synthetic construct
SSYTSSSTYVV

SEQ ID NO: 188 - protein, 10 aa, synthetic construct
VGGYNYVSWY

SEQ ID NO: 189 - protein, 10 aa, synthetic construct
LMIYEVSYRP

SEQ ID NO: 190 - protein, 10 aa, synthetic construct
SSYTSSSTYV

SEQ ID NO: 191 - protein, 121 aa, synthetic construct
EVQLVESGGGLVQPGGSLRLSCAASGFTFRSYWMHWVRQAPGKGLVWVSRISSGGSTTNYADSVKGRFTISRDNAKNTLYLQMNSLRAEDTAIYYCARGDNYYDSPYFDYWGCGTLVTVSS

SEQ ID NO: 192 - protein, 111 aa, synthetic construct
QSALTQPASVSGSPGQSITISCTGTSSDVGGYNYVSWYQQHPGCAPKLMIYEVSYRPSGVSNRFSGSKSGNTASLTISGLQAEDEADYYCSSYTSSSTYVVFGGGTKLTVL

SEQ ID NO: 193 - protein, 482 aa, synthetic construct
DIQMTQSPSSLSASVGDRVTITCRSSQSLVHSNRNTYLHWYQQKPGCAPKLLIYKVSNRFSGVPSRFSGSGSGTDFTLTISSLQPEDFATYYCSQSTHVPFTFGQGTKLEIKGGGSGGSGGCPPCGGSGGQVQLQESGPGLVKPSETLSLTCTVSGYSITSGYFWNWIRQFPGKGLEWMGYISYDGSNNYNPSLKSRITISRDTSKNQFSLKLSSVTAADTAVYYCASPSPGTGYAVDYWGCGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 448 aa, synthetic construct
EVQLVQSGGGLVKPGGSLRLSCVASGFIFRSYSMNWVRQAPGKGLEWVSSISSSSSYIYYADSVKGRFTISRDNAKNSLYLQMDSLRAEDTAVYYCARREYGGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 195 - protein, 217 aa, synthetic construct
QSVLTQPPSVFAAPGEKVTISCSGSSSNIGNDYVSWYQQLPGTAPKLLIYDNNKRPSGIPDRFSGSKSGTSATLGITGLQTGDEADYYCGTWDSSLSAGYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 196 - protein, 451 aa, synthetic construct
EVQLVESGGGLVQPGGSLRLSCAASGFTFSSYWMHWVRQVPGKGLVWVSRISSHGSSTSYADSVKGRFTISRDNAKNALYLQMNSLRAEDTAVYYCARGGNYYDSPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 197 - protein, 217 aa, synthetic construct
QSALTQPASVSGSPGQSITISCTGTSSDVGGYNYVSWYQQHPGKAPKLMIYEVSYRPSGISNRFSGSKSGDTASLTISGLQAEDEADYYCSSYTSSSTYVVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 198 - protein, 447 aa, synthetic construct
EVQLVESGGGLVQPGGSLRLSCTASGFTFSRYSMNWVRQAPGEGLEWVSYISSSSNTIYYADSVKGRFTISRDNAKNSLYLQMNSLRDEDTAVYYCARDQGTPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 199 - protein, 214 aa, synthetic construct
EIVLTQSPSSVSASIGDRVTITCRASQDISSWLGWYQQKPGKAPKLLIYAAISLQSGVPSRFSGSESGTDFTLTISSLQPEDFATYYCHQANSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 450 aa, synthetic construct
QVQLQESGPGLVKPSETLSLTCTVSGYSITSGYFWNWIRQPPGKGLEWIGYISYDGSNNYNPSLKSRVTISRDTSKNQFSLKLSSVTAADTAVYYCASPSPGTGYAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 201 - protein, 219 aa, synthetic construct
DIQMTQSPSSLSASVGDRVTITCRSSQSLVHSSGNTYLHWYQQKPGKAPKFLIYKVSNRFSGVPSRFSGSGSGTDFTLTISSLQPEDFATYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - protein, 482 aa, synthetic construct
QSALTQPASVSGSPGQSITISCTGTSSDVGGYNYVSWYQQHPGCAPKLMIYEVSYRPSGVSNRFSGSKSGNTASLTISGLQAEDEADYYCSSYTSSSTYVVFGGGTKLTVLGGGSGGSGGCPPCGGSGGEVQLVESGGGLVQPGGSLRLSCAASGFTFRSYWMHWVRQAPGKGLVWVSRISSGGSTTNYADSVKGRFTISRDNAKNTLYLQMNSLRAEDTAIYYCARGDNYYDSPYFDYWGCGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 203 - protein, 334 aa, synthetic construct
MARKSALLALALLLLGFPGAWGDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQKVLCVFWEHGQNGCGHWATTGCSTIGTRDTSTICRCTHLSSFAVLMAHYDVQEEDPVLTVITGGGGSGLNDIFEAQKIEWHEGGGGSHHHHHHG

SEQ ID NO: 204 - protein, 272 aa, synthetic construct
MARKSALLALALLLLGFPGAWGDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQGGGGSGLNDIFEAQKIEWHEGGGGSHHHHHHG

SEQ ID NO: 205 - protein, 823 aa, Homo sapiens
MGGRVFLVFLAFCVWLTLPGAETQDSRGCARWCPQDSSCVNATACRCNPGFSSFSEIITTPMETCDDINECATLSKVSCGKFSDCWNTEGSYDCVCSPGYEPVSGAKTFKNESENTCQDVDECQQNPRLCKSYGTCVNTLGSYTCQCLPGFKLKPEDPKLCTDVNECTSGQNPCHSSTHCLNNVGSYQCRCRPGWQPIPGSPNGPNNTVCEDVDECSSGQHQCDSSTVCFNTVGSYSCRCRPGWKPRHGIPNNQKDTVCEDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQKVLCVFWEHGQNGCGHWATTGCSTIGTRDTSTICRCTHLSSFAVLMAHYDVQEEDPVLTVITYMGLSVSLLCLLLAALTFLLCKAIQNTSTSLHLQLSLCLFLAHLLFLVAIDQTGHKVLCSIIAGTLHYLYLATLTWMLLEALYLFLTARNLTVVNYSSINRFMKKLMFPVGYGVPAVTVAISAASRPHLYGTPSRCWLQPEKGFIWGFLGPVCAIFSVNLVLFLVTLWILKNRLSSLNSEVSTLRNTRMLAFKATAQLFILGCTWCLGILQVGPAARVMAYLFTIINSLQGVFIFLVYCLLSQQVREQYGKWSKGIRKLKTESEMHTLSSSAKADTSKPSTVN

SEQ ID NO: 206 - protein, 585 aa, Homo sapiens
MARKSALLALALLLLGFPGAWGDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQKVLCVFWEHGQNGCGHWATTGCSTIGTRDTSTICRCTHLSSFAVLMAHYDVQEEDPVLTVITYMGLSVSLLCLLLAALTFLLCKAIQNTSTSLHLQLSLCLFLAHLLFLVAIDQTGHKVLCSIIAGTLHYLYLATLTWMLLEALYLFLTARNLTVVNYSSINRFMKKLMFPVGYGVPAVTVAISAASRPHLYGTPSRCWLQPEKGFIWGFLGPVCAIFSVNLVLFLVTLWILKNRLSSLNSEVSTLRNTRMLAFKATAQLFILGCTWCLGILQVGPAARVMAYLFTIINSLQGVFIFLVYCLLSQQVREQYGKWSKGIRKLKTESEMHTLSSSAKADTSKPSTVN

SEQ ID NO: 207-210 - skipped (4 entries)

SEQ ID NO: 211 - protein, 449 aa, synthetic construct
MAWVWTLLFLMAAAQSIQAQLLEQSPQFLSIQEGENLTVYCNSSSVFSSLQWYRQEPGEGPVLLVTVVTGGEVKKLKRLTFQFGDARKDSSLHITAAQPGDTGLYLCAGAGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 212 - protein, 493 aa, synthetic construct
MAWVWTLLFLMAAAQSIQAVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSSRSSYEQYFGPGTRLTVTEDLKNVFPPEVAVFEPSEAEISHTQKATLVCLATGFYPDHVELSWWVNGKEVHSGVSTDPQPLKEQPALNDSRYSLSSRLRVSATFWQNPRNHFRCQVQFYGLSENDEWTQDRAKPVTQIVSAEAWGRADEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 213 - protein, 450 aa, synthetic construct
MAWVWTLLFLMAAAQSIQATQLLEQSPQFLSIQEGENLTVYCNSSSVFSSLQWYRQEPGEGPVLLVTVVTGGEVKKLKRLTFQFGDARKDSSLHITAAQPGDTGLYLCAGAGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 214 - protein, 494 aa, synthetic construct
MARKSALLALALLLLFGPAWADGGITQSPKYLFRKEGQNVTLSCEQNLNHDAMYWYRQDPGQGLRLIYYSQIVNDFQKGDIAEGYSVSREKKESFPLTVTSAQKNPTAFYLCASSSRSSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 215 - protein, 218 aa, Homo sapiens
DMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQ

SEQ ID NO: 216 - protein, 51 aa, Homo sapiens
KVLCVFWEHGQNGCGHWATTGCSTIGTRDTSTICRCTHLSSFAVLMAHYDV

SEQ ID NO: 217 - protein, 9 aa, Homo sapiens
NQAVMQLDW

SEQ ID NO: 218 - protein, 24 aa, Homo sapiens
HGQNGCGHWATTGCSTIGTRDTST

SEQ ID NO: 219 - protein, 4 aa, Homo sapiens
IVND

SEQ ID NO: 220 - skipped

SEQ ID NO: 221 - protein, 18 aa, synthetic construct
GGGSGGSGGCPPCGGSGG

SEQ ID NO: 222 - protein, 107 aa, synthetic construct [sequence omitted]

SEQ ID NO: 223 - protein, 95 aa, synthetic construct
FPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 224 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 225 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 6 aa, synthetic construct
HHHHHH

SEQ ID NO: 227 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0361317 A1
(43) Pub. Date: Nov. 27, 2025

(54) BISPECIFIC ANTIBODY TARGETING EMR2 (CD312) AND THE T-CELL RECEPTOR TRBV19

(71) Applicant: JANSSEN BIOTECH, INC., HORSHAM, PA (US)

(72) Inventors: Nicholas Andrew MANIERI, NORTH WALES, PA (US); Mehabaw Getahun DEREBE, BURLINGAME, CA (US); Sara EL ASHKAR, EDEGEM (BE); Ulrike PHILIPPAR, ANTWERPEN (BE)

(21) Appl. No.: 19/215,918

(22) Filed: May 22, 2025

Related U.S. Application Data
(60) Provisional application No. 63/651,800, filed on May 24, 2024.

(51) Int. Cl. C07K 16/28; A61K 39/00; A61P 35/00

(52) U.S. Cl. CPC C07K 16/2896; A61P 35/00; C07K 16/2809; A61K 2039/505; C07K 2317/31; C07K 2317/33; C07K 2317/52; C07K 2317/92

(57) ABSTRACT

Provided herein are multispecific antibodies, including bispecific antibodies that specifically bind to EMR2 and TRBV19 (also known as Vβ17), and monospecific antibodies that specifically bind to EMR2, and multispecific antigen-binding fragments thereof. Also described are related polynucleotides capable of encoding the provided multispecific antibodies or multispecific antigen-binding fragments, cells expressing the provided multispecific antibodies or multispecific antigen-binding fragments, as well as associated vectors and detectably labeled multispecific antibodies or multispecific antigen-binding fragments. In addition, methods of producing and using the provided multispecific antibodies and multispecific antigen-binding fragments are described.

### CROSS REFERENCE TO RELATED APPLICATIONS

This application claims priority to U.S. Provisional Application No. 63/651,800, filed on May 24, 2024, the disclosure of which is herein incorporated by reference in its entirety.

### SEQUENCE LISTING

The instant application contains a Sequence Listing which has been submitted electronically in XML format and is hereby incorporated by reference in its entirety. Said XML copy, is named JBI6897USNP1_SL.xml created on May 5, 2025 and is 199,843 bytes in size.

### TECHNICAL FIELD

The disclosure provided herein relates to bispecific antibodies that specifically bind epidermal-growth-factor-like module-containing mucin-like hormone receptor-like 2 (EMR2) and the TRBV19 receptor (also known as Vβ17) on T cells and monospecific antibodies that specifically bind EMR2.

### BACKGROUND

Acute myeloid leukemia (AML) and myelodysplastic neoplasms (MDS; also known as myelodysplastic syndrome) are highly aggressive hematological malignancies characterized by clonal expansion of stem and myeloid progenitor cells. The current standard of care (SoC) for fit patients with AML is aggressive induction combination chemotherapy followed by high-dose chemotherapy and/or allogeneic transplantation. For AML patients that relapse or are refractory to SoC, targeted therapy is often combined with alternative regimens. While some patients achieve complete remission, 50% to 70% eventually relapse within 3 years and succumb to the disease. The current SoC for fit patients with MDS depends on the risk stratification; higher risk (HR) disease is managed with decitabine or azacytidine followed by allogeneic transplantation while lower-risk disease is managed with growth factors and supportive care until progression. Efforts to develop immunotherapies for AML and HR MDS have been challenging due to limited clinical activity, severe cytokine release syndrome (CRS), and significant toxicities owing to the broader expression of AML targets on healthy myeloid cells and nonhematopoietic tissues.

### SUMMARY

In one aspect, the disclosure provides an antibody or an antigen-binding fragment thereof, that specifically binds the G-protein-coupled receptor auto-proteolysis inducing (GAIN) domain and/or the GPCR proteolytic site (GPS) motif of epidermal-growth-factor-like module-containing mucin-like hormone receptor 2 (EMR2).

In some embodiments, the GAIN domain comprises amino acid residues D261-Q478 of SEQ ID NO: 205.

In some embodiments, the GAIN domain comprises the amino acid sequence SEQ ID NO: 215.

In any of the foregoing embodiments, the GPS motif comprises the amino acid sequence SEQ ID NO: 216.

In any of the foregoing embodiments, the antibody or the antigen-binding fragment thereof, binds to an epitope comprising SEQ ID NO: 217 or SEQ ID NO: 218.

In any of the foregoing embodiments, the antibody or the antigen-binding fragment thereof, binds to human EMR2 with a dissociation constant (K_{D}) between about 0.01 nM to about 5 nM.

In any of the foregoing embodiments, the antibody or the antigen-binding fragment thereof, binds to human EMR2 with an half maximal effective concentration (EC₅₀) between about 0.1 nM to about 15 nM.

In any of the foregoing embodiments, the antibody or the antigen-binding fragment thereof, is or comprises a fragment antigen-binding (Fab), a F(ab′)2 fragment, F(ab)′3 fragments, a single-chain variable fragment (scFv), a bis-scFv, a (scFv)2, a stapled scFv (spFv), a diabody, a minibody, a nanobody, a triabody, a tetrabody, a disulfide stabilized Fv protein (dsFv), a single-domain antibody (sdAb), an Immunoglobulin New Antigen Receptor (Ig NAR), a single heavy chain antibody, a camelid antibody, a shark antibody, or a chemically modified derivative thereof.

In any of the foregoing embodiments, the antibody or the antigen-binding fragment thereof, comprises a Fab.

In any of the foregoing embodiments, the antibody or the antigen-binding fragment thereof, further comprises a first constant Ig domain of the heavy chain (CH1) domain.

In any of the foregoing embodiments, the antibody or the antigen-binding fragment thereof, does not comprise a CH1 domain.

In any of the foregoing embodiments, the antibody or the antigen-binding fragment thereof, comprises a scFv or a spFv.

In some embodiments, the scFv or spFv comprises a signal sequence, a heavy chain variable sequence, a GS-Linker, and a light chain variable sequence.

In any of the foregoing embodiments, the antibody or the antigen-binding fragment thereof, further comprises a fragment crystallizable (Fc) domain.

In some embodiments, the Fc domain of the antibody or the antigen-binding fragment thereof, is an IgA, an IgG, an IgE, or an IgM. In some embodiments, the Fc domain of the antibody or the antigen-binding fragment thereof, is an IgG. In some embodiments, the IgG is IgG1 or IgG4.

In some of the foregoing embodiments, the Fc domain comprises one or more different mutations which promote heterodimerization.

In some of the foregoing embodiments, the Fc domain comprises mutations T366S, L368A and Y407V (EU numbering) or mutation T366W (EU numbering).

In some of the foregoing embodiments, the Fc domains of the first heavy chain (HC1) and/or the second heavy chain (HC2) further comprise one or more mutations which reduce Fc binding to a Fc receptor. In some embodiments, the Fc receptor is FcγRI, FcγRIIA, FcγRIIB, FcγRIIIA, and/or FcγRIIIB. In some of the foregoing embodiments, the Fc domain comprises one or more mutations selected from L234A, L235A, and D265S (EU numbering). In some of the foregoing embodiments, the Fc domain comprises mutations L234A, L235A, and D265S (EU numbering).

In some of the foregoing embodiments, the Fc domain further comprises one or more mutations which reduce Fc binding to protein A. In some of the foregoing embodiments, the Fc domain comprises mutations H435R and/or Y436F (EU numbering). In some of the foregoing embodiments, the Fc domain comprises mutations H435R and Y436F (EU numbering).

In some of the foregoing embodiments, the antibody or the antigen-binding fragment thereof, comprises a humanized antibody, or an antigen binding fragment thereof, a human antibody or an antigen binding fragment thereof, a murine antibody or an antigen binding fragment thereof, a chimeric antibody or an antigen binding fragment thereof, a monospecific antibody or a monospecific antigen binding fragment thereof, a bispecific antibody or a bispecific antigen binding fragment thereof, or a multispecific antibody or a multispecific antigen binding fragment thereof.

In any of the foregoing embodiments, the antibody or the antigen-binding fragment thereof, comprises: a) a heavy chain complementarity determining region (HCDR) 1, a HCDR2 and a HCDR3 of the heavy chain variable region (VH) of SEQ ID NO: 63, and a light chain complementarity determining region (LCDR) 1, a LCDR2 and a LCDR3 of the light chain variable region (VL) of SEQ ID NO: 64; b) a HCDR1, a HCDR2, and a HCDR3 of the VH of SEQ ID NO: 95, and a LCDR1, a LCDR2, and a LCDR3 of the VL of SEQ ID NO: 96; c) a HCDR1, a HCDR2, and a HCDR3 of the VH of SEQ ID NO: 127, and a LCDR1, a LCDR2, and a LCDR3 of the VL of SEQ ID NO: 128; or d) a HCDR1, a HCDR2, and a HCDR3 of the VH of SEQ ID NO: 191, and a LCDR1, a LCDR2, and a LCDR3 of the VL of SEQ ID NO: 192.

In any of the foregoing embodiments, the antibody or the antigen-binding fragment thereof, comprises the HCDR1, the HCDR2, the HCDR3, the LCDR1, the LCDR2 and the LCDR3 of: a) SEQ ID NO: 33, 34, 35, 48, 49, and 50, respectively; b) SEQ ID NO: 36, 37, 38, 51, 52, and 53, respectively; c) SEQ ID NO: 39, 40, 41, 54, 55, and 56, respectively; d) SEQ ID NO: 42, 43, 44, 57, amino acid sequence EVS, and SEQ ID NO: 59, respectively; e) SEQ ID NO: 45, 46, 47, 60, 61, and 62, respectively; f) SEQ ID NO: 65, 66, 67, 80, 81, and 82, respectively; g) SEQ ID NO: 68, 69, 70, 83, 84, and 85, respectively; h) SEQ ID NO: 71, 72, 73, 86, 87, and 88, respectively; i) SEQ ID NO: 74, 75, 76, 89, amino acid sequence AAI, and SEQ ID NO: 91, respectively; j) SEQ ID NO: 77, 78, 79, 92, 93, and 94, respectively; k) SEQ ID NO: 97, 98, 99, 112, 113, and 114, respectively; l) SEQ ID NO: 100, 101, 102, 115, 116, and 117, respectively; m) SEQ ID NO: 103, 104, 105, 118, 119, and 120, respectively; n) SEQ ID NO: 106, 107, 108, 121, amino acid sequence DNN, and SEQ ID NO: 123, respectively; o) SEQ ID NO: 109, 110, 111, 124, 125, and 126, respectively; p) SEQ ID NO: 161, 162, 163, 176, 177, and 178, respectively; q) SEQ ID NO: 164, 165, 166, 179, 180, and 181, respectively; r) SEQ ID NO: 167, 168, 169, 182, 18, and 184, respectively; s) SEQ ID NO: 170, 171, 172, 185, amino acid sequence EVS, and SEQ ID NO: 187, respectively; or t) SEQ ID NO: 173, 174, 175, 188, 189, and 190, respectively.

In any of the foregoing embodiments, the antibody or the antigen-binding fragment thereof, comprises a variable heavy chain region (VH) comprising an amino acid sequence that is at least 90%, at least 91%, at least 92%, at least 93%, at least 94%, at least 95%, at least 96%, at least 97%, at least 98%, at least 99% or 100% identical to: a) the VH of SEQ ID NO: 63; b) the VH of SEQ ID NO: 95; c) the VH of SEQ ID NO: 127; or d) the VH of SEQ ID NO: 191.

In any of the foregoing embodiments, the antibody or the antigen-binding fragment thereof, comprises or further comprises a variable light chain region (VL) comprising an amino acid sequence that is at least 90%, at least 91%, at least 92%, at least 93%, at least 94%, at least 95%, at least 96%, at least 97%, at least 98%, at least 99% or 100% identical to: a) the VL of SEQ ID NO: 64; b) the VL of SEQ ID NO: 96; c) the VL of SEQ ID NO: 128; or d) the VL of SEQ ID NO: 192.

In any of the foregoing embodiments, the antibody or the antigen-binding fragment thereof, comprises: a) the VH of SEQ ID NO: 63 and the VL of SEQ ID NO: 64; b) the VH of SEQ ID NO: 95 and the VL of SEQ ID NO: 96; c) the VH of SEQ ID NO: 127 and the VL of SEQ ID NO: 128; or d) the VH of SEQ ID NO: 191 and the VL of SEQ ID NO: 192.

In any of the foregoing embodiments, the antibody or the antigen-binding fragment thereof, comprises a scFv or a spFV which comprises, from the N- to C-terminus, a VH, a linker (L) and a in the format VH-L-VL or the VL, a linker (L) and a VH in the format VL-L-VH.

In some embodiments, the VH comprises the amino acid sequence of SEQ ID NO: 191, the VL comprises the amino acid sequence of SEQ ID NO: 192, and the L comprises SEQ ID NO: 221.

In some embodiments, the linker comprises SEQ ID NO: 221.

In any of the foregoing embodiments, the heavy chain (HC) comprises the amino acid sequence of SEQ ID NO: 194, 196, 198, or 202.

In any of the foregoing embodiments, the light chain (LC) comprises the amino acid sequence of SEQ ID NO: 195, 197 or 199.

In some of the foregoing embodiments, a) the HC1 comprises the amino acid sequence of SEQ ID NO: 194 and the first light chain (LC1) comprises the amino acid sequence of SEQ ID NO: 195; b) the HC1 comprises the amino acid sequence of SEQ ID NO: 196 and the LC1 comprises the amino acid sequence of SEQ ID NO: 197; or c) the HC1 comprises the amino acid sequence of SEQ ID NO: 198 and the LC1 comprises the amino acid sequence of SEQ ID NO: 199.

In another general aspect, the disclosure provides an antibody or an antigen-binding fragment thereof, that binds to the same epitope of EMR2 as the antibody or the antigen-binding fragment thereof, according to any of the embodiments described herein.

In another general aspect, the disclosure provides an antibody or an antigen-binding fragment thereof, that competes for binding to the same epitope of EMR2 with the antibody or the antigen-binding fragment thereof, according to any of the embodiments described herein.

In any of the foregoing embodiments, the antibody or the antigen-binding fragment thereof, is a monospecific antibody.

In any of the foregoing embodiments, the antibody or the antigen-binding fragment thereof, is a bispecific antibody which specifically binds to EMR2 and to a second antigen.

In some embodiments, the second antigen is T cell receptor beta variable 19 (TRBV19).

In an aspect, the disclosure provides an isolated polynucleotide encoding the antibody or the antigen-binding fragment thereof, according to any of the embodiments described herein.

In some embodiments, the isolated polynucleotide comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: a) the VH of SEQ ID NO: 63; b) the VH of SEQ ID NO: 95; c) the VH of SEQ ID NO: 127; or d) the VH of SEQ ID NO: 191.

In some embodiments, the isolated polynucleotide comprises or further comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: a) the VL of SEQ ID NO: 64; b) the VL of SEQ ID NO: 96; c) the VL of SEQ ID NO: 128; or d) the VL of SEQ ID NO: 192.

In any of the foregoing embodiments, the isolated polynucleotide comprises a sequence comprising a nucleotide sequence encoding: a) the amino acid sequence of SEQ ID NO: 63 and/or SEQ ID NO: 64; b) the amino acid sequence of SEQ ID NO: 95 and/or SEQ ID NO: 96; c) the amino acid sequence of SEQ ID NO: 127 and/or SEQ ID NO: 128; or d) the amino acid sequence of SEQ ID NO: 191 and/or the SEQ ID NO: 192.

In some embodiments, the isolated polynucleotide comprises a sequence encoding a heavy chain (HC) comprising the amino acid sequence of SEQ ID NO: 194, 196, 198, or 202.

In some embodiments, the isolated polynucleotide comprises a sequence encoding a light chain (LC) comprising the amino acid sequence of SEQ ID NO: 195, 197, or 199.

In any of the foregoing embodiments, the isolated polynucleotide comprises a nucleotide sequence encoding: a) the amino acid sequence of SEQ ID NO: 194 and/or SEQ ID NO: 195; b) the amino acid sequence of SEQ ID NO: 196 and/or SEQ ID NO: 197; c) the amino acid sequence of SEQ ID NO: 198 and/or the amino acid sequence of SEQ ID NO: 199; or d) the amino acid sequence of SEQ ID NO: 202.

In another general aspect, the disclosure provides a vector comprising the isolated polynucleotide according to any of the foregoing embodiments.

In some embodiments, the isolated polynucleotide is operably linked to an expression control sequence.

In some embodiments, the vector is a viral vector. In some embodiments, the viral vector is selected from an adenoviral vector, an adeno-associated viral vector, a retroviral vector, a lentiviral vector, a herpes simplex virus vector, and a poxvirus vector.

In another general aspect, the disclosure provides a pharmaceutical composition comprising (i) the antibody or the antigen-binding fragment thereof, according to any of the embodiments described herein, or the isolated polynucleotide according to any of the embodiments described herein, or the vector according to any of the embodiments described herein, and (ii) a pharmaceutically acceptable carrier or excipient.

In an aspect, the disclosure provides a host cell expressing the antibody or the antigen-binding fragment thereof, according to any of the embodiments described herein.

In some embodiments, the cell is a hybridoma.

In some embodiments, the antibody, or the antigen-binding fragment thereof, is recombinantly produced.

In another general aspect, the disclosure provides a host cell comprising the isolated polynucleotide according to any of the embodiments described herein or the vector according to of the embodiments described herein.

In another general aspect, the disclosure provides a method for treating a cancer in a subject in need thereof, the method comprising administering to the subject a therapeutically effective amount of the antibody or the antigen-binding fragment thereof, according to any of the embodiments described herein, or the isolated polynucleotide according to any of the embodiments described herein, or the vector according to any of the embodiments described herein, or the pharmaceutical composition according to any of the embodiments described herein, or the host cell according to any of the embodiments described herein.

In another general aspect, the disclosure provides a method for inducing cytotoxicity of a cancer cell or redirecting immune or T cells to a cancer cell, said method comprising administering to the cell an effective amount of the antibody or the antigen-binding fragment thereof, according to any of the embodiments described herein, or the polynucleotide according to any of the embodiments described herein, or the vector according to any of the embodiments described herein, or the pharmaceutical composition of any of the embodiments described herein, or the host cell according to any of the embodiments described herein, wherein said effective amount is sufficient to inhibit the growth or proliferation of the cancer cell.

In some embodiments, the cancer cell is in a subject and the antibody or the binding fragment, the polynucleotide, the vector, the pharmaceutical composition, or the host cell is administered to the subject.

In some embodiments, said administration is conducted ex vivo.

In some embodiments, the cancer is an EMR2-expressing cancer. In some embodiments, the EMR2-expressing cancer is a hematological cancer. In some embodiments, the hematological cancer is a myeloid malignancy. In some embodiments, the hematological cancer is an acute myeloid leukemia (AML), a chronic myelogenous leukemia (CML), or myelodysplastic neoplasms (MDS).

In any of the foregoing embodiments, the methods further comprise administering a second therapeutic agent.

In some embodiments, the second therapeutic agent is a surgery, a chemotherapy, an androgen deprivation therapy, a radiation, or any combination thereof.

In another general aspect, the disclosure provides an antibody according to any of the embodiments described herein for use in the method according to any of the embodiments described herein.

In another general aspect, the disclosure provides an isolated polynucleotide according to any of the embodiments described herein for use in the method according to any of the embodiments described herein.

In another general aspect, the disclosure provides a vector according to any of the embodiments described herein for use in the method according to any of the embodiments described herein.

In another general aspect, the disclosure provides a pharmaceutical composition according to the embodiments described herein for use in the method according to any of the embodiments described herein.

In another general aspect, the disclosure provides a host cell according to any of the embodiments described herein for use in the method according to any of the embodiments described herein.

In another general aspect, the disclosure provides a method for generating the antibody or the antigen-binding fragment thereof, according to any of the embodiments described herein, wherein said method comprises culturing the host cell according to any of the embodiments described herein and isolating said antibody or the antigen-binding fragment thereof.

In another general aspect, the disclosure provides a kit comprising (i) the antibody or the antigen-binding fragment thereof, according to any of the embodiments described herein and/or the polynucleotide according to any of the embodiments described herein, and/or the vector according to any of the embodiments described herein, and/or the pharmaceutical composition of any of the embodiments described herein, and/or the host cell according to any of the embodiments described herein, and (ii) packaging for the same and/or instructions for use.

In another general aspect, the disclosure provides a bispecific antibody or a bispecific antigen-binding fragment thereof, that specifically binds (i) TRBV19 with a first antigen-binding site and (ii) GAIN domain and/or GPS motif of EMR2 with a second antigen-binding site.

In some embodiments, the bispecific antibody or the bispecific antigen-binding fragment comprises: a) a first heavy chain (HC1); b) optionally a first light chain (LC1); c) a second heavy chain (HC2); and d) optionally a second light chain (LC2), wherein (i) the HC1 and the LC1 form a first antigen-binding site that specifically binds a first antigen, (ii) the HC2 and the LC2 form a second antigen-binding site that specifically binds a second antigen, (iii) the HC1 and HC2 each comprise a Fc domain comprising a CH2-CH3 domain; and wherein the first antigen is TRBV19, and the second antigen is EMR2.

In some embodiments, the second antigen-binding site specifically binds the GAIN domain and/or GPS motif of EMR2.

In some embodiments, the GAIN domain comprises amino acid residues D261-Q478 of human EMR2 (SEQ ID NO: 205).

In some embodiments, the GAIN domain comprises the amino acid sequence of SEQ ID NO: 215.

In any of the foregoing embodiments, the GPS motif comprises the amino acid sequence SEQ ID NO: 216.

In any of the foregoing embodiments, the antibody or the antigen-binding fragment thereof, binds to an epitope comprising SEQ ID NO: 217 or SEQ ID NO: 218.

In any of the foregoing embodiments, the first antigen-binding site comprises a Fab or a scFv or a spFv.

In any of the foregoing embodiments, the Fc of the first antigen-binding site comprises a CH1 domain.

In any of the foregoing embodiments, the second antigen-binding site comprises a Fab or a scFv or a spFv.

In any of the foregoing embodiments, the Fc of the second antigen-binding site further comprises a CH1 domain.

In any of the foregoing embodiments, the first antigen-binding site comprises a Fab and the second antigen-binding site comprises a scFv or a spFv.

In any of the foregoing embodiments, the first antigen-binding site comprises a scFv or a spFv and the second antigen-binding site comprises a Fab.

In any of the foregoing embodiments, the Fc domain of the antibody or the antigen-binding fragment thereof, is an IgA, an IgG, an IgE, or an IgM.

In some embodiments, the Fc domain of the antibody or the antigen-binding fragment thereof, is an IgG. In some embodiments, the IgG is IgG1 or IgG4.

In any of the foregoing embodiments, the Fc domains of HC1 and HC2 comprise one or more different mutations which promote heterodimerization.

In any of the foregoing embodiments, the Fc domain of the HC1 comprises mutations T366S, L368A and Y407V (EU numbering) and the Fc domain of the HC2 comprises mutation T366W (EU numbering). In any of the foregoing embodiments, the Fc domain of the HC2 comprises mutations T366S, L368A and Y407V (EU numbering) and the Fc domain of the HC1 comprises mutation T366W (EU numbering).

In any of the foregoing embodiments, the Fe domains of HC1 and/or HC2 further comprise one or more mutations which reduce Fc binding to a Fcγ receptor. In any of the foregoing embodiments, the Fcγ receptor is FcγRI, FcγRIIA, FcγRIIB, FcγRIIIA, and/or FcγRIIIB. In any of the foregoing embodiments, the Fe domains of HC1 and/or HC2 each comprise one or more mutations selected from L234A, L235A, and D265S (EU numbering). In any of the foregoing embodiments, the Fe domains of HC1 and HC2 each comprise mutations L234A, L235A, and D265S (EU numbering).

In any of the foregoing embodiments, the Fe domains of HC1 or HC2 further comprises one or more mutations which reduce Fc binding to protein A. In any of the foregoing embodiments, the Fe domains of HC1 or HC2 comprise mutations H435R and/or Y436F (EU numbering). In any of the foregoing embodiments, the Fc domain of HC1 comprises mutations H435R and Y436F (EU numbering). In any of the foregoing embodiments, the Fc domain of HC2 comprises mutations H435R and Y436F (EU numbering).

In any of the foregoing embodiments, the HC1 or HC2 comprise mutation C220S (EU numbering). In any of the foregoing embodiments, the HC1 comprises mutation C220S (EU numbering). In any of the foregoing embodiments, the HC2 comprises mutation C220S (EU numbering).

In any of the foregoing embodiments, the antibody or the antigen-binding fragment thereof, is a humanized antibody or an antigen binding fragment thereof, a human antibody or an antigen binding fragment thereof, a murine antibody or an antigen binding fragment thereof, a chimeric antibody or an antigen binding fragment thereof, or a chemically modified derivative thereof.

In any of the foregoing embodiments, the first antigen-binding site that specifically binds TRBV19 comprises: a) a HCDR1, a HCDR2 and a HCDR3 of the VH of SEQ ID NO: 31 and a LCDR1, a LCDR2 and a LCDR3 of the VL of SEQ ID NO: 32; or b) a HCDR1, a HCDR2, and a HCDR3 of the VH of SEQ ID NO: 159 and a LCDR1, LCDR2, and LCDR3 of the VL of SEQ ID NO: 160.

In any of the foregoing embodiments, the first antigen-binding site that specifically binds TRBV19 comprises the HCDR1, the HCDR2, the HCDR3, the LCDR1, the LCDR2 and the LCDR3 of: a) SEQ ID NOs: 1, 2, 3, 16, 17, and 18, respectively; b) SEQ ID NOs: 4, 5, 6, 19, 20, and 21, respectively; c) SEQ ID NOs: 7, 8, 9, 22, 23, and 24, respectively; d) SEQ ID NOs: 10, 11, 12, 25, amino acid sequence KVS, and SEQ ID NO: 27, respectively; e) SEQ ID NOs: 13, 14, 15, 28, 29, and 30, respectively; f) SEQ ID NOs: 129, 130, 131, 144, 145, and 146, respectively; g) SEQ ID NOs: 132, 133, 134, 147, 148, and 149, respectively; h) SEQ ID NOs: 135, 136, 137, 150, 151, and 152, respectively; i) SEQ ID NOs: 138, 139, 140, 153, 154, and 155, respectively, for IMGT numbering; or j) SEQ ID NOs: 141, 142, 143, 156, 157, and 158, respectively.

In any of the foregoing embodiments, the first antigen-binding site that specifically binds TRBV19 comprises a VH comprising an amino acid sequence that is at least 90%, at least 91%, at least 92%, at least 93%, at least 94%, at least 95%, at least 96%, at least 97%, at least 98%, at least 99% or 100% identical to: the VH of SEQ ID NO: 31; or the VH of SEQ ID NO: 159.

In any of the foregoing embodiments, the first antigen-binding site that specifically binds TRBV19 comprises or further comprises a VL comprising an amino acid sequence that is at least 90%, at least 91%, at least 92%, at least 93%, at least 94%, at least 95%, at least 96%, at least 97%, at least 98%, at least 99% or 100% identical to: the VL of SEQ ID NO: 32; or the VL of SEQ ID NO: 160.

In any of the foregoing embodiments, the first antigen-binding site that specifically binds TRBV19 comprises: the VH of SEQ ID NO: 31 and the VL of SEQ ID NO: 32; or the VH of SEQ ID NO: 159 and the VL of SEQ ID NO: 160.

In any of the foregoing embodiments, the second antigen-binding site that specifically binds EMR2 comprises: a) a HCDR1, a HCDR2 and a HCDR3 of the VH of SEQ ID NO: 63, and a LCDR1, a LCDR2 and a LCDR3 of the VL of SEQ ID NO: 64; b) a HCDR1, a HCDR2, and a HCDR3 of the VH of SEQ ID NO: 95, and a LCDR1, a LCDR2, and a LCDR3 of the VL of SEQ ID NO: 96; c) a HCDR1, a HCDR2, and a HCDR3 of the VH of SEQ ID NO: 127, and a LCDR1, a LCDR2, and a LCDR3 of the VL of SEQ ID NO: 128; or d) a HCDR1, a HCDR2, and a HCDR3 of the VH of SEQ ID NO: 191, and a LCDR1, a LCDR2, and a LCDR3 of the VL of SEQ ID NO: 192.

In any of the foregoing embodiments, the second antigen-binding site that specifically binds EMR2 comprises the HCDR1, the HCDR2, the HCDR3, the LCDR1, the LCDR2 and the LCDR3 of: a) SEQ ID NO: 33, 34, 35, 48, 49, and 50, respectively; b) SEQ ID NO: 36, 37, 38, 51, 52, and 53, respectively; c) SEQ ID NO: 39, 40, 41, 54, 55, and 56, respectively; d) SEQ ID NO: 42, 43, 44, 57, amino acid sequence EVS, and SEQ ID NO: 59, respectively; e) SEQ ID NO: 45, 46, 47, 60, 61, and 62, respectively; f) SEQ ID NO: 65, 66, 67, 80, 81, and 82, respectively; g) SEQ ID NO: 68, 69, 70, 83, 84, and 85, respectively; h) SEQ ID NO: 71, 72, 73, 86, 87, and 88, respectively; i) SEQ ID NO: 74, 75, 76, 89, amino acid sequence AAI, and SEQ ID NO: 91, respectively; j) SEQ ID NO: 77, 78, 79, 92, 93, and 94, respectively; k) SEQ ID NO: 97, 98, 99, 112, 113, and 114, respectively; l) SEQ ID NO: 100, 101, 102, 115, 116, and 117, respectively; m) SEQ ID NO: 103, 104, 105, 118, 119, and 120, respectively; n) SEQ ID NO: 106, 107, 108, 121, amino acid sequence DNN, and SEQ ID NO: 123, respectively; o) SEQ ID NO: 109, 110, 111, 124, 125, and 126, respectively; p) SEQ ID NO: 161, 162, 163, 176, 177, and 178, respectively; q) SEQ ID NO: 164, 165, 166, 179, 180, and 181, respectively; r) SEQ ID NO: 167, 168, 169, 182, 18, and 184, respectively; s) SEQ ID NO: 170, 171, 172, 185, amino acid sequence EVS, and SEQ ID NO:187, respectively; or t) SEQ ID NO: 173, 174, 175, 188, 189, and 190, respectively.

In any of the foregoing embodiments, the bispecific antibody or the bispecific antigen-binding fragment thereof, comprises a VH comprising an amino acid sequence that is at least 90%, at least 91%, at least 92%, at least 93%, at least 94%, at least 95%, at least 96%, at least 97%, at least 98%, at least 99% or 100% identical to: a) the VH of SEQ ID NO: 63; b) the VH of SEQ ID NO: 95; c) the VH of SEQ ID NO: 127; or d) the VH of SEQ ID NO: 191.

In any of the foregoing embodiments, the bispecific antibody or the bispecific antigen-binding fragment thereof, comprises or further comprises a VL comprising an amino acid sequence that is at least 90%, at least 91%, at least 92%, at least 93%, at least 94%, at least 95%, at least 96%, at least 97%, at least 98%, at least 99% or 100% identical to: a) the VL of SEQ ID NO: 64; b) the VL of SEQ ID NO: 96; c) the VL of SEQ ID NO: 128; or d) the VL of SEQ ID NO: 192.

In any of the foregoing embodiments, the first antigen-binding site and/or the second antigen-binding site that specifically binds EMR2 comprises: a) the VH of SEQ ID NO: 63 and the VL of SEQ ID NO: 64; b) the VH of SEQ ID NO: 95 and the VL of SEQ ID NO: 96; c) the VH of SEQ ID NO: 127 and the VL of SEQ ID NO: 128; or d) the VH of SEQ ID NO: 191 and the VL of SEQ ID NO: 192.

In any of the foregoing embodiments, the bispecific antibody or the bispecific antigen-binding fragment thereof, comprises a single-chain variable fragment (scFv) or a spFV which scFv or spFV comprises, from the N- to C-terminus, a VH, a linker (L) and a VL in the format VH-L-VL or a VL, a linker and a VH in the format VL-L-VH.

In any of the foregoing embodiments, the bispecific antibody or the bispecific antigen-binding fragment thereof, comprises the VH comprising the amino acid sequence of SEQ ID NO: 159, the VL comprises the amino acid sequence of SEQ ID NO: 160, and the L comprises SEQ ID NO: 221.

In any of the foregoing embodiments, the bispecific antibody or the bispecific antigen-binding fragment thereof, comprises the VH comprising the amino acid sequence of SEQ ID NO: 191, the VL comprises the amino acid sequence of SEQ ID NO: 192, and the L comprises SEQ ID NO: 221.

In any of the foregoing embodiments, the bispecific antibody or the bispecific antigen-binding fragment thereof, comprises the L comprising SEQ ID NO: 221.

In any of the foregoing embodiments, the first antigen-binding site specifically binds to TRBV19 with a dissociation constant (K_{D}) that is between about 15 nM to about 200 nM.

In any of the foregoing embodiments, the first antigen-binding site specifically binds to TRBV19 with an EC₅₀ between about 1 nM to about 100 nM.

In any of the foregoing embodiments, the second antigen-binding site specifically binds to EMR2 with a dissociation constant (K_{D}) that is between about 0.01 nM to about 5 nM.

In any of the foregoing embodiments, the second antigen-binding site specifically binds to EMR2 with an EC₅₀ between about 0.1 nM to about 15 nM.

In any of the foregoing embodiments, the bispecific antibody or the bispecific antigen-binding fragment thereof, comprises the HC1 comprising the amino acid sequence of SEQ ID NO: 193 or 200.

In any of the foregoing embodiments, the bispecific antibody or the bispecific antigen-binding fragment thereof, comprises the LC1 comprising the amino acid sequence of SEQ ID NO: 201.

In any of the foregoing embodiments, the bispecific antibody or the bispecific antigen-binding fragment thereof, comprises a) the HC1 comprising the amino acid sequence of SEQ ID NO: 193; or b) the HC1 comprising the amino acid sequence of SEQ ID NO: 200 and the LC1 comprising the amino acid sequence of SEQ ID NO: 201.

In any of the foregoing embodiments, the bispecific antibody or the bispecific antigen-binding fragment thereof, comprises the HC2 comprising the amino acid sequence of SEQ ID NO: 194, 196, 198, or 202.

In any of the foregoing embodiments, the bispecific antibody or the bispecific antigen-binding fragment thereof, comprises the LC2 comprising the amino acid sequence of SEQ ID NO: 195, 197, or 199.

In any of the foregoing embodiments, the bispecific antibody or the bispecific antigen-binding fragment thereof, comprises a) the HC2 comprising the amino acid sequence of SEQ ID NO: 194 and the LC2 comprising the amino acid sequence of SEQ ID NO: 195; b) the HC2 comprising the amino acid sequence of SEQ ID NO: 196 and the LC1 comprising the amino acid sequence of SEQ ID NO: 197; c) the HC2 comprising the amino acid sequence of SEQ ID NO: 198 and the LC1 comprising the amino acid sequence of SEQ ID NO: 199; or d) the HC2 comprising the amino acid sequence of SEQ ID NO: 202.

In any of the foregoing embodiments, the bispecific antibody or the bispecific antigen-binding fragment thereof, comprises a) the HC1 comprising the amino acid sequence of SEQ ID NO: 193, the HC2 comprising the amino acid sequence of SEQ ID NO: 194, and the LC2 comprising the amino acid sequence of SEQ ID NO 195; b) the HC1 comprising the amino acid sequence of SEQ ID NO: 193, the HC2 comprising the amino acid sequence of SEQ ID NO: 196, and the LC2 comprising the amino acid sequence of SEQ ID NO: 197; c) the HC1 comprising the amino acid sequence of SEQ ID NO: 193, the HC2 comprising the amino acid sequence of SEQ ID NO: 198, and the LC2 comprising the amino acid sequence of SEQ ID NO: 199; or d) the HC1 comprising the amino acid sequence of SEQ ID NO: 200, the LC1 comprising the amino acid sequence of SEQ ID NO: 201, and the HC2 comprising the amino acid sequence of SEQ ID NO: 202.

In another general aspect, the disclosure provides a bispecific antibody or a bispecific antigen-binding fragment thereof, that specifically binds (i) TRBV19 with a first antigen-binding site and (ii) the GAIN domain and/or the GPS motif of EMR2 with a second antigen-binding site, wherein the first antigen-binding site that specifically binds TRBV19 comprises a HCDR1, a HCDR2 and a HCDR3 of the VH of SEQ ID NO: SEQ ID NO: 159 and a LCDR1, LCDR2, and LCDR3 of the VL of SEQ ID NO: 160 and wherein the second antigen-binding site that specifically binds EMR2 comprises a HCDR1, a HCDR2, and a HCDR3 of the VH of SEQ ID NO: 95, and a LCDR1, a LCDR2, and a LCDR3 of the VL of SEQ ID NO: 96.

In any of the foregoing embodiments, wherein the first antigen-binding site that specifically binds TRBV19 comprises the HCDR1, the HCDR2, the HCDR3, the LCDR1, the LCDR2 and the LCDR3 of: SEQ ID NOs: 129, 130, 131, 144, 145, and 146, respectively; SEQ ID NOs: 132, 133, 134, 147, 148, and 149, respectively; SEQ ID NOs: 135, 136, 137, 150, 151, and 152, respectively; SEQ ID NOs: 138, 139, 140, 153, 154, and 155, respectively; or SEQ ID NOs: 141, 142, 143, 156, 157, and 158, respectively; and wherein the second antigen-binding site that specifically binds EMR2 comprises the HCDR1, the HCDR2, the HCDR3, the LCDR1, the LCDR2 and the LCDR3 of SEQ ID NO: 65, 66, 67, 80, 81, and 82, respectively; SEQ ID NO: 68, 69, 70, 83, 84, and 85, respectively; SEQ ID NO: 71, 72, 73, 86, 87, and 88, respectively; SEQ ID NO: 74, 75, 76, 89, amino acid sequence AAI, and SEQ ID NO: 91, respectively; or SEQ ID NO: 77, 78, 79, 92, 93, and 94, respectively.

In any of the foregoing embodiments, the first antigen-binding site that specifically binds TRBV19 comprises the VH of SEQ ID NO: 159 and the VL of SEQ ID NO: 160 and the second antigen-binding site that specifically binds EMR2 comprises the VH of SEQ ID NO: 95 and the VL of SEQ ID NO: 96.

In any of the foregoing embodiments, the bispecific antibody or the bispecific antigen-binding fragment thereof, comprises an HC1 comprising the amino acid sequence of SEQ ID NO: 193, an HC2 comprising the amino acid sequence of SEQ ID NO: 198, and an LC2 comprising the amino acid sequence of SEQ ID NO: 199.

In another general aspect, the disclosure provides a bispecific antibody or a bispecific antigen-binding fragment thereof, that binds to the same epitope as the bispecific antibody or the bispecific antigen-binding fragment thereof, according to any of the embodiments described herein.

In another general aspect, the disclosure provides a bispecific antibody or a bispecific antigen-binding fragment thereof, that competes for binding to the same epitope with the bispecific antibody or the bispecific antigen-binding fragment thereof, according to any of the embodiments described herein.

In another general aspect, the disclosure provides a bispecific antibody, or a bispecific antigen-binding fragment thereof, wherein the bispecific antibody, or the bispecific antigen-binding fragment thereof, comprises an HC1 comprising the amino acid sequence of SEQ ID NO: 193, an HC2 comprising the amino acid sequence of SEQ ID NO: 198, and an LC2 comprising the amino acid sequence of SEQ ID NO: 199.

In another general aspect, the disclosure provides an isolated polynucleotide encoding the bispecific antibody or the bispecific binding fragment of any of the embodiments described herein.

In some embodiments, the isolated polynucleotide comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: a) the VH of SEQ ID NO: 63; b) the VH of SEQ ID NO: 95; c) the VH of SEQ ID NO: 127; or d) the VH of SEQ ID NO: 191.

In any of the foregoing embodiments, the isolated polynucleotide comprises or further comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: a) the VL of SEQ ID NO: 64; b) the VL of SEQ ID NO: 96; c) the VL of SEQ ID NO: 128; or d) the VL of SEQ ID NO: 192.

In any of the foregoing embodiments, the isolated polynucleotide comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: a) the VH of SEQ ID NO: 31; or b) the VH of SEQ ID NO: 159.

In any of the foregoing embodiments, the isolated polynucleotide comprises or further comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: a) the VL of SEQ ID NO: 32; or b) the VL of SEQ ID NO: 160.

In any of the foregoing embodiments, the isolated polynucleotide comprises a sequence encoding a first antigen-binding site that specifically binds TRBV19, the sequence comprising a nucleotide sequence encoding: a) the amino acid sequence of SEQ ID NO: 31 and/or the amino acid sequence of SEQ ID NO: 32; or b) the amino acid sequence of SEQ ID NO: 159 and/or the amino acid sequence of SEQ ID NO: 160.

In any of the foregoing embodiments, the isolated polynucleotide comprises a sequence encoding a second antigen-binding site that specifically binds EMR2, the sequence comprising a nucleotide sequence encoding: a) the amino acid sequence of SEQ ID NO: 63 and/or the amino acid sequence of SEQ ID NO: 64; b) the amino acid sequence of SEQ ID NO: 95 and/or the amino acid sequence of SEQ ID NO: 96; c) the amino acid sequence of SEQ ID NO: 127 and/or the amino acid sequence of SEQ ID NO: 128; or d) the amino acid sequence of SEQ ID NO: 191 and/or the amino acid sequence of SEQ ID NO: 192.

In any of the foregoing embodiments, the isolated polynucleotide comprises a sequence encoding an HC1 comprising the amino acid sequence of SEQ ID NO: 193 or 200.

In any of the foregoing embodiments, the isolated polynucleotide comprises a sequence encoding a LC1 comprising the amino acid sequence of SEQ ID NO: 201.

In any of the foregoing embodiments, the isolated polynucleotide comprises a sequence encoding: a) the amino acid sequence of SEQ ID NO: 193; or b) the amino acid sequence of SEQ ID NO: 200 and the amino acid sequence of SEQ ID NO: 201.

In any of the foregoing embodiments, the isolated polynucleotide comprises a sequence encoding an HC2 comprising the nucleotide sequence of SEQ ID NO: 194, 196, 198, or 202.

In any of the foregoing embodiments, the isolated polynucleotide comprises a sequence encoding a LC2 comprising the nucleotide sequence of SEQ ID NO: 195, 197, or 199.

In any of the foregoing embodiments, the isolated polynucleotide comprises a sequence encoding: a) the amino acid sequence of SEQ ID NO: 194 and/or the amino acid sequence of SEQ ID NO: 195; b) the amino acid sequence of SEQ ID NO: 196 and/or the amino acid sequence of SEQ ID NO: 197; c) the amino acid sequence of SEQ ID NO: 198 and/or the amino acid sequence of SEQ ID NO: 199; or d) the amino acid sequence of SEQ ID NO: 202.

In any of the foregoing embodiments, the isolated polynucleotide comprises a sequence encoding: a) the amino acid sequence of SEQ ID NO: 193, the amino acid sequence of SEQ ID NO: 194 and the amino acid of SEQ ID NO: 195; b) the amino acid sequence of SEQ ID NO: 193, the amino acid sequence of SEQ ID NO: 196 and the amino acid sequence of SEQ ID NO: 197; c) the amino acid sequence of SEQ ID NO: 193, the amino acid sequence of SEQ ID NO: 198 and the amino acid sequence of SEQ ID NO: 199; or d) the amino acid sequence of SEQ ID NO: 200, the amino acid sequence of SEQ ID NO: 201, and the amino acid sequence of SEQ ID NO: 202.

In another general aspect, the disclosure provides a vector comprising the polynucleotide according to any of the embodiments described herein.

In some embodiments, the isolated polynucleotide is operably linked to an expression control sequence.

In some embodiments, the vector is a viral vector. In some embodiments, the viral vector is selected from an adenoviral vector, an adeno-associated viral vector, a retroviral vector, a lentiviral vector, a herpes simplex virus vector, and a poxvirus vector.

In another general aspect, the disclosure provides a pharmaceutical composition comprising (i) the bispecific antibody or the bispecific antigen-binding fragment thereof, according to any of the foregoing embodiments, or the polynucleotide according to any of the foregoing embodiments, or the vector according to any of the foregoing embodiments, and (ii) a pharmaceutically acceptable carrier or excipient.

In another general aspect, the disclosure provides a host cell expressing the bispecific antibody or the bispecific antigen-binding fragment thereof, according to any of the embodiments described herein.

In some embodiments, the cell is a hybridoma.

In some embodiments, the bispecific antibody or the bispecific antigen-binding fragment thereof, is recombinantly produced.

In another general aspect, the disclosure provides a host cell comprising the isolated polynucleotide according to any of the embodiments described herein or the vector according to any of the embodiments described herein.

In another general aspect, the disclosure provides a method for treating cancer in a subject in need thereof, the method comprising administering to the subject a therapeutically effective amount of the bispecific antibody or the bispecific antigen-binding fragment thereof, according to any of the embodiments described herein, or the polynucleotide according to any of the embodiments described herein, or the vector according to any of the foregoing embodiments, or the pharmaceutical composition according to any of the embodiments described herein.

In another general aspect, the disclosure provides a method for inducing cytotoxicity of a cancer cell or redirecting immune or T cells to a cancer cell, said method comprising administering to the cell an effective amount of the bispecific antibody or the bispecific antigen-binding fragment thereof, according to any of the embodiments described herein, or the isolated polynucleotide according to any of the embodiments described herein, or the vector according to any of the embodiments described herein, or the pharmaceutical composition according to any of the embodiments described herein, or the host cell according to any of the embodiments described herein, wherein the effective amount is sufficient to inhibit the growth or proliferation of the cancer cell.

In some embodiments, the cancer cell is in a subject and the bispecific antibody or the bispecific antigen-binding fragment thereof, the polynucleotide, the vector, the pharmaceutical composition, or the host cell is administered to the subject.

In some embodiments, the administration is conducted ex vivo.

In another general aspect, the disclosure provides a method of redirecting a T cell to EMR2-expressing cancer cells in a subject in need thereof, the method comprising administering to the subject a therapeutically effective amount of the bispecific antibody or the bispecific antigen-binding fragment thereof, according to any of the embodiments described herein, or the polynucleotide according to any of the embodiments described herein, or the vector according to any of the embodiments described herein, or the pharmaceutical composition according to any of the embodiments described herein, or the host cell according to any of the embodiments described herein.

In some embodiments, the therapeutically effective amount is sufficient to direct said T cell response to the cancer cells.

In any of the foregoing embodiments, the cancer is an EMR2-expressing cancer. In some embodiments, the EMR2-expressing cancer is a hematological cancer. In some embodiments, the hematological cancer is a myeloid malignancy. In some embodiments, the cancer is AML, chronic myelogenous leukemia (CML), or MDS.

In any of the foregoing embodiments, the method further comprises administering a second therapeutic agent.

In some embodiments, the second therapeutic agent is a surgery, a chemotherapy, an androgen deprivation therapy, or a radiation, or any combination thereof.

In another general aspect, the disclosure provides a bispecific antibody or a bispecific antigen-binding fragment thereof, according to any of the embodiments described herein for use in the method according to any of the embodiments described herein.

In another general aspect, the disclosure provides an isolated polynucleotide according to any of the embodiments described herein for use in the method according to any of the embodiments described herein.

In another general aspect, the disclosure provides a vector according to any of the embodiments described herein for use in the method according to any of the embodiments described herein.

In another general aspect, the disclosure provides a pharmaceutical composition according to any of the embodiments described herein for use in the method according to any of the embodiments described herein.

In another general aspect, the disclosure provides a host cell according to any of the embodiments described herein for use in the method according to any of the embodiments described herein.

In another general aspect, the disclosure provides a method for generating the bispecific antibody or the bispecific antigen-binding fragment thereof, according to any of the embodiments described herein, wherein said method comprises culturing the host cell according to any of the embodiments described herein, and isolating the bispecific antibody or the bispecific binding fragment.

In another general aspect, the disclosure provides a kit comprising (i) the bispecific antibody or the bispecific antigen-binding fragment thereof, according to any of the embodiments described herein, or the isolated polynucleotide according to any of the embodiments described herein, or the vector according to any of the embodiments described herein, or the pharmaceutical composition according to any of the embodiments described herein, or the host cell according to any of the embodiments described herein, and (ii) packaging for the same and/or instructions for use.

### BRIEF DESCRIPTION OF THE DRAWINGS

FIG. 1. Frequency of TRBV19⁺ T cells. AML, acute myeloid leukemia; CD, cluster of differentiation; HD, healthy donor; PBMC, peripheral blood mononuclear cells. Healthy-donor PBMCs (n=20) and AML PBMCs (n=16) were commercially purchased and phenotyped for the frequency of TRBV19+ T cells. Data shown as population percentage from total CD3-expressing cells.

FIG. 2. Depiction of EMR2 Structure. EMR2 receptor features a 7-pass transmembrane domain (shown in a box), a GAIN domain, and 5 EGF-like domains (labeled as E1 to E5). EGF, epidermal growth factor; EMR2, GAIN.

FIG. 3. EMR2 expression in hematological malignancies (mRNA levels). The InforMe database was used to quantitate the relative mRNA levels of EMR2 across hematological malignancies using the GeneLogic Heme Plus 2.0 data set. AML, acute myeloid leukemia; CML, chronic myelogenous leukemia; DLBCL, diffused large B-cell lymphoma; EMR2, epidermal-growth-factor-like module containing mucin-like hormone receptor 2; FL, follicular lymphoma; MCL, mantle cell lymphoma; MDS, myelodysplastic syndrome (also known as myelodysplastic neoplasms); MGUS, monoclonal gammopathy of undetermined significance; MM, multiple myeloma.

FIG. 4. EMR2 expression on healthy hematopoietic cells and primary AML blasts. Heat maps showing EMR2 expression percentage as assessed by flow cytometry on healthy-donor CD34+ HSCs and progenitor cells (n=8), healthy-donor PBMCs (n=8), healthy-donor peripheral blood (n=2), and AML patient-derived blast detected in the BM (n=14) or PBMCs (n=9). Expression is indicated as EMR2 percentage within each population that was identified based on its respective specific surface markers. For each population receptor density was calculated and depicted. AML, acute myeloid leukemia; BM, bone marrow; CD, cluster of differentiation; CMP, common myeloid progenitor; EMR2, GMP, granulocyte-monocyte progenitor; HD, healthy donor; HSC, hematopoietic stem cell; MEP, megakaryocyte erythrocyte progenitor; MPP, multi-potent progenitor; PB, peripheral blood; PBMC

FIG. 5. Concentration of soluble EMR2 in HD and AML patient sera. Soluble EMR2 levels in HD and AML patient serum via ELISA showed no significant difference between populations. There was a median soluble EMR2 concentration of 5.00 pM in HD serum and 4.03 pM in AML patient serum. AML, ELISA: enzyme-linked immunosorbent assay; EMR2, HD, healthy donors; NS, no significant difference.

FIG. 6. Bispecific antibody 1-hour, 37° C. binding on AML cell lines. Binding of a bispecific or EMR2xNull control or TRBV19xNull control were incubated with tumor cells for 1 hour at 37° C. at a starting concentration of 2,000 nM, diluted serially 1:3 for 11 points. After secondary detection with AF647-labeled material, the MFI signal was analyzed to generate an EC50 value. Curves are representative of 3 independent experiments. AML, acute myeloid leukemia; EC₅₀, 50% effective concentration; EMR2, MFI, mean fluorescence intensity; Vβ17, variable domain of T-cell receptor beta chain 17, also referred to as TRBV19.

FIG. 7. 1-, 3-, 5-, and 24-hour, 37° C. kinetic binding of the bispecific antibody and TRBV19xNull on OCI-AML3 cells. A bispecific antibody or TRBV19xNull control was incubated with OCI-AML-3 cells for 1, 3, 5, and 24 hours at 37° C. at concentrations of 3, 30, and 300 nM. After secondary detection with AF647-labeled material, the MFI signal was analyzed to show stable binding of the molecule at various concentrations over time.

FIG. 8. Bispecific antibody 1-hour, 37° C. binding on TRBV19+ T cells. Bispecific antibody or EMR2xNull control were incubated with pan-T cells from 6 healthy donors for 1 hour at 37° C. at a starting concentration of 2,000 nM. After secondary detection, the percent TRBV19+ (AF647+) of live cells was analyzed to generate an EC50 value. EC₅₀, 50% effective concentration.

FIGS. 9A-9B. A representative data set for the EMR2xTRBV19 cytotoxicity assays using OCI-AML3 AML cell line: (FIG. 9A) T-cell cytotoxicity assay was performed with increasing concentrations of antibody for 72 hours at E:T ratio of 10:1. (FIG. 9B) PBMC cytotoxicity assay was performed with increasing concentrations of antibody for 96 hours at E:T ratio of 5:1.

FIGS. 10A-10C. Exemplary bispecific amino acid sequences. CDRs using AbM definition shown in bold. Linker connecting TRBV19 (TRBV19) VL and VL are underlined. Mature amino acid sequence of an exemplary Heavy Chain 1 (FIG. 10A). Mature amino acid sequence of an exemplary Light Chain 2 (FIG. 10B). Mature amino acid sequence of an exemplary Heavy Chain 2 (FIG. 10C).

FIG. 11. T-cell-mediated cytotoxicity. Cancer cell cytotoxicity was assessed in a flow-cytometry-based assay at 3 and 6 days with pan-T cells as effector cells (n=4-5 different healthy donors) cocultured with EMR2+ (i.e., OCI-AML3, OCI-AML5, OCI-AML2) or EMR2− (i.e., OCI-LY10, n=3) AML cell lines. Absolute E:T ratio shown is 10:1 (relative E:T ratio 0.5:1). EMR2, E:T ratio, effector-to-target ratio

FIGS. 12A-12B. The bispecific antibody induced T-cell activation and expansion in the presence of different cancer cell lines. (FIG. 12A) T-cell activation and (FIG. 12 B) T-cell expansion were assessed by flow cytometry at Day 3 and Day 6 upon coculture with a panel of antigen-positive or -negative cancer cell lines at an absolute E:T ratio of 10:1 (relative E:T ratio of 0.5:1). T-cell activation was determined through the expression (%) of the late T-cell activation marker CD25. T-cell expansion was assessed by T-cell absolute counts, normalized to the untreated wells. Data from 4 to 5 different healthy T-cell donors were pooled and represented as mean±SEM. CD, cluster of differentiation; EMR2, E:T ratio, effector-to-target ratio; SEM, standard error of the mean.

FIGS. 13A-13C. The bispecific antibody induced selective TRBV19⁺ T-cell activation in the presence of different cancer cell lines. T-cell activation was assessed by flow cytometry at Day 3 and Day 6 upon coculture with a panel of antigen-positive or -negative cancer cell lines at an absolute E:T ratio of 10:1 (relative E:T ratio of 0.5:1). (FIG. 13A) TRBV19⁺ T-cell expansion was assessed by measuring the percentage of TRBV19⁺ T cells. T-cell activation was determined for both (FIG. 13B) TRBV19⁺ and (FIG. 13C) TRBV19⁻ T-cell subpopulations. The expression (%) of the late T-cell activation marker CD25 was used to identify the activated T cells. Data from 4 to 5 different healthy T-cell donors for EMR2⁺ cell lines and 3 healthy donors for EMR2-cell lines were pooled and represented as mean±SEM. CD, cluster of differentiation; E:T ratio, effector-to-target ratio; SEM, standard error of the mean.

FIGS. 14A-14D. Intracellular cytokine production upon stimulation with the bispecific antibody. Characterization of TRBV19⁺ T-cell profile upon the bispecific antibody treatment was assessed in a flow-cytometry-based assay. OCI-AML3 cells and pan-T cells (n=6 different healthy donors) were cocultured at an absolute E:T ratio of 10:1 (relative E:T ratio for TRBV19⁺ T cells of 0.5:1) for 48 hours. (FIG. 14A) Efficacy was evaluated by determining cytotoxicity to cancer cells and granzyme B expression on pan-T cells and TRBV19⁺ T cells. Profiling of cytokine expression was determined for (FIG. 14B) IFN-γ, (FIG. 14C) IL-2, and (FIG. 14D) TNF-α by assessing the expression of the respective cytokine on pan-T cells and TRBV19⁺ T cells. The intensity of cytokine expression is indicated as signal MFI and normalized to untreated conditions. CD, cluster of differentiation; E:T ratio, effector-to-target ratio; IFN, interferon; IL, interleukin; MFI, mean fluorescence intensity; TNF, tumor necrosis factor.

FIGS. 15A-15D. Intracellular cytokine production upon stimulation with the bispecific antibody compared to a control comparator antibody A. Characterization of TRBV19⁺ T-cell profile upon bispecific antibody or control comparator antibody A treatments was assessed in a flow-cytometry-based assay. OCI-AML3 cells and pan-T cells (n=6 different healthy donors) were cocultured at a relative E:T ratio for TRBV19⁺ T cells of 0.5:1 for 48 hours. (FIG. 15A) T-cell-mediated cytotoxicity was evaluated by determining granzyme B expression on pan-T cells and TRBV19⁺ T cells. Profiling of cytokine expression was determined for (FIG. 15B) IFN-γ, (FIG. 15C) IL-2, and (FIG. 15D) TNF-α by assessing the expression of the respective cytokine on pan-T cells and TRBV19⁺ T cells. The intensity of cytokine expression is indicated as signal MFI in the positive population and normalized to untreated conditions. CD, cluster of differentiation; EMR2, E:T ratio, effector-to-target ratio; Gzmb, granzyme B; IFN, interferon; IL, interleukin; MFI, mean fluorescence intensity; TNF, tumor necrosis factor;

FIGS. 16A-16F. Therapeutic efficacy (in vitro cytotoxicity assay) of the bispecific antibody in a time-course PBMC-based cytotoxicity assay. (FIG. 16A) Cancer cell cytotoxicity of the bispecific antibody was assessed by flow cytometry at 24, 48, 72, 96, and 120 hours. PBMCs were cocultured with OCI-AML3 cells at an E:T ratio of 5:1 with total PBMCs as effector cells (relative E:T ratio of ˜0.19:1 for the bispecific antibody and ˜3.3:1 for the control comparator antibody A). Cancer cell cytotoxicity was calculated by normalizing the absolute target cell count in the treated conditions to the counts of the untreated wells. T-cell activation was assessed by evaluating the expression of CD25 on either (FIG. 16B) total CD3 T cells or (FIG. 16C) TRBV19⁺ T cells. (FIGS. 16D-16F) NullxTRBV19 control was assessed in parallel. Graphing of data was done in GraphPad Prism 10. Data from 5 healthy donors were pooled and represented as mean±SEM. CD, cluster of differentiation; E:T ratio, effector-to-target ratio; PBMC, peripheral blood mononuclear cell; SEM, standard error of the mean.

FIGS. 17A-17C. Cytotoxicity of control comparator antibody A in PBMC assays. As a comparison for assessing the cancer cell cytotoxicity of the bispecific antibody, a control comparator antibody A was used in parallel. Readout of the flow-cytometry-based assay was performed at 24, 48, 72, 96, and 120 hours with OCI-AML3 as target cancer cells and healthy PBMCs as effector cells (n=5 donors) at an E:T ratio of 5:1. (FIG. 17A) Cytotoxicity was calculated by normalizing the absolute target cell count in the treated conditions to the counts of the untreated wells. T-cell activation was assessed by evaluating the expression of CD25 on either (FIG. 17B) total CD3 T cells or (FIG. 17C) TRBV19⁺ T cells. CD, cluster of differentiation; E:T ratio, effector-to-target ratio; LH, light-heavy; PBMC, peripheral blood mononuclear cell; spFv, single-chain fragment variable featuring a ‘stapled’ linker.

FIG. 18. Time-course evaluation of cytokine production upon bispecific antibody treatment in a PBMC-based cytotoxicity assay. Supernatant were collected at the different time points and analyzed for inflammatory cytokines using MSD Proinflammatory kit. Graphing of data was done in GraphPad Prism 10. Data from 5 different donors were pooled and represented as mean±SEM. IFN, interferon; IL, interleukin; MSD, Meso Scale Discovery; PBMC, peripheral blood mononuclear cell; SEM, standard error of the mean; TNF, tumor necrosis factor.

FIG. 19. Time-course evaluation of cytokine production upon control comparator antibody A treatment in a PBMC-based cytotoxicity assay. Supernatant were collected at the different time points and analyzed for inflammatory cytokines using MSD Proinflammatory kit. Graphing of data was done in GraphPad Prism 10. Data from 5 different donors were pooled and represented as mean±SEM. CD, cluster of differentiation; EMR2, IFN, interferon; IL, interleukin; MSD, Meso Scale Discovery; PBMC, peripheral blood mononuclear cell; SEM, standard error of the mean; TNF, tumor necrosis factor.

FIGS. 20A-20D. Bispecific antibody cytotoxicity assays on primary AML BM cells. (FIG. 20A) Cytotoxicity of primary AML BM cells was evaluated in a flow-cytometry-based assay for 24 hours at a relative E:T ratio of 2:1. Two different T-cell donors were used with different AML BM donors (n=3). (FIG. 20B) Pan-T-cell activation, (FIG. 20C) specific TRBV19⁺ T-cell activation, and (FIG. 20D) TRBV19⁻ T-cell activation were evaluated by measuring the expression of the activation markers CD69 and/or CD25. AML, acute myeloid leukemia; BM, bone marrow; CD, cluster of differentiation; DN, donor; E:T ratio, effector-to-target ratio. FIGS. 20A-20D. Bispecific antibody cytotoxicity assays on primary AML BM cells. (FIG. 20A) Cytotoxicity of primary AML BM cells was evaluated in a flow-cytometry-based assay for 24 hours at a relative E:T ratio of 2:1. Two different T-cell donors were used with different AML BM donors (n=3). (FIG. 20B) Pan-T-cell activation, (FIG. 20C) specific TRBV19⁺ T-cell activation, and (FIG. 20D) TRBV19⁻ T-cell activation were evaluated by measuring the expression of the activation markers CD69 and/or CD25. AML, acute myeloid leukemia; BM, bone marrow; CD, cluster of differentiation; DN, donor; E:T ratio, effector-to-target ratio.

FIGS. 21A-21B. Bispecific antibody on-target/off-tumor assessment on primary HSPC and myeloid cells. (FIG. 21A) CFU assays were performed to evaluate on-target/off-tumor toxicity of the bispecific antibody on healthy-donor CD34⁺ HSPC. Healthy-donor pan-T cells were used as effector cells at a relative E:T ratio of 1:1 with CD34⁺ cells (n=5) or OCI-AML3 cells (n=5) as target cells. After 48 hours of preincubation with a dose range of the bispecific antibody, cells were seeded in semisolid methylcellulose medium for 14 days to support colony formation. The viability of either CD34⁺ cells or OCI-AML3 cells was assessed by enumerating the number of colonies formed compared to the untreated controls. As shown in the bar graph, the bars alternate CD34+ HSPC cells followed by OCI-AML3 cells from left to right. (FIG. 21B) Viability of monocytes upon the bispecific antibody treatment was assessed by flow cytometry at 72 hours. PBMCs were cocultured with OCI-AML3 cells at an E:T ratio of 5:1 with total PBMCs as effector cells. Monocyte cytotoxicity was calculated by normalizing the absolute target cell count in the treated conditions to the counts of the untreated wells. CD, cluster of differentiation; CFU, colony forming unit; E:T, effector to target; HSCP, hematopoietic stem and progenitor cells; PBMC, peripheral blood mononuclear cell.

FIG. 22. On-target/off-tumor assessment of control comparator antibody A. Viability of monocytes upon control comparator antibody treatment A was assessed by flow cytometry at 72 hours. PBMCs were cocultured with OCI-AML3 cells at an E:T ratio of 5:1 with total PBMCs as effector cells (n=5). Monocyte cytotoxicity was calculated by normalizing the absolute target cell count in the treated conditions to the counts of the untreated wells. CD, cluster of differentiation; EMR2, E:T, effector to target; PBMC, peripheral blood mononuclear cell.

FIGS. 23A-23D. Effect of the bispecific antibody on MOLM-13-luc disseminated human AML progression in T-cell-humanized mice. Tumor cells were implanted on Day 0. T cells were implanted on Day 3, as indicated by an asterisk. Mice (n=10/group) bearing established MOLM-13-luc disseminated AML were IP dosed the bispecific antibody or control DPBS as indicated by triangles and an arrow pointing out the triangles on Days 4, 7, 10, 13, 17, 20, 24, 27, 31, and 34. (FIG. 23A) Logarithmic scale and (FIG. 23 B) linear scale group BLI is graphed as average radiance mean expressed in p/s/cm²/sr±SEM when at least 7 of 10 animals remained in each group. * Denotes statistically significant difference of the bispecific antibody-treated groups over time versus DPBS-treated control group. (FIG. 23C) Percent survival. * Denotes statistically significant difference in survival curves of the bispecific antibody-treated groups versus DPBS-treated control group. (FIG. 23D) Individual BLI plots with number of CRs in each group. Ab, antibody; AML, acute myeloid leukemia; BLI, bioluminescent imaging; CR, complete response; DPBS, Dulbecco's phosphate-buffered saline; IP, intraperitoneal; luc, luciferase; M, million; p, photons; SEM, standard error of the mean; sr, steradian.

FIG. 24. Effect of the bispecific antibody on MOLM-13-luc disseminated human AML progression in T-cell-humanized mice; representative ventral BLI images. Representative ventral BLI images from 5 mice per group on the indicated study days. Tumor cells were implanted on Day 0. Mice bearing established MOLM-13-luc disseminated AML were IP dosed with the bispecific antibody at the indicated doses. AML, acute myeloid leukemia; BLI, bioluminescent imaging; DPBS, Dulbecco's phosphate-buffered saline; luc, luciferase; M, million; p, photons; sr, steradian.

FIGS. 25A-25B. In vivo efficacy of control comparator antibody A on OCI-AML3-luc disseminated human AML progression in T-cell-humanized mice. Tumor cells were implanted on Day 0 and T cells on Day 6, as indicated by an asterisk. Mice (n=10/group) bearing established OCI-AML3-luc disseminated AML were IP dosed with control comparator antibody A (1 mg/kg) or control DPBS as indicated by triangles on Days 7, 11, 14, 18, 21, 25, 28, 32, 36, 40, 43, 47, 50, and 54. (FIG. 25A) Group BLI is graphed as average radiance mean expressed in p/s/cm²/sr±SEM when at least 7 of 10 animals remained in each group. * Denotes statistically significant difference of control comparator antibody A-treated groups on Day 33 versus DPBS-treated control group (100% ΔTGI). (FIG. 25B) Percent survival. * Denotes statistically significant difference in survival curves of control comparator antibody A-treated groups versus DPBS-treated control group (>65% ILS).

FIGS. 26A-26C. Effect of the bispecific antibody on OCI-AML3-luc disseminated human AML progression in T-cell-humanized mice. Tumor cells were implanted on Day 0 and T cells on Day 6, as indicated by an asterisk. Mice (n=10/group) bearing established OCI-AML3-luc disseminated AML were IP dosed the bispecific antibody or control DPBS as indicated by solid triangles on Days 7, 11, 14, 18, 21, 25, 28, 32, 36, 40, 43, 47, 50 and 54. (FIG. 26A) Logarithmic scale and (FIG. 26B) linear scale group BLI is graphed as average radiance mean expressed in p/s/cm²/sr±SEM when at least 7 of 10 animals remained in each group. * Denotes statistically significant difference of the bispecific antibody-treated groups on Day 33 versus DPBS-treated control group. (FIG. 26C) Percent survival. * Denotes statistically significant difference in survival curves of the bispecific antibody-treated groups versus DPBS-treated control group. Ab, antibody; AML, acute myeloid leukemia; BLI, bioluminescent imaging; DPBS, Dulbecco's phosphate-buffered saline; IP, intraperitoneal; luc, luciferase; M, million; p, photons; SEM, standard error of the mean; sr, steradian.

FIG. 27. Effect of the bispecific antibody on OCI-AML3-luc disseminated human AML progression in T-cell-humanized mice; representative dorsal BLI images. Representative dorsal BLI images from 5 mice per group on the indicated study days. Tumor cells were implanted on Day 0. Mice bearing established MOLM-13-luc disseminated AML were IP dosed with the bispecific antibody at the indicated doses. AML, acute myeloid leukemia; BLI, bioluminescent imaging; DPBS, Dulbecco's phosphate-buffered saline; luc, luciferase; M, million; p, photons; sr, steradian

FIG. 28. Table of the Biophysical Properties of the bispecific antibodies.

FIGS. 29A-29C. Binding activity of the bispecific antibodies. Percent Relative Activity of TRBV19 binding (FIG. 29A). Binding to TRBV19+ T cells (FIG. 29B). EMR2 arm binding affinity (FIG. 29C).

FIG. 30. Amino acid sequences of an exemplary bispecific antibody.

FIG. 31. Amino acid sequences of an exemplary bispecific antibody.

FIG. 32. Amino acid sequences of an exemplary bispecific antibody.

FIG. 33. Amino acid sequences of an exemplary bispecific antibody.

FIG. 34. Amino acid sequences of exemplary Kappa (SEQ ID NO: 222), Lambda (SEQ ID NO: 223), CH (SEQ ID NO: 225) and CH1 (SEQ ID NO: 224) regions. Figure discloses the full “huIgG1_Glm(17)” sequence as SEQ ID NO: 227.

### DETAILED DESCRIPTION OF ILLUSTRATIVE EMBODIMENTS

The bispecific molecules of the present disclosure, bind the variable domain of T-cell receptor beta chain 17 (Vβ17; also referred to as TRBV19) expressed on a subtype of T cells and epidermal-growth-factor-like module-containing mucin-like hormone receptor-like 2 (EMR2; also known as adhesion G protein-coupled receptor E2 or ADGRE2) on AML blast cells. The mechanism of action (MoA) of the bispecific molecules of the present disclosure is to selectively engage TRBV19+ T cells and act as a bridge between cancer cells and T cells. TRBV19+ T cells are a subtype of mature and primed viral-specific T lymphocytes with memory phenotype found in approximately 5% of T cells in the peripheral blood of healthy and AML patients. Binding to the TRBV19 receptor on a subset of T cells and the specific antigen on cancer cells (i.e., EMR2) results in TRBV19+ T-cell activation and lysis of cancer cells. This approach differentiates from CD3 T-cell engagers (TCEs) through the recruitment of a selected T-cell subpopulation (i.e., TRBV19+) with the potential to increase the therapeutic index by lowering the risk of severe cytokine release syndrome (CRS).

Examples of bispecific (EMR2xTRBV19) molecules of the present disclosure include fully human immunoglobulin (Ig)G1-L234A, L235A, D265S (AAS) bispecific antibodies specifically targeting TRBV19⁺ T cells with one binding arm and EMR2 with the other binding arm. The antibodies feature the AAS mutations in the Fc region (fragment crystallizable [Fc]) to abolish interaction with Fc receptors. The TRBV19-engaging arm bound with an affinity of 94±13 nM to recombinant TRBV19 protein by surface plasmon resonance (SPR) and detected TRBV19⁺ T cells with a 50% effective concentration (EC50) of 7.8 nM. The EMR2-binding arm bound EMR2-expressing cells with an affinity range of 27 to 55 nM and showed a stable binding profile over 24 hours. The TRBV19-binding arm is a stabilized single-chain fragment variable featuring a ‘stapled’ linker (spFv), while the EMR2-binding arm is a fragment antigen-binding (Fab).

The bispecific molecules of the present disclosure exhibited good biophysical properties. In vitro, the bispecific molecules of the present disclosure induced T-cell mediated cytotoxicity to EMR2⁺ AML cancer cell lines. No impact on the viability of tumor-associated antigen (TAA)-negative cells was observed. The bispecific molecules of the present disclosure induced selective activation and expansion of TRBV19⁺ T cells with no/minimal impact on the TRBV19⁻ population.

The bispecific molecules of the present disclosure showed potent cancer cell cytotoxicity in peripheral blood mononuclear cell (PBMC)-based assays. The bispecific molecules of the present disclosure led to low overall T-cell activation associated with slower and lower cytokine secretion (i.e., interleukin [IL]-1β, IL-10, and tumor necrosis factor [TNF]-α) than the comparator antibody A.

The bispecific molecules of the present disclosure induced potent T-cell-mediated cytotoxicity to primary AML bone marrow (BM) cells with minimal overall T-cell activation.

The bispecific molecules of the present disclosure induced higher cytotoxic activity on AML cell lines than on healthy hematopoietic stem and progenitor cells (HSPCs) or monocytes.

In vivo, the bispecific molecules of the present disclosure showed robust antitumor efficacy leading to increased survival in MOLM-13-luc and OCI-AML3-luc established models. EMR2 expression was high on macrophage lineages (i.e., monocyte/macrophages/dendritic cells) across various tissues and low on granulocytes (i.e., basophils, eosinophils, and neutrophils).

In vitro data showed a moderate level of activation of monocytes and neutrophils, and to a lesser extent natural killer (NK) cells. No activation of basophils was observed. No off-target liabilities were observed based on the human cell microarray platform screen (Retrogenix) and an in vitro functional assay on TAA-negative cell lines.

### Definitions

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dubel eds., 2d ed. 2010).

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated.

As used in this specification and the appended claims, the singular forms “a,” “an,” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of up to ±10% from the specified value, as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

“Isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids. An “isolated” antibody or an antigen-binding fragment, as used herein, is intended to refer to an antibody or an antigen-binding fragment which is substantially free of other antibodies or antigen-binding fragments having different antigenic specificities.

“Polynucleotide,” synonymously referred to as “nucleic acid molecule,” “nucleotides” or “nucleic acids,” refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

The meaning of “substantially the same” can differ depending on the context in which the term is used. Because of the natural sequence variation likely to exist among heavy and light chains and the genes encoding them, one would expect to find some level of variation within the amino acid sequences or the genes encoding the antibodies or antigen-binding fragments described herein, with little or no impact on their unique binding properties (e.g., specificity and affinity). Such an expectation is due in part to the degeneracy of the genetic code, as well as to the evolutionary success of conservative amino acid sequence variations, which do not appreciably alter the nature of the encoded protein. Accordingly, in the context of nucleic acid sequences, “substantially the same” means at least 65% identity between two or more sequences. Preferably, the term refers to at least 70% identity between two or more sequences, more preferably at least 75% identity, more preferably at least 80% identity, more preferably at least 85% identity, more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, and more preferably at least 99% or greater identity. The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm.

The degree of variation that may occur within the amino acid sequence of a protein without having a substantial effect on protein function is much lower than that of a nucleic acid sequence, since the same degeneracy principles do not apply to amino acid sequences. Accordingly, in the context of an antibody or an antigen-binding fragment, “substantially the same” means antibodies or antigen-binding fragments having 90%, 91%, 92%, 93%, 94%, 95%, 96%, 97%, 98%, or 99% identity to the antibodies or antigen-binding fragments described. Other embodiments include antibodies, or antigen-binding fragments, that have framework, scaffold, or other non-binding regions that do not share significant identity with the antibodies and antigen-binding fragments described herein, but do incorporate one or more CDRs or other sequences needed to confer binding that are 90%, 91%, 92%, 93%, 94%, 95%, 96%, 97%, 98%, or 99% identical to such sequences described herein.

A “clone” is a population of cells derived from a single cell or common ancestor by mitosis. A “cell line” is a clone of a primary cell that is capable of stable growth in vitro for many generations. In some examples provided herein, cells are transformed by transfecting the cells with DNA.

The terms “express” and “produce” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications. The expression or production of an antibody or an antigen-binding fragment thereof, may be within the cytoplasm of the cell, or into the extracellular milieu such as the growth medium of a cell culture.

The terms “treating” or “treatment” refer to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, neurological examination, or psychiatric evaluations.

An “effective amount” or “therapeutically effective amount” refers to an amount effective, at dosages and for periods of time necessary, to achieve a desired therapeutic result. A therapeutically effective amount of an antibody described herein may vary according to factors such as the disease state, age, sex, and weight of the individual, and the ability of the antibody to elicit a desired response in the individual. A therapeutically effective amount is also one in which any toxic or detrimental effects of the antibody or the antibody portion are outweighed by the therapeutically beneficial effects.

“Antibody” is understood in accordance with its ordinary meaning in the field and encompasses all isotypes of immunoglobulins (IgG, IgA, IgE, IgM, IgD, and IgY) including various monomeric, polymeric and chimeric forms, unless otherwise specified. Specifically encompassed by the term “antibody” are polyclonal antibodies, monoclonal antibodies (mAbs), and antibody-like polypeptides, such as chimeric antibodies and humanized antibodies or human antibodies, as well as multispecific antibodies, bispecific antibodies, single-domain antibodies (sdAb), Immunoglobulin New Antigen Receptor (Ig NARs), single heavy chain antibodies, camelid antibodies, shark antibodies, or chemically modified derivatives thereof. The term “antibody” encompasses the meaning of non-naturally occurring antibody or of an engineered antibody. “Antigen-binding fragments” are any proteinaceous structure that may exhibit binding affinity for a particular antigen. Antigen-binding fragments include those provided by any known technique, such as enzymatic cleavage, peptide synthesis, and recombinant techniques. Some antigen-binding fragments are composed of portions of intact antibodies that retain antigen-binding specificity of the parent antibody molecule. For example, antigen-binding fragments may comprise at least one variable region (either a heavy chain or light chain variable region) or one or more CDRs of an antibody known to bind a particular antigen. Examples of suitable antigen-binding fragments include, without limitation diabodies and single-chain molecules as well as Fab, F(ab′)2, F(ab)′3, Fc, Fabc, and Fv molecules, single chain (Sc) antibodies, single-chain variable fragments (scFv), bis-scFvs, (scFv)2, stapled scFvs (spFv) (see, e.g., Boucher, L E et al., “Stapling” scFv for multispecific biotherapeutics of superior properties, MAbs. 2023; 15(1): 2195517; PCT Int. Publ. No. WO 2023/089587; PCT Int. Publ. No. WO 2021/030657), individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, a monovalent fragment consisting of the VL, VH, CL and CH1 domains, or a monovalent antibody as described in WO2007059782, bivalent fragments comprising two Fab fragments linked by a disulfide bridge at the hinge region, a Fd fragment consisting essentially of the VH and CH1 domains; a Fv fragment consisting essentially of the VL and VH domains of a single arm of an antibody, a dAb fragment (see, e.g., Ward et al., Nature 341, 544-546 (1989)), which consists essentially of a VH domain and also called domain antibodies (see, e.g., Holt et al; Trends Biotechnol. 2003 November; 21(11):484-90); camelid or nanobodies (see, e.g., Revets et al; Expert Opin Biol Ther. 2005 January; 5(1):111-24); an isolated complementarity determining region (CDR), a diabody; a minibody; a triabody; a tetrabody; a disulfide stabilized Fv protein (dsFv); and the like. All antibody isotypes may be used to produce antigen-binding fragments. Additionally, antigen-binding fragments may include non-antibody proteinaceous frameworks that may successfully incorporate polypeptide segments in an orientation that confers affinity for a given antigen of interest, such as protein scaffolds. Antigen-binding fragments may be recombinantly produced or produced by enzymatic or chemical cleavage of intact antibodies. The phrase “an antibody or an antigen-binding fragment thereof” may be used to denote that a given antigen-binding fragment incorporates one or more amino acid segments of the antibody referred to in the phrase.

The terms “CDR”, and its plural “CDRs”, refer to a complementarity determining region (CDR) of which three make up the binding character of a light chain variable region (CDRL1, CDRL2 and CDRL3) and three make up the binding character of a heavy chain variable region (CDRH1, CDRH2 and CDRH3). CDRs contribute to the functional activity of an antibody molecule and are separated by amino acid sequences that comprise scaffolding or framework regions. The exact definitional CDR boundaries and lengths are subject to different classification and numbering systems. CDRs may therefore be referred to by Kabat, Chothia, Contact, IMTG, AbM, or any other boundary definitions. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat et al., Sequences of Proteins of Immunological Interest, 5^{th} ed. NIH Publication No. 91-3242 (1991); Chothia et al., “Canonical Structures For the Hypervariable Regions of Immunoglobulins,” J. Mol. Biol. 196:901 (1987); and MacCallum et al., “Antibody-Antigen Interactions: Contact Analysis and Binding Site Topography,” J. Mol. Biol. 262:732 (1996)), each of which is hereby incorporated by reference in its entirety.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia et al., “Canonical Structures For the Hypervariable Regions of Immunoglobulins,” J. Mol. Biol. 196:901 (1987); Chothia et al., “Conformations of Immunoglobulin Hypervariable Regions,” I 342:877 (1989); Martin and Thornton, “Structural Families in Loops of Homologous Proteins: Automatic Classification, Modelling and Application to Antibodies,” J. Mol. Biol. 263:800 (1996), each of which is incorporated by reference in its entirety). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “polypeptide” is used interchangeably with the term “protein” and in its broadest sense refers to a compound of two or more subunit amino acids, amino acid analogs or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein.

“Specifically binds” or “binds specifically” or derivatives thereof when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules. Typically, an antibody binds to a cognate antigen with a K_{d} of less than about 1×10⁻⁸ M, as measured by a surface plasmon resonance assay or a cell-binding assay. Phrases such as “[antigen]-specific” antibody (e.g., EMR2-specific antibody or TRBV19-specific antibody) are meant to convey that the recited antibody specifically binds the recited antigen. As used herein, the term “chimeric” refers to an antibody, or antigen-binding fragment thereof, having at least some portion of at least one variable domain derived from the antibody amino acid sequence of a non-human mammal, a rodent, or a reptile, while the remaining portions of the antibody, or antigen-binding fragment thereof, are derived from a human.

“Polynucleotide,” synonymously referred to as “nucleic acid molecule,” “nucleotides” or “nucleic acids,” refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

A “vector” is a replicon, such as plasmid, phage, cosmid, or virus in which another nucleic acid segment may be operably inserted so as to bring about the replication or expression of the segment.

As used herein, the term “host cell” can be any type of cell, e.g., a primary cell, a cell in culture, or a cell from a cell line. In specific embodiments, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome. The terms “expression” and “production” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications.

The term “subject” refers to human and non-human animals, including all vertebrates, e.g., mammals and non-mammals, such as non-human primates, mice, rabbits, sheep, dogs, cats, horses, cows, chickens, amphibians, and reptiles. In many embodiments of the described methods, the subject is a human.

The term “redirect” or “redirecting” as used herein refers to the ability of the described multispecific antibody (e.g., a EMR2xTRBV19 antibody) to traffic the activity of T cells effectively, from its inherent cognate specificity toward reactivity against EMR2-expressing cells.

The term “sample” as used herein refers to a collection of similar fluids, cells, or tissues (e.g., surgically resected tumor tissue, biopsies, including fine needle aspiration), isolated from a subject, as well as fluids, cells, or tissues present within a subject. In some embodiments the sample is a biological fluid. Biological fluids are typically liquids at physiological temperatures and may include naturally occurring fluids present in, withdrawn from, expressed or otherwise extracted from a subject or biological source. Certain biological fluids derive from particular tissues, organs or localized regions and certain other biological fluids may be more globally or systemically situated in a subject or biological source. Examples of biological fluids include blood, serum and serosal fluids, plasma, lymph, urine, saliva, cystic fluid, tear drops, feces, sputum, mucosal secretions of the secretory tissues and organs, vaginal secretions, ascites fluids such as those associated with non-solid tumors, fluids of the pleural, pericardial, peritoneal, abdominal and other body cavities, fluids collected by bronchial lavage and the like. Biological fluids may also include liquid solutions contacted with a subject or biological source, for example, cell and organ culture medium including cell or organ conditioned medium, lavage fluids and the like. The term “sample,” as used herein, encompasses materials removed from a subject or materials present in a subject.

A “EMR2xTRBV19 antibody” is a multispecific antibody, optionally a bispecific antibody, which comprises two different antigen-binding regions, one of which binds specifically to the antigen EMR2 and one of which binds specifically to TRBV19 (also known as Vβ17). The term “multispecific antibody” is used herein in the broadest sense and specifically covers an antibody that has polyepitopic specificity. Multispecific antibodies include, but are not limited to, an antibody comprising a heavy chain variable domain (V_{H}) and a light chain variable domain (V_{L}), where the V_{H}VL unit has polyepitopic specificity, antibodies having two or more V_{L} and VH domains where each V_{H}V_{L} unit binds to a different epitope, antibodies having two or more single variable domains with each single variable domain binding to a different epitope, full length antibodies, and antibodies comprising one or more antibody fragments as well as antibodies comprising antibody fragments that have been linked covalently or non-covalently.

A multispecific antibody can be a bispecific antibody, diabody, or similar molecule (see for instance PNAS USA 90(14), 6444-8 (1993) for a description of diabodies). The bispecific antibodies, diabodies, and the like, provided herein may bind any suitable target in addition to a portion of epidermal-growth-factor-like module-containing mucin-like hormone receptor 2 (EMR2) or T cell receptor (TCR) TRBV19. The term “bispecific antibody” is to be understood as an antibody having two different antigen-binding regions defined by different antibody sequences. This can be understood as different target binding but includes as well as binding to different epitopes in one target.

A “reference sample” is a sample that may be compared against another sample, such as a test sample, to allow for characterization of the compared sample. The reference sample will have some characterized property that serves as the basis for comparison with the test sample. For instance, a reference sample may be used as a benchmark for EMR2 levels that are indicative of a subject having cancer. The reference sample does not necessarily have to be analyzed in parallel with the test sample, thus in some instances the reference sample may be a numerical value or range previously determined to characterize a given condition, such as EMR2 levels that are indicative of cancer in a subject.

The term “progression,” as used in the context of progression of EMR2-expressing cancer, includes the change of a cancer from a less severe to a more severe state. This may include an increase in the number or severity of tumors, the degree of metastasis, the speed with which the cancer is growing or spreading, and the like. For example, “the progression of colon cancer” includes the progression of such a cancer from a less severe to a more severe state, such as the progression from stage I to stage II, from stage II to stage III, etc.

The term “regression,” as used in the context of regression of EMR2-expressing cancer, includes the change of a cancer from a more severe to a less severe state. This could include a decrease in the number or severity of tumors, the degree of metastasis, the speed with which the cancer is growing or spreading, and the like. For example, “the regression of colon cancer” includes the regression of such a cancer from a more severe to a less severe state, such as the progression from stage III to stage II, from stage II to stage I, etc.

The term “stable” as used in the context of stable EMR2-expressing cancer, is intended to describe a disease condition that is not, or has not, changed significantly enough over a clinically relevant period of time to be considered a progressing cancer or a regressing cancer.

The embodiments described herein are not limited to particular methods, reagents, compounds, compositions or biological systems, which can, of course, vary.

### Multispecific Antibodies

Multispecific antibodies that bind to EMR2 and/or TRBV19, and multispecific binding fragments thereof are provided herein. Such antibodies or antibody fragments may allow for more specific targeting to particular subsets of cells as compared to antibodies targeting only one or two of these targets.

### Bispecific Antibodies

In some embodiments, provided herein are bispecific antibodies that bind to EMR2 and TRBV19, and bispecific binding fragments thereof. This can be achieved by, for example, making a molecule which comprises a first region binding specifically to EMR2, and a second binding region binding specifically to TRBV19. The antigen-binding regions can take any form that allows specific recognition of the target, for example the binding region may be or may include a heavy chain variable domain, an Fv (combination of a heavy chain variable domain and a light chain variable domain), an single-chain Fv (scFv), a stapled scFv (“spFv”), an Fab, a binding domain based on a fibronectin type III domain (such as from fibronectin, or based on a consensus of the type III domains from fibronectin, or from tenascin or based on a consensus of the type III domains from tenascin, such as the Centyrin molecules from Janssen Biotech, Inc., see e.g. WO2010/051274 and WO2010/093627). Accordingly, bispecific molecules comprising three different antigen-binding regions which bind EMR2 and TRBV19, respectively, are provided.

In some embodiments, the EMR2 x TRBV19-multispecific antibody comprises a first heavy chain (HC1) and a light chain (LC) that pair to form a first antigen-binding site that specifically binds a first antigen and a second heavy chain (HC2) comprises a second antigen-binding site that specifically binds a second antigen. The HC1 and HC2 may each comprise a Fragment crystallizable (Fc) domain comprising a CH2-CH3 domain. In some embodiments, the EMR2 x TRBV19-bispecific antibody comprises a EMR2-specific arm comprising a first heavy chain (HC1) and a light chain (LC) that pair to form a first antigen-binding site that specifically binds EMR2, a second heavy chain (HC2) that comprises a second antigen-binding site that specifically binds TRBV19 (TRBV19). In some embodiments, the EMR2 x TRBV19-bispecific antibody comprises a EMR2-specific arm comprising a first heavy chain (HC1) and a light chain (LC) that pair to form a first antigen-binding site that specifically binds TRBV19 (Vβ17), a second heavy chain (HC2) that comprises a second antigen-binding site that specifically binds EMR2.

In some embodiments, the first antigen-binding site comprises a fragment antigen-binding (Fab) region. In some embodiments, the second antigen-binding site comprises a single-chain variable fragment (scFv) or a stapled scFv (spFv). In some embodiments, the first antigen-binding site comprises a scFv or a spFv. In some embodiments, the second antigen-binding site comprises a Fab. In some embodiments, the spFv comprises a C220S mutation.

In one embodiment, the EMR2-binding arm comprises a fragment antigen-binding (Fab) region, and the TRBV19-binding arm comprises a single-chain variable fragment (scFv) or spFv. In some embodiments, the spFv comprises a C220S mutation.

In one embodiment, the TRBV19-binding arm comprises a fragment antigen-binding (Fab) region, and the EMR2-binding arm comprises a single-chain variable fragment (scFv) or spFv. In some embodiments, the spFv comprises a C220S mutation.

In some embodiments, the multispecific antibodies (e.g., bispecific antibodies) of the disclosure include antibodies having a full length antibody structure. “Full length antibody” as used herein refers to an antibody having two full length antibody heavy chains and two full length antibody light chains. A full length antibody heavy chain (HC) includes heavy chain variable and constant domains VH, CH1, CH2, and CH3. A full length antibody light chain (LC) includes light chain variable and constant domains VL and CL. The full length antibody may be lacking the C-terminal lysine (K) in either one or both heavy chains. The term “Fab-arm” or “half molecule” refers to one heavy chain-light chain pair that specifically binds an antigen. In some embodiments, one of the antigen-binding domains is a non-antibody based binding domain, e.g. a binding domain of based on a fibronectin type 3 domain, e.g. Centyrin.

### EMR2-Binding Arm

The multispecific antibodies (e.g., bispecific antibodies) described herein comprise an antigen-binding site specific for EMR2. In some embodiments, the EMR2-binding arm binds human EMR2. In some embodiments, the EMR2-binding arm binds human EMR2 and cynomolgus monkey EMR2. In some embodiments, the EMR2-binding arm binds human EMR2 but not to cynomolgus monkey EMR2. In some embodiments, the EMR2-binding arm binds bind to an epitope including one or more residues from the EMR2 extracellular domain (ECD). In some embodiments, the EMR2-binding arm binds to one or more residues of a polypeptide having the amino acid sequence of SEQ ID NO: 215 (G-protein-coupled receptor auto-proteolysis inducing (GAIN) domain and/or GPS motif of epidermal-growth-factor-like module-containing mucin-like hormone receptor 2 (EMR2)). In some embodiments, the EMR2-binding arm binds to residues D261-Q478 of human EMR2. In some embodiments, the EMR2-binding arm binds to the GAIN domain comprising the amino acid sequence SEQ ID NO: 215. In some embodiments, the EMR2-binding arm binds to the GPS motif comprising the amino acid sequence SEQ ID NO: 216. In some embodiments, the EMR2-binding arm binds to SEQ ID NO: 217 or SEQ ID NO: 218. Such EMR2-binding arms may bind to EMR2 with an affinity of 5×10⁻⁷M or less, such as 1×10⁻⁷M or less, 5×10⁻⁸M or less, 1×10⁻⁸M or less, 5×10⁻⁹M or less, 1×10⁻⁹M, or 5×10⁻¹⁰ M or less. In one embodiment, the EMR2-binding arm binds to the EMR2 with an affinity of about 1×10⁻¹¹M to 1×10⁻⁹M. In one embodiment, the EMR2-binding arm binds to the EMR2 with an affinity of about 1×10⁻¹¹M, about 2×10⁻¹¹M, about 3×10⁻¹¹M, about 4×10⁻¹¹M, about 5×10⁻¹¹M, about 6×10⁻¹¹M, about 7×10⁻¹¹M, about 8×10⁻¹¹M, about 9×10⁻¹¹M, 1×10⁻¹⁰M, about 2×10⁻¹⁰M, about 3×10⁻¹⁰M, about 4×10⁻¹⁰M, about 5×10⁻¹⁰M, about 6×10⁻¹⁰M, about 7×10⁻¹⁰M, about 8×10⁻¹⁰M, about 9×10⁻¹⁰M or about 1×10⁻⁹M. In some embodiments, the EMR2-binding arm binds to EMR2 with a dissociation constant (K_{D}) between about 0.01 nM to about 5 nM. In some embodiments, the EMR2-binding arm binds to EMR2 with an EC₅₀ between about 0.1 nM to about 15 nM.

Table 1-1 to 1-5 and Table 1-6 provide a summary of examples of some EMR2-specific antibodies described herein:

TABLE 1-1
CDR sequences (AbM) of exemplary mAbs generated against human EMR2

| Binder | | | | | | |
|---|---|---|---|---|---|---|
| Name | HC-CDR1 | HC-CDR2 | HC-CDR3 | LC-CDR1 | LC-CDR2 | LC-CDR3 |
| EMR2B410 | GFTFSSYW | RISSHGSST | GGNYYDSP | TGTSSDVG | EVSYRPS | SSYTSSSTY |
| | MH (SEQ ID | S (SEQ ID | YFDY (SEQ | GYNYVS | (SEQ ID NO: | VV (SEQ ID |
| | NO: 33) | NO: 34) | ID NO: 35) | (SEQ ID | 49) | NO: 50) |
| | | | | NO: 48) | | |
| EMR2B454 | GFTFSRYS | YISSSSNTIY | DQGTPFDY | RASQDISSW | AAISLQS | HQANSFPLT |
| | MN (SEQ ID | (SEQ ID NO: | (SEQ ID | LG (SEQ ID | (SEQ ID NO: | (SEQ ID NO: |
| | NO: 65) | 66) | NO: 67) | NO: 80) | 81) | 82) |
| EMR2B361 | GFIFRSYSM | SISSSSSYIY | REYGGPFD | SGSSSNIGN | DNNKRPS | GTWDSSLS |
| | N (SEQ ID | (SEQ ID NO: | Y (SEQ ID | DYVS (SEQ | (SEQ ID NO: | AGYV (SEQ |
| | NO: 97) | 98) | NO: 99) | ID NO: 112) | 113) | ID NO: 114) |
| EMR2B57- | GFTFRSYW | RISSGGSTT | GDNYYDSP | TGTSSDVG | EVSYRPS | SSYTSSSTY |
| LH-spFv | MH (SEQ ID | N (SEQ ID | YFDY (SEQ | GYNYVS | (SEQ ID NO: | VV (SEQ ID |
| | NO: 161) | NO: 162) | ID NO: 163) | (SEQ ID | 177) | NO: 178) |
| | | | | NO: 176) | | |

TABLE 1-2
CDR sequences (KABAT) of exemplary mAbs generated against human

| Binder | | | | | | |
|---|---|---|---|---|---|---|
| Name | HC-CDR1 | HC-CDR2 | HC-CDR3 | LC-CDR1 | LC-CDR2 | LC-CDR3 |
| EMR2B410 | SYWM (SEQ | RISSHGSST | GGNYYDSP | TGTSSDVG | EVSYRPS | SSYTSSSTY |
| | ID NO: 36) | SYADSVKG | YFDY (SEQ | GYNYVS | (SEQ ID NO: | VV (SEQ ID |
| | | (SEQ ID NO: | ID NO: 38) | (SEQ ID NO: | 52) | NO: 53) |
| | | 37) | | 51) | | |
| EMR2B454 | RYSMN | YISSSSNTIY | DQGTPFDY | RASQDISSW | AAISLQS | HQANSFPLT |
| | (SEQ ID NO: | YADSVKG | (SEQ ID NO: | LG (SEQ ID | (SEQ ID NO: | (SEQ ID NO: |
| | 68) | (SEQ ID NO: | 70) | NO: 83) | 84) | 85) |
| | | 69) | | | | |
| EMR2B361 | SYSMN | SISSSSSYIY | REYGGPFD | SGSSSNIGN | DNNKRPS | GTWDSSLS |
| | (SEQ ID NO: | YADSVKG | Y (SEQ ID | DYVS (SEQ | (SEQ ID NO: | AGYV (SEQ |
| | 100) | (SEQ ID NO: | NO: 102) | ID NO: 115) | 116) | ID NO: 117) |
| | | 101) | | | | |
| EMR2B57- | SYWMH | RISSGGSTT | GDNYYDSP | TGTSSDVG | EVSYRPS | SSYTSSSTY |
| LH-spFv | (SEQ ID NO: | NYADSVKG | YFDY (SEQ | GYNYVS | (SEQ ID NO: | VV (SEQ ID |
| | 164) | (SEQ ID NO: | ID NO: 166) | (SEQ ID NO: | 180) | NO: 181) |
| | | 165) | | 179) | | |

TABLE 1-3
CDR sequences (CHOTHIA) of exemplary mAbs generated against human

| Binder | | | | | | |
|---|---|---|---|---|---|---|
| Name | HC-CDR1 | HC-CDR2 | HC-CDR3 | LC-CDR1 | LC-CDR2 | LC-CDR3 |
| EMR2B410 | GFTFSSY | SSHGSS | GGNYYDSP | TGTSSDVG | EVSYRPS | SSYTSSSTY |
| | (SEQ ID NO: | (SEQ ID NO: | YFDY (SEQ | GYNYVS | (SEQ ID NO: | VV (SEQ ID |
| | 39) | 40) | ID NO: 41) | (SEQ ID NO: | 55) | NO: 56) |
| | | | | 54) | | |
| EMR2B454 | GFTFSRY | SSSSNT | DQGTPFDY | RASQDISSW | AAISLQS | HQANSFPLT |
| | (SEQ ID NO: | (SEQ ID NO: | (SEQ ID | LG (SEQ ID | (SEQ ID NO: | (SEQ ID NO: |
| | 71) | 72) | NO: 73) | NO: 86) | 87) | 88) |
| EMR2B361 | GFIFRSY | SSSSSY | REYGGPFD | SGSSSNIGN | DNNKRPS | GTWDSSLS |
| | (SEQ ID NO: | (SEQ ID NO: | Y (SEQ ID | DYVS (SEQ | (SEQ ID NO: | AGYV (SEQ |
| | 103) | 104) | NO: 105) | ID NO: 118) | 119) | ID NO: 120) |
| EMR2B57- | GFTFRSY | SSGGST | GDNYYDSP | TGTSSDVG | EVSYRPS | SSYTSSSTY |
| LH-spFv | (SEQ ID NO: | (SEQ ID NO: | YFDY (SEQ | GYNYVS | (SEQ ID NO: | VV (SEQ ID |
| | 167) | 168) | ID NO: 169) | (SEQ ID NO: | 183) | NO: 184) |
| | | | | 182) | | |

TABLE 1-4
CDR sequences (IMGT) of exemplary mAbs generated against human EMR2

| Binder | | | | | | |
|---|---|---|---|---|---|---|
| Name | HC-CDR1 | HC-CDR2 | HC-CDR3 | LC-CDR1 | LC-CDR2 | LC-CDR3 |
| EMR2B410 | GFTFSSYW | ISSHGSST | ARGGNYYD | SSDVGGYN | EVS | SSYTSSSTY |
| | (SEQ ID NO: | (SEQ ID NO: | SPYFDY | Y (SEQ ID | | VV (SEQ ID |
| | 42) | 43) | (SEQ ID NO: | NO: 57) | | NO: 59) |
| | | | 44) | | | |
| EMR2B454 | GFTFSRYS | ISSSSNTI | ARDQGTPF | QDISSW | AAI | HQANSFPLT |
| | (SEQ ID NO: | (SEQ ID NO: | DY (SEQ ID | (SEQ ID NO: | | (SEQ ID NO: |
| | 74) | 75) | NO: 76) | 89) | | 91) |
| EMR2B361 | GFIFRSYS | ISSSSSYI | ARREYGGP | SSNIGNDY | DNN | GTWDSSLS |
| | (SEQ ID NO: | (SEQ ID NO: | FDY (SEQ ID | (SEQ ID NO: | | AGYV (SEQ |
| | 106) | 107) | NO: 108) | 121) | | ID NO: 123) |
| EMR2B57- | GFTFRSYW | ISSGGSTT | ARGDNYYD | SSDVGGYN | EVS | SSYTSSSTY |
| LH-spFv | (SEQ ID NO: | (SEQ ID NO: | SPYFDY | Y (SEQ ID | | VV (SEQ ID |
| | 170) | 171) | (SEQ ID NO: | NO: 185) | | NO: 187) |
| | | | 172) | | | |

TABLE 1-5
CDR sequences (CONTACT) of exemplary mAbs generated against human

| Binder | | | | | | |
|---|---|---|---|---|---|---|
| Name | HC-CDR1 | HC-CDR2 | HC-CDR3 | LC-CDR1 | LC-CDR2 | LC-CDR3 |
| EMR2B410 | SSYWMH | WVSRISSHG | ARGGNYYD | VGGYNYVS | LMIYEVSY | SSYTSSSTY |
| | (SEQ ID NO: | SSTS (SEQ | SPYFD (SEQ | WY (SEQ ID | RP (SEQ ID | V (SEQ ID |
| | 45) | ID NO: 46) | ID NO: 47) | NO: 60) | NO: 61) | NO: 62) |
| EMR2B454 | SRYSMN | WVSYISSSS | ARDQGTPF | SSWLGWY | LLIYAAISL | HQANSFPL |
| | (SEQ ID NO: | NTIY (SEQ | D (SEQ ID | (SEQ ID | Q (SEQ ID | (SEQ ID NO: |
| | 77) | ID NO: 78) | NO: 79) | NO: 92) | NO: 93) | 94) |
| EMR2B361 | RSYSMN | WVSSISSSS | ARREYGGP | IGNDYVSW | LLIYDNNK | GTWDSSLS |
| | (SEQ ID NO: | SYIY (SEQ | FD (SEQ ID | Y (SEQ ID | RP (SEQ ID | AGY (SEQ |
| | 109) | ID NO: 110) | NO: 111) | NO: 124) | NO: 125) | ID NO: 126) |
| EMR2B57- | RSYWMH | WVSRISSGG | ARGDNYYD | VGGYNYVS | LMIYEVSY | SSYTSSSTY |
| LH-spFv | (SEQ ID NO: | STTN (SEQ | SPYFD (SEQ | WY (SEQ ID | RP (SEQ ID | V (SEQ ID |
| | 173) | ID NO: 174) | ID NO: 175) | NO: 188) | NO: 189) | NO: 190) |

TABLE 1-6
VH and VL sequences of exemplary
mAbs generated against human EMR2

| | VH amino acid | VL amino acid |
|---|---|---|
| | sequence SEQ | sequence SEQ |
| Binder Name | ID NO | ID NO |
| EMR2B410 | 63 | 64 |
| EMR2B454 | 95 | 96 |
| EMR2B361 | 127 | 128 |
| EMR2B57-LH-spFv | 191 | 192 |

In some embodiments, the EMR2-binding arm comprises a heavy chain variable domain comprising a CDR1, a CDR2, and a CDR3 of any one of the antibodies described in Tables 1-1 to 1-5. In some embodiments, the EMR2-binding arm comprises a light chain variable region comprising a CDR1, a CDR2, and a CDR3 of any one of the antibodies described in Tables 1-1 to 1-5. In some embodiments, the EMR2-binding arm comprises a heavy chain variable domain comprising a CDR1, a CDR2, and a CDR3 of any one of the antibodies described in Tables 1-1 to 1-5 and a light chain variable region comprising a CDR1, a CDR2, and a CDR3 of any one of the antibodies described in Tables 1-1 to 1-5. In some embodiments, the EMR2-binding arm competes for binding to EMR2 with an antibody or an antigen-binding comprising a heavy chain comprising a CDR1, a CDR2, and a CDR3 of any one of the antibodies described in Tables 1-1 to 1-5 and a light chain comprising a CDR1, a CDR2, and a CDR3 of any one of the antibodies described in Tables 1-1 to 1-5.

In some embodiments, the EMR2-binding arm comprises a heavy chain variable domain of any one of the antibodies described in Table 1-6. In some embodiments, the EMR2-binding arm comprises a light chain variable region of any one of the antibodies described in Table 1-6. In some embodiments, the EMR2-binding arm comprises a heavy chain variable domain of any one of the antibodies described in Table 1-6 and a light chain variable region of any one of the antibodies described in Table 1-6. In some embodiments, the EMR2-binding arm competes for binding to EMR2 with an antibody or an antigen-binding comprising a heavy chain variable domain of any one of the antibodies described in Table 1-6 and a light chain variable domain of any one of the antibodies described in Table 1-6.

In some embodiments, the EMR2-binding arm comprises a heavy chain comprising a CDR1, a CDR2, and a CDR3 of any one of the antibodies described in Tables 1-1 to 1-5. In some embodiments, the EMR2-binding arm comprises a light chain comprising a CDR1, a CDR2, and a CDR3 of any one of the antibodies described in Tables 1-1 to 1-5. In some embodiments, the EMR2-binding arm comprises a heavy chain comprising a CDR1, a CDR2, and a CDR3 of any one of the antibodies described in Tables 1-1 to 1-5 and a light chain comprising a CDR1, a CDR2, and a CDR3 of any one of the antibodies described in Tables 1-1 to 1-5.

In some embodiments, the EMR2-binding arm comprises a heavy chain comprising a heavy chain variable domain of any one of the antibodies described in Table 1-6. In some embodiments, the EMR2-binding arm comprises a light chain comprising a light chain variable domain of any one of the antibodies described in Table 1-6. In some embodiments, the EMR2-binding arm comprises a heavy chain comprising a heavy chain variable domain of any one of the antibodies described in Table 1-6 and a light chain comprising a light chain variable domain of any one of the antibodies described in Table 1-6.

In some embodiments, the antibody or the antigen-binding fragment thereof, comprises: a) a heavy chain complementarity determining region (HCDR) 1, a HCDR2 and a HCDR3 of the heavy chain variable region (VH) of SEQ ID NO: 63, and a light chain complementarity determining region (LCDR) 1, a LCDR2 and a LCDR3 of the light chain variable region (VL) of SEQ ID NO: 64; b) a HCDR1, a HCDR2, and a HCDR3 of the VH of SEQ ID NO: 95, and a LCDR1, a LCDR2, and a LCDR3 of the VL of SEQ ID NO: 96; c) a HCDR1, a HCDR2, and a HCDR3 of the VH of SEQ ID NO: 127, and a LCDR1, a LCDR2, and a LCDR3 of the VL of SEQ ID NO: 128; or d) a HCDR1, a HCDR2, and a HCDR3 of the VH of SEQ ID NO: 191, and a LCDR1, a LCDR2, and a LCDR3 of the VL of SEQ ID NO: 192. In some embodiments, the antibody or the antigen-binding fragment thereof, comprises a HCDR1, a HCDR2, and a HCDR3 of the VH of SEQ ID NO: 95, and a LCDR1, a LCDR2, and a LCDR3 of the VL of SEQ ID NO: 96.

In some embodiments, the antibody or the antigen-binding fragment thereof, comprises the HCDR1, the HCDR2, the HCDR3, the LCDR1, the LCDR2 and the LCDR3 of: a) SEQ ID NO: 33, 34, 35, 48, 49, and 50, respectively; b) SEQ ID NO: 36, 37, 38, 51, 52, and 53, respectively; c) SEQ ID NO: 39, 40, 41, 54, 55, and 56, respectively; d) SEQ ID NO: 42, 43, 44, 57, amino acid sequence EVS, and SEQ ID NO: 59, respectively; e) SEQ ID NO: 45, 46, 47, 60, 61, and 62, respectively; f) SEQ ID NO: 65, 66, 67, 80, 81, and 82, respectively; g) SEQ ID NO: 68, 69, 70, 83, 84, and 85, respectively; h) SEQ ID NO: 71, 72, 73, 86, 87, and 88, respectively; i) SEQ ID NO: 74, 75, 76, 89, amino acid sequence AAI, and SEQ ID NO: 91, respectively; j) SEQ ID NO: 77, 78, 79, 92, 93, and 94, respectively; k) SEQ ID NO: 97, 98, 99, 112, 113, and 114, respectively; l) SEQ ID NO: 100, 101, 102, 115, 116, and 117, respectively; m) SEQ ID NO: 103, 104, 105, 118, 119, and 120, respectively; n) SEQ ID NO: 106, 107, 108, 121, 122, and 123, respectively; o) SEQ ID NO: 109, 110, 111, 124, 125, and 126, respectively; p) SEQ ID NO: 161, 162, 163, 176, 177, and 178, respectively; q) SEQ ID NO: 164, 165, 166, 179, 180, and 181, respectively; r) SEQ ID NO: 167, 168, 169, 182, 18, and 184, respectively; s) SEQ ID NO: 170, 171, 172, 185, amino acid sequence EVS, and SEQ ID NO:187, respectively; or t) SEQ ID NO: 173, 174, 175, 188, 189, and 190, respectively. In some embodiments, the antibody or the antigen-binding fragment thereof, comprises the HCDR1, the HCDR2, the HCDR3, the LCDR1, the LCDR2 and the LCDR3 of SEQ ID NO: 65, 66, 67, 80, 81, and 82, respectively; SEQ ID NO: 68, 69, 70, 83, 84, and 85, respectively; SEQ ID NO: 71, 72, 73, 86, 87, and 88, respectively; SEQ ID NO: 74, 75, 76, 89, amino acid sequence AAI, and SEQ ID NO: 91, respectively; or SEQ ID NO: 77, 78, 79, 92, 93, and 94, respectively

In some embodiments, the antibody or an antigen-binding fragment thereof, comprises: a) the HC1 comprises the amino acid sequence of SEQ ID NO: 194 and the LC1 comprises the amino acid sequence of SEQ ID NO: 195; b) the HC1 comprises the amino acid sequence of SEQ ID NO: 196 and the LC1 comprises the amino acid sequence of SEQ ID NO: 197; or c) the HC1 comprises the amino acid sequence of SEQ ID NO: 198 and the LC1 comprises the amino acid sequence of SEQ ID NO: 199. In some embodiments, the antibody or an antigen-binding fragment thereof, comprises an HC1 comprising the amino acid sequence of SEQ ID NO: 198 and a LC1 comprising the amino acid sequence of SEQ ID NO: 199.

In some embodiments, the antibody or an antigen-binding fragment thereof, comprises a variable heavy chain region (VH) comprising an amino acid sequence that is at least 90%, at least 91%, at least 92%, at least 93%, at least 94%, at least 95%, at least 96%, at least 97%, at least 98%, at least 99% or 100% identical to: a) the VH of SEQ ID NO: 63; b) the VH of SEQ ID NO: 95; c) the VH of SEQ ID NO: 127; or to d) the VH of SEQ ID NO: 191. In some embodiments, the antibody or an antigen-binding fragment thereof, comprises a variable heavy chain region (VH) comprising an amino acid sequence that is at least 90%, at least 91%, at least 92%, at least 93%, at least 94%, at least 95%, at least 96%, at least 97%, at least 98%, at least 99% or 100% identical to the VH of SEQ ID NO: 95.

In some embodiments, the antibody or the antigen-binding fragment thereof, comprises or further comprises a VL comprising an amino acid sequence that is at least 90%, at least 91%, at least 92%, at least 93%, at least 94%, at least 95%, at least 96%, at least 97%, at least 98%, at least 99% or 100% identical to: a) the VL of SEQ ID NO: 64; b) the VL of SEQ ID NO: 96; c) the VL of SEQ ID NO: 128; or to d) the VL of SEQ ID NO: 192. In some embodiments, the antibody or the antigen-binding fragment thereof, comprises or further comprises a VL comprising an amino acid sequence that is at least 90%, at least 91%, at least 92%, at least 93%, at least 94%, at least 95%, at least 96%, at least 97%, at least 98%, at least 99% or 100% identical to the VL of SEQ ID NO: 96.

In some embodiments, the antibody or the antigen-binding fragment thereof, comprises or further comprises the antibody or the antigen-binding fragment thereof, comprises: a) the VH of SEQ ID NO: 63 and the VL of SEQ ID NO: 64; b) the VH of SEQ ID NO: 95 and the VL of SEQ ID NO: 96; c) the VH of SEQ ID NO: 127 and the VL of SEQ ID NO: 128; or d) the VH of SEQ ID NO: 191 and the VL of SEQ ID NO: 192. In some embodiments, the antibody or the antigen-binding fragment thereof, comprises or further comprises the antibody or the antigen-binding fragment thereof, comprises the VH of SEQ ID NO: 95 and the VL of SEQ ID NO: 96.

In some embodiments, the present disclosure provides a polynucleotide comprising a sequence encoding an amino acid sequence that is at least 90%, at least 91%, at least 92%, at least 93%, at least 94%, at least 95%, at least 96%, at least 97%, at least 98%, at least 99% or 100% identical to: a) the VH of SEQ ID NO: 63; b) the VH of SEQ ID NO: 95; c) the VH of SEQ ID NO: 127; or d) the VH of SEQ ID NO: 191. In some embodiments, the present disclosure provides an isolated polynucleotide comprising a sequence encoding an amino acid sequence that is at least 90%, at least 91%, at least 92%, at least 93%, at least 94%, at least 95%, at least 96%, at least 97%, at least 98%, at least 99% or 100% identical to: the VH of SEQ ID NO: 95.

In some embodiments, the present disclosure provides a polynucleotide comprising a sequence encoding an amino acid sequence that is at least 90%, at least 91%, at least 92%, at least 93%, at least 94%, at least 95%, at least 96%, at least 97%, at least 98%, at least 99% or 100% identical to: a) the VL of SEQ ID NO: 64; b) the VL of SEQ ID NO: 96; c) the VL of SEQ ID NO: 128; or d) the VL of SEQ ID NO: 192. In some embodiments, the present disclosure provides an isolated polynucleotide comprising a sequence encoding an amino acid sequence that is at least 90%, at least 91%, at least 92%, at least 93%, at least 94%, at least 95%, at least 96%, at least 97%, at least 98%, at least 99% or 100% identical to the VL of SEQ ID NO: 96.

In some embodiments, the present disclosure provides an isolated polynucleotide comprising a sequence encoding: a) the amino acid sequence of SEQ ID NO: 63 and/or the amino acid sequence of SEQ ID NO: 64; b) the amino acid sequence of SEQ ID NO: 95 and/or the amino acid sequence of SEQ ID NO: 96; c) the amino acid sequence of SEQ ID NO: 127 and/or the amino acid sequence of SEQ ID NO: 128; or d) the amino acid sequence of SEQ ID NO: 191 and/or the amino acid sequence of SEQ ID NO: 192. In some embodiments, the present disclosure provides an isolated polynucleotide comprising a sequence encoding: the amino acid sequence of SEQ ID NO: 95 and/or the amino acid sequence of SEQ ID NO: 96.

In some embodiments, the polynucleotide comprises a sequence encoding a heavy chain comprising the amino acid sequence of SEQ ID NO: 194, 196, 198, or 202. In some embodiments, the polynucleotide comprises a sequence encoding a light chain comprising the amino acid sequence of SEQ ID NO: 195, 197, or 199. In some embodiments, the polynucleotide comprises a nucleotide sequence encoding: a) the amino acid sequence of SEQ ID NO: 194 and/or SEQ ID NO: 195; b) the amino acid sequence of SEQ ID NO: 196 and/or SEQ ID NO: 197; c) the amino acid sequence of SEQ ID NO: 198 and/or the amino acid sequence of SEQ ID NO: 199; or d) the amino acid sequence of SEQ ID NO: 202. In some embodiments, the isolated polynucleotide comprises a sequence encoding a heavy chain comprising the amino acid sequence of SEQ ID NO: 198. In some embodiments, the isolated polynucleotide comprises a sequence encoding a light chain comprising the amino acid sequence of SEQ ID NO: 199. In some embodiments, the polynucleotide comprises a nucleotide sequence encoding: the amino acid sequence of SEQ ID NO: 198 and/or the amino acid sequence of SEQ ID NO: 199.

The EMR2-binding arm may be derived from any species by recombinant means. For example, the EMR2 antigen-binding region may be derived from mouse, rat, goat, horse, swine, bovine, chicken, rabbit, camelid, donkey, human, or chimeric versions thereof. For use in administration to humans, non-human derived antigen-binding fragments may be genetically or structurally altered to be less antigenic upon administration to a human patient. In some embodiments, the EMR2-binding arm comprises antigen-binding fragments which is chimeric.

In some embodiments, the EMR2-binding arm comprises humanized antigen-binding fragments. Humanized antigen-binding fragments may be derived from chimeric immunoglobulins, immunoglobulin chains or fragments thereof (such as Fv, scFv, spFv, Fab, Fab′, F(ab′)2, or other antigen-binding subsequences of antibodies) that contain minimal sequence derived from non-human immunoglobulin. For the most part, humanized antibodies or antigen-binding fragments are human immunoglobulins (recipient antibody) or antigen-binding fragments in which residues from a complementary-determining region (CDR) of the recipient are replaced by residues from a CDR of a non-human species (donor antibody) such as mouse, rat or rabbit having the desired specificity, affinity, and capacity. In general, the humanized antibody antigen-binding fragments will comprise substantially all of at least one, and typically two, variable domains, in which all or substantially all of the CDR regions correspond to those of a non-human immunoglobulin and all or substantially all of the framework regions are those of a human immunoglobulin sequence. The humanized antibody antigen-binding fragments may include at least a portion of an immunoglobulin constant region (Fc), typically that of a human immunoglobulin.

### TRBV19-Binding Arm

The multispecific antibodies (e.g., bispecific antibodies) described herein may comprise an antigen-binding site specific for TRBV19. In some embodiments, the TRBV19-binding arm binds human TRBV19. In some embodiments, TRBV19-binding arm binds human TRBV19 and cynomolgus monkey TRBV19, preferably the extracellular domain thereof. In some embodiments, TRBV19-binding arm binds human TRBV19 but not to cynomolgus monkey TRBV19.

In some embodiments, the TRBV19-binding arm comprises a heavy chain CDR1, CDR2, and CDR3 derived from an antibody clone as described in Tables 2-1 to 2-5. In some embodiments, the TRBV19-binding arm comprises a light chain CDR1, CDR2, and CDR3 derived from an antibody clone as described in Tables 2-1 to 2-5. In some embodiments, the TRBV19-binding arm comprises heavy chain CDR1, CDR2, and CDR3 and light chain CDR1, CDR2, and CDR3 derived from an antibody clone as described in Tables 2-1 to 2-5.

In some exemplary embodiments, the TRBV19-binding arm comprises a heavy chain variable domain derived from an antibody clone as described in Table 2-6. In some exemplary embodiments, the TRBV19-binding arm comprises heavy chain variable domain and light chain variable domain derived from an antibody clone as described in Table 2-6.

Tables 2-1 to 2-5 and Table 2-6 provide a summary of examples of some TRBV19-specific antibodies described herein:

TABLE 2-1
CDR sequences (AbM) of exemplary mAbs generated against human TRBV19

| Binder | | | | | | |
|---|---|---|---|---|---|---|
| Name | HC-CDR1 | HC-CDR2 | HC-CDR3 | LC-CDR1 | LC-CDR2 | LC-CDR3 |
| B17B21- | GYSITSGYF | YISYDGSNN | PSPGTGYA | RSSQSLVHS | KVSNRFS | SQSTHVPFT |
| VL-N33S | WN (SEQ ID | (SEQ ID NO: | VDY (SEQ | SGNTYLH | (SEQ ID NO: | (SEQ ID NO: |
| | NO: 1) | 2) | ID NO: 3) | (SEQ ID NO: | 17) | 18) |
| | | | | 16) | | |
| B17B852- | GYSITSGYF | YISYDGSNN | PSPGTGYA | RSSQSLVHS | KVSNRFS | SQSTHVPFT |
| G34R- | WN (SEQ ID | (SEQ ID NO: | VDY (SEQ | NRNTYLH | (SEQ ID NO: | (SEQ ID NO: |
| LH-spFv | NO: 129) | 130) | ID NO: 131) | (SEQ ID NO: | 145) | 146) |
| | | | | 144) | | |

TABLE 2-2
CDR sequences (KABAT) of exemplary mAbs generated against human TRBV19

| Binder | | | | | | |
|---|---|---|---|---|---|---|
| Name | HC-CDR1 | HC-CDR2 | HC-CDR3 | LC-CDR1 | LC-CDR2 | LC-CDR3 |
| B17B21- | SGYFWN | YISYDGSNN | PSPGTGYA | RSSQSLVHS | KVSNRFS | SQSTHVPFT |
| VL-N33S | (SEQ ID NO: | YNPSLKS | VDY (SEQ | SGNTYLH | (SEQ ID NO: | (SEQ ID NO: |
| | 4) | (SEQ ID NO: | ID NO: 6) | (SEQ ID NO: | 20) | 21) |
| | | 5) | | 19) | | |
| B17B852- | SGYFWN | YISYDGSNN | PSPGTGYA | RSSQSLVHS | KVSNRFS | SQSTHVPFT |
| G34R- | (SEQ ID NO: | YNPSLKS | VDY (SEQ | NRNTYLH | (SEQ ID NO: | (SEQ ID NO: |
| LH-spFv | 132) | (SEQ ID NO: | ID NO: 134) | (SEQ ID NO: | 148) | 149) |
| | | 133) | | 147) | | |

TABLE 2-3
CDR sequences (CHOTHIA) of exemplary mAbs generated against human TRBV19

| Binder | | | | | | |
|---|---|---|---|---|---|---|
| Name | HC-CDR1 | HC-CDR2 | HC-CDR3 | LC-CDR1 | LC-CDR2 | LC-CDR3 |
| B17B21- | GYSITSGY | SYDGS (SEQ | PSPGTGYA | RSSQSLVHS | KVSNRFS | SQSTHVPFT |
| VL-N33S | (SEQ ID NO: | ID NO: 8) | VDY (SEQ | SGNTYLH | (SEQ ID NO: | (SEQ ID NO: |
| | 7) | | ID NO: 9) | (SEQ ID NO: | 23) | 24) |
| | | | | 22) | | |
| B17B852- | GYSITSGY | SYDGS (SEQ | PSPGTGYA | RSSQSLVHS | KVSNRFS | SQSTHVPFT |
| G34R- | (SEQ ID NO: | ID NO: 136) | VDY (SEQ | NRNTYLH | (SEQ ID NO: | (SEQ ID NO: |
| LH-spFv | 135) | | ID NO: 137) | (SEQ ID NO: | 151) | 152) |
| | | | | 150) | | |

TABLE 2-4
CDR sequences (IMGT) of exemplary mAbs generated against human TRBV19

| Binder | | | | | | |
|---|---|---|---|---|---|---|
| Name | HC-CDR1 | HC-CDR2 | HC-CDR3 | LC-CDR1 | LC-CDR2 | LC-CDR3 |
| B17B21- | GYSITSGYF | ISYDGSN | ASPSPGTGY | QSLVHSSG | KVS | SQSTHVPFT |
| VL-N33S | (SEQ ID NO: | (SEQ ID NO: | AVDY (SEQ | NTY (SEQ | | (SEQ ID NO: |
| | 10 | 11) | ID NO: 12) | ID NO: 25) | | 27) |
| B17B852- | GYSITSGYF | ISYDGSN | ASPSPGTGY | QSLVHSNR | KVS | SQSTHVPFT |
| G34R- | (SEQ ID NO: | (SEQ ID NO: | AVDY (SEQ | NTY (SEQ | | (SEQ ID NO: |
| LH-spFv | 138) | 139) | ID NO: 140) | ID NO: 153) | | 155) |

TABLE 2-5
CDR sequences (CONTACT) of exemplary mAbs generated against human TRBV19

| Binder | | | | | | |
|---|---|---|---|---|---|---|
| Name | HC-CDR1 | HC-CDR2 | HC-CDR3 | LC-CDR1 | LC-CDR2 | LC-CDR3 |
| B17B21- | TSGYFWN | WIGYISYDG | ASPSPGTGY | VHSSGNTY | FLIYKVSNR | SQSTHVPF |
| VL-N33S | (SEQ ID NO: | SNN (SEQ ID | AVD (SEQ | LHWY (SEQ | F (SEQ ID | (SEQ ID NO: |
| | 13) | NO: 14) | ID NO: 15) | ID NO: 28) | NO: 29) | 30) |
| B17B852- | TSGYFWN | WMGYISYD | ASPSPGTGY | VHSNRNTY | LLIYKVSNR | SQSTHVPF |
| G34R- | (SEQ ID NO: | GSNN (SEQ | AVD (SEQ | LHWY (SEQ | F (SEQ ID | (SEQ ID NO: |
| LH-spFv | 141) | ID NO: 142) | ID NO: 143) | ID NO: 156) | NO: 157) | 158) |

TABLE 2-6
VH and VL sequences of exemplary mAbs
generated against human TRBV19

| | VH amino acid | VL amino acid |
|---|---|---|
| | sequence SEQ | sequence SEQ |
| Binder Name | ID NO | ID NO |
| B17B21-VL-N33S | 31 | 32 |
| B17B852-G34R-LH- | 159 | 160 |
| spFv | | |

In some embodiments, the TRBV19-binding arm comprises a heavy chain variable domain comprising a CDR1, a CDR2, and a CDR3 of any one of the antibodies described in Tables 2-1 to 2-5. In some embodiments, the TRBV19-binding arm comprises a light chain variable domain comprising a CDR1, a CDR2, and a CDR3 of any one of the antibodies described in Tables 2-1 to 2-5. In some embodiments, the TRBV19-binding arm comprises a heavy chain variable domain comprising a CDR1, a CDR2, and a CDR3 of any one of the antibodies described in Table 2a and a light chain variable domain comprising a CDR1, a CDR2, and a CDR3 of any one of the antibodies described in Tables 2-1 to 2-5. In some embodiments, the TRBV19-binding arm competes for binding to TRBV19 with an antibody or an antigen-binding comprising a heavy chain comprising a CDR1, a CDR2, and a CDR3 of any one of the antibodies described in Tables 2-1 to 2-5 and a light chain comprising a CDR1, a CDR2, and a CDR3 of any one of the antibodies described in Tables 2-1 to 2-5.

In some embodiments, the TRBV19-binding arm comprises a heavy chain variable domain of any one of the antibodies described in Table 2-6. In some embodiments, the TRBV19-binding arm comprises a light chain variable region of any one of the antibodies described in Table 2b. In some embodiments, the TRBV19-binding arm comprises a heavy chain variable domain of any one of the antibodies described in Table 2-6 and a light chain variable region of any one of the antibodies described in Table 2-6. In some embodiments, the TRBV19-binding arm competes for binding to TRBV19 with an antibody or an antigen-binding comprising a heavy chain variable domain of any one of the antibodies described in Table 2-6 and a light chain variable domain of any one of the antibodies described in Table 2-6.

In some embodiments, the TRBV19-binding arm comprises a heavy chain comprising a CDR1, a CDR2, and a CDR3 of any one of the antibodies described in Tables 2-1 to 2-5. In some embodiments, the TRBV19-binding arm comprises a light chain comprising a CDR1, a CDR2, and a CDR3 of any one of the antibodies described in Tables 2-1 to 2-5. In some embodiments, the TRBV19-binding arm comprises a heavy chain comprising a CDR1, a CDR2, and a CDR3 of any one of the antibodies described in Tables 2-1 to 2-5 and a light chain comprising a CDR1, a CDR2, and a CDR3 of any one of the antibodies described in Tables 2-1 to 2-5.

In some embodiments, the TRBV19-binding arm comprises a heavy chain comprising a heavy chain variable domain of any one of the antibodies described in Table 2-6. In some embodiments, the TRBV19-binding arm comprises a light chain comprising a light chain variable domain of any one of the antibodies described in Table 2-6. In some embodiments, the TRBV19-binding arm comprises a heavy chain comprising a heavy chain variable domain of any one of the antibodies described in Table 2-6 and a light chain comprising a light chain variable domain of any one of the antibodies described in Table 2-6.

In some embodiments, the antibody or the antigen-binding fragment thereof, comprises: a) a heavy chain complementarity determining region (HCDR) 1, a HCDR2 and a HCDR3 of the heavy chain variable region (VH) of SEQ ID NO: 31 and a light chain complementarity determining region (LCDR) 1, a LCDR2 and a LCDR3 of the light chain variable region (VL) of SEQ ID NO: 32; or b) a HCDR1, a HCDR2, and a HCDR3 of the VH of SEQ ID NO: 159 and a LCDR1, LCDR2, and LCDR3 of the VL of SEQ ID NO: 160. In some embodiments, the antibody or the antigen-binding fragment thereof, comprises: a HCDR1, a HCDR2, and a HCDR3 of the VH of SEQ ID NO: 159 and a LCDR1, LCDR2, and LCDR3 of the VL of SEQ ID NO: 160.

In some embodiments, the bispecific antibody or the bispecific antigen-binding fragment thereof, comprises the HCDR1, the HCDR2, the HCDR3, the LCDR1, the LCDR2 and the LCDR3 of: a) SEQ ID NOs: 1, 2, 3, 16, 17, and 18, respectively; b) SEQ ID NOs: 4, 5, 6, 19, 20, and 21, respectively; c) SEQ ID NOs: 7, 8, 9, 22, 23, and 24, respectively; d) SEQ ID NOs: 10, 11, 12, 25, amino acid sequence KVS, and SEQ ID NO: 27, respectively; e) SEQ ID NOs: 13, 14, 15, 28, 29, and 30, respectively; f) SEQ ID NOs: 129, 130, 131, 144, 145, and 146, respectively; g) SEQ ID NOs: 132, 133, 134, 147, 148, and 149, respectively; h) SEQ ID NOs: 135, 136, 137, 150, 151, and 152, respectively; i) SEQ ID NOs: 138, 139, 140, 153, 154, and 155, respectively; or j) SEQ ID NOs: 141, 142, 143, 156, 157, and 158, respectively. In some embodiments, the bispecific antibody or the bispecific antigen-binding fragment thereof, comprises the HCDR1, the HCDR2, the HCDR3, the LCDR1, the LCDR2 and the LCDR3 of SEQ ID NOs: 129, 130, 131, 144, 145, and 146, respectively; SEQ ID NOs: 132, 133, 134, 147, 148, and 149, respectively; SEQ ID NOs: 135, 136, 137, 150, 151, and 152, respectively; SEQ ID NOs: 138, 139, 140, 153, 154, and 155, respectively; or SEQ ID NOs: 141, 142, 143, 156, 157, and 158, respectively.

In some embodiments, the bispecific antibody or the bispecific antigen-binding fragment thereof, comprises a heavy chain variable region (VH) comprising an amino acid sequence that is at least 90%, at least 91%, at least 92%, at least 93%, at least 94%, at least 95%, at least 96%, at least 97%, at least 98%, at least 99% or 100% identical to: a) the VH of SEQ ID NO: 31; or b) the VH of SEQ ID NO: 159. In some embodiments, the bispecific antibody or the bispecific antigen-binding fragment thereof, comprises a VH comprising an amino acid sequence that is at least 90%, at least 91%, at least 92%, at least 93%, at least 94%, at least 95%, at least 96%, at least 97%, at least 98%, at least 99% or 100% identical to: the VH of SEQ ID NO: 159.

In some embodiments, the bispecific antibody or the bispecific antigen-binding fragment thereof, comprises or further comprises a light chain variable region (VL) comprising an amino acid sequence that is at least 90%, at least 91%, at least 92%, at least 93%, at least 94%, at least 95%, at least 96%, at least 97%, at least 98%, at least 99% or 100% identical to: a) the VL of SEQ ID NO: 32; or b) the VL of SEQ ID NO: 160. In some embodiments, the bispecific antibody or the bispecific antigen-binding fragment thereof, comprises or further comprises a VL comprising an amino acid sequence that is at least 90%, at least 91%, at least 92%, at least 93%, at least 94%, at least 95%, at least 96%, at least 97%, at least 98%, at least 99% or 100% identical to: the VL of SEQ ID NO: 160.

In some embodiments, the bispecific antibody or the bispecific antigen-binding fragment thereof, comprises: a) the VH of SEQ ID NO: 31 and the VL of SEQ ID NO: 32; or b) the VH of SEQ ID NO: 159 and the VL of SEQ ID NO: 160. In some embodiments, the bispecific antibody or the bispecific antigen-binding fragment thereof, comprises: the VH of SEQ ID NO: 159 and the VL of SEQ ID NO: 160.

In some embodiments, the bispecific antibody or the bispecific antigen-binding fragment thereof, comprises: a) a heavy chain complementarity determining region (HCDR) 1, a HCDR2 and a HCDR3 of the heavy chain variable region (VH) of SEQ ID NO: 63, and a light chain complementarity determining region (LCDR) 1, a LCDR2 and a LCDR3 of the light chain variable region (VL) of SEQ ID NO: 64; b) a HCDR1, a HCDR2, and a HCDR3 of the VH of SEQ ID NO: 95, and a LCDR1, a LCDR2, and a LCDR3 of the VL of SEQ ID NO: 96; c) a HCDR1, a HCDR2, and a HCDR3 of the VH of SEQ ID NO: 127, and a LCDR1, a LCDR2, and a LCDR3 of the VL of SEQ ID NO: 128; or d) a HCDR1, a HCDR2, and a HCDR3 of the VH of SEQ ID NO: 191, and a LCDR1, a LCDR2, and a LCDR3 of the VL of SEQ ID NO: 192. In some embodiments, the bispecific antibody or the bispecific antigen-binding fragment thereof, comprises: a) a HCDR1, a HCDR2, and a HCDR3 of the VH of SEQ ID NO: 95, and a LCDR1, a LCDR2, and a LCDR3 of the VL of SEQ ID NO: 96.

In some embodiments, the bispecific antibody or the bispecific antigen-binding fragment thereof, comprises a HC1 comprises the amino acid sequence of SEQ ID NO: 193 or 200. In some embodiments, the bispecific antibody or the bispecific antigen-binding fragment thereof, comprises a HC1 comprises the amino acid sequence of SEQ ID NO: 193.

In some embodiments, the bispecific antibody or the bispecific antigen-binding fragment thereof, comprises a LC1 comprises the amino acid sequence of SEQ ID NO: 201.

In some embodiments, the bispecific antibody or the bispecific antigen-binding fragment thereof, comprises: a) the HC1 comprises the amino acid sequence of SEQ ID NO: 193; or b) the HC1 comprises the amino acid sequence of SEQ ID NO: 200 and the LC1 comprises the amino acid sequence of SEQ ID NO: 201. In some embodiments, the HC2 comprises the amino acid sequence of SEQ ID NO: 194, 196, 198, or 202. In some embodiments, the bispecific antibody or the bispecific antigen-binding fragment thereof, comprises the HC1 comprises the amino acid sequence of SEQ ID NO: 193. In some embodiments, the HC2 comprises the amino acid sequence of SEQ ID NO: 198. In some embodiments, the LC2 comprises the amino acid sequence of SEQ ID NO: 195, 197, or 199. In some embodiments, the LC2 comprises the amino acid sequence of SEQ ID NO: 199. In some embodiments, a) the HC2 comprises the amino acid sequence of SEQ ID NO: 194 and the LC2 comprises the amino acid sequence of SEQ ID NO: 195; b) the HC2 comprises the amino acid sequence of SEQ ID NO: 196 and the LC1 comprises the amino acid sequence of SEQ ID NO: 197; c) the HC2 comprises the amino acid sequence of SEQ ID NO: 198 and the LC1 comprises the amino acid sequence of SEQ ID NO: 199; or d) the HC2 comprises the amino acid sequence of SEQ ID NO: 202. In some embodiments, the HC2 comprises the amino acid sequence of SEQ ID NO: 198 and the LC1 comprises the amino acid sequence of SEQ ID NO: 199. In some embodiments, a) the HC1 comprises the amino acid sequence of SEQ ID NO: 193, the HC2 comprises the amino acid sequence of SEQ ID NO: 194, and the LC2 comprises the amino acid sequence of SEQ ID NO 195; b) the HC1 comprises the amino acid sequence of SEQ ID NO: 193, the HC2 comprises the amino acid sequence of SEQ ID NO: 196, and the LC2 comprises the amino acid sequence of SEQ ID NO: 197; c) the HC1 comprises the amino acid sequence of SEQ ID NO: 193, the HC2 comprises the amino acid sequence of SEQ ID NO: 198, and the LC2 comprises the amino acid sequence of SEQ ID NO: 199; or d) the HC1 comprises the amino acid sequence of SEQ ID NO: 200, the LC1 comprises the amino acid sequence of SEQ ID NO: 201, and the HC2 comprises the amino acid sequence of SEQ ID NO: 202. In some embodiments, the HC1 comprises the amino acid sequence of SEQ ID NO: 193, the HC2 comprises the amino acid sequence of SEQ ID NO: 198, and the LC2 comprises the amino acid sequence of SEQ ID NO: 199.

In some embodiments, the bispecific antibody or the bispecific antigen-binding fragment thereof, is encoded by an isolated polynucleotide, wherein the isolated polynucleotide comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: a) the VH of SEQ ID NO: 63; b) the VH of SEQ ID NO: 95; c) the VH of SEQ ID NO: 127; or d) the VH of SEQ ID NO: 191. In some embodiments, the bispecific antibody or the bispecific antigen-binding fragment thereof, is encoded by a polynucleotide, wherein the polynucleotide comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: the VH of SEQ ID NO: 95.

In some embodiments, the bispecific antibody or the bispecific antigen-binding fragment thereof, is encoded by a polynucleotide, wherein the polynucleotide comprises or further comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: a) the VL of SEQ ID NO: 64; b) the VL of SEQ ID NO: 96; c) the VL of SEQ ID NO: 128; or d) the VL of SEQ ID NO: 192. In some embodiments, the bispecific antibody or the bispecific antigen-binding fragment thereof, is encoded by a polynucleotide, wherein the polynucleotide comprises or further comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: the VL of SEQ ID NO: 96.

In some embodiments, the bispecific antibody or the bispecific antigen-binding fragment thereof, is encoded by an isolated polynucleotide, wherein the isolated polynucleotide comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: a) the VH of SEQ ID NO: 31; or b) the VH of SEQ ID NO: 159. In some embodiments, the bispecific antibody or the bispecific antigen-binding fragment thereof, is encoded by an isolated polynucleotide, wherein the isolated polynucleotide comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to the VH of SEQ ID NO: 159.

In some embodiments, the bispecific antibody or the bispecific antigen-binding fragment thereof, is encoded by an isolated polynucleotide, wherein the isolated polynucleotide comprises or further comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: a) the VL of SEQ ID NO: 32; or b) the VL of SEQ ID NO: 160. In some embodiments, the bispecific antibody or the bispecific antigen-binding fragment thereof, is encoded by an isolated polynucleotide, wherein the isolated polynucleotide comprises or further comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: the VL of SEQ ID NO: 160.

In some embodiments, the bispecific antibody or the bispecific antigen-binding fragment thereof, is encoded by an isolated polynucleotide, wherein the isolated polynucleotide comprises a sequence encoding a first antigen-binding site that specifically binds TRBV19, the sequence comprising a nucleotide sequence encoding: a) the amino acid sequence of SEQ ID NO: 31 and/or the amino acid sequence of SEQ ID NO: 32; or b) the amino acid sequence of SEQ ID NO: 159 and/or the amino acid sequence of SEQ ID NO: 160. In some embodiments, the bispecific antibody or the bispecific antigen-binding fragment thereof, is encoded by an isolated polynucleotide, wherein the isolated polynucleotide comprises a sequence encoding a first antigen-binding site that specifically binds TRBV19, the sequence comprising a nucleotide sequence encoding: the amino acid sequence of SEQ ID NO: 159 and/or the amino acid sequence of SEQ ID NO: 160.

In some embodiments, the bispecific antibody or the bispecific antigen-binding fragment thereof, is encoded by an isolated polynucleotide, wherein the isolated polynucleotide comprises a sequence encoding a second antigen-binding site that specifically binds EMR2, the sequence comprising a nucleotide sequence encoding: a) the amino acid sequence of SEQ ID NO: 63 and/or the amino acid sequence of SEQ ID NO: 64; b) the amino acid sequence of SEQ ID NO: 95 and/or the amino acid sequence of SEQ ID NO: 96; c) the amino acid sequence of SEQ ID NO: 127 and/or the amino acid sequence of SEQ ID NO: 128; or d) the amino acid sequence of SEQ ID NO: 191 and/or the amino acid sequence of SEQ ID NO: 192. In some embodiments, the bispecific antibody or the bispecific antigen-binding fragment thereof, is encoded by a polynucleotide, wherein the polynucleotide comprises a sequence encoding a second antigen-binding site that specifically binds EMR2, the sequence comprising a nucleotide sequence encoding the amino acid sequence of SEQ ID NO: 95 and/or the amino acid sequence of SEQ ID NO: 96.

In some embodiments, the bispecific antibody or the bispecific antigen-binding fragment thereof, is encoded by an isolated polynucleotide, wherein the isolated polynucleotide comprises a sequence encoding an HC1 comprising the amino acid sequence of SEQ ID NO: 193 or 200. In some embodiments, the bispecific antibody or the bispecific antigen-binding fragment thereof, is encoded by an isolated polynucleotide, wherein the isolated polynucleotide comprises a sequence encoding an HC1 comprising the amino acid sequence of SEQ ID NO: 193.

In some embodiments, the bispecific antibody or the bispecific antigen-binding fragment thereof, is encoded by a polynucleotide, wherein the polynucleotide comprises a sequence encoding a LC1 comprising the amino acid sequence of SEQ ID NO: 201.

In some embodiments, the bispecific antibody or the bispecific antigen-binding fragment thereof, is encoded by an isolated polynucleotide, wherein the isolated polynucleotide comprises a nucleotide sequence encoding: a) the amino acid sequence of SEQ ID NO: 193; or b) the amino acid sequence of SEQ ID NO: 200 and the amino acid sequence of SEQ ID NO: 201. In some embodiments, the bispecific antibody or the bispecific antigen-binding fragment thereof, is encoded by an isolated polynucleotide, wherein the isolated polynucleotide comprises a nucleotide sequence encoding the amino acid sequence of SEQ ID NO: 193.

In some embodiments, the bispecific antibody or the bispecific antigen-binding fragment thereof, is encoded by an isolated polynucleotide, wherein the isolated polynucleotide comprises a sequence encoding an HC2 comprising the nucleotide sequence of SEQ ID NO: 194, 196, 198, or 202. In some embodiments, the bispecific antibody or the bispecific antigen-binding fragment thereof, is encoded by an isolated polynucleotide, wherein the isolated polynucleotide comprises a sequence encoding an HC2 comprising the nucleotide sequence of SEQ ID NO: 198.

In some embodiments, the bispecific antibody or the bispecific antigen-binding fragment thereof, is encoded by an polynucleotide, wherein the isolated polynucleotide comprises a sequence encoding a LC2 comprising the nucleotide sequence of SEQ ID NO: 195, 197, or 199. In some embodiments, the bispecific antibody or the bispecific antigen-binding fragment thereof, is encoded by an polynucleotide, wherein the isolated polynucleotide comprises a sequence encoding a LC2 comprising the nucleotide sequence of SEQ ID NO: 199.

In some embodiments, the bispecific antibody or the bispecific antigen-binding fragment thereof, is encoded by an isolated polynucleotide, wherein the isolated polynucleotide comprises a sequence encoding: a) the amino acid sequence of SEQ ID NO: 194 and/or the amino acid sequence of SEQ ID NO: 195; b) the amino acid sequence of SEQ ID NO: 196 and/or the amino acid sequence of SEQ ID NO: 197; c) the amino acid sequence of SEQ ID NO: 198 and/or the amino acid sequence of SEQ ID NO: 199; or d) the amino acid sequence of SEQ ID NO: 202. In some embodiments, the bispecific antibody or the bispecific antigen-binding fragment thereof, is encoded by an isolated polynucleotide, wherein the isolated polynucleotide comprises a sequence encoding: the amino acid sequence of SEQ ID NO: 198 and/or the amino acid sequence of SEQ ID NO: 199.

In some embodiments, the bispecific antibody or the bispecific antigen-binding fragment thereof, is encoded by an isolated polynucleotide, wherein the isolated polynucleotide comprises a sequence encoding: a) the amino acid sequence of SEQ ID NO: 193, the amino acid sequence of SEQ ID NO: 194 and the amino acid of SEQ ID NO: 195; b) the amino acid sequence of SEQ ID NO: 193, the amino acid sequence of SEQ ID NO: 196 and the amino acid sequence of SEQ ID NO: 197; c) the amino acid sequence of SEQ ID NO: 193, the amino acid sequence of SEQ ID NO: 198 and the amino acid sequence of SEQ ID NO: 199; or d) the amino acid sequence of SEQ ID NO: 200, the amino acid sequence of SEQ ID NO: 201, and the amino acid sequence of SEQ ID NO: 202. In some embodiments, the bispecific antibody or the bispecific antigen-binding fragment thereof, is encoded by an isolated polynucleotide, wherein the isolated polynucleotide comprises a sequence encoding: the amino acid sequence of SEQ ID NO: 193, the amino acid sequence of SEQ ID NO: 198 and the amino acid sequence of SEQ ID NO: 199

The TRBV19-binding arm may be derived from any species by recombinant means. For example, the TRBV19 antigen-binding region may be derived from mouse, rat, goat, horse, swine, bovine, chicken, rabbit, camelid, donkey, human, or chimeric versions thereof. For use in administration to humans, non-human derived antigen-binding fragments may be genetically or structurally altered to be less antigenic upon administration to a human patient. In some embodiments, the TRBV19-binding arm comprises antigen-binding fragments which is chimeric.

In some embodiments, the TRBV19-binding arm comprises humanized antigen-binding fragments. Humanized antigen-binding fragments may be derived from chimeric immunoglobulins, immunoglobulin chains or fragments thereof (such as Fv, scFv, spFv, Fab, Fab′, F(ab′)2 or other antigen-binding subsequences of antibodies) that contain minimal sequence derived from non-human immunoglobulin. For the most part, humanized antibodies or antigen-binding fragments are human immunoglobulins (recipient antibody) or antigen-binding fragments in which residues from a complementary-determining region (CDR) of the recipient are replaced by residues from a CDR of a non-human species (donor antibody) such as mouse, rat or rabbit having the desired specificity, affinity, and capacity. In general, the humanized antibody antigen-binding fragments will comprise substantially all of at least one, and typically two, variable domains, in which all or substantially all of the CDR regions correspond to those of a non-human immunoglobulin and all or substantially all of the framework regions are those of a human immunoglobulin sequence. The humanized antibody antigen-binding fragments may include at least a portion of an immunoglobulin constant region (Fc), typically that of a human immunoglobulin.

In some embodiments, the multispecific antibodies described herein may adopt any format which has been described in the art for multispecific antibodies. In some embodiments, the multispecific antibody comprises a bispecific antibody which is a diabody, a cross-body, or a bispecific antibody obtained via a controlled Fab arm exchange as those described in the present disclosure.

In some embodiments, the multispecific antibodies include IgG-like molecules with complementary CH3 domains to force heterodimerization; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or spFv or stabilized diabodies are fused to heavy-chain constant-domains, Fc-regions or parts thereof; Fab fusion molecules, wherein different Fab-fragments are fused together; scFv-, spFv-, and diabody-based and heavy chain antibodies (e.g., domain antibodies, nanobodies) wherein different single chain Fv molecules or spFv molecules or different diabodies or different heavy-chain antibodies (e.g., domain antibodies, nanobodies) are fused to each other or to another protein or carrier molecule.

In some embodiments, IgG-like molecules with complementary CH3 domains molecules include the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (SEEDbody) (EMD Serono), the Biclonic (Merus), the DuoBody (Genmab A/S), and other asymmetric mutations (e.g., Zymeworks).

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (InnClone/Eli Lilly), Ts2Ab (Medlmmune/AZ) and BsAb (Zymogenetics), HERCULES (Biogen Idec) and TvAb (Roche).

In some embodiments, Fc fusion molecules include ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv).sub.2-Fab (National Research Center for Antibody Medicine—China).

In some embodiments, Fab fusion bispecific antibodies include F(ab)2 (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based and domain antibodies include but are not limited to Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

Full length multispecific antibodies of the present disclosure may be generated for example using Fab arm exchange (or half molecule exchange) between two mono specific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent mono specific antibodies are reduced. The resulting free cysteines of one of the parent monospecific antibodies form an inter heavy-chain disulfide bond with cysteine residues of a second parent mono specific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope, e.g., an epitope on EMR2 and an epitope on TRBV19.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Int. Publ. No. WO 2006/028936) may be used to generate full length multispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

In some embodiments of the multispecific antibody or the multispecific binding fragment described herein, the Fc domain of the first heavy chain (HC1) comprise mutations T366S, L368A and Y407V and the Fc domain of the second heavy chain (HC2) comprises mutation T366W. In some embodiments, the Fc domain of the second heavy chain (HC2) comprise mutations T366S, L368A and Y407V and the Fc domain of the first heavy chain (HC1) comprises mutation T366W.

Other strategies such as promoting heavy chain heterodimerization using electrostatic interactions by substituting positively charged residues at one CH3 surface and negatively charged residues at a second CH3 surface may be used, as described in, e.g., US Pat. Publ. No. US2010/0015133; US Pat. Publ. No. US2009/0182127; US Pat. Publ. No. US2010/028637 or US Pat. Publ. No. US2011/0123532. In other strategies, heterodimerization may be promoted by the following substitutions (expressed as modified position in the first CH3 domain of the first heavy chain/modified position in the second CH3 domain of the second heavy chain): L351Y_F405AY407V/T394W, T3661_K392M_T394W/F405A_Y407V, T366L_K392M_T394W/F405A_Y407V, L351Y_Y407A/T366A_K409F, L351Y_Y407A/T366V K409F Y407A/T366A_K409F, or T350V_L351Y_F405A Y407V/T350V_T366L_K392L_T394W as described in, e.g, U.S. Pat. Publ. No. US2012/0149876 or U.S. Pat. Publ. No. US2013/0195849 (Zymeworks).

In addition to methods described above, multispecific antibodies of the disclosure may be generated in vitro in a cell-free environment by introducing asymmetrical mutations in the CH3 regions of two mono specific homodimeric antibodies and forming the multispecific heterodimeric antibody from two parent monospecific homodimeric antibodies in reducing conditions to allow disulfide bond isomerization according to methods described in, e.g., Int. Pat. Publ. No. WO2011/131746. In the methods, the first monospecific bivalent antibody (e.g., anti-EMR2 antibody) and the second monospecific bivalent antibody (e.g., anti-TRBV19 antibody) are engineered to have certain substitutions at the CH3 domain that promotes heterodimer stability; the antibodies are incubated together under reducing conditions sufficient to allow the cysteines in the hinge region to undergo disulfide bond isomerization; thereby generating the multispecific antibody by Fab arm exchange. The incubation conditions may optimally be restored to non-reducing conditions. Exemplary reducing agents that may be used are 2-mercaptoethylamine (2-MEA), dithiothreitol (DTT), dithioerythritol (DTE), glutathione, tris (2-carboxyethyl) phosphine (TCEP), L-cysteine and beta-mercaptoethanol, preferably a reducing agent selected from the group consisting of: 2-mercaptoethylamine, dithiothreitol and tris (2-carboxyethyl) phosphine. For example, incubation for at least 90 min at a temperature of at least 20° C. in the presence of at least 25 mM 2-MEA or in the presence of at least 0.5 mM dithiothreitol at a pH from 5-8, for example at pH of 7.0 or at pH of 7.4 may be used.

In some embodiments, the multispecific antibodies or antigen-binding fragments are IgG, or derivatives thereof. The IgG class is divided in four isotypes: IgG1, IgG2, IgG3 and IgG4 in humans. They share more than 95% homology in the amino acid sequences of the Fc regions but show major differences in the amino acid composition and structure of the hinge region. The Fc region mediates effector functions, such as antibody-dependent cellular cytotoxicity (ADCC) and complement-dependent cytotoxicity (CDC). In ADCC, the Fc region of an antibody binds to Fc receptors (FcγRs) on the surface of immune effector cells such as natural killers and macrophages, leading to the phagocytosis or lysis of the targeted cells. In CDC, the antibodies kill the targeted cells by triggering the complement cascade at the cell surface. The antibodies described herein include antibodies with the described features of the variable domains in combination with any of the IgG isotypes, including modified versions in which the Fc sequence has been modified to effect different effector functions.

For many applications of therapeutic antibodies, Fc-mediated effector functions are not part of the mechanism of action. These Fc-mediated effector functions can be detrimental and potentially pose a safety risk by causing off-mechanism toxicity. Modifying effector functions can be achieved by engineering the Fc regions to reduce their binding to FcγRs or the complement factors. The binding of IgG to the activating (FcγRI, FcγRIIa, FcγRIIIa and FcγRIIIb) and inhibitory (FcγRIIb) FcγRs or the first component of complement (C1q) depends on residues located in the hinge region and the CH2 domain. Mutations have been introduced in IgG1, IgG2 and IgG4 to reduce or silence Fc functionalities. The antibodies described herein may include these modifications.

In one embodiment, the antibody comprises an Fc region with one or more of the following properties: (a) reduced effector function when compared to the parent Fc; (b) reduced affinity to FcγRI, FcγRIIa, FcγRIIb, FcγRIIIb and/or FcγRIIIa, (c) reduced affinity to FcγRI (d) reduced affinity to FcγRIIa (e) reduced affinity to FcγRIIb, (f) reduced affinity to FcγRIIIb or (g) reduced affinity to FcγRIIIa.

In some embodiments, the antibodies or antigen-binding fragments are IgG, or derivatives thereof, e.g., IgG1, IgG2, IgG3, and IgG4 isotypes. In some embodiments wherein the antibody has an IgG1 isotype, the antibody contains L234A, L235A, D265S and/or K409R substitution(s) in its Fc region. In some embodiments wherein the antibody has an IgG4 isotype, the antibody contains S228P, L234A, and L235A substitutions in its Fc region. The antibodies described herein may include these modifications.

In some embodiments, the Fc domains of HC1 and/or HC2 of a multispecific antibody described herein each comprise one or more mutations selected from L234A, L235A, and D265S. In some embodiments, the Fc domains of HC1 and HC2 each comprise mutations L234A, L235A, and D265S.

In some embodiments, the Fc domains of HC1 or HC2 of a multispecific antibody described herein further comprises one or more mutations which reduce Fc binding to protein A. In some embodiments, the Fc domains of HC1 or HC2 comprises mutations H435R and/or Y436F. In some embodiments, the Fc domain of HC1 comprises mutations H435R and/or Y436F. In some embodiments, the Fc domain of HC2 comprises mutations H435R and/or Y436F.

In some embodiments, the HC1 comprises, from the N- to C-terminus, a heavy chain variable domain (VH) associated with the first antigen-binding site, a CH1 domain, the Fc domain, a linker, and the third antigen-binding site.

In some embodiments, the HC2 comprises, from the N- to C-terminus, the second antigen-binding site, the Fc domain, a linker, and the third antigen-binding site.

In various embodiments, the scFv or spFv used in multispecific antibodies described herein comprises, from the N- to C-terminus, a VH, a linker and a VL in the format VH-L-VL or a VL, a linker and a VH in the format VL-L-VH. In some embodiments, the scFv or spFV comprises, from the N- to C-terminus, a VL, a linker and a VH in the format VL-L-VH. In some embodiments, the scFv or spFv comprises, from the N- to C-terminus, a VH, a linker and a VH in the format VL-L-VH.

Linkers used in the present disclosure may be about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. In some embodiments, the linker is 6 amino acids long. In some embodiments, the linker is 7 amino acids long. In some embodiments, the linker is 8 amino acids long. In some embodiments, the linker is 9 amino acids long. In some embodiments, the linker is 10 amino acids long. In some embodiments, the linker is 11 amino acids long. In some embodiments, the linker is 12 amino acids long. In some embodiments, the linker is 13 amino acids long. In some embodiments, the linker is 14 amino acids long. In some embodiments, the linker is 15 amino acids long. In some embodiments, the linker is 16 amino acids long. In some embodiments, the linker is 17 amino acids long. In some embodiments, the linker is 18 amino acids long. In some embodiments, the linker is 19 amino acids long. In some embodiments, the linker is 20 amino acids long. In some embodiments, the linker is 21 amino acids long. In some embodiments, the linker is 22 amino acids long. In some embodiments, the linker is 23 amino acids long. In some embodiments, the linker is 24 amino acids long. In some embodiments, the linker is 25 amino acids long. In some embodiments, the linker is 26 amino acids long. In some embodiments, the linker is 27 amino acids long. In some embodiments, the linker is 28 amino acids long. In some embodiments, the linker is 29 amino acids long. In some embodiments, the linker is 30 amino acids long. In some embodiments, the linker is 31 amino acids long. In some embodiments, the linker is 32 amino acids long. In some embodiments, the linker is 33 amino acids long. In some embodiments, the linker is 34 amino acids long. In some embodiments, the linker is 35 amino acids long. In some embodiments, the linker is 36 amino acids long. In some embodiments, the linker is 37 amino acids long. In some embodiments, the linker is 38 amino acids long. In some embodiments, the linker is 39 amino acids long. In some embodiments, the linker is 40 amino acids long. Exemplary linkers that may be used are Gly rich linkers, Gly and Ser containing linkers, Gly and Ala containing linkers, Ala and Ser containing linkers, and other flexible linkers.

In some embodiments, the linker comprises SEQ ID NO: 221.

In some embodiments, a bispecific antibody, or a bispecific antigen-binding fragment of the present disclosure comprises a HC1/LC1 and/or LC2/HC2 of any one of the antibodies described in Table 3.

Table 3 provides a summary of examples of some EMR2 x TRBV19 bispecific antibodies described herein:

TABLE 3
Exemplary EMR2 × TRBV19 bispecific antibodies

| | | HC1 | LC1 | | HC2 | LC2 |
|---|---|---|---|---|---|---|
| | | Amino acid | Amino acid | | Amino acid | Amino acid |
| | HC1/LC1 | sequence | sequence | HC2/LC2 | sequence | sequence |
| Name | (Vb17 arm) | SEQ ID NO | SEQ ID NO | (EMR2 arm) | SEQ ID NO | SEQ ID NO |
| EMVBB6 | B17B852- | 193 | — | EMR2B361 | 194 | 195 |
| | G34R-LH- | | | | | |
| | spFv | | | | | |
| EMVBB7 | B17B852- | 193 | — | EMR2B410 | 196 | 197 |
| | G34R-LH- | | | | | |
| | spFv | | | | | |
| EMVBB8 | B17B852- | 193 | — | EMR2B454 | 198 | 199 |
| | G34R-LH- | | | | | |
| | spFv | | | | | |
| EMVBB27 | B17B21- | 200 | 201 | EMR2B57- | 202 | — |
| | LC-N33S | | | LH-spFv | | |

In some embodiments, the antibody or the antigen-binding fragment thereof, comprises a heavy chain comprising the amino acid sequence of SEQ ID NO: 194, 196, 198, or 202. In some embodiments, the antibody or the antigen-binding fragment thereof, comprises a heavy chain comprising the amino acid sequence of SEQ ID NO: 198.

In some embodiments, the antibody or the antigen-binding fragment thereof, comprises a light chain comprises the amino acid sequence of SEQ ID NO: 195, 197 or 199. In some embodiments, the antibody or the antigen-binding fragment thereof, comprises a light chain comprises the amino acid sequence of SEQ ID NO: 199.

In some embodiments, the antibody or the antigen-binding fragment thereof, comprises: a) the HC1 comprising the amino acid sequence of SEQ ID NO: 194 and the LC1 comprising the amino acid sequence of SEQ ID NO: 195; b) the HC1 comprising the amino acid sequence of SEQ ID NO: 196 and the LC1 comprising the amino acid sequence of SEQ ID NO: 197; or c) the HC1 comprising the amino acid sequence of SEQ ID NO: 198 and the LC1 comprising the amino acid sequence of SEQ ID NO: 199. In some embodiments, the antibody or the antigen-binding fragment thereof, comprises: the HC1 comprising the amino acid sequence of SEQ ID NO: 198 and the LC1 comprising the amino acid sequence of SEQ ID NO: 199.

In some embodiments, the present disclosure provides an isolated polynucleotide comprising a sequence encoding the amino acid sequence of SEQ ID NO: 194, 196, 198, or 202.

In some embodiments, the present disclosure provides an isolated polynucleotide comprising a sequence encoding the amino acid sequence of SEQ ID NO: 198.

In some embodiments, the present disclosure provides an isolated polynucleotide comprising a sequence encoding the amino acid sequence of SEQ ID NO: 195, 197 or 199.

In some embodiments, the present disclosure provides an isolated polynucleotide comprising a sequence encoding the amino acid sequence of SEQ ID NO: 199.

In some embodiments, the present disclosure provides an isolated polynucleotide comprising a sequence encoding a) the amino acid sequence of SEQ ID NO: 194 and/or the amino acid sequence of SEQ ID NO: 195; b) the amino acid sequence of SEQ ID NO: 196 and/or the amino acid sequence of SEQ ID NO: 197; or c) the amino acid sequence of SEQ ID NO: 198 and/or the amino acid sequence of SEQ ID NO: 199. In some embodiments, the present disclosure provides an isolated polynucleotide comprising a sequence encoding the amino acid sequence of SEQ ID NO: 198 and/or the amino acid sequence of SEQ ID NO: 199.

In addition to the described multispecific antibodies or antigen-binding fragments, also provided are polynucleotide sequences capable of encoding the described multispecific antibodies or antigen-binding fragments. Vectors comprising the described polynucleotides are also provided, as are cells expressing the multispecific antibodies or antigen-binding fragments provided herein. Also described are cells capable of expressing the disclosed vectors. These cells may be mammalian cells (such as 293F cells, CHO cells), insect cells (such as Sf7 cells), yeast cells, plant cells, or bacteria cells (such as E. coli). The described antibodies may also be produced by hybridoma cells. The described antibodies may also be recombinantly produced.

Polynucleotides encoding recombinant antigen-binding proteins also are within the scope of the disclosure. In some embodiments, the polynucleotides described (and the peptides they encode) include a leader sequence. Any leader sequence known in the art may be employed. The leader sequence may include, but is not limited to, a restriction site or a translation start site.

The multispecific antibodies or antigen-binding fragments described herein include variants having single or multiple amino acid substitutions, deletions, or additions that retain the biological properties (e.g., binding affinity or immune effector activity) of the described multispecific antibodies or antigen-binding fragments. In the context of the present disclosure the following notations are, unless otherwise indicated, used to describe a mutation; i) substitution of an amino acid in a given position is written as e.g. K409R which means a substitution of a Lysine in position 409 with an Arginine; and ii) for specific variants the specific three or one letter codes are used, including the codes Xaa and X to indicate any amino acid residue. Thus, the substitution of Arginine for Lysine in position 409 is designated as: K409R, or the substitution of any amino acid residue for Lysine in position 409 is designated as K409X. In case of deletion of Lysine in position 409 it is indicated by K409*. The skilled person may produce variants having single or multiple amino acid substitutions, deletions, or additions.

These variants may include: (a) variants in which one or more amino acid residues are substituted with conservative or nonconservative amino acids, (b) variants in which one or more amino acids are added to or deleted from the polypeptide, (c) variants in which one or more amino acids include a substituent group, and (d) variants in which the polypeptide is fused with another peptide or polypeptide such as a fusion partner, a protein tag or other chemical moiety, that may confer useful properties to the polypeptide, such as, for example, an epitope for an antibody, a polyhistidine sequence, a biotin moiety and the like. Antibodies or antigen-binding fragments described herein may include variants in which amino acid residues from one species are substituted for the corresponding residue in another species, either at the conserved or nonconserved positions. In other embodiments, amino acid residues at nonconserved positions are substituted with conservative or nonconservative residues. The techniques for obtaining these variants, including genetic (deletions, mutations, etc.), chemical, and enzymatic techniques, are known to persons having ordinary skill in the art.

The multispecific antibodies or antigen-binding fragments described herein may embody several antibody isotypes, such as IgM, IgD, IgG, IgA and IgE. In some embodiments, the antibody isotype is IgG. In some embodiments the antibody isotype is IgG1, IgG2, IgG3, or IgG4 isotype, preferably IgG1 or IgG4 isotype. In some embodiments, the antibody isotype is IgG1 or IgG4. Antibody or antigen-binding fragment thereof, specificity is largely determined by the amino acid sequence, and arrangement, of the CDRs. Therefore, the CDRs of one isotype may be transferred to another isotype without altering antigen specificity. Alternatively, techniques have been established to cause hybridomas to switch from producing one antibody isotype to another (isotype switching) without altering antigen specificity. Accordingly, such antibody isotypes are within the scope of the described antibodies or antigen-binding fragments.

Also provided are vectors comprising the polynucleotides described herein. The vectors can be expression vectors. Recombinant expression vectors containing a sequence encoding a polypeptide of interest are thus contemplated as within the scope of this disclosure. The expression vector may contain one or more additional sequences such as but not limited to regulatory sequences (e.g., promoter, enhancer), a selection marker, and a polyadenylation signal. Vectors for transforming a wide variety of host cells are well known and include, but are not limited to, plasmids, phagemids, cosmids, baculoviruses, bacmids, bacterial artificial chromosomes (BACs), yeast artificial chromosomes (YACs), as well as other bacterial, yeast and viral vectors. Viral vectors can include an adenoviral vector, an adeno-associated viral vector, a retroviral vector, a lentiviral vector, a herpes simplex virus vector, and a poxvirus vector.

Recombinant expression vectors within the scope of the description include synthetic, genomic, or cDNA-derived nucleic acid fragments that encode at least one recombinant protein which may be operably linked to suitable regulatory elements. Such regulatory elements may include a transcriptional promoter, sequences encoding suitable mRNA ribosomal binding sites, and sequences that control the termination of transcription and translation. Expression vectors, especially mammalian expression vectors, may also include one or more nontranscribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host may also be incorporated.

The transcriptional and translational control sequences in expression vectors to be used in transforming vertebrate cells may be provided by viral sources. Exemplary vectors may be constructed as described by Okayama and Berg, 3 Mol. Cell. Biol. 280 (1983).

In some embodiments, the multispecific antibody- or antigen-binding fragment-coding sequence is placed under control of a powerful constitutive promoter, such as the promoters for the following genes: hypoxanthine phosphoribosyl transferase (HPRT), adenosine deaminase, pyruvate kinase, beta-actin, human myosin, human hemoglobin, human muscle creatine, and others. In addition, many viral promoters function constitutively in eukaryotic cells and are suitable for use with the described embodiments. Such viral promoters include without limitation, Cytomegalovirus (CMV) immediate early promoter, the early and late promoters of SV40, the Mouse Mammary Tumor Virus (MMTV) promoter, the long terminal repeats (LTRs) of Maloney leukemia virus, Human Immunodeficiency Virus (HIV), Epstein Barr Virus (EBV), Rous Sarcoma Virus (RSV), and other retroviruses, and the thymidine kinase promoter of Herpes Simplex Virus. In one embodiment, the multispecific antibody or the antigen-binding fragment thereof, coding sequence is placed under control of an inducible promoter such as the metallothionein promoter, tetracycline-inducible promoter, doxycycline-inducible promoter, promoters that contain one or more interferon-stimulated response elements (ISRE) such as protein kinase R 2′,5′-oligoadenylate synthetases, Mx genes, ADAR1, and the like.

Vectors described herein may contain one or more Internal Ribosome Entry Site(s) (IRES). Inclusion of an IRES sequence into fusion vectors may be beneficial for enhancing expression of some proteins. In some embodiments the vector system will include one or more polyadenylation sites (e.g., SV40), which may be upstream or downstream of any of the aforementioned nucleic acid sequences. Vector components may be contiguously linked, or arranged in a manner that provides optimal spacing for expressing the gene products (i.e., by the introduction of “spacer” nucleotides between the ORFs), or positioned in another way. Regulatory elements, such as the IRES motif, may also be arranged to provide optimal spacing for expression.

The vectors may comprise selection markers, which are well known in the art. Selection markers include positive and negative selection markers, for example, antibiotic resistance genes (e.g., neomycin resistance gene, a hygromycin resistance gene, a kanamycin resistance gene, a tetracycline resistance gene, a penicillin resistance gene, a puromycin resistance gene, a blasticidin resistance gene), glutamate synthase genes, HSV-TK, HSV-TK derivatives for ganciclovir selection, or bacterial purine nucleoside phosphorylase gene for 6-methylpurine selection (Gadi et al., 7 Gene Ther. 1738-1743 (2000)). A nucleic acid sequence encoding a selection marker or the cloning site may be upstream or downstream of a nucleic acid sequence encoding a polypeptide of interest or cloning site.

The vectors described herein may be used to transform various cells with the genes encoding the described antibodies or antigen-binding fragments. For example, the vectors may be used to generate multispecific antibody or an antigen-binding fragment-producing cells. Thus, another aspect features host cells transformed with vectors comprising a nucleic acid sequence encoding an antibody or an antigen-binding fragment thereof, that specifically binds EMR2 and/or TRBV19, such as the antibodies or antigen-binding fragments described and exemplified herein.

Numerous techniques are known in the art for the introduction of foreign genes into cells and may be used to construct the recombinant cells for purposes of carrying out the described methods, in accordance with the various embodiments described and exemplified herein. The technique used should provide for the stable transfer of the heterologous gene sequence to the host cell, such that the heterologous gene sequence is heritable and expressible by the cell progeny, and so that the necessary development and physiological functions of the recipient cells are not disrupted. Techniques which may be used include but are not limited to chromosome transfer (e.g., cell fusion, chromosome mediated gene transfer, micro cell mediated gene transfer), physical methods (e.g., transfection, spheroplast fusion, microinjection, electroporation, liposome carrier), viral vector transfer (e.g., recombinant DNA viruses, recombinant RNA viruses) and the like (described in Cline, 29 Pharmac. Ther. 69-92 (1985)). Calcium phosphate precipitation and polyethylene glycol (PEG)-induced fusion of bacterial protoplasts with mammalian cells may also be used to transform cells.

Cells suitable for use in the expression of the multispecific antibodies or antigen-binding fragments described herein are preferably eukaryotic cells, more preferably cells of plant, rodent, or human origin, for example but not limited to NSO, CHO, CHOK1, perC.6, Tk-ts13, BHK, HEK293 cells, COS-7, T98G, CV-1/EBNA, L cells, C127, 3T3, HeLa, NS1, Sp2/0 myeloma cells, and BHK cell lines, among others. In addition, expression of antibodies may be accomplished using hybridoma cells. Methods for producing hybridomas are well established in the art.

Cells transformed with expression vectors described herein may be selected or screened for recombinant expression of the antibodies or antigen-binding fragments described herein. Recombinant-positive cells are expanded and screened for subclones exhibiting a desired phenotype, such as high level expression, enhanced growth properties, or the ability to yield proteins with desired biochemical characteristics, for example, due to protein modification or altered post-translational modifications. These phenotypes may be due to inherent properties of a given subclone or to mutation. Mutations may be effected through the use of chemicals, UV-wavelength light, radiation, viruses, insertional mutagens, inhibition of DNA mismatch repair, or a combination of such methods.

### Exemplary Monospecific Antibodies

In some embodiments, the disclosure provides an antibody or an antigen-binding fragment thereof, that specifically binds the GAIN domain and/or GPS motif of epidermal-growth-factor-like module-containing mucin-like hormone receptor 2 (EMR2).

In some embodiments, the GAIN domain comprises amino acid residues D261-Q478 of human EMR2. In some embodiments, the GAIN domain comprises the amino acid sequence SEQ ID NO: 215.

In some embodiments, the GPS motif comprises the amino acid sequence SEQ ID NO: 216.

In some embodiments, the antibody or the antigen-binding fragment thereof, binds to an epitope comprising SEQ ID NO: 217 or SEQ ID NO: 218.

In some embodiments, the antibody or the antigen-binding fragment thereof, binds to human EMR2 with a dissociation constant (K_{D}) between about 0.01 nM to about 5 nM. In some embodiments, the antibody or the antigen-binding fragment thereof, binds to human EMR2 with an EC₅₀ between about 0.1 nM to about 15 nM.

In some embodiments, the antibody or the antigen-binding fragment thereof, is or comprises a, a Fab fragment, a F(ab′)2 fragment, F(ab)′3 fragments, a single-chain variable fragment (scFv), a bis-scFv, a (scFv)2, a stapled scFv (spFv), a diabody, a minibody, a nanobody, a triabody, a tetrabody, a disulfide stabilized Fv protein (dsFv), a single-domain antibody (sdAb), an Ig NAR, a single heavy chain antibody, a camelid antibody, a shark antibody, or a chemically modified derivative thereof.

In some embodiments, the antibody or the antigen-binding fragment thereof, comprises a Fab. In some embodiments, the antibody or the antigen-binding fragment thereof, further comprises a CH1 domain.

In some embodiments, the antibody or the antigen-binding fragment thereof, does not comprise a CH1 domain. In some embodiments, the antibody or the antigen-binding fragment thereof, comprises a scFv or a spFv. In some embodiments, the scFv or spFv comprises a signal sequence, a heavy chain variable sequence, a GS-Linker, and a light chain variable sequence.

In some embodiments, the antibody or the antigen-binding fragment thereof, further comprises an Fc domain. In some embodiments, the Fc domain of the antibody or the antigen-binding fragment thereof, is an IgA, an IgG, an IgE, or an IgM. In some embodiments, the Fc domain of the antibody or the antigen-binding fragment thereof, is an IgG. In some embodiments, the IgG is IgG1 or IgG4.

In some embodiments, the Fc domain comprises one or more different mutations which promote heterodimerization. In some embodiments, the Fc domain comprises mutations T366S, L368A and Y407V (EU numbering) or mutation T366W (EU numbering).

In some embodiments, the Fc domains of HC1 and/or HC2 further comprise one or more mutations which reduce Fc binding to a Fcγ receptor. In some embodiments, the Fcγ receptor is FcγRI, FcγRIIA, FcγRIIB, FcγRIIIA, and/or FcγRIIIB. In some embodiments, the Fc domain comprises one or more mutations selected from L234A, L235A, and D265S (EU numbering). In some embodiments, the Fc domain comprises mutations L234A, L235A, and D265S (EU numbering).

In some embodiments, the Fc domain further comprises one or more mutations which reduce Fc binding to protein A. In some embodiments, the Fc domain comprises mutations H435R and/or Y436F (EU numbering). In some embodiments, the Fc domain comprises mutations H435R and Y436F (EU numbering).

In some embodiments, the antibody or the antigen-binding fragment thereof, comprises a humanized antibody or an antigen binding fragment thereof, a human antibody or an antigen binding fragment thereof, a murine antibody or an antigen binding fragment thereof, a chimeric antibody or an antigen binding fragment thereof, a monospecific antibody or a monospecific antigen binding fragment thereof, a bispecific antibody or a bispecific antigen binding fragment thereof, a multispecific antibody or a multispecific antigen binding fragment thereof.

In any of the foregoing embodiments, the antibody or the antigen-binding fragment thereof, comprises the amino acid sequences of the EMR2-binding arm as described herein. In any of the foregoing embodiments, the antibody or the antigen-binding fragment thereof, is encoded by a polynucleotide comprising a nucleotide sequence that encodes the amino acid sequences of the EMR2-binding arm as described herein.

Therapeutic composition and methods of treatment using multispecific antibodies and multispecific antigen-binding fragments thereof and/or monospecific antibodies and monospecific antigen-binding fragments thereof

The multispecific antibodies discussed above, for example the EMR2 x TRBV19 bispecific antibodies and the EMR2 monospecific antibodies discussed above, are useful in therapy. In particular, the multispecific antibodies are useful in treating cancer. Also provided herein are therapeutic compositions for the treatment of a hyperproliferative disorder in a mammal which comprises a therapeutically effective amount of a multispecific antibody or a multispecific antigen-binding fragment described herein or a monospecific antibody or a monospecific antigen-binding fragment as described herein and a pharmaceutically acceptable carrier. In some embodiments, the bispecific antibody is a EMR2 x TRBV19 bispecific antibody as described herein, or a EMR2 x TRBV19-bispecific antigen-binding fragment thereof. In some embodiments, the monospecific antibody is a EMR2 monospecific antibody as described herein, or a EMR2-monospecific antigen binding fragment thereof. In one embodiment, said pharmaceutical composition is for the treatment of a EMR2-expressing cancer, including (but not limited to) hematological cancers such as, e.g., myeloid malignancies. In one embodiment said pharmaceutical composition is for the treatment of a EMR2-expressing cancer, including (but not limited to) the following: AML, CML, or myelodysplastic neoplasms (MDS).

The pharmaceutical compositions provided herein comprise: a) an effective amount of a multispecific antibody or the antibody fragment of the present disclosure, and b) a pharmaceutically acceptable excipient and/or carrier, which may be inert or physiologically active. In some embodiments, the bispecific antibody is a EMR2 x TRBV19 bispecific antibody as described herein, or a EMR2 x TRBV19-bispecific antigen-binding fragment thereof. In some embodiments, the monospecific antibody is a EMR2 monospecific antibody as described herein, or a EMR2-monospecific antigen binding fragment thereof.

Also provided herein are methods for inhibiting cytotoxicity of a cancer cell or redirecting immune or T cells against cancer cells expressing EMR2. Any of the multispecific antibodies or antibody fragments of the disclosure may be used therapeutically. For example, in one embodiment the EMR2 x TRBV19-multispecific antibody or the EMR2 monospecific antibody may be used therapeutically to treat cancer in a subject. The method for inhibiting cytotoxicity of a cancer cell or redirecting immune or T cells against cancer cells can be practiced in vitro, in vivo, or ex vivo.

In an exemplary embodiment, multispecific antibodies or antibody fragments of the disclosure are used for the treatment of a hyperproliferative disorder in a mammal. In a further exemplary embodiment, one of the pharmaceutical compositions disclosed above, and which contains a multispecific antibody or an antibody fragment of the disclosure, is used for the treatment of a hyperproliferative disorder in a mammal. In one embodiment, the disorder is a cancer, e.g., an EMR2-expressing cancer. In particular, the EMR2-expressing cancer is a hematological cancer, e.g., myeloid malignancies. In one embodiment said pharmaceutical composition is for the treatment of a EMR2-expressing cancer, including (but not limited to) the following: AML, CML, or MDS. In exemplary embodiments, the multispecific antibody is a EMR2 x TRBV19-multispecific antibody as described herein, or a multispecific antigen-binding fragment thereof, and more preferably a EMR2 x TRBV19-bispecific antibody as described herein, or a EMR2 x TRBV19-bispecific antigen-binding fragment thereof. In some embodiments, the monospecific antibody is a EMR2 monospecific antibody as described herein, or a EMR2-monospecific antigen binding fragment thereof.

Accordingly, the pharmaceutical compositions of the disclosure are useful in the treatment or prevention of a variety of cancers, e.g., EMR2-expressing cancers. In particular, the EMR2-expressing cancer is a hematological cancer, e.g., myeloid malignancies. In one embodiment said pharmaceutical composition is for the treatment of a EMR2-expressing cancer, including (but not limited to) the following: AML, CML, or MDS.

Similarly, further provided herein is a method for redirecting a T cell to EMR2-expressing cancer cells in a subject in need thereof, said method comprising administering to said subject a therapeutically effective amount of the bispecific antibody or the bispecific antigen-binding fragment, with an effective amount of a multispecific antibody or the antibody fragment of the present disclosure, either alone or in combination with other cytotoxic or therapeutic agents. In exemplary embodiments, the multispecific antibody is a EMR2 x TRBV19-multispecific antibody as described herein, or a multispecific antigen-binding fragment thereof, and more preferably a EMR2 x TRBV19-bispecific antibody as described herein, or a EMR2 x TRBV19-bispecific antigen-binding fragment thereof. In some embodiments, the monospecific antibody is a EMR2 monospecific antibody as described herein, or a EMR2-monospecific antigen binding fragment thereof.

In some embodiments, the multispecific antibody or the multispecific antigen-binding fragment, polynucleotide, vector, pharmaceutical composition, or host cell is administered to the subject. In some embodiments, the administration of the multispecific antibody or the multispecific antigen-binding fragment, polynucleotide, vector, pharmaceutical composition, or host cell is ex vivo.

Examples of in vitro uses include treatments of autologous bone marrow prior to their transplant into the same patient in order to kill diseased or malignant cells; and prevent graft-versus-host-disease (GVHD); treatments of cell cultures in order to kill all cells except for desired variants that do not express the target antigen; or to kill variants that express undesired antigen. The conditions of non-clinical in vitro use are readily determined by one of ordinary skill in the art.

Examples of clinical ex vivo use are to remove tumor cells from bone marrow prior to autologous transplantation in cancer treatment. Treatment can be carried out as follows. Bone marrow is harvested from the patient or other individual and then incubated in medium containing serum to which is added the cytotoxic agent of the disclosure. Concentrations range from about 10 μM to 1 μM, for about 30 min to about 48 hours at about 37° C. The exact conditions of concentration and time of incubation, i.e., the dose, are readily determined by one of ordinary skill in the art. After incubation, the bone marrow cells are washed with medium containing serum and returned to the patient by i.v. infusion according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

For clinical in vivo use, a therapeutically effective amount of the multispecific antibody or the antigen-binding fragment is administered to a subject in need thereof. For example, the EMR2 x TRBV19-multispecific antibodies and multispecific antigen-binding fragments thereof or the EMR2-specific antibodies and antigen-binding fragments thereof may be useful in the treatment of a cancer in a subject in need thereof. In some embodiments, the cancer is AML, CML, or MDS. In exemplary embodiments, the multispecific antibody is a EMR2 x TRBV19-multispecific antibody as described herein, or a multispecific antigen-binding fragment thereof, and more preferably a EMR2 x TRBV19-bispecific antibody as described herein, or a EMR2 x TRBV19-bispecific antigen-binding fragment thereof. In some embodiments, the monospecific antibody is a EMR2-monospecific antibody as described herein, or a EMR2-monospecific antigen binding fragment thereof. In some embodiments, the subject is a mammal, preferably a human. In some embodiments, the multispecific antibody or the antigen-binding fragment will be administered as a solution that has been tested for sterility.

In some embodiments, the methods describe herein further comprise administering a second therapeutic agent. In some embodiments, the second therapeutic agent is a surgery, chemotherapy, androgen deprivation therapy, radiation, or any combination thereof.

In one embodiment, a method for treating a disorder involving cells expressing EMR2 and/or TRBV19 in a subject, which method comprises administration of a therapeutically effective amount of a multispecific antibody or the fragment, such as a EMR2 x TRBV19 multispecific antibody described herein, and radiotherapy to a subject in need thereof is provided. In one embodiment is provided a method for treating or preventing cancer, which method comprises administration of a therapeutically effective amount of a multispecific antibody or the fragment, such as a EMR2 x TRBV19 antibody described herein, and radiotherapy to a subject in need thereof. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, actinium-225, and indium-111.

### EXAMPLES

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### Materials and Methods

### EMR2 Recombinant Proteins

Human EMR2 protein is composed of a large extracellular N-terminal domain and a Class B GPCR 7-transmembrane (7TM) C-terminal portion. Located in the ECD proximal to the cell membrane resides a GPS motif that contains a protease site that is cleaved to activate the formation of a non-covalent active receptor complex of the large N-terminal domain and the GPCR-7TM domain halves. For antibody discovery purposes, the present efforts were focused on the antibody accessible extracellular N-terminal domain specifically the GAIN domain and the GPS motif proximal to the cell membrane to avoid cross reactivity to CD97, another EGF-TM7 receptor family member. CD97/AGRE5 (Uniprot P48960), is 97% similar in sequence to EMR2 in the first 260 amino acids where the 5 EGF-like tandem sequences reside in the N-terminal ECD. The EMR2 GAIN domain portion, however is only 49.7% identical to the CD97 GAIN domain which would facilitate generating antibodies specific for EMR2 and not to CD97.

Two human EMR2 GAIN domain constructs were designed with and without the GPS motif. The first construct, EMR2W4, contains the GAIN domain plus GPS motif (EMR2 D261-T540, UniprotKB Q9UHX3 numbering), was expressed with an Avitag at the C-terminus followed by a 6×His tag (SEQ ID NO: 226). The second construct EMR2W6 was designed without the GPS (EMR2 D261-Q478) and tagged with the Avitag and 6×His (SEQ ID NO: 226) at the C-terminus.

The EMR2 constructs were transiently expressed in HEK293-6E or Expi293 cells and purified by utilizing the C-terminal 6×His tag (SEQ ID NO: 226) followed by size exclusion chromatography. The purified proteins were used as immunization antigens to generate antibodies against the EMR2 GAIN domain. These antibodies were used to derive the EMR2 binding arm of the bispecific antibodies of the present disclosure.

### EMR2 Cell Lines

Full length human EMR2 with its native signal sequence was overexpressed in NS0 cells.

An N-terminal ECD truncated EMR2, human EMR2 GAIN+GPCR-7TM, (D261-N823, EMR2W13) with a non-native signal sequence was also overexpressed in NS0 cells.

### Vb17 Proteins

The first TCR Va10.2-Vb17 knob in hole Fc protein (B17W38) was designed based on the crystal structure PDB 2VLR and used to test binding of the Vb17 arm of the bispecific antibodies of the present disclosure.

Because the B17W38 Vb17 chain demonstrated ragged processing of the signal sequence, a second construct B17W40 was produced. Changes were introduced to obtain correct processing of the signal sequence and to facilitate simpler mass spectrometry analysis of the purified protein. These changes included using a different signal sequence, removal of the N-terminal valine and a Ser to Cys mutation was made. Also in the Va10.2 chain, an extra threonine was introduced at the N-terminus.

### EMR2 and Related Protein Sequences

huEMR2 GAIN domain + GPS, D261-T540 UniprotKB Q9UHX3
with C-terminal Avi/6x His tag (“6x His tag”
disclosed as SEQ ID NO: 226), EMR2W4
(SEQ ID NO: 203)
MARKSALLALALLLLGFPGAWGDMTFSTWTPPPGVHSQTLSRFFDKVQDLGRD
YKPGLANNTIQSILQALDELLEAPGDLETLPRLQQHCVASHLLDGLEDVLRGLSKNLSNG
LLNFSYPAGTELSLEVQKQVDRSVTLRQNQAVMQLDWNQAQKSGDPGPSVVGLVSIPG
MGKLLAEAPLVLEPEKQMLLHETHQGLLQDGSPILLSDVISAFLSNNDTQNLSSPVTFTF
SHRSVIPRQKVLCVFWEHGQNGCGHWATTGCSTIGTRDTSTICRCTHLSSFAVLMAHYD
VQEEDPVLTVITGGGGSGLNDIFEAQKIEWHEGGGGSHHHHHHG
huEMR2 GAIN domain, D261-Q478 UniprotKB Q9UHX3 with C-terminal
Avi/6x His tag (“6x His tag” disclosed as SEQ ID NO: 226),
EMR2W6
(SEQ ID NO: 204)
MARKSALLALALLLLGFPGAWGDMTFSTWTPPPGVHSQTLSRFFDKVQDLGRD
YKPGLANNTIQSILQALDELLEAPGDLETLPRLQQHCVASHLLDGLEDVLRGLSKNLSNG
LLNFSYPAGTELSLEVQKQVDRSVTLRQNQAVMQLDWNQAQKSGDPGPSVVGLVSIPG
MGKLLAEAPLVLEPEKQMLLHETHQGLLQDGSPILLSDVISAFLSNNDTQNLSSPVTFTF
SHRSVIPRQGGGGSGLNDIFEAQKIEWHEGGGGSHHHHHHG
huEMR2 full length, M1-N823 UniprotKB Q9UHX3-1, EMR2W12
(SEQ ID NO: 205)
MGGRVFLVFLAFCVWLTLPGAETQDSRGCARWCPQDSSCVNATACRCNPGFSS
FSEIITTPMETCDDINECATLSKVSCGKFSDCWNTEGSYDCVCSPGYEPVSGAKTFKNES
ENTCQDVDECQQNPRLCKSYGTCVNTLGSYTCQCLPGFKLKPEDPKLCTDVNECTSGQ
NPCHSSTHCLNNVGSYQCRCRPGWQPIPGSPNGPNNTVCEDVDECSSGQHQCDSSTVCF
NTVGSYSCRCRPGWKPRHGIPNNQKDTVCEDMTFSTWTPPPGVHSQTLSRFFDKVQDL
GRDYKPGLANNTIQSILQALDELLEAPGDLETLPRLQQHCVASHLLDGLEDVLRGLSKN
LSNGLLNFSYPAGTELSLEVQKQVDRSVTLRQNQAVMQLDWNQAQKSGDPGPSVVGL
VSIPGMGKLLAEAPLVLEPEKQMLLHETHQGLLQDGSPILLSDVISAFLSNNDTQNLSSP
VTFTFSHRSVIPRQKVLCVFWEHGQNGCGHWATTGCSTIGTRDTSTICRCTHLSSFAVL
MAHYDVQEEDPVLTVITYMGLSVSLLCLLLAALTFLLCKAIQNTSTSLHLQLSLCLFLAH
LLFLVAIDQTGHKVLCSIIAGTLHYLYLATLTWMLLEALYLFLTARNLTVVNYSSINRFM
KKLMFPVGYGVPAVTVAISAASRPHLYGTPSRCWLQPEKGFIWGFLGPVCAIFSVNLVL
FLVTLWILKNRLSSLNSEVSTLRNTRMLAFKATAQLFILGCTWCLGILQVGPAARVMAY
LFTIINSLQGVFIFLVYCLLSQQVREQYGKWSKGIRKLKTESEMHTLSSSAKADTSKPSTV
N
huEMR2 GAIN + GPCR 7TM, D261-N823 UniprotKB Q9UHX3, EMR2W13
(SEQ ID NO: 206)
MARKSALLALALLLLGFPGAWGDMTESTWTPPPGVHSQTLSRFFDKVQDLGRD
YKPGLANNTIQSILQALDELLEAPGDLETLPRLQQHCVASHLLDGLEDVLRGLSKNLSNG
LLNFSYPAGTELSLEVQKQVDRSVTLRQNQAVMQLDWNQAQKSGDPGPSVVGLVSIPG
MGKLLAEAPLVLEPEKQMLLHETHQGLLQDGSPILLSDVISAFLSNNDTQNLSSPVTFTF
SHRSVIPRQKVLCVFWEHGQNGCGHWATTGCSTIGTRDTSTICRCTHLSSFAVLMAHYD
VQEEDPVLTVITYMGLSVSLLCLLLAALTFLLCKAIQNTSTSLHLQLSLCLFLAHLLFLVA
IDQTGHKVLCSIIAGTLHYLYLATLTWMLLEALYLFLTARNLTVVNYSSINRFMKKLMF
PVGYGVPAVTVAISAASRPHLYGTPSRCWLQPEKGFIWGFLGPVCAIFSVNLVLFLVTL
WILKNRLSSLNSEVSTLRNTRMLAFKATAQLFILGCTWCLGILQVGPAARVMAYLFTIIN
SLQGVFIFLVYCLLSQQVREQYGKWSKGIRKLKTESEMHTLSSSAKADTSKPSTVN
TCR Va10.2 x Vb17 -KiHFc (knob-in-hole Fc), B17W38
Va10.2 -knob-in-hole Fc chain 1
(SEQ ID NO: 211)
MAWVWTLLFLMAAAQSIQAQLLEQSPQFLSIQEGENLTVYCNSSSVFSSLQWYR
QEPGEGPVLLVTVVTGGEVKKLKRLTFQFGDARKDSSLHITAAQPGDTGLYLCAGAGS
QGNLIFGKGTKLSVKPNIQNPDPAVYQLRDSKSSDKSVCLFTDFDSQTNVSQSKDSDVYI
TDKTVLDMRSMDFKSNSAVAWSNKSDFACANAFNNSIIPEDTFFPSEPKSCDKTHTCPPC
PAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQ
VYVYPPSREEMTKNQVSLTCLVKGFYPSDIAVEWESNGQPENNYKTTPPVLDSDGSFAL
VSKLTVDKSRWQQGNVFSCSVMHEALHNHYTQKSLSLSPG
Vb17 -knob-in-hole Fc chain 2
(SEQ ID NO: 212)
MAWVWTLLFLMAAAQSIQAVDGGITQSPKYLFRKEGQNVTLSCEQNLNHDAM
YWYRQDPGQGLRLIYYSQIVNDFQKGDIAEGYSVSREKKESFPLTVTSAQKNPTAFYLC
ASSSRSSYEQYFGPGTRLTVTEDLKNVFPPEVAVFEPSEAEISHTQKATLVCLATGFYPD
HVELSWWVNGKEVHSGVSTDPQPLKEQPALNDSRYSLSSRLRVSATFWQNPRNHFRCQ
VQFYGLSENDEWTQDRAKPVTQIVSAEAWGRADEPKSCDKTHTCPPCPAPELLGGPSVF
LFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYVLPPSREE
MTKNQVSLLCLVKGFYPSDIAVEWESNGQPENNYLTWPPVLDSDGSFFLYSKLTVDKSR
WQQGNVFSCSVMHEALHNHYTQKSLSLSPG
TCR Va10.2 x Vb17 -KiHFc (knob-in-hole Fc), B17W40
Va10.2-knob-in-hole Fc Chain 1
(SEQ ID NO: 213)
MAWVWTLLFLMAAAQSIQATQLLEQSPQFLSIQEGENLTVYCNSSSVFSSLQWY
RQEPGEGPVLLVTVVTGGEVKKLKRLTFQFGDARKDSSLHITAAQPGDTGLYLCAGAGS
QGNLIFGKGTKLSVKPNIQNPDPAVYQLRDSKSSDKSVCLFTDFDSQTNVSQSKDSDVYI
TDKTVLDMRSMDFKSNSAVAWSNKSDFACANAFNNSIIPEDTFFPSEPKSCDKTHTCPPC
PAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQ
VYVYPPSREEMTKNQVSLTCLVKGFYPSDIAVEWESNGQPENNYKTTPPVLDSDGSFAL
VSKLTVDKSRWQQGNVFSCSVMHEALHNHYTQKSLSLSPG
Vb17-knob-in-hole-Fc Chain 2
(SEQ ID NO: 214)
MARKSALLALALLLLFGPAWADGGITQSPKYLFRKEGQNVTLSCEQNLNHDAM
YWYRQDPGQGLRLIYYSQIVNDFQKGDIAEGYSVSREKKESFPLTVTSAQKNPTAFYLC
ASSSRSSYEQYFGPGTRLTVTEDLKNVFPPEVAVFEPSEAEISHTQKATLVCLATGFYPD
HVELSWWVNGKEVHSGVSTDPQPLKEQPALNDSRYCLSSRLRVSATFWQNPRNHFRCQ
VQFYGLSENDEWTQDRAKPVTQIVSAEAWGRADEPKSCDKTHTCPPCPAPELLGGPSVF
LFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYVLPPSREE
MTKNQVSLLCLVKGFYPSDIAVEWESNGQPENNYLTWPPVLDSDGSFFLYSKLTVDKSR
WQQGNVFSCSVMHEALHNHYTQKSLSLSPG

Single underlined sequences are the signal sequences for the expressed proteins, protein tags are double underlined, linker sequences are wave underlined, and Avitag is dotted underlined.

### Immunization

Twenty-four (24) Ablexis mice were immunized with Human EMR2 alone or combined with Rhesus EMR2 using 4 different protocols (AB298, AB299, AB300, AB301). Prior to injection, Human and Rhesus EMR2 GAIN Avi 6×His (SEQ ID NO: 226) were produced at Syngene (batches EMR2W4.001 and EMR2W8.001). AB298, AB299 received CFA/Sigma adjuvant and AB300, AB301 received CL413 adjuvant. The mice received weekly RIMMS+IP injections for 5 weeks and final boost on Day 42. The final boost contained 10 ug Human EMR2 and 50 ug mouse CD40 monoclonal antibody. Sera was collected at Day 36 and assessed for circulating IgG specific antibodies to human EMR2. Titers were determined via cell based FACS with OCI-AML-3 and OCI-AML-5 cells.

### Hybridoma Fusion

On day 45 and day 46 of the immunization schedule, lymph nodes and spleen were harvested from the mice for B lymphocytes fusion. Lymph nodes and spleens were homogenized by group into single-cell suspensions and fused with FO multiple myeloma cell line for hybridoma generation. A total of four separate fusions were performed as follows: Fusion 1 (lymph nodes) and fusion 2 (spleen) from mouse #'s 1, 5, 6, 9, 11, 12, 15, 17, 18. Fusion 3 (lymph nodes) and fusion 4 (spleen) from mouse #'s 2, 14, 19, 20, 21, 22, 23. The hybridoma cells were then plated and cultured for 7-14 days.

scFv Conversion of EMR2 Hybridomas (EMR2-00030, EMR2-00031)

This is a summary of the process used to generate and identify the EMR2 binder EMR2B57 which was used on EMR2xVb17 EMVBB27. This binder was generated from ‘direct PCR’ HTP scFv screening based approach. cDNA from V-gene recovery was used to pair the VH/VL wells directly with the scFv linker and then the generated scFv pairs were tested in E. coli-based expression and screening platform.

Four 96-well plates were used for the EMR2 scFv conversion from V-region cloning group. In total, there were 358 unique scFv PCR wells and screened ˜2300 colonies. Identified 511 samples from the 2300 colonies as positive binders and sent these off to sequencing. Upon receiving sequencing results, SL performed alignments and clustering to identify 156 unique scFv consensus sequences. These sequences then moved into thermal stability screening to identify rank.

### Summary

Total sequences: 512
HC: 491
LC: 493
Assembled scFv 477
Clusters by CDR: 156

### Hybridoma Screening

Hybridoma supernatants were screened by cell MSD on OCI-AML-3 cells (AML cell line with high EMR2-expression). Primary hits were defined as samples giving assay signal greater than 9.33 times the negative control average. A total of 616 samples meeting this criterion were subsequently scaled up and re-screened by FACS and ELISA. FACs assay was done on primary EMR2-expressing OCI-AML-3, OCI-AML-5, SIG-M5 (very low expression) and negative CARNAVAL cells. Samples were also screened by ELISA for human or mouse light chains. A total of 348 hits met the desired binding profile (binding to OCI-AML-3 cells >5×S:B by flow). Selected hits from the combined confirmatory ELISA and FACS screen are expanded in 48-well plate format. From these hits, frozen cell stocks, cell lysates and culture supernatant were made. The selected hybridoma lysates were sent to the molecular biology team for variable region cloning. The recovered variable regions were cloned into a human IgG1 vector and recombinantly expressed. The human IgG1 antibodies were tested by flow cytometry for cell binding to OCI3 cells with negative counter screen against Carnaval cells. 92 clones meeting the criteria were sequenced, expressed, and tested for additional characterization.

A total of 134 mAbs were recovered and recombinantly expressed.

### EMR2-00061

128 out of 134 EMR2 tested samples showed S/B>5 on OciAml-3 cells at 50 nM or 10 nM concentration

### EMR2-00064

Biophysical testing: Affinity kinetics and Thermal stability was completed for 128 mAbs using hu EMR2 on Biacore 8K+

### EMR2 Affinity

51% of IgG mAbs and 98% VHHs bound Hu-EMR2.

### Thermostability

In both mAb panels, roughly 30% of binders have TM1 >69° C.
30% (19 of 65) of EMR2 binders from affinity screen have a TM1 >69° C.
8% of the anti-EMR2 VHH binders were >55° C.
8% (5 of 61) of binders from affinity screen have a TM1 >55° C.

The Fab arm of molecule EMVBB8 was derived from Hybridoma clone GDB #EMR2HB26SC1147_174C09 (CL002044637).

The Fab arm of EMVBB7 was derived from hybridoma clone GDB #EMR2HB25SC1147_026G09 (CL002031617).

The Fab arm of EMVBB6 was derived from hybridoma clone GDB #EMR2SB1SC1147_207P22 (CL002297775).

The scFv arm of EMVBB27 was generated from a direct PCR high-throughput scFv screening based approach. cDNA from V-gene recovery was used to pair the VH/VL wells directly with the scFv linker and then the generated scFv pairs were tested in an E. coli-based expression and screening platform.

### T-Cell Engagement Cytotoxicity Assay Using EMR2xTRBV19 Leads

EMR2-positive AML cell line OCI-AML3, were incubated with EMR2xTRBV19 Abs with either pan human T-cells for 72 h at 37° C. at the effector to target (E:T) ratio of 10:1 or with healthy human peripheral blood mononuclear cells (PBMCs) with E:T ration of 5:1 for 96 h at 37° C. Cell cytotoxicity was measured at by flow cytometry.

### Example 1. Target Validation

Targeted immunotherapy in AML remains a challenge due to the heterogenous nature of the AML cancer cells (blasts) and the lack of AML-specific antigens. While several immunotherapies targeting CD33, CD123, or CLL1, including chimeric antigen receptor (CAR)-T and CAR-NK cell therapies, are being evaluated clinically, challenges have been observed with CD3 TCE therapies due to lack of efficacy and tolerability as well as an unacceptable safety profile. EMR2xTRBV19 TCEs represent a treatment that may address unmet medical needs of patients with AML whose disease no longer responds to SoC. Bispecific TRBV19 antibodies could provide potent cytotoxicity to cancer cells by engaging a small population of cytotoxic T cells while avoiding pan-T-cell activation that is known to cause CRS.

TRBV19+ T cell

During T-cell development, T-cell receptor (TCR) diversity results from variable diversity joining (VDJ)-domain recombination, leading to unique TCR Vα and Vβ features in specific T-cell clones. The TCRβ locus comprises a cluster of 52 functional Vβ gene segments, a single D gene segment, thirteen J gene segments, and 2 TRBC genes. There are 24 families of Vβ gene segments in humans. Consequently, each TCR Vβ family is used by only 0.58% to 10.84% of the TCR repertoire (van der Geest K S, Abdulahad W H, Horst G, et al. Quantifying distribution of flow cytometric TCR-VP usage with economic statistics. PLoS One. 2015; 10(4):e0125373). Extensive repertoire analysis has shown preferential skewing of distinct TCR Vβ genes for selective expansion of T cells in certain diseases such as Vβ6 and Vβ15 in rheumatoid arthritis (Jenkins R N, Nikaein A, Zimmermann A, Meek K, Lipsky P E. T cell receptor V beta gene bias in rheumatoid arthritis. J Clin Invest. 1993; 92(6):2688-2701), Vβ8 in IgA nephropathy (Muro K, Yamagata K, Kobayashi M, Hirayama K, Koyama A. Usage of T cell receptor variable segments of the beta-chain in IgA nephropathy. Nephron. 2002; 92(1):56-63), and Vβ5, Vβ8, Vβ15, Vβ16, and Vβ18 in sarcoidosis (Forman J D, Klein J T, Silver R F, Liu M C, Greenlee B M, Moller D R. Selective activation and accumulation of oligoclonal V beta-specific T cells in active pulmonary sarcoidosis. J Clin Invest. 1994; 94(4):1533-1542).

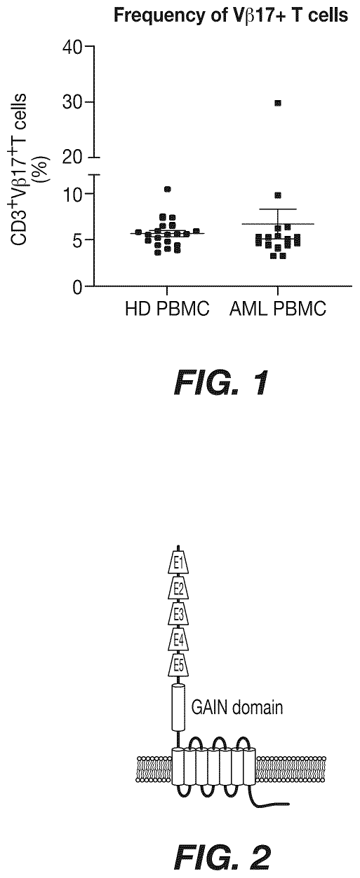

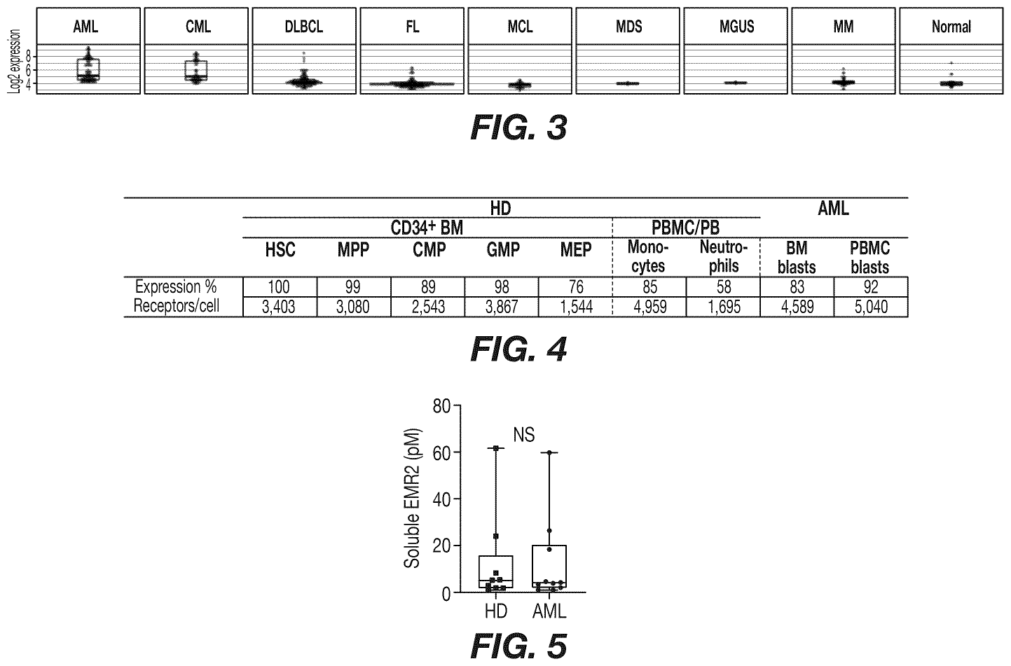

Similarly, after influenza A infection, M158-66 peptide is presented in the context of major histocompatibility complex (MHC)-allele human leukocyte antigen (HLA)-A*0201 and the resulting cytotoxic T-lymphocytes (CTLs) predominantly express TRBV19 (Lawson T M, Man S, Williams S, Boon A C, Zambon M, Borysiewicz L K. Influenza A antigen exposure selects dominant Vbeta17+ TCR in human CD8+ cytotoxic T cell responses. Int Immunol. 2001; 13(11):1373-1381). Expansion of CD8⁺TRBV19⁺ correlates with M158-66-specific lysis while TRBV19 depletion from PBMCs abrogates CTL response to the influenza infection (Lehner P J, Wang E C, Moss P A, et al. Human HLA-A0201-restricted cytotoxic T lymphocyte recognition of influenza A is dominated by T cells bearing the V beta 17 gene segment. J Exp Med. 1995; 181(1):79-91). Upon clearance of infection, a highly focused memory T-cell repertoire—wherein TRBV19 cells prevail—provides efficient recall response in future exposures. Since influenza A is a common viral infection of humans and most adults have serological evidence of previous influenza A exposure, the frequency of TRBV19⁺ T cells was relatively stable in the peripheral blood of healthy donors and AML patients with a mean value of 5.7% and 6.7%, respectively (FIG. 1).

The strong cytotoxic potential and retained memory phenotype of TRBV19⁺ T cells make them an attractive candidate for immune-cell-engaging approaches. Leveraging a small and relatively stable population of T cells with strong cytotoxic potential would potentially reduce the risk of CRS while inducing potent antitumor efficacy over time.

### EMR2 as Tumor-Associated Antigen

The adhesion G-protein-coupled receptors (aGPCRs) constitute an evolutionarily membrane protein family with emerging roles in many important biological processes. They are uniquely characterized by the chimeric composition of a large extracellular domain (ECD) and a 7-pass transmembrane (7TM) region. The aGPCRs are further classified into subfamilies based on the nature of their N-terminal domains (i.e., lectin-like, Ig-like, epidermal growth factor [EGF]-like, or cadherin-like motifs). One subfamily, the adhesion G-protein-coupled receptor E (ADGRE) family (i.e., EMR1, 2, 3, 4, and CD97) is characterized by the presence of several tandem EGF-like domains in their N-terminus (McKnight A J, Gordon S. EGF-TM7: a novel subfamily of seven-transmembrane-region leukocyte cell-surface molecules. Immunol Today. 1996; 17(6):283-287). Within this family, the EGF domains of EMR2 and CD97 demonstrate the most homology, differing by only 6 amino acids (Lin H H, Stacey M, Hamann J, Gordon S, McKnight A J. Human EMR2, a novel EGF-TM7 molecule on chromosome 19p13.1, is closely related to CD97. Genomics. 2000; 67(2):188-200), whereas EMR2 and EMR3 are the only members that lack mouse orthologs.

A defining feature of EMR2, similar to aGPCRs, is the presence of a GAIN domain, which is capable of self-catalytic cleavage, resulting in the generation of an extracellular N-terminal fragment and a 7TM C-terminal fragment that is involved in the cellular adhesion and signaling functions (FIG. 2) (Huang Y S, Chiang N Y, Chang G W, Lin H H. Membrane-association of EMR2/ADGRE2-NTF is regulated by site-specific N-glycosylation. Sci Rep. 2018; 8(1):4532).

EMR2 expression is restricted to myeloid cells including mature monocytes, macrophages, and BDCA-3⁺ myeloid dendritic cells, whereas minimal expression is found on granulocytes (Kwakkenbos M J, Chang G W, Lin H H, et al. The human EGF-TM7 family member EMR2 is a heterodimeric receptor expressed on myeloid cells. J Leukoc Biol. 2002; 71(5):854-862). Expression is highly regulated during monocyte/macrophage differentiation (Chang G W, Davies J Q, Stacey M, et al. CD312, the human adhesion-GPCR EMR2, is differentially expressed during differentiation, maturation, and activation of myeloid cells. Biochem Biophys Res Commun. 2007; 353(1):133-138; Boyden S E, Desai A, Cruse G, et al. Vibratory urticaria associated with a missense variant in ADGRE2. N Engl J Med. 2016; 374(7):656-663). Unlike CD97, no expression of EMR2 is reported on resting or activated lymphocytes.

Extensive studies of EMR2 are hampered by the lack of mouse orthologues. Functionally, EMR2 has also been implicated in autoimmune disease and neutrophil function. EMR2⁺ macrophages and dendritic cells are increased in the synovium of patients with rheumatoid arthritis (Kop E N, Kwakkenbos M J, Teske G J, et al. Identification of the epidermal growth factor-TM7 receptor EMR2 and its ligand dermatan sulfate in rheumatoid synovial tissue. Arthritis Rheum. 2005; 52(2):442-450). Additionally, EMR2 is implicated in neutrophil function by mediating activation and cytokine secretion in the presence of lipopolysaccharide and IL-10 (Chang G W, Davies J Q, Stacey M, et al. CD312, the human adhesion-GPCR EMR2, is differentially expressed during differentiation, maturation, and activation of myeloid cells. Biochem Biophys Res Commun. 2007; 353(1):133-138). Circulating neutrophils in patients with systemic inflammation also exhibit elevated EMR2 expression (Chen T Y, Hwang T L, Lin C Y, et al. EMR2 receptor ligation modulates cytokine secretion profiles and cell survival of lipopolysaccharide-treated neutrophils. Chang Gung Med J. 2011; 34(5):468-477.). Additionally, foamy macrophages in atherosclerotic vessels and splenocytes in patients with Gaucher's disease express EMR2 (van Eijk M, Aust G, Brouwer M S, et al. Differential expression of the EGF-TM7 family members CD97 and EMR2 in lipid-laden macrophages in atherosclerosis, multiple sclerosis and Gaucher disease. Immunol Lett. 2010; 129(2):64-71).

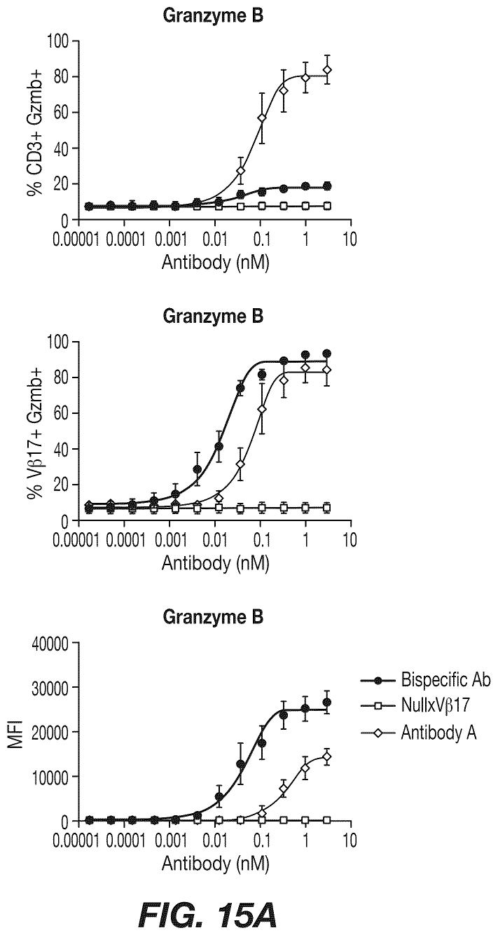

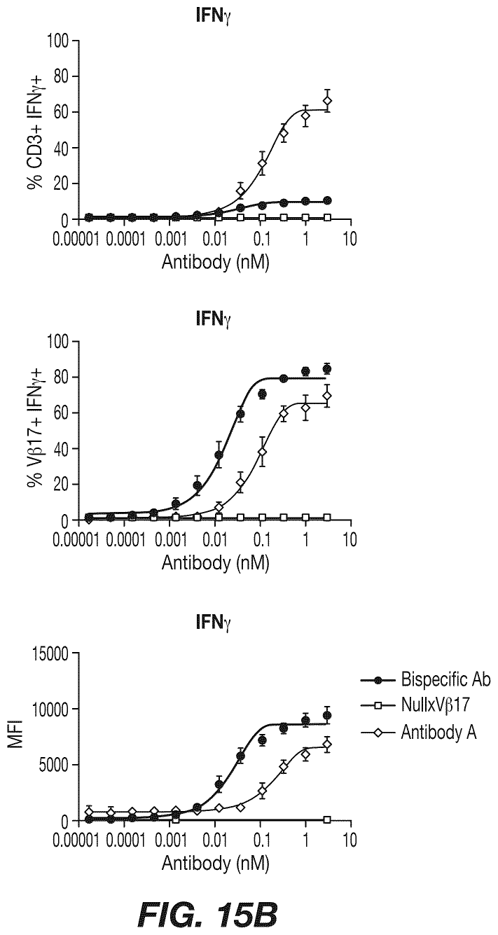

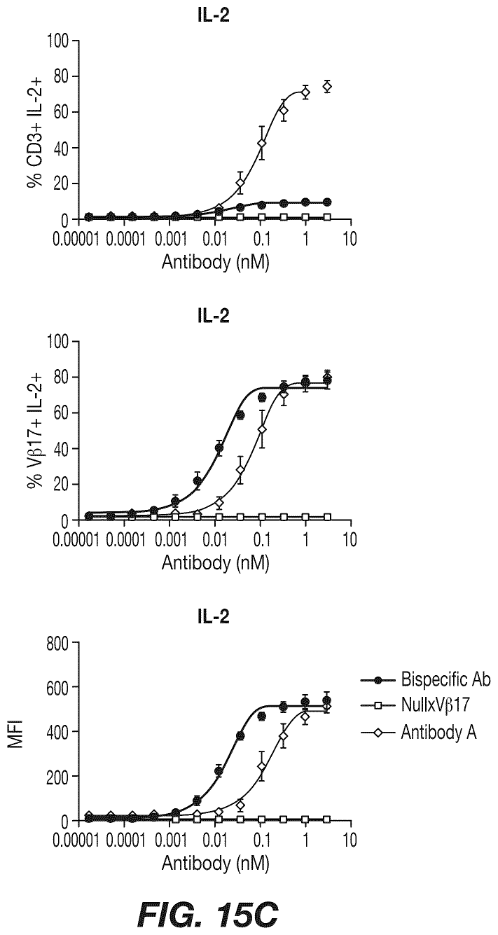

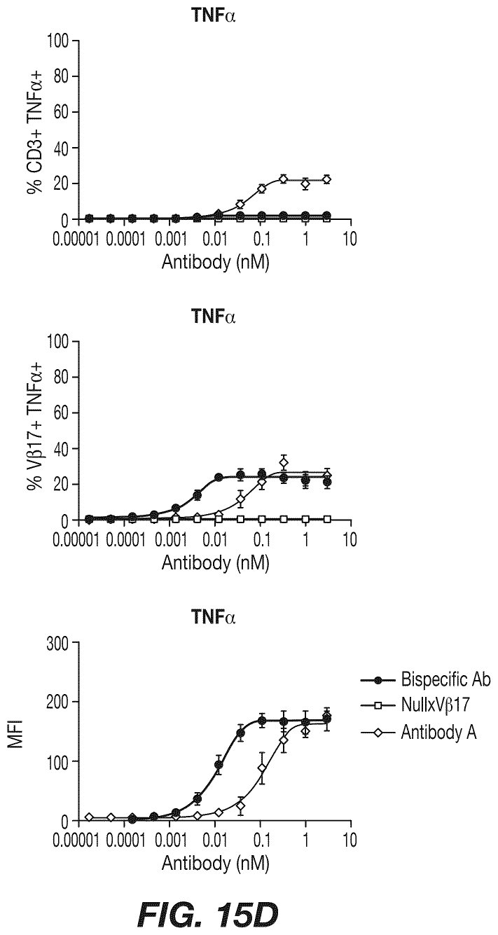

Several reports have identified EMR2 in human neoplasms (Aust G, Steinert M, Schlitz A, et al. CD97, but not its closely related EGF-TM7 family member EMR2, is expressed on gastric, pancreatic, and esophageal carcinomas. Am J Clin Pathol. 2002; 118(5):699-707). EMR2 mRNA expression in AML was reported to be high when compared to normal cells (FIG. 15).

In an extensive AML surfaceome dataset, EMR2 was found to be expressed at low levels in the gut, ovary, and spleen, and the fluorescence-activated cell sorting (FACS) analyses in that study detected EMR2 in ˜93% of cells in AML patient samples (Perna F, Berman S H, Soni R K, et al. Integrating proteomics and transcriptomics for systematic combinatorial chimeric antigen receptor therapy of AML. Cancer Cell. 2017; 32(4):506-519.e5). To confirm the protein expression of EMR2 and receptor density, FACS analysis was performed on AML cell lines (Table 4) and primary AML patient samples as well as the different healthy hematopoietic cells (FIG. 4). In addition to the myeloid populations, low/limited EMR2 expression was detected on lymphocytes, corresponding to mean values of 1.4% on B cells, 3.6% on NK cells, and 1% on T cells (CD4⁺ and CD8⁺ T cells). Furthermore, receptor densities of the positive populations of lymphocytes (B, T, and NK cells) were found in a range of 100 to 200 receptors, representing 3% of that on monocytes (4,959 receptors/cell).

TABLE 4
Calculated receptor counts for EMR2
on different myeloid cell lines.

| | EMR2 receptor | |
|---|---|---|
| Cell line | density | Cell line origin |
| ELF153 | 15,719 | Acute myeloid leukemia - M7 |
| OCI-AML3 | 14,753 | Acute myeloid leukemia - M4 |
| YHN-1 | 12,944 | Acute myeloid leukemia |
| MUTZ-3 | 11,482 | Acute myelomonocytic leukemia - M4 |
| FKH-1 | 11,411 | Acute myeloid leukemia - M4 |
| KG-1 | 10,314 | Acute myeloid leukemia |
| MOLM-13 | 9,530 | Acute myeloid leukemia - M5a |
| HNT-34 | 9,096 | Acute myeloid leukemia - M4 |
| OCI-AML5 | 8,644 | Acute myeloid leukemia - M4 |
| Kasumi 3 | 7,583 | Acute myeloblastic leukemia - M0 |
| OCI-M1 | 4,854 | Erythroleukemia - M6 |
| MOLM-14 | 4,764 | Acute myeloid leukemia - M5a |
| EOL1 | 4,026 | Acute myeloid (eosinophilic) leukemia |
| KO52 | 3,987 | Acute myeloid leukemia |
| SET-2 | 3,922 | Essential thrombocythemia |
| OCI-AML1 | 3,245 | Acute myelomonocytic leukemia -M4 |
| HEL92.1.7 | 3,115 | Erythroleukemia |
| OCI-AML2 | 2,584 | Acute myeloid leukemia - M4 |
| TF1 | 2,374 | Erythroleukemia - M6 |
| THP-1 | 2,152 | Acute monocytic leukemia |
| NOMO-1 | 2,053 | Acute myeloid leukemia - M5a |
| P815 | −271 | Mastocytoma |
| M-NSF-60 | −190 | Myelogenous leukemia |

EMR2, epidermal-growth-factor-like module-containing mucin-like hormone receptor-like 2; FAB, French- American-British.
Receptor density of EMR2 was measured in different malignant myeloid cell lines. FAB classification of leukemia subtypes is depicted when available.

Alternatively spliced transcripts (transmembrane and soluble) of EMR2 were detected in colorectal carcinoma cell lines due to partial or complete deletion of Exon 13 of the canonical transcript that leads to the soluble isoform (Lin H H, Stacey M, Yona S, Chang G W. GPS proteolytic cleavage of adhesion-GPCRs. Adv Exp Med Biol. 2010; 706:49-58). Physiological soluble EMR2 levels were assessed in sera of healthy donors (n=9) and AML patients (n=10) by enzyme-linked immunosorbent assay (ELISA) detecting the ECD domain. Soluble EMR2 concentrations were low and comparable between healthy donors and AML patients, ranging from 1.08 pM (based on reported ECD size of 65 kDa [9]) to 61.54 pM with a median of 5.00 pM in healthy donors and ranging from 1.08 pM to 59.97 pM with a median of 4.03 pM in AML patients (FIG. 5).

Overall, the high expression of EMR2 on AML blast and the distinctive features of TRBV19⁺ T cells together provide a novel opportunity for immunotherapy.

### Putative Target Liabilities

Due to expression on myeloid progenitors and downstream myeloid cells, AML-targeted therapies have been associated with hematological adverse events such as myelosuppression and cytopenias (Maakaron J E, Rogosheske J, Long M, Bachanova V, Mims A S. CD33-targeted therapies: beating the disease or beaten to death? J Clin Pharmacol. 2021; 61(1):7-17; Uckun F M, Lin T L, Mims A S, et al. A clinical phase 1B study of the CD3xCD123 bispecific antibody APVO436 in patients with relapsed/refractory acute myeloid leukemia or myelodysplastic syndrome. Cancers (Basel). 2021; 13(16):4113). However preclinical data presented below indicates a therapeutic window between targeting AML cells and healthy myeloid cells with the bispecific molecules of the present disclosure.

### Example 2. Therapeutic Profile

The bispecific molecules of the present example are an IgG1 bispecific antibody that simultaneously binds to the R subunit (TRBV19, herein called TRBV19) of TCR (Uniprot ID: A0A5B3) on T cells and to EMR2 on AML cells. The antibody features the AAS mutations in the constant region to abolish interaction with Fc receptors and heterodimerization is enhanced using the knobs-into-holes platform mutations (Ridgway J B, Presta L G, Carter P. ‘Knobs-into-holes’ engineering of antibody CH3 domains for heavy chain heterodimerization. Protein Eng. 1996; 9(7):617-621). The molecule comprises an anti-TRBV19 spFv fused onto the N-terminus of the ‘knob’ Fc region (i.e., T366W). The ‘hole’ chain (i.e., T366S, L368A, Y407V) features an anti-EMR2 Fab at the N-terminus of the Fc. The hole chain also contains the ‘RF’ mutations (i.e., H435R, Y436F) to disrupt protein A binding of monomeric and homodimerized hole chains (Tustian A D, Endicott C, Adams B, Mattila J, Bak H. Development of purification processes for fully human bispecific antibodies based upon modification of protein A binding avidity. MAbs. 2016; 8(4):828-838). The bispecific was developed to evaluate the therapeutic potential of tumor-targeting EMR2 and of TRBV19 for T-cell engagement.

### Intrinsic Design Properties of the Bispecific Antibodies

TABLE 5
Biophysical assessment and results.

| mAb TMP characteristic | Result | Comments |
|---|---|---|
| SPR protein binding (human) | EMR2 K_{D} = 193 ± 8 pM | Binding to EMR2W4 |
| SPR protein binding (human) | TRBV19 K_{D} = 94 ± 13 nM | Binding to B17W38 |
| Cyno cross-reactivity (SPR) | No binding up to 0.1 μM | Binding to EMR2W14 |
| Fc receptor binding | Not determined. | No binding expected due to AAS |
| | | Fc mutation. 20 different IgG1- |
| | | AAS molecules tested showed no |
| | | binding to human and cyno Fcγ |
| | | receptors. |
| FcRn binding (human, pH 6) | K_{D} = 306 ± 217 nM | Fast dissociation at pH 7.4. |
| FcRn binding (cyno, pH 6) | K_{D} = 454 ± 311 nM | Fast dissociation at pH 7.4. |
| Epitope for EMR2 (HDX- MS) | Epitope encompassing Residues | EMR2 construct used for epitope |
| | 120-128 | mapping was EMR2W4. |
| | (SEQ ID NO: 217), and 227- | |
| | 250 (SEQ ID NO: 218) | |
| Epitope for TRBV19 (HDX- | Residues 53-56 (IVND) (SEQ ID | TRBV19 construct used for |
| MS) | NO: 219) of beta chain formed | epitope mapping was B17W40. |
| | strongest epitope. Residues 50-52 | Epitope determined for |
| | (YSQ) formed the weak epitope. | B17B881, the parent anti- |
| | | TRBV19 antibody. |
| Intact mAb (MS), release | 127,380.1 Da | N/A |
| Conformational stability | Tₒₙ = 59.93° C. | N/A |
| (DSC) | Tₒₙ = 59.93° C. | N/A |
| | Tₘ1 = 70.17° C. | |
| | Tₘ2 = 76.08° C. | |
| % Purity (2-step | 97.6% monomer | N/A |
| purification) (AUC) | | |
| Serum stability over 7 days, | 94.4% monomer | N/A |
| 37° C. (SEC-FDS) | | |
| Non-specific binding (SPR) | None observed | N/A |
| Isoelectric point (cIEF) | pI = 8.83 | N/A |
| | % acidic = 25.8 | |
| | basic = 15.5 | |
| Relative hydrophobicity | Hydrophobic index = 0.54 | N/A |
| (aHIC) | | |
| Concentration achieved | 191.4 mg/mL | N/A |
| Percent recovery | 80.30% | N/A |
| Viscosity | 6.5 cP | N/A |

AAS, L234A, L235A, D265S; AUC, analytical ultracentrifugation; aHIC, analytical hydrophobic interaction chromatography; B17W38, Vα10.2-TRBV19-Fc fusion protein; cIEF, capillary isoelectric focusing; cyno, cynomolgus monkey; Cys, cysteine; EMR2, epidermal-growth-factor-like module-containing mucin-like hormone receptor-like 2; DSC, differential scanning calorimetry; EMR2W14, Cyno EMR2 GAIN domain protein; EMR2W4, Human EMR2 GAIN domain protein; Fc, fragment crystallizable; FcRn, neonatal Fc receptor; FDS, fluorescence detection system; gly, glycosylation; HDX-MS, hydrogen-deuterium exchange mass spectrometry; hu, human; Ig, immunoglobulin; KD, equilibrium dissociation constant; mAb, monoclonal antibody; MS, mass spectrometry; N/A, not assessed; pI, isoelectric point; ref., reference; SEC, size-exclusion chromatography; SPR, surface plasmon resonance; Tm, melting temperature; TMP, target medicinal product; Ton, temperature of onset of melting.

TABLE 6
Chemical and PTM stability in forced degradation conditions.

| Assay | Release | Physiological | pH 8.5 | pH 5.0 | Chem Ox | Thermal |
|---|---|---|---|---|---|---|
| Intact mass (Da) | 127,380.1 | 127,381.6 | 127,382.3 | 127,381.7 | 127,476 | 127,379.8 |
| % Monomer (aSEC) | 99.4 | 98.7 | 97.4 | 98.4 | 97.9 | 98.4 |
| % Purity | 100/99.5 | 98.6/99.3 | 94.4/96.9 | 98.8/99.4 | 94.3/99.6 | 99/99.4 |
| (GXII NR/R) | | | | | | |
| Clipping | None | None | None | None | Trace | None |
| (intact mass) | | | | | clipping | |
| Conformational | Tₒₙ = 63.61° C. | Tₒₙ = 63.56° C. | Tₒₙ = 63.03° C. | Tₒₙ = 62.70° C. | Tₒₙ = 50.13° C. | Tₒₙ = 61.15° C. |
| stability (DSF) | Tₘ1 = 73.81° C. | Tₘ1 = 72.63° C. | Tₘ1 = 73.45° C. | Tₘ1 = 73.85° C. | Tₘ1 = 60.72° C. | Tₘ1 = 73.77° C. |
| | Tₘ2 = N/A | Tₘ2 = N/A | Tₘ2 = N/A | Tₘ2 = N/A | Tₘ2 = 74.73° C. | Tₘ2 = N/A |
| | Tₘ3 = N/A | Tₘ3 = N/A | Tₘ3 = N/A | Tₘ3 = N/A | Tₘ3 = N/A | Tₘ3 = N/A |
| Binding (EMR2 | 1.9 × 10⁻¹⁰ / 100 | 2.1 × 10⁻¹⁰ / 100 | 2.5 × 10⁻¹⁰ / 97 | 2.0 × 10⁻¹⁰ / 98 | 2.0 × 10⁻¹⁰ / 100 | 2.2 × 10⁻¹⁰ / 98 |
| SPR K_{D} [M] / % | | | | | | |
| active) | | | | | | |
| Binding (TRBV19 | 9.4 × 10⁻⁸ / 100 | 1.0 × 10⁻⁷ / 106 | 8.9 × 10⁻⁸ / 101 | 9.7 × 10⁻⁸ / 101 | 9.3 × 10⁻⁸ / 104 | 9.4 × 10⁻⁸ / 106 |
| SPR K_{D} [M] / % | | | | | | |
| active) | | | | | | |
| CDR oxidation | ≤1.0% | ≤1.0% | ≤1.0% | ≤1.0% | ≤1.0% | ≤1.0% |
| CDR | 1.9% N35 | 1.7% N33 | 1.5% N56 | 1.8% N35 | 2.2% N35 | 2.1% N35 |
| Deamidation | LCDR1 of LC2 | LCDR1 of LC2 | HCDR2 of HC2 | LCDR1 of LC2 | LCDR1 of LC2 | LCDR1 of LC2 |
| | | 3.0% N35 | 4.3% N33 | | | 1.4% N92 |
| | | LCDR1 of LC2 | LCDR1 of LC2 | | | LCDR3 of LC2 |
| | | | 3.7% N35 | | | |
| | | | LCDR1 of LC2 | | | |
| CDR | 2.1% D185 | 2.1% D185 | 2.1% D185 | 2.0% D185 | 1.9% D185 | 2.0% D185 in |
| Isomerization | HCDR2 of HC1 | HCDR2 of HC1 | HCDR2 of HC1 | HCDR2 of HC1 | HCDR2 of HC1 | HCDR2 of HC1 |
| | 1.2% D99 | 1.5% D99 | 1.4% D99 | 2.0% D99 | 1.3% D99 | 2.1% D99 |
| | HCDR3 of HC2 | HCDR3 of HC2 | HCDR3 of HC2 | HCDR3 of HC2 | HCDR3 of HC2 | HCDR3 of HC2 |

aSEC, analytical size-exclusion chromatography; CDR, complementarity determining region; Chem Ox, chemical oxidation; DSF, differential scanning fluorimetry; EMR2, epidermal-growth-factor-like module-containing mucin-like hormone receptor-like 2; GXII, capillary electrophoresis; HC, heavy chain; HCDR, heavy-chain complementarity determining region; KD, equilibrium dissociation constant; LC, light chain; LCDR, light-chain complementarity determining region; N/A, not assessed; NR/R, non-reduced/reduced; PTM, post-translational modification; SPR, surface plasmon resonance; Tm, melting temperature; Ton, temperature of onset of melting.

### EMR2 Target Arm and TRBV19 Arm 37° C. Binding Characterization of the Bispecific

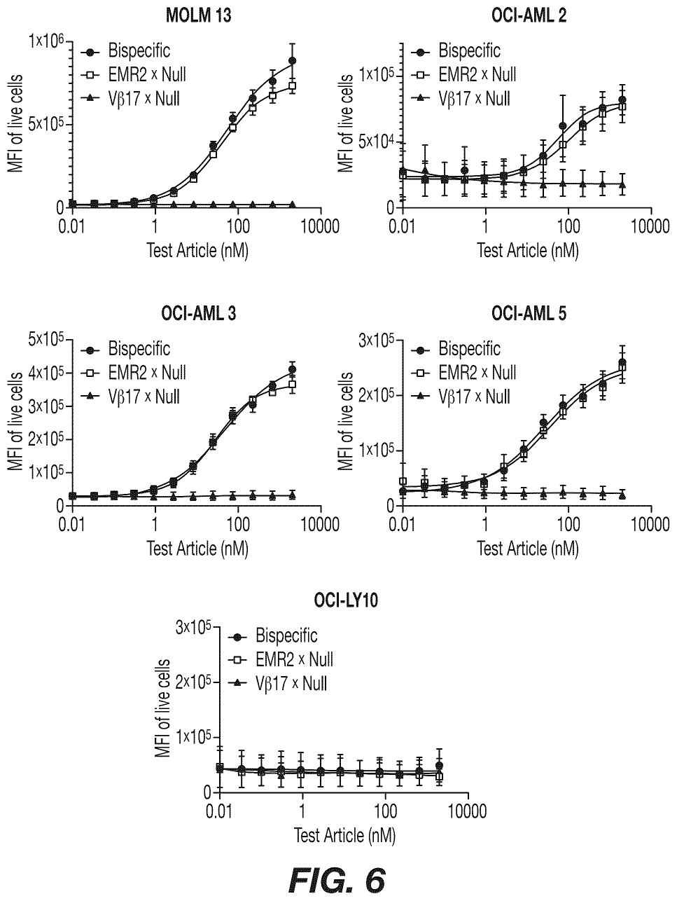

Flow cytometry was used to measure EMR2 arm binding affinity of the bispecific to AML cell lines MOLM-13, OCI-AML2, OCI-AML3, and OCI-AML5 that endogenously express EMR2 at varying receptor densities. Bispecific antibody binding to OCI-LY10, a B-cell lymphoma line that does not express EMR2, was also tested to confirm target specificity. Cell lines were administered increasing concentrations of a bispecific antibody, EMR2xNull, and TRBV19xNull for 1 hour at 37° C. A bispecific antibody showed concentration-dependent binding with an EC50 range of 27 to 55 nM on all EMR2-expressing cell lines (Table 7, FIG. 6). The TRBV19xNull negative control had no specific binding to any of the 5 tumor cell lines tested.

TABLE 7
EMR2 arm binding affinity to AML cell lines.

| | MOLM- | OCI- | OCI- | OCI- | OCI- |
|---|---|---|---|---|---|
| | 13 | AML2 | AML3 | AML5 | LY10 |
| Binding EC₅₀ | 53 ± 20 | 55 ± 54 | 48 ± 30 | 27 ± 7 | ND |
| (nM) ± SEM | | | | | |

AML, acute myeloid leukemia; EC50, 50% effective concentration; EMR2, epidermal-growth-factor-like module- containing mucin-like hormone receptor-like 2; ND, not detected; SEM, standard error of the mean.

### Bispecific Antibody Kinetic Binding to OCI-AML3 Cells

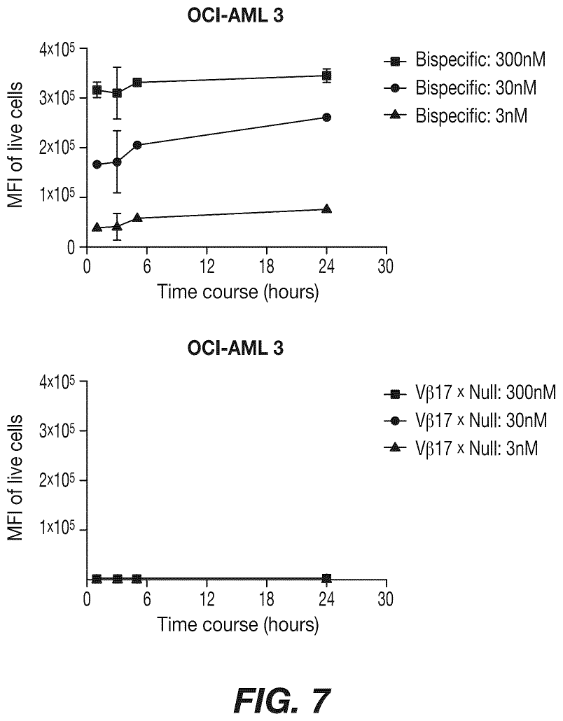

Flow cytometry was used to assess stability of EMR2 binding of a bispecific antibody to the OCI-AML3 cell line, which endogenously expresses EMR2. When OCI-AML3 cells were administered a bispecific antibody for 1, 3, 5, and 24 hours at 37° C., stable binding was observed at all concentrations tested (3, 30, and 300 nM; FIG. 7). TRBV19xNull, which lacks the EMR2 binding arm present in the bispecific antibody, displayed no binding to OCI-AML3 cells at any concentration or timepoint.

### Bispecific Antibody Binding to TRBV19+ T Cells

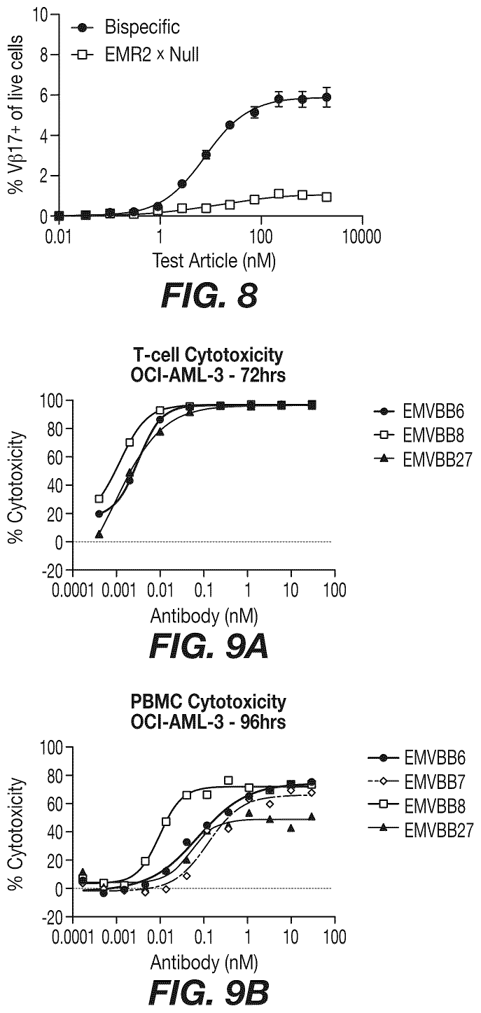

Flow cytometry was used to measure specific binding of the TRBV19 arm of the bispecific antibody to TRBV19⁺ T cells from 6 different healthy human pan-T-cell donors. When pan-T cells were administered increasing concentrations of the bispecific antibody at 1 hour, 37° C., concentration-dependent binding was observed (FIG. 8, Table 8). The EC50 value of detection of TRBV19⁺ cells for the bispecific antibody was 7.8±0.9 nM. Specific binding to TRBV19⁺ cells was not detected when stained with EMR2xNull, which lacks the TRBV19-spFv

TABLE 8
EMR2 arm binding affinity to AML cell lines.

| Parameter | Donor 1 | Donor 2 | Donor 3 | Donor 4 | Donor 5 | Donor 6 |
|---|---|---|---|---|---|---|
| EC₅₀ (nM) | 7.8 | 9.7 | 6.2 | 10.6 | 9.5 | 4.9 |

EC50, 50% effective concentration.
Data from 6 pan-T-cell donors. Values are calculated as log (agonist) versus variable slope (4 parameters).

The bispecific antibody displayed good intrinsic biophysical properties.

The bispecific antibody as described in the example is a fully human bispecific monoclonal antibody targeting the TRBV19 TCR with one binding arm and tumor cell-surface antigen EMR2 on the other binding arm. The bispecific antibody showed good intrinsic biophysical properties and bound to all tested EMR2-expressing cell lines. The bispecific antibody showed stable tumor cell binding profiles over 24 hours. The bispecific antibody also showed binding to a small subpopulation of primary human T cells expressing TRBV19 on the cell surface.

### Example 3. Cytotoxicity Assays for the EMR2xTRBV19 Abs

The EMR2 antibodies were assessed for cell cytotoxicity of OCI-AML3 AML cell line in T-cell or PBMC cytotoxicity assays. The results from one T-cell donor or one PBMC donor are shown in FIG. 9A (T-cell cytotoxicity assay) and 9B (PBMC cytotoxicity assay), where a dose range of EMR2xTRBV19 antibodies was used.

### Example 4. Human IgG1 Glm(17), Kappa Bispecific Knobs-into-Holes Antibody Targeting TCR TRBV19 and ERM2

The present disclosure provides an immunoglobulin (Ig) G1 bispecific antibodies that simultaneously bind to the T cell receptor (TCR) TRBV19 on T lymphocytes cells, and to EMR2 (Adhesion G protein-coupled receptor E2, Uniprot ID: Q9UHX3) on tumor cells. The exemplary antibody of the present disclosure features mutations of L234A, L235A, and D265S (AAS) in the constant region (Fc) to abolish interaction with Fc receptors and heterodimerization is enhanced using the knobs-into-holes platform mutations (Ridgway, J. B., Presta, L. G. & Carter, P. ‘Knobs-into-holes’ engineering of antibody CH3 domains for heavy chain heterodimerization. Protein Eng 9, 617-621 (1996)). The molecule comprises an anti-TRBV19 spFv fused onto the N-terminus of the “Knob” Fc region (T366W). The “hole” chain (T366S, L368A, Y407V) features an anti-ERM2 Fab at the N-terminus. The hole chain also contains the “RF” mutations (H435R, Y436F) to disrupt protein A binding of monomeric and homodimerized hole chains (Tustian, A. D., Endicott, C., Adams, B., Mattila, J. & Bak, H. Development of purification processes for fully human bispecific antibodies based upon modification of protein A binding avidity. MAbs 8, 828-838 (2016)).

### Source of Coding Sequence

The bispecific was generated by co-expression of the anti-TRBV19 spFv-Fc “knob” Heavy Chain (HC1) with the anti-EMR2 Fab Heavy Chain containing the “hole” and RF mutations (HC2) and paired with the EMR2 Light Chain 2 (LC2).

The anti-TCR Vb17 variable region VR000071196 was derived from murine clone E17.5F3.15.13 obtained from Beckman Coulter. The parental murine variable sequences were humanized and a deamidation site was mitigated. Briefly, the murine complementarity determination regions (CDRs) according to the AbM definitions were grafted into the human IGHV4-61*02-IGHJ6-01 and IGKV1-39*01-IGKJ2-01 human germlines. A human/murine binary library of VH variants in the frameworks (sequential positions 41, 45, 49, 68, 72 and 98) were generated and screened against Vb17⁺ cells and the best binder with two back mutations (V72R and R98S) was selected as the humanized variant. The parental Light Chain CDR1 contained a deamidation PTM-risk NG sequence motif at positions 33(N)-34(G), and this risk was eliminated by mutation of G34R after all possible mutations in both N and G positions were tested. The Vb17 binder B17B852-G34R was formatted as spFv in the LH orientation (light chain-linker-heavy chain) as described in Boucher et al. (Boucher, L. E, Prinslow, E. G., et al. “Stapling” scFv for multispecific biotherapeutics of superior properties. mAbs 15(1): 2195517. (2023)) and fused to the N-terminus of Fc for the final molecule.

The anti-ERM2 variable region VR000049625 featured in the bispecific is derived from the human IgG1, Kappa antibody named EMR2B454, discovered by immunizing transgenic humanized mice [Ablexis] with recombinant EMR2. The anti-EMR2 binder was formatted as a Fab at the N-terminus.

Generation of the expression plasmids used to generate the manufacturing cell line was performed as previously known.

### Amino Acid Sequence of an Exemplary Bispecific Molecule

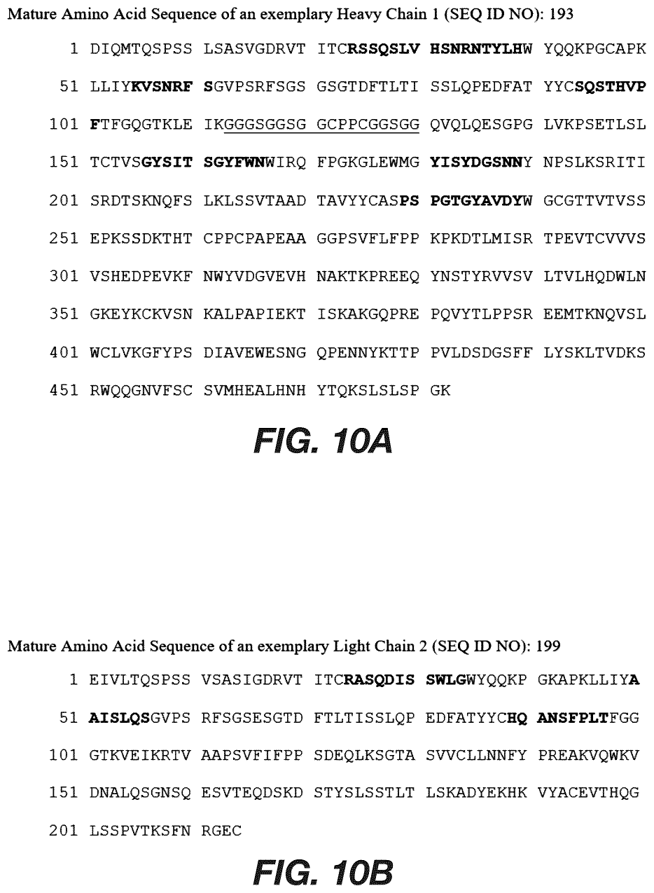

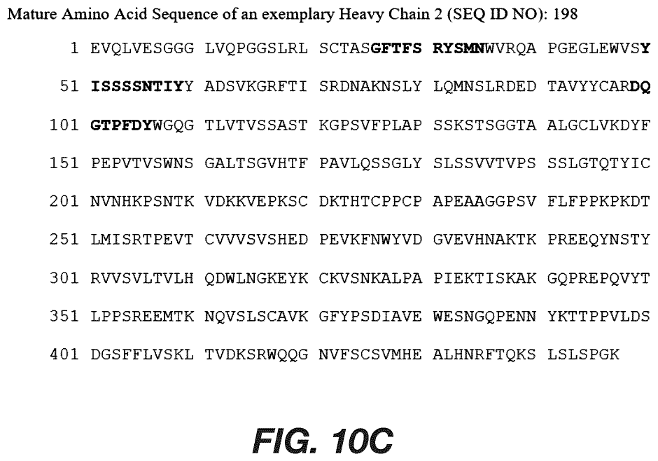

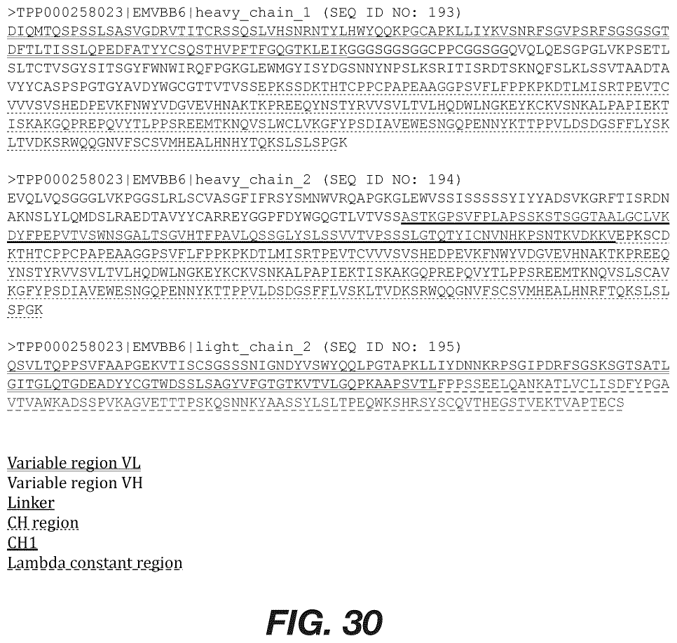

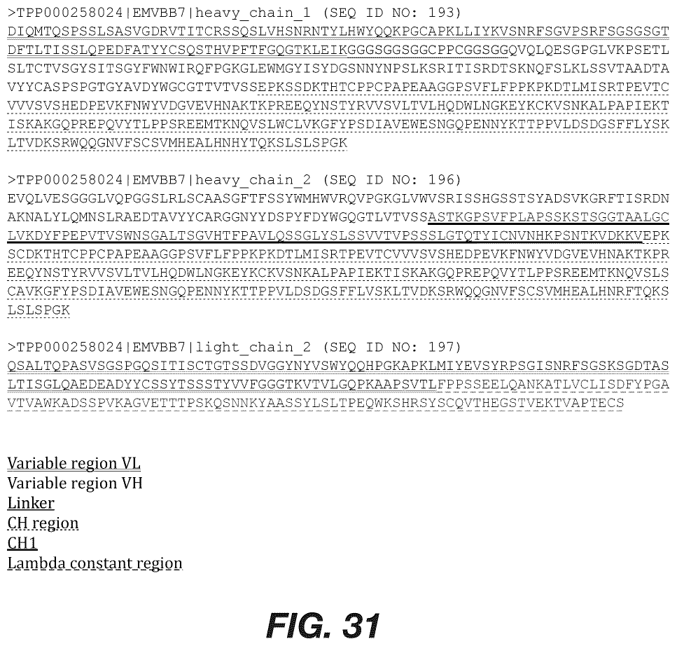

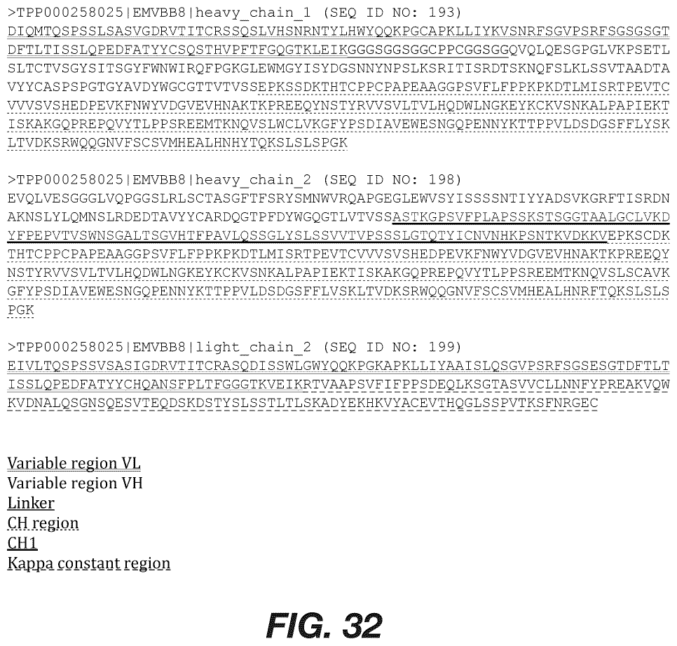

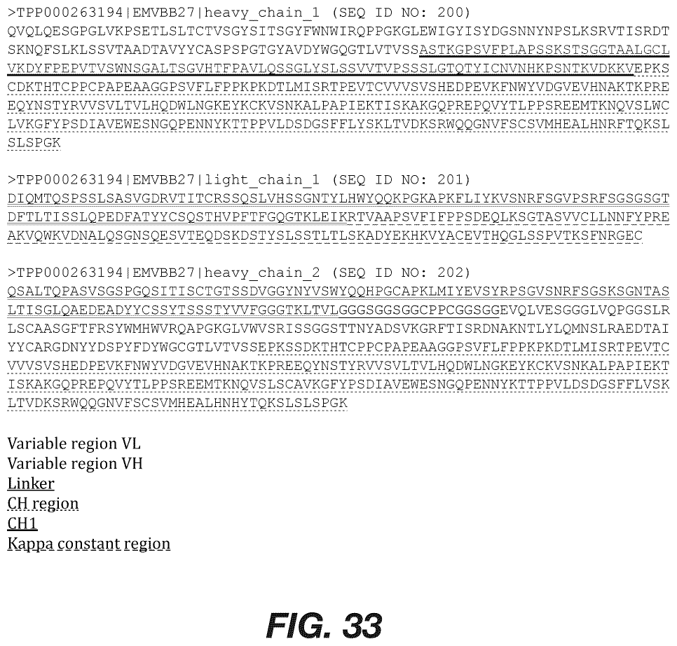

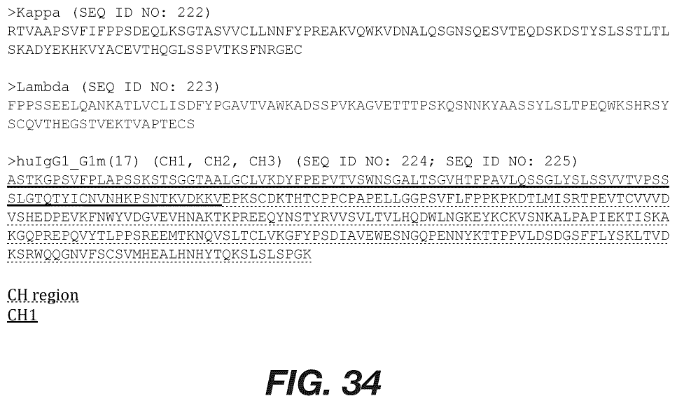

The amino acid sequence for exemplary bispecific Heavy Chain 1, Heavy Chain 2, and Light Chain 2 are shown in FIG. 10A-10C. The sequences were confirmed by peptide mapping and mass spectrometry. The complementarity-determining regions (CDRs) using AbM definition are shown in bold. Amino acid sequences of exemplary bispecific antibodies are also shown in FIGS. 30-33. Amino acid sequences of exemplary Kappa, Lambda, CH and CH1 regions are shown in FIG. 34.

The Asp residue at position 1 of exemplary bispecific Heavy Chain 1, Glu at position 1 of exemplary bispecific Heavy Chain 2, and Glu residue at position 1 of exemplary bispecific Light Chain 2 constitute the N-termini of the mature chains.

The following mutations (Eu numbering) were introduced:

L234A L235A D265S (AAS) to eliminate binding to Fe gamma receptors FcγRI, FcγRIIA, FcγRIIb, and FcγRIIIa (Fc receptor silencing).

With the purpose of enabling formation of the bispecific antibody, T366W (knob) was introduced into Heavy Chain 1, and T366S L368A Y407V (hole) was introduced into the Heavy Chain 2 (Ridgway, J. B., Presta, L. G. & Carter, P. ‘Knobs-into-holes’ engineering of antibody CH3 domains for heavy chain heterodimerization. Protein Eng 9, 617-621 (1996)).

H435R Y436F (RF) mutation set introduced to disrupt protein A binding of monomeric and homodimerized HC2/LC2 chains (Tustian, A. D., Endicott, C., Adams, B., Mattila, J. & Bak, H. Development of purification processes for fully human bispecific antibodies based upon modification of protein A binding avidity. MAbs 8, 828-838 (2016)).

C220S in Heavy Chain 1 introduced to prevent unpaired cysteine in the scFv binder.

### Example 5. In Vitro Pharmacology Studies

### Bispecific Antibody T-Cell-Mediated Cytotoxicity Assays

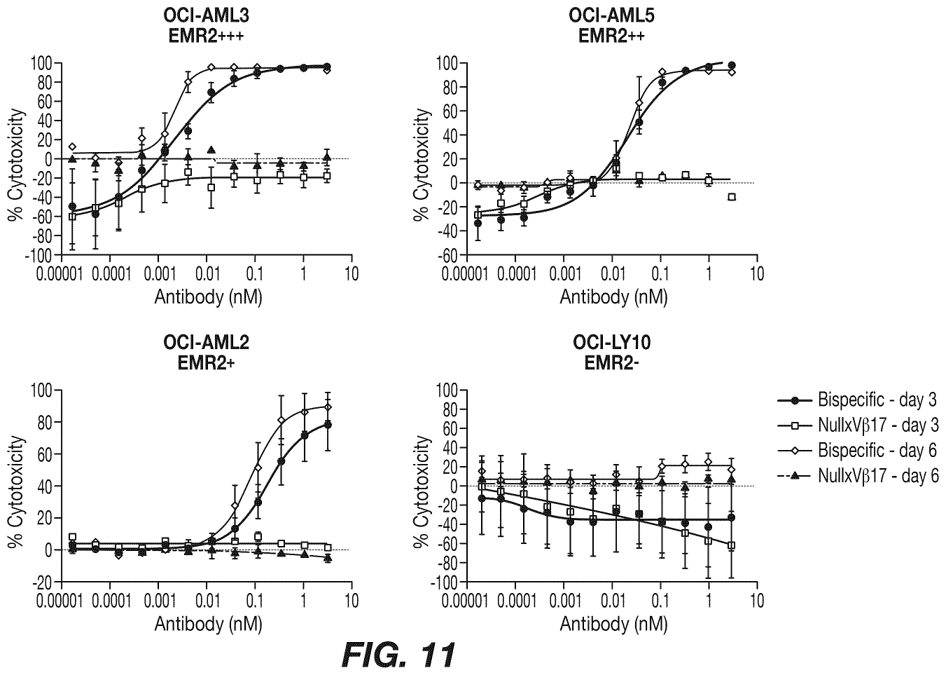

The activity of the bispecific antibody in T-cell-mediated cytotoxicity was evaluated using coculture assays of healthy-donor pan-T cells and a panel of EMR2+ AML cell lines with different expression levels (i.e., OCI-AML3, OCI-AML5, and OCI-AML2) or an EMR2− cell line (i.e., OCI-LY10). Cytotoxicity to cancer cells was assessed at 3 and 6 days using flow cytometry. The bispecific antibody was able to elicit potent cytotoxicity to all EMR2+ cell lines at both time points (FIG. 11). No cytotoxicity was induced in presence of the TAA-negative cell line or with the NullxTRBV19 negative control.

### Bispecific Antibody Mediated TRBV19+ T-Cell Activation In Vitro

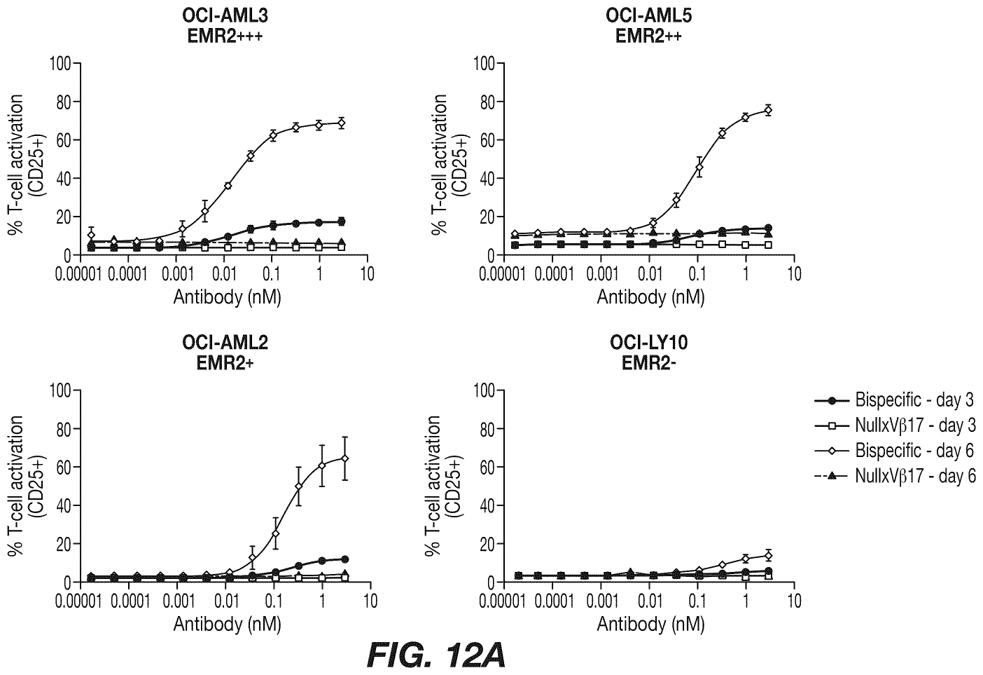

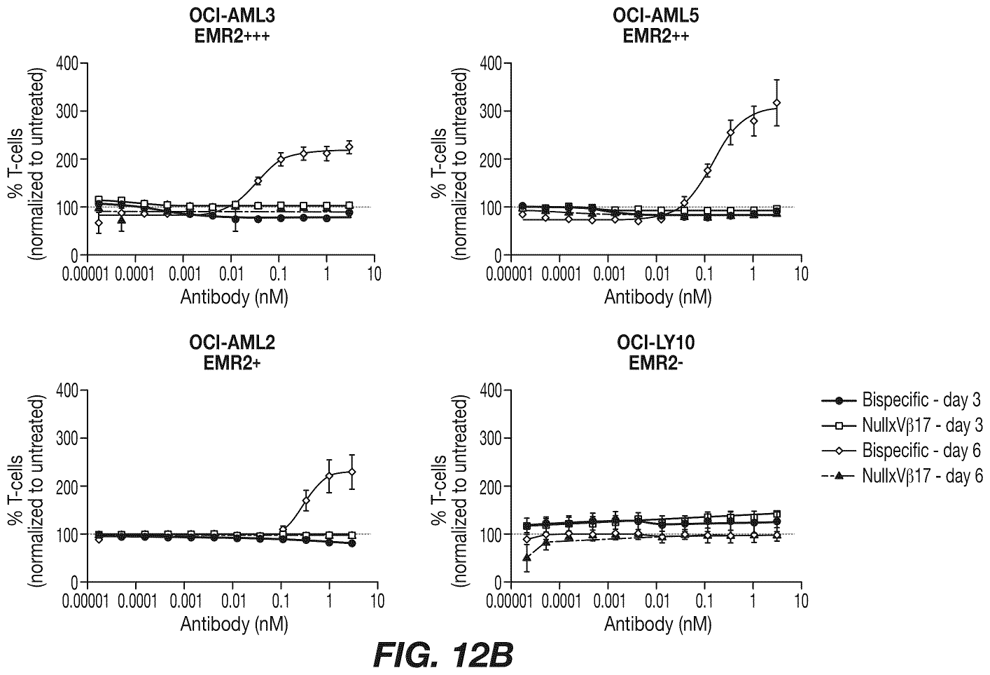

To assess the T-cell activation and expansion profile upon treatment, the expression of a late activation marker (i.e., CD25) was measured on T cells. T-cell activation and expansion (˜2- to 3-fold in pan-T cells at Day 6) were induced in the presence of the bispecific antibody only when incubated with EMR2⁺ and not in the presence of the EMR2⁻ cell lines, demonstrating antigen specificity of its activity (FIGS. 12A-12B). While potent cytotoxicity could be readily observed at Day 3 (FIG. 11), minimal T-cell activation and expansion were detected at the same timepoint. The effect of the bispecific antibody was both dose and time dependent, leading to a greater induction of T-cell activation and expansion at Day 6. The negative control NullxTRBV19 antibody did not induce significant T-cell activation in any of the cell lines tested.

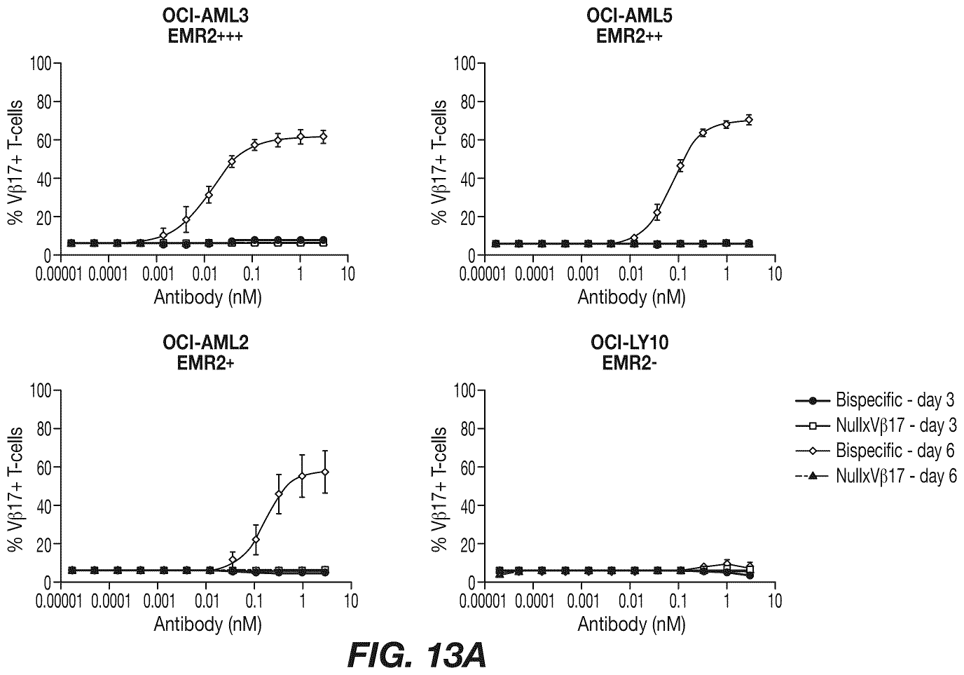

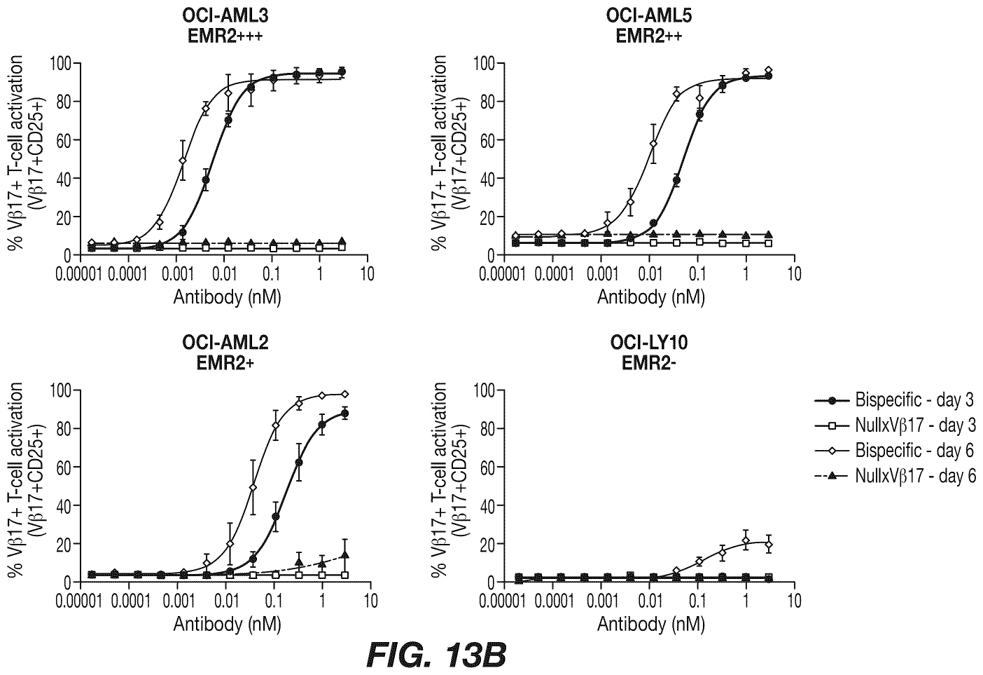

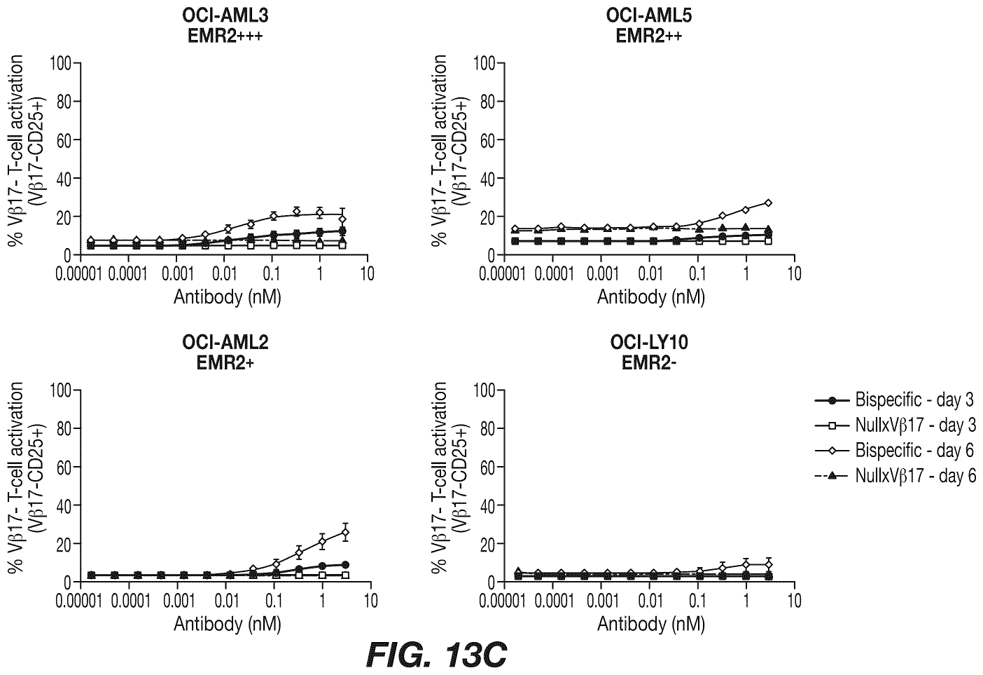

When looking more specifically within the TRBV19⁺ and TRBV19⁻ T-cell subpopulations, it could be demonstrated that the bispecific antibody-induced expansion and activation was highly restricted to the TRBV19⁺ T cells (FIGS. 13A-13C). No significant expression of the CD25 activation marker could be detected within the TRBV19⁻ T cells at Day 3 for all cell lines tested. Minimal levels of T-cell activation were observed at the top doses of the bispecific antibody within TRBV19⁻ T cells at Day 6, which could potentially be due to allogeneic reaction after a long coculture time leading to non-specific T-cell activation.

### Measurement of Bispecific Antibody-Induced Cytokine Production in a Flow-Cytometry-Based Assay

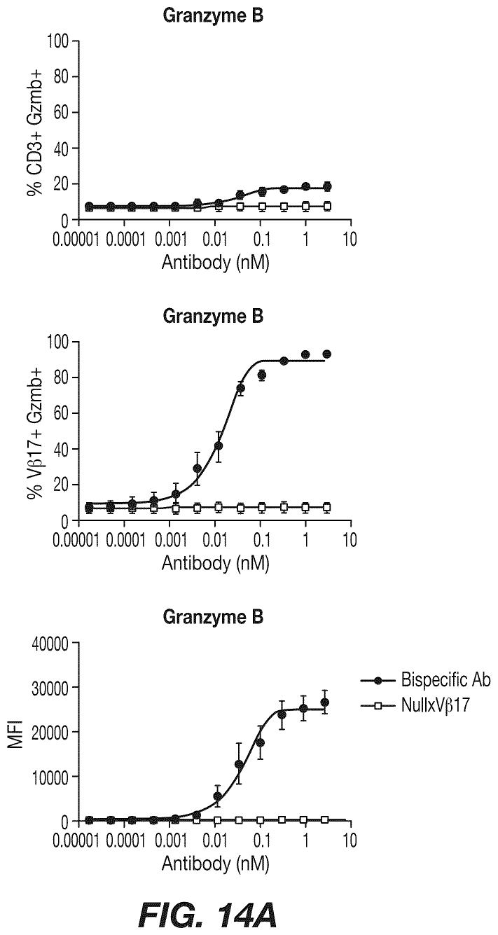

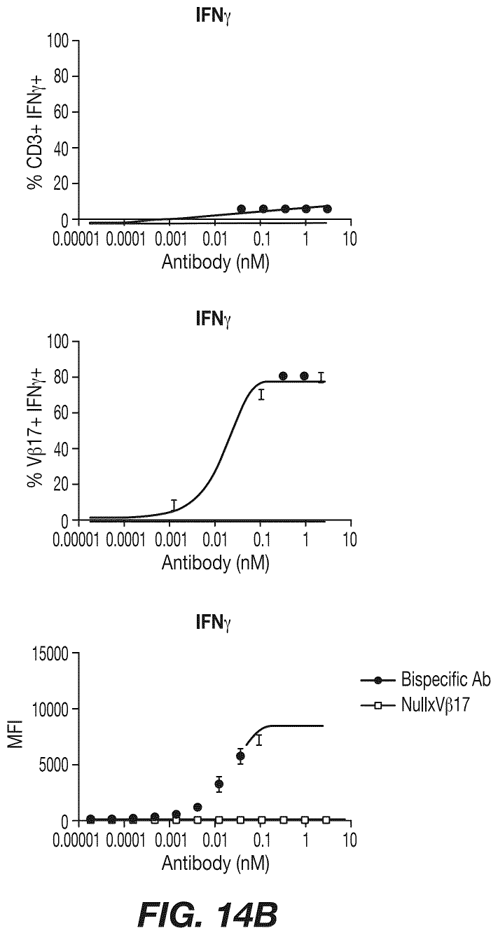

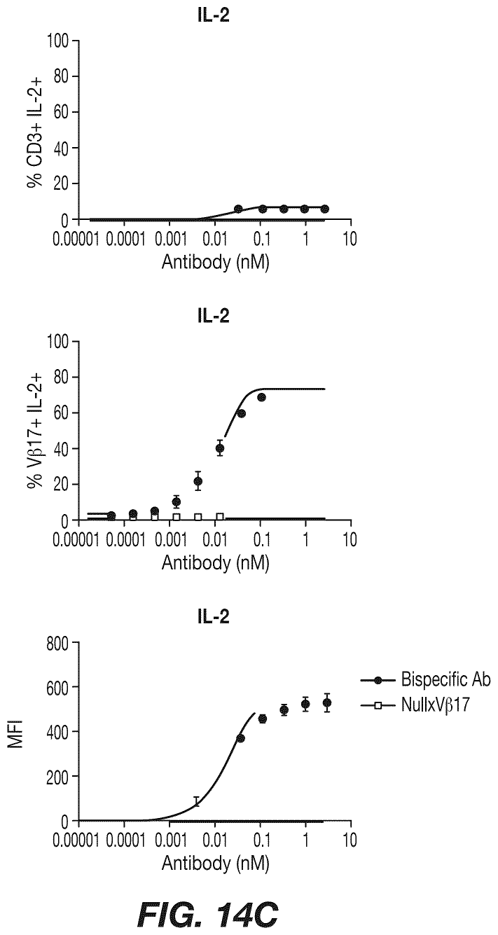

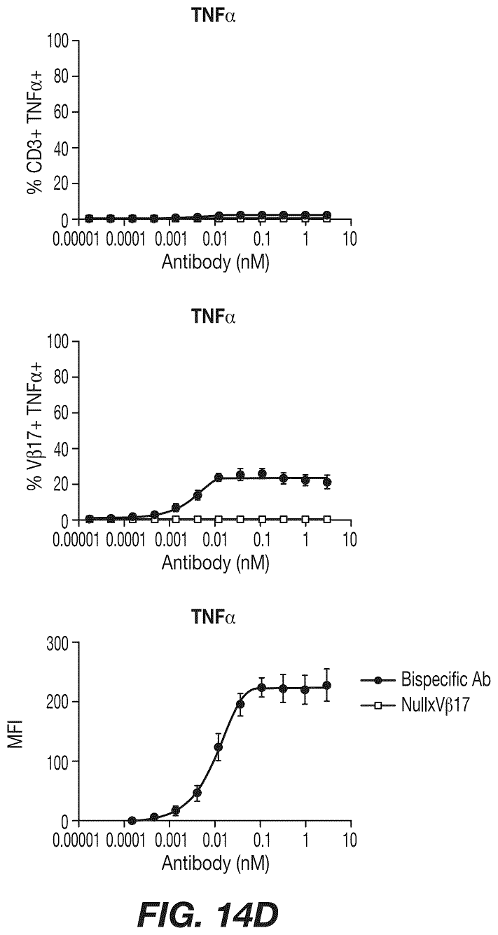

To better understand the TRBV19⁺ T-cell profile upon activation, a flow-cytometry-based assay was set up to look at the intracellular production of interferon (IFN)-γ, IL-2, and TNF-α at different timepoints. OCI-AML3 cells and pan-T cells (n=6 different healthy donors) were cocultured at an effector-to-target (E:T) ratio of 10:1 (relative E:T ratio for TRBV19⁺ T cells of 0.5:1) and analyzed by flow cytometry after 48 and 72 hours of incubation with the bispecific antibody or the NullxTRBV19 control. The comparator antibody A was assessed in parallel. The percentage of cytotoxicity as well as cytokine-producing T cells and the intensity of the signal (i.e., mean fluorescence intensity [MFI]) for each cytokine was evaluated. While potent cytotoxicity was observed readily at 48 hours, the percentages of granzyme-B-expressing cells was minimal in the total T-cell population (FIG. 14A). This was specifically detected in the TRBV19⁺ population and not in the TRBV19⁻ population. Further evaluation of cytokine expression (IFN-γ, IL-2, and TNF-α) showed similar effects, where minimal expression was detected within the total T-cell population that was specific for the TRBV19+ population (FIG. 14B-14D). IL-6 expression on pan-T cells and TRBV19+ cells was low, corresponding to a maximum of ˜20% expression at the highest concentrations. It is noteworthy that the intensity of cytokine expression on single-cell level within the TRBV19+ population engaged by the bispecific antibody was slightly higher than the signal detected within pan-T cells engaged with the control comparator antibody A (FIGS. 15A-15D). Similar data were observed at 72 hours. These data could indicate the potential of the TRBV19+ T cells to induce a potent response despite representing only ˜5% of pan-T cells, further validating the hypothesis that TRBV19+ T cells are characterized by a memory/primed phenotype.

### Bispecific Antibody PBMC-Mediated Cytotoxicity Assays

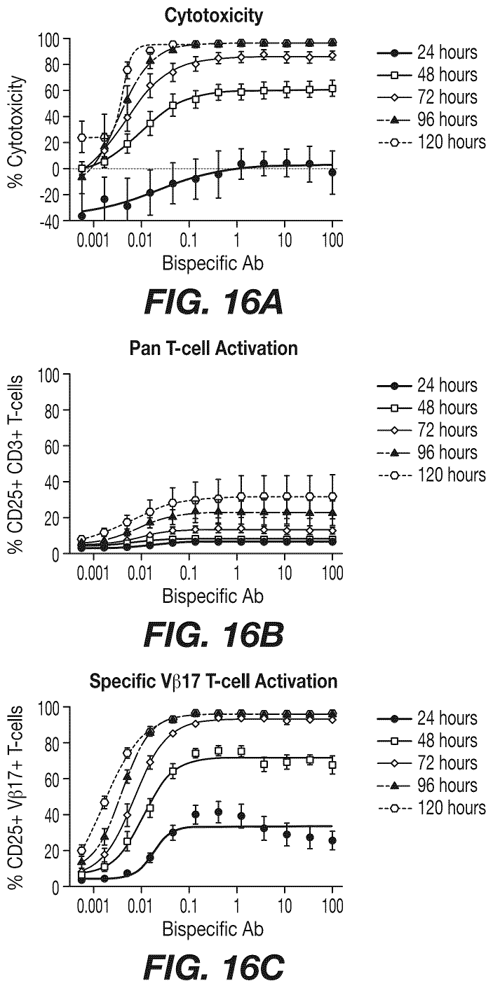

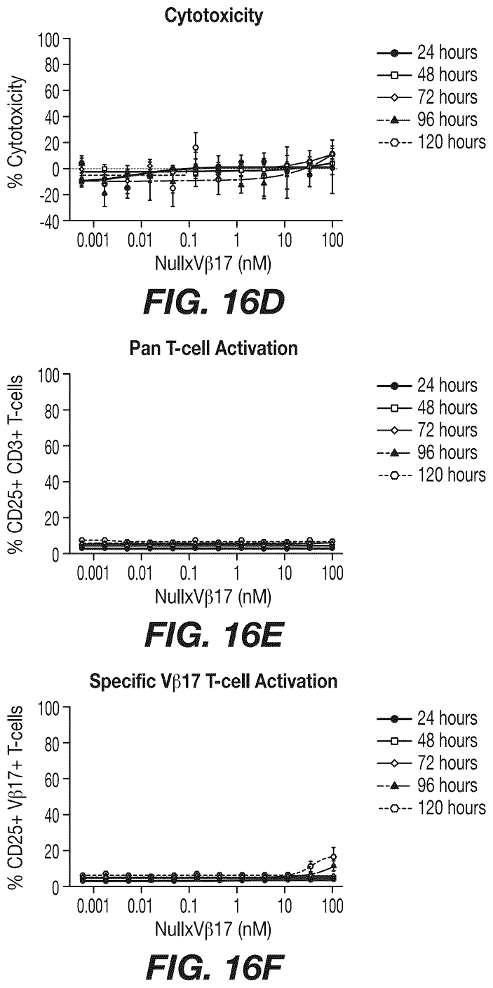

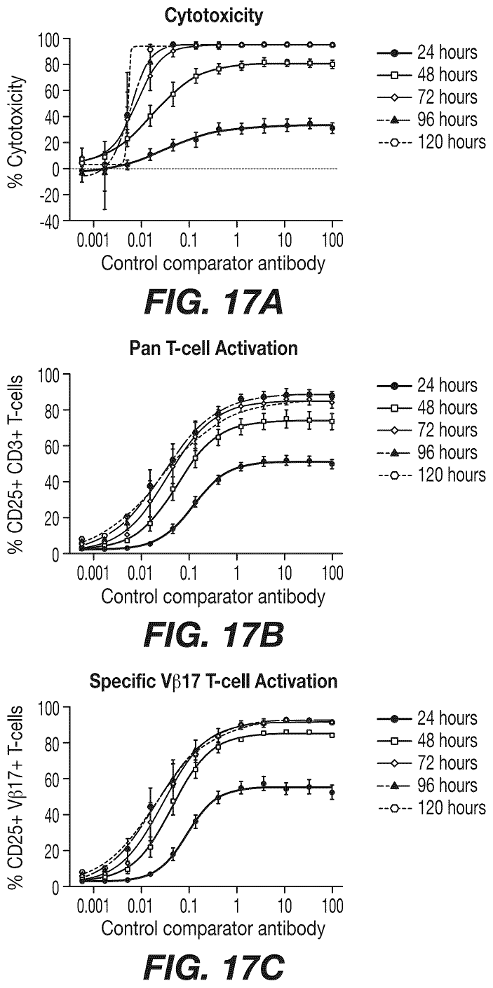

To characterize the kinetics of the bispecific antibody in a more physiological setting, a time course PBMC-based cytotoxicity assay was optimized to determine cancer cell cytotoxicity as well as T-cell activation. PBMCs from a total of 5 healthy donors were cocultured at a ratio of 5:1 with the OCI-AML3 cell line as target cells. Treatment with a dose titration of the bispecific antibody or NullxTRBV19 antibodies was performed at 24, 48, 72, 96, and 120 hours. Cell cytotoxicity and T-cell activation analysis were assessed by flow cytometry. The bispecific antibody showed dose- and time-dependent induction of cytotoxicity against OCI-AML3 cells where maximum cytotoxicity was reached between 72 and 96 hours (FIGS. 16A-16F, Table 9). T-cell activation analysis showed a maximum of ˜30% pan-T-cell activation at 120 hours as measured by the expression of the late activation marker CD25. Among the TRBV19⁺ T cells, maximum specific T-cell activation was detected between 72 and 96 hours, corelating with the cytotoxicity data. No impact on cancer cell cytotoxicity nor T-cell activation was observed in presence of the NullxTRBV19 control. As a reference, the control comparator antibody A antibody was included in the assay. While maximum cytotoxicity was reached at earlier timepoints with the control comparator antibody A, the bispecific antibody led to comparable cytotoxicity over time (FIGS. 17A-17C). Additionally, significantly higher overall pan-T-cell activation was observed as early as 48 hours (Table 10).

TABLE 9
Maximum values and EC50 values of cytotoxicity and
T-cell activation from PBMC cytotoxicity assays.

| | | | | |
|---|---|---|---|---|
| 24 | 48 | 72 | 96 | 120 |
| hours | hours | hours | hours | hours |

Cytotoxicity

| | | | | | |
|---|---|---|---|---|---|
| Max value (%) | 3.2 | 60.7 | 86.2 | 96.6 | 95.7 |
| EC₅₀ (nM) | 0.02 | 0.01 | 0.004 | 0.003 | 0.004 |

Pan-T-cell activation

| | | | | | |
|---|---|---|---|---|---|
| Max value (%) | 6.7 | 8.2 | 13.4 | 23.0 | 31.5 |
| EC₅₀ (nM) | 0.02 | 0.01 | 0.01 | 0.01 | 0.01 |

EC50, 50% effective concentration; max, maximum; PBMC, peripheral blood mononuclear cell.
Data from 5 healthy donors were pooled and represented as mean. A nonlinear regression model was used for EC50 and max mean value estimation across the donors. The model was fit using log-transformed data in GraphPad Prism 10.

TABLE 10
Maximum values and EC50 values of cytotoxicity and
T-cell activation from PBMC cytotoxicity assays.

| | | | | |
|---|---|---|---|---|
| 24 hours | 48 hours | 72 hours | 96 hours | 120 hours |

Cytotoxicity

| | | | | | | |
|---|---|---|---|---|---|---|
| Bispecific | Max value (%) | 3.2 | 60.7 | 86.2 | 96.6 | 96.7 |
| Antibody | EC₅₀ (nM) | 0.02 | 0.01 | 0.004 | 0.003 | 0.004 |
| Control | Max value (%) | 33.5 | 81.2 | 95.4 | 95.8 | 94.2 |
| comparator | EC₅₀ (nM) | 0.03 | 0.02 | 0.01 | 0.01 | 0.01 |
| antibody A | | | | | | |

Pan-T-cell activation

| | | | | | | |
|---|---|---|---|---|---|---|
| Bispecific | Max value (%) | 6.7 | 8.2 | 13.4 | 23.0 | 31.5 |
| Antibody | EC₅₀ (nM) | 0.02 | 0.01 | 0.01 | 0.01 | 0.01 |
| Control | Max value (%) | 51.3 | 74.2 | 85.4 | 89.0 | 85.5 |
| comparator | EC₅₀ (nM) | 0.12 | 0.06 | 0.04 | 0.03 | 0.03 |
| antibody A | | | | | | |

CD, cluster of differentiation; EC50, 50% effective concentration; EMR2, epidermal-growth-factor-like module-containing mucin-like hormone receptor-like 2; max, maximum; PBMC, peripheral blood mononuclear cell.
Data from 5 healthy donors were pooled and represented as mean. A nonlinear regression model was used for EC50 and max mean value estimation across the donors. The model was fit using log-transformed data in GraphPad Prism 10.

### Cytokine Release Profiling of the Bispecific Antibody

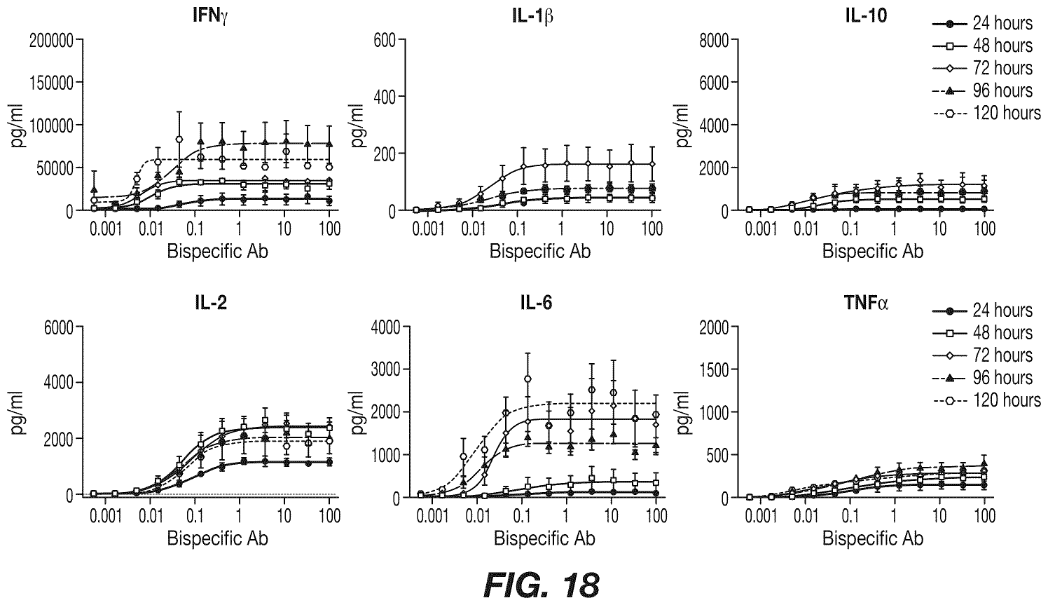

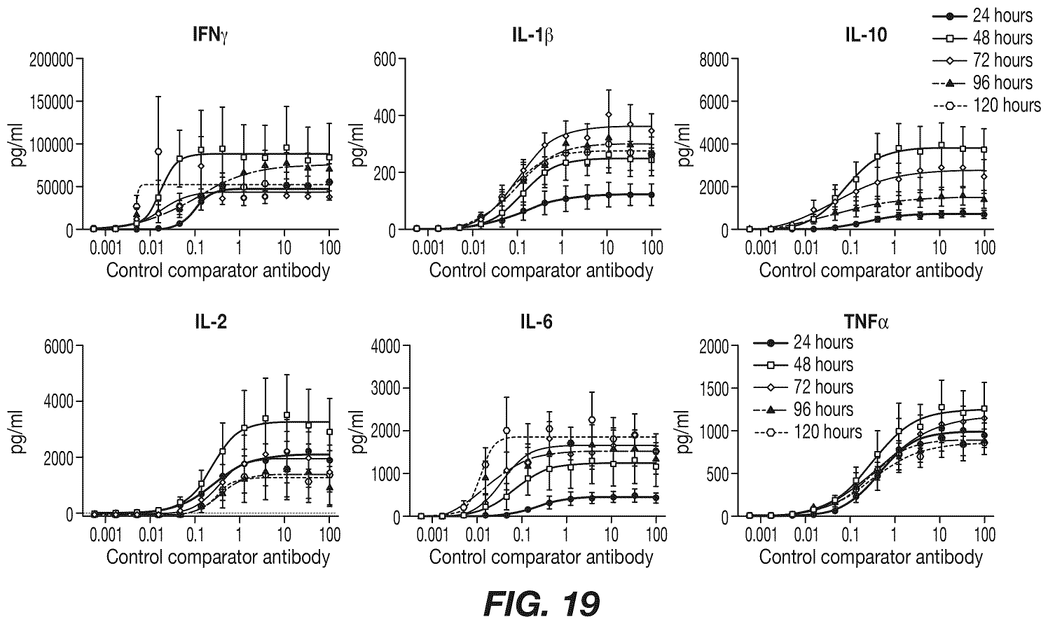

To characterize the cytokine release profile of the bispecific antibody, cytokine levels were measured using the Meso Scale Discovery (MSD) ELISA Proinflammatory Panel 1 in the supernatants of the PBMC assays. Supernatants were collected at each time point and data was normalized to untreated control wells. Similarly, the control comparator antibody A was used in parallel as a reference for cytokine production. Overall, the kinetics of cytokine secretion were different between the bispecific antibody and control comparator antibody A. For most cytokines, peak cytokine production was detected between 72 and 96 hours for the bispecific antibody (FIG. 18) while for the control comparator antibody A, maximum productions were readily detected at 48 hours (FIG. 19). Moreover, there was a significant difference in the highest cytokine concentrations between both antibodies, where the bispecific antibody led to significantly lower production of IL-1β, IL-10, and TNF-α (Table 11 and Table 12). While IL-6 expression on T cells was low as indicated above, high production levels were observed at 72 hours in the PBMC assays, potentially indicating that IL-6 could be secreted by other immune cells or the cancer cells. This is in line with previous data showing that monocytes and AML leukemic blasts release IL-6. Interestingly, single-cell RNA sequencing (RNAseq) as well as bulk RNAseq indicated that OCI-AML3 cell lines express some levels of IL-6 and IFN-γ at baseline and after TCE treatment. Altogether these data further validated the hypothesis that the bispecific antibody could lead to a lower risk of T-cell-mediated CRS by selectively engaging and activating a specific T-cell subpopulation.

TABLE 11
Bispecific Antibody-induced inflammatory cytokines in the presence
of OCI-AML3 cells: EC50 and max cytokine release values.

| | | |
|---|---|---|
| Cytokine | Max value (pg/mL)-time point | EC₅₀ (nM) |

| | | | |
|---|---|---|---|
| IFN-γ | 77,827-96 | hours | 0.03 |
| IL-1β | 162-72 | hours | 0.02 |
| IL-10 | 1,199-72 | hours | 0.02 |
| IL-2 | 2,463-72 | hours | 0.08 |
| IL-6 | 2,189-120 | hours | 0.01 |
| TNF-α | 368.7-72 | hours | 0.07 |

EC₅₀, 50% effective concentration; IFN, interferon; IL, interleukin; max, maximum; MSD, Meso Scale Discovery; NE, not estimable; PBMC, peripheral blood mononuclear cell; SEM, standard error of the mean; TNF, tumor necrosis factor.
Supernatant were collected at the different time points and analyzed for inflammatory cytokines using MSD Proinflammatory kit. Graphing of data was done in GraphPad Prism 10. Data from 5 different donors were pooled and represented as mean ± SEM.

TABLE 12
Inflammatory cytokines induced by the control comparator
antibody A in the presence of OCI-AML3 cells:
EC50 and max cytokine release values.

| | | |
|---|---|---|
| Cytokine | Max value (pg/mL)-time point | EC₅₀ (nM) |

| | | | |
|---|---|---|---|
| IFN-γ | 88,580-48 | hours | 0.02 |
| IL-1β | 362.5-72 | hours | 0.1 |
| IL-10 | 3,825-48 | hours | 0.07 |
| IL-2 | 3,269-48 | hours | 0.2 |
| IL-6 | 1,868-120 | hours | 0.01 |
| TNF-α | 1,254-48 | hours | 0.3 |

CD, cluster of differentiation; EC₅₀, 50% effective concentration; EMR2, epidermal-growth-factor-like module- containing mucin-like hormone receptor-like 2; IFN, interferon; IL, interleukin; max, maximum; MSD, Meso Scale Discovery; SEM, standard error of the mean; TNF, tumor necrosis factor.
Supernatant were collected at the different time points and analyzed for inflammatory cytokines using MSD Proinflammatory kit. Graphing of data was done in GraphPad Prism 10. Data from 5 different donors were pooled and represented as mean ± SEM

### Bispecific Antibody-Mediated Cytotoxicity on Primary AML BM Blast

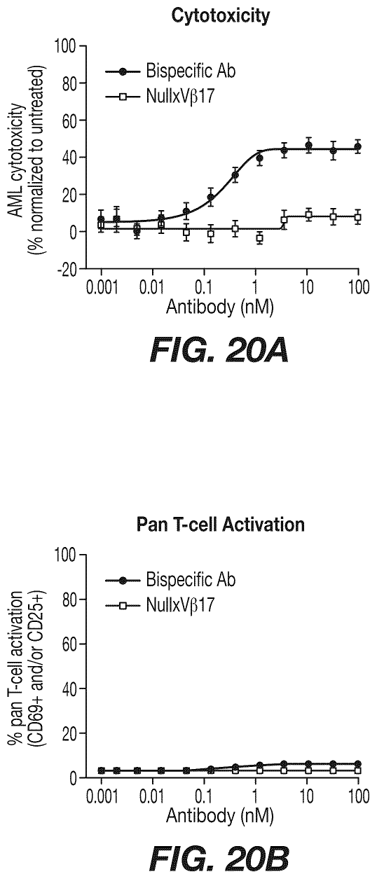

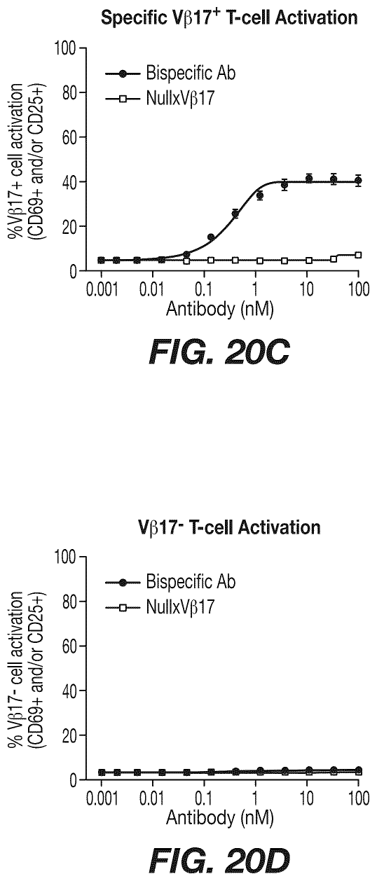

To further assess the cytotoxic potential of the bispecific antibody in a relevant AML setting, cytotoxicity assays were performed using AML BM samples as target cells. Since the viability of primary AML BM cells is compromised at 48 hours in vitro, the assay was performed for 24 hours at a relative E:T ratio of 2:1 with pan-T cells as effectors. the bispecific antibody promoted a dose-dependent reduction of blasts in the tested AML BM donors (n=3) (FIGS. 20A-20D). While no pan-T-cell activation was detected, specific activation of TRBV19⁺ T cells was observed. No activation was detected in the TRBV19⁻ T cells. The NullxTRBV19 control did not show appreciable cancer cell cytotoxicity nor T-cell activation.

### Activity of the Bispecific Antibody on Healthy HSPCs and Myeloid Cells

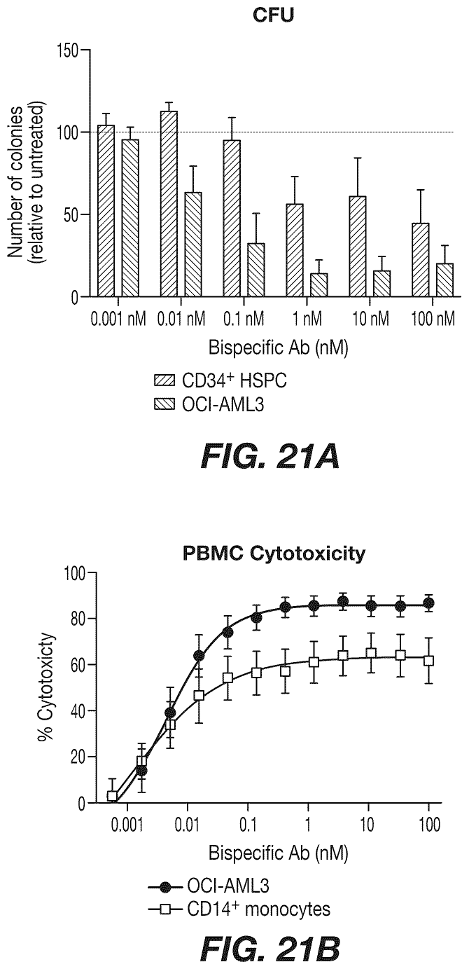

Similar to other targets pursued in AML, EMR2 is also expressed on healthy myeloid cells (FIG. 4). To determine the activity of the bispecific antibody on cancer cells versus healthy HSPCs, colony-forming unit (CFU) assays were performed with healthy CD34⁺ HSPCs. A coculture assay of pan-T cells and CD34⁺ cells or OCI-AML3 cells was set up. The inhibition of colony formation of OCI-AML3 cells induced by the bispecific antibody could be readily seen at doses between 0.01 and 0.1 nM. At the same concentrations, no significant effects were observed on the clonogenic potential of CD34⁺ HSPCs (FIG. 21A). While increasing concentrations of the bispecific antibody (up to 100 nM) did not cause >˜50% reduction in the CD34⁺ HSPC colony formation, ˜85% inhibition of OCI-AML3 colonies was observed.

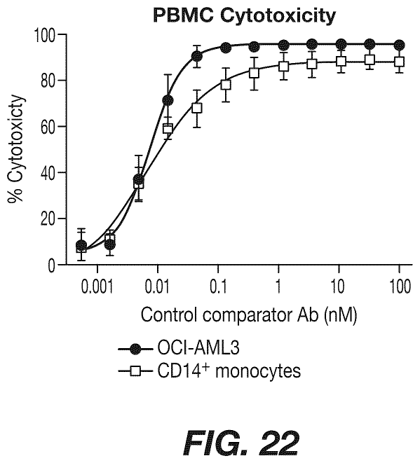

To evaluate the effect of the bispecific antibody treatment on healthy monocytes, PBMC cytotoxicity assays with OCI-AML3 cocultures were performed as described above and cytotoxicity of monocytes was assessed. At 72 hours, the bispecific antibody induced >85% cytotoxicity on OCI-AML3 cells while monocyte cytotoxicity showed a plateau effect at 60% (FIG. 21B). In contrast, comparable cytotoxicity to cancer cells and monocytes was observed with the control comparator A antibody (FIG. 22).

### Example 6. In Vivo Pharmacology Studies

The in vivo antitumor activity of the bispecific antibody was evaluated in 2 disseminated EMR2⁺ AML models labelled with luciferase (luc): MOLM-13-luc and OCI-AML3-luc. Tumor-bearing female NSG (i.e., non-obese diabetic [NOD] severe combined immunodeficiency [scid] gamma or NOD.Cg Prkdc^{scid} Il-2rg^{tm1Wjl}/SzJ) mice were humanized with either CD3⁺ pan-T cells or isolated TRBV19⁺ T cells from healthy donors.

The tolerability of the bispecific antibody could not be assessed with respect to EMR2 or TRBV19 binding to host tissues due to the lack of cross-reactivity to corresponding mouse antigens; the engrafted human T cells did bind the bispecific antibody. Engraftment of human T cells can lead to body weight loss due to eventual graft-versus-host disease (GvHD), however treatment with the bispecific antibody did not result in significant body weight loss as compared to the Dulbecco's phosphate-buffered saline (DPBS)-treated control group. Disseminated AML xenograft models including MOLM-13 and OCI-AML3 generally home to the BM of the hind limb as well as the spinal column ultimately leading to hind limb paralysis. Animals were monitored daily for negative clinical signs related to excessive tumor burden and monitored for body weight loss twice per week. When individual animals exhibited negative clinical signs or reached ≥20% body weight loss as compared to initial body weights, they were removed from the study and humanely euthanized. Whole-body in vivo imaging was performed twice weekly for the MOLM-13 disseminated model and weekly for the OCI-AML3 disseminated model according to rate of disease progression.

### Efficacy of the Bispecific Antibody in MOLM-13-Luc Established Disseminated Model in T-Cell-Humanized Mice

MOLM-13-luc cells (1×10⁵) were injected IV on Day 0. Mice (n=10/group) were randomized by bioluminescence intensity (BLI) and humanized with either pan CD3⁺ T cells at 2 different concentrations (1×10⁷ or 4×10⁷) or TRBV19⁺ T cells at 2 different concentrations (2×10⁶ or 1×10⁷) per mouse on Day 3 to evaluate the efficacy of the bispecific antibody under varied conditions of T-cell humanization. Starting on Day 4, mice were intraperitoneally (IP) dosed with the bispecific antibody twice weekly at 0.05 and 1 mg/kg, or DPBS for a total of 10 doses.

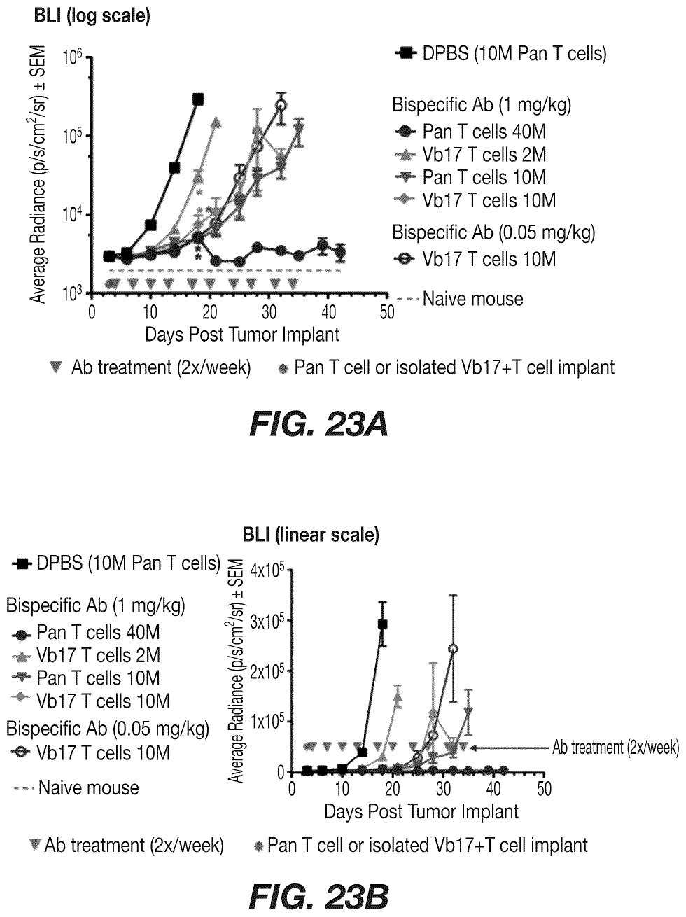

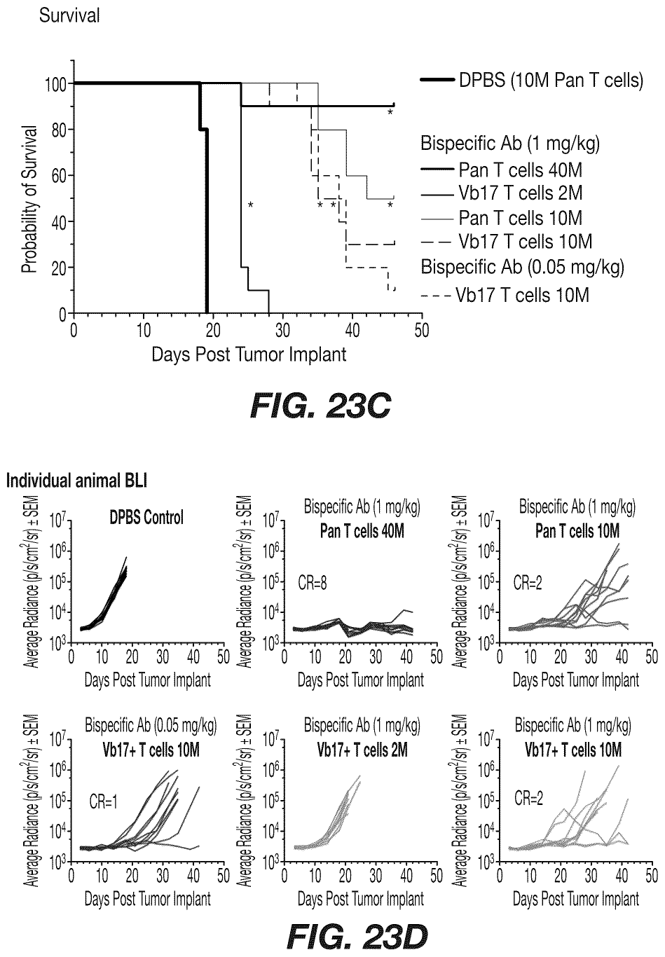

Significant antitumor efficacy was observed with the bispecific antibody treatment at 1 mg/kg in mice humanized with all the various conditions of T cells as assessed by growth rate over time (p<0.0001), as compared to the DPBS-treated control group (humanized with 1×10⁷ pan CD3⁺ T cells), with 99% Δ tumor growth inhibition (TGI) in groups humanized with pan CD3⁺ T cells, and 91% and 98% ΔTGI in groups humanized with 2×10⁶ or 1×10⁷ TRBV19⁺ T cells, respectively, on Day 18 post tumor implantation (FIG. 23A and FIG. 23B). Significant antitumor efficacy was also observed in mice treated with the bispecific antibody at 0.05 mg/kg humanized with 1×10⁷ TRBV19⁺ cells as assessed by growth rate over time (p<0.0001), with 99% ΔTGI on Day 18 post tumor implantation. Treatment with the bispecific antibody at 1 mg/kg elicited 8 and 2 complete responses (CR) in the groups humanized with 4×10⁷ and 1×10⁷ pan CD3⁺ T cells, respectively, and 2 CRs in the group humanized with 1×10⁷ TRBV19⁺ T cells (FIG. 23D). Treatment with the bispecific antibody at 0.05 mg/kg of mice humanized with 1×10⁷ TRBV19⁺ T cells resulted in 1 CR.

The bispecific antibody at 1 mg/kg in mice humanized with 4×10⁷ or 1×10⁷ pan CD3⁺ T cells resulted in median survival values of >46 and 44 days, respectively, compared to the median survival of 19 days in the DPBS-treated control group resulting in a biologically significant percent increased life span (ILS; i.e., >25%) of >142% and 132%, respectively (FIG. 23C). the bispecific antibody at 1 mg/kg in mice humanized with 2×10⁶ or 1×10⁷ TRBV19⁺ T cells resulted in median survival values of 24 and 37 days, respectively, resulting in a biologically significant 26% and 92% ILS, respectively, compared to the DPBS control. The bispecific antibody at 0.05 mg/kg in mice humanized with 1×10⁷ TRBV19⁺ T cells resulted in median survival of 39 days, resulting in a biologically significant 103% ILS compared to the DPBS control. Statistical differences in survival curves were observed with the bispecific antibody treatment as compared to DPBS (p<0.0001).

It was expected that mice humanized with 4×10⁷ pan CD3⁺ T cells or 2×10⁶ TRBV19⁺ T cells would have similar antitumor responses due to being humanized with theoretically equivalent numbers of TRBV19⁺ T cells; however, greater TGI and CRs were observed with 4×10⁷ pan CD3⁺ T cells (FIG. 23D). Presence of different T-cell subtypes may have contributed to a greater antitumor response in mice humanized with 4×10⁷ pan CD3⁺ T cells, which is more physiologically relevant. Alternatively, 2×10⁶ T cells might not be a sufficient number of T cells to humanize the mice.

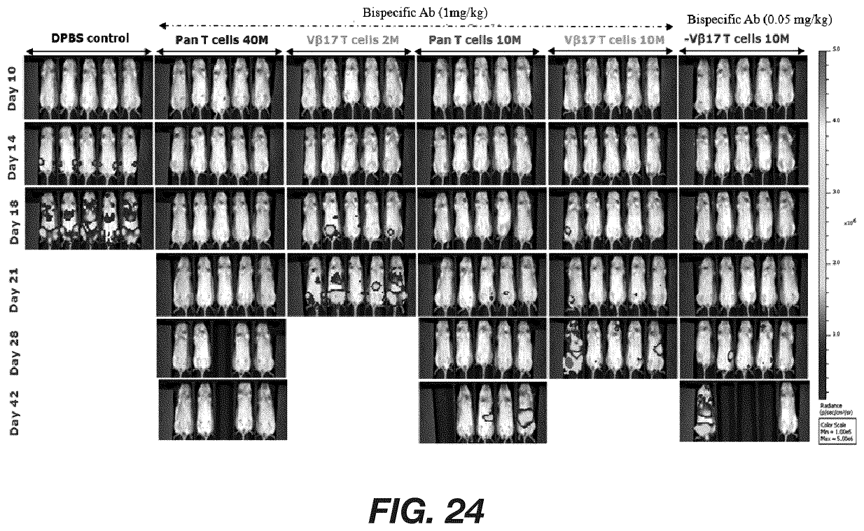

The observed AML-derived bioluminescence signals demonstrated a characteristic distribution pattern of MOLM-13-luc cells, homing to the hind limbs (ventral image, FIG. 24) and spinal column (dorsal image, data not shown) of the mice. Treatment with the bispecific antibody reduced tumor burden visually, with a more pronounced effect in the groups receiving higher numbers of T-cell humanization.

### Efficacy of the Bispecific Antibody in OCI-AML3-Luc Established Disseminated Model in T-Cell-Humanized Mice

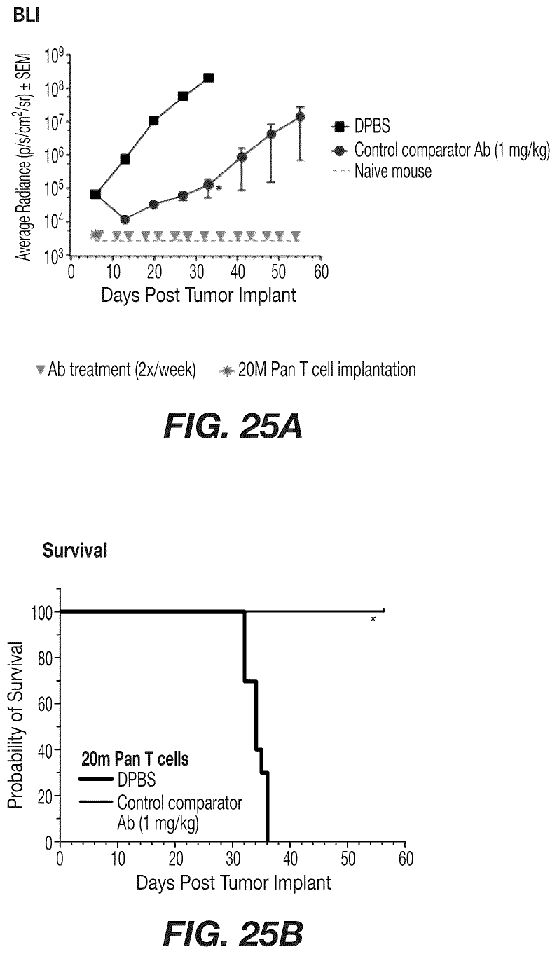

The ability of the bispecific antibody to control AML disseminated disease was evaluated in a second xenograft model, i.e., OCI-AML3-luc. In Study ONC2023-210, mice bearing established OCI-AML3-luc xenografts were IP dosed with DPBS or the bispecific antibody twice weekly at 0.01, 0.1, and 1 mg/kg for a total of 14 doses (n=10/group). Mice were humanized with either 2×10⁷ pan CD3+ or 1×10⁷ TRBV19⁺ T cells. As a reference, mice humanized with 2×10⁷ pan CD3⁺ T cells were treated with the control comparator antibody A at 1 mg/kg (FIGS. 25A and 25B).

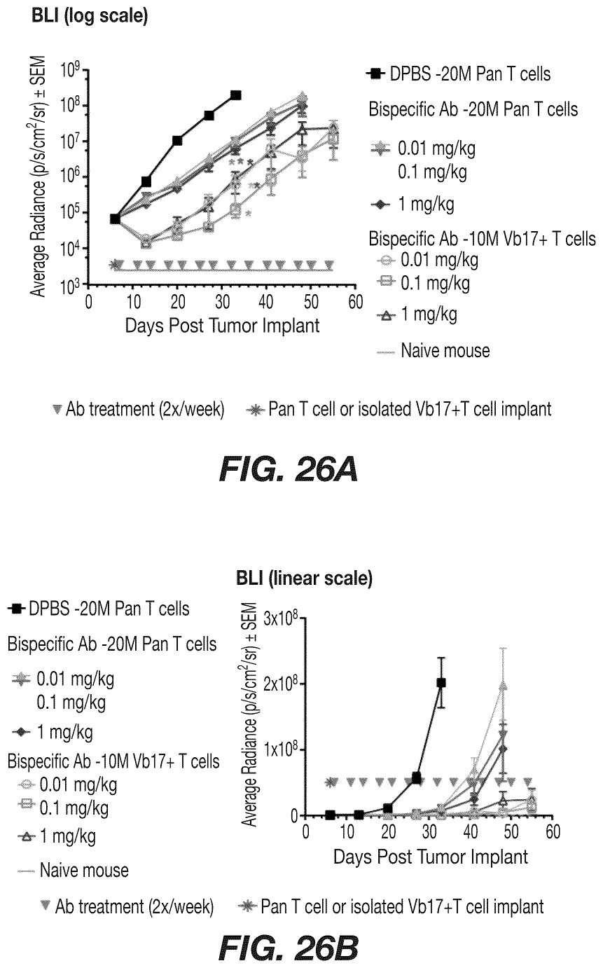

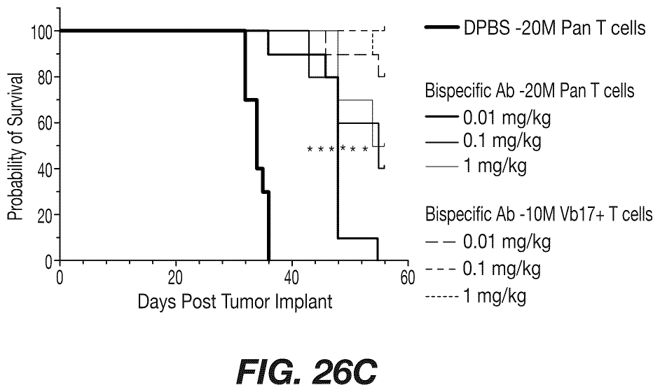

In mice humanized with 1×10⁷ TRBV19⁺ T cells, significant antitumor efficacy was observed with the bispecific antibody treatment at all tested doses (0.01, 0.1, and 1 mg/kg) assessed by change in mean tumor burden on Day 33 post tumor implantation (p<0.001), as compared to the DPBS-treated group with 100% ΔTGI in all treated groups (FIGS. 26A and 26B). At Day 33, partial regression was observed in 2 mice in the 0.01 mg/kg group and in 4 mice in both the 0.1 and 1 mg/kg treatment groups. In mice humanized with 2×10⁷ pan CD3⁺ T cells (corresponding to ˜1×10⁶ TRBV19⁺ T cells), significant antitumor efficacy was observed with the bispecific antibody 2 treatment at all tested doses (0.01, 0.1, and 1 mg/kg) on Day 33 (p<0.001), as compared to the DPBS-treated group, with 94%, 95%, and 97% ΔTGI, respectively (FIG. 26A).

The bispecific antibody at all tested doses (0.01, 0.1, and 1 mg/kg) in mice humanized with 1×10⁷ TRBV19⁺ T cells resulted in median survival of >56 days compared to the median survival of 34 days in the DPBS-treated control group resulting in a biologically significant ILS (i.e., >25%) of >65% (FIG. 26C). In mice humanized with 2×10⁷ pan CD3⁺ T cells, treatment with the bispecific antibody at 0.01 mg/kg resulted in a median survival of 48 days with a biologically significant 41% ILS, while treatment with 0.1 and 1 mg/kg the bispecific antibody resulted in a median survival of 55 days, resulting in a biologically significant 62% ILS, compared to DPBS control. Statistical differences in survival curves were observed with the bispecific antibody treatment as compared to DPBS (p<0.0001).

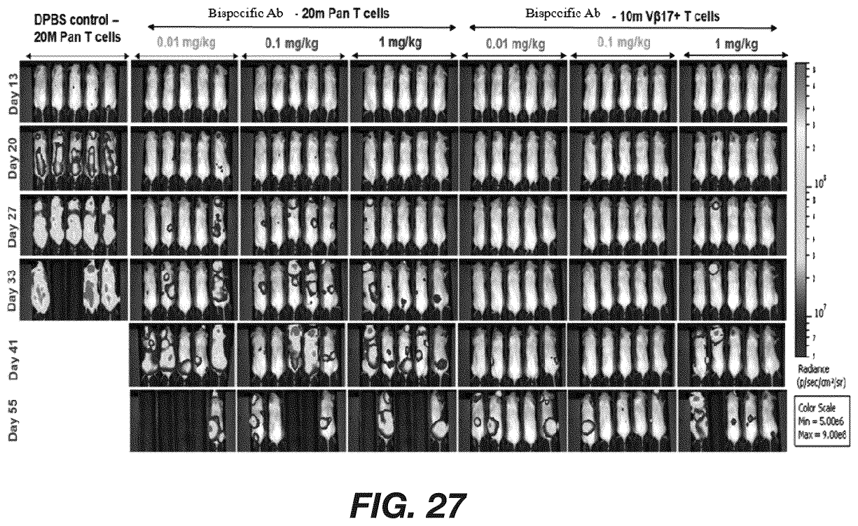

The observed AML-derived bioluminescence signals demonstrated a characteristic distribution pattern of OCI-AML3-luc cells, homing with the highest degree to the spinal column (dorsal image, FIG. 27) and also to the hind limbs. Treatment with the bispecific antibody reduced tumor burden visually, with a more pronounced effect in the groups receiving higher numbers of T-cell humanization. Although the bispecific antibody reduced or eliminated tumor burden in the primary locations (spine and hind limb), BLI was also observed in secondary tumor locations due to homing of the cancer cells in immune-privileged sites such as the ovaries, lymph nodes, and central nervous system, areas not typically associated with AML. BLI imaging coupled with micro-computed tomography analysis performed on Day 32 showed that the bispecific antibody completely eradicated primary disease as evidenced by the lack of signal in the spinal cord of the treated mice compared to DPBS-treated group. Further analysis on Day 57 continued to show lack of tumor burden in the spinal cord and hind limbs of the mice while BLI signals were detected in the lymph nodes, ovaries, and SC tissue, highlighting the efficacy of the bispecific antibody in eradicating primary AML progression.

Taken together, the present in vitro and in vivo results document the bispecific antibody's ability to induce potent and TAA-specific cytotoxicity, while leading to low levels of T-cell activation and cytokine release.

In vitro, the bispecific antibody led to cytotoxicity in a panel of AML cancer cell models showing different levels of EMR2 surface expression. No impact on cancer cell viability of TAA-negative cell lines was reported. T-cell activation was exclusively observed within the TRBV19⁺ T-cell subpopulation, leading to low levels of pan-T-cell activation. Most importantly, selective engagement and activation of TRBV19⁺ T cells was associated with low levels of cytokine production, further validating the hypothesis of lowering the risk of CRS with specific TRBV19⁺ T-cell engagement.

In vivo, treatment with the bispecific antibody demonstrated robust efficacy in 2 different AML models (i.e., MOLM-13-luc and OCI-AML3-luc). The results from the present in vivo studies demonstrated that the bispecific antibody can inhibit AML progression leading to a significant increase in the animal's life span.

Overall, the present results are in line with the present hypothesis of the TRBV19⁺ platform aiming at selectively recruiting a small population of TRBV19⁺ T cells by the bispecific antibody, to develop a TCE with an improved therapeutic window and a potential for less CRS.

### Example 7. Potential for On-Target/Off-Tumor Efficacy

Although EMR2 expression is predominantly observed in myeloid lineages (monocytes/macrophages/dendritic cells/mast cells) across various tissues, the target is also expressed in healthy HSCs and to low/limited levels in granulocytes (basophils, eosinophils, and neutrophils), immature NK cells, B cells, and T cells. The potential for on-target effects of the bispecific antibody on HSCs, granulocytes (basophils and neutrophils), and on immature NK cells was evaluated to determine on-target/off-tumor toxicity of the bispecific antibody.

### Normal Tissue Expression of EMR2 in Humans

With TCE antibodies, on-target/off-tumor toxicity has been identified as a risk in cases where expression of the targeted TAA(s) is not restricted to the tumor. Normal human expression data in the public domain on EMR2-related immune cell and tissue expression were reviewed. Propriety databases including DICE database, FANTOM5, Blueprint, and bulk RNA-Seq showed EMR2 expression is enriched in HSCs, progenitor cells, and myeloid lineage cells, and weakly or not expressed in lymphoid lineage cells. Peer-reviewed literature shows that EMR2 expression is enriched in myeloid cells, primarily monocytic and to a lesser extent granulocytic and lineages, including mast cells, and absent or very weakly expressed by the lymphoid cell lines Ramos (B-lymphoblastic) and Jurkat (T-lymphoblastic), respectively. Based on internal data as well as publicly available databases, low level of EMR2 expression was observed in small subset of B cells, NK cells and T cells.

In addition, EMR2 target expression profiling by immunohistochemistry (IHC) was performed using normal human formalin-fixed, paraffin-embedded (FFPE) tissue samples and human mast cell pellet. The IHC assay relied on the mouse monoclonal anti-EMR2 antibody clone [2A1](ThermoFisher, MA5-28205; 8) that was qualified for FFPE matrix on suitable controls. Assay development included confirmation of specificity of this reagent to EMR2 (ADGRE2) based on no cross-reactivity to CD97 (ADGRE5). EMR2 IHC positive labeling was confirmed in the human mast cell pellet and, in tissues, predominantly observed in mononuclear leukocytes with morphology most consistent with myeloid lineages (monocytes/macrophages/dendritic cells/mast cells) across various tissues: BM, colon and small intestine (lamina propria), gallbladder (lamina propria and muscle layer), liver (macrophages and Kupffer cells), spleen (predominantly leukocyte in the red pulp), lymph nodes, tonsil, appendix, lung (subepithelium and interstitium), skin (dermis), kidney (interstitium and glomeruli), and interstitium of ovary and pancreas. Granulocytes (neutrophils and eosinophils) were weakly labeled across tissues; neutrophils labeled in muscle layer in one of the three gallbladders. EMR2 IHC was negative in cerebellum, cerebrum, and meninges.

### Whole-Blood Neutrophil, Monocyte, NK Cell, and Basophil Activation Assay

A flow-cytometry-based human whole-blood assay was performed to assess if the bispecific antibody has a functional impact on peripheral neutrophils, monocytes, and/or NK cells. Preliminary results demonstrated that the bispecific antibody, but not a NullxTRBV19 negative control antibody, resulted in a moderate level of activation of monocytes and neutrophils, and to a lesser extent NK cells, after an overnight culture, albeit with variability in responses between donors. Further characterization of whether leukocyte activation is a result of direct, or indirect target engagement will be determined.

To assess the bispecific antibody results in activation of peripheral blood basophils, a flow cytometric assay was conducted in human whole blood. No basophil cell activation was detected (n=3 donors, t≤40 minutes), indicating a low risk of anaphylactoid reactions due to direct target engagement on basophils.

### Effect on HSPCs

To assess whether the bispecific antibody has an effect on the ability of HSPCs to proliferate and differentiate appropriately, in vitro CFU cell assays were conducted by setting up a coculture assay of pan-T cells and CD34⁺ cells or OCI-AML3 cells with the bispecific antibody at 0.001 to 100 nM. Results demonstrated that the bispecific antibody impacted clonogenic potential of HSPCs (≥1 nM), however in this in vitro system there was a greater inhibition of the colony formation potential of OCI-AML3 cancer cells (≥0.01 nM).

### Potential for Off-Target Binding

### Off-Target Binding by Retrogenix Screen

The potential for off-target binding of the bispecific antibody was evaluated in a human cell microarray platform (Retrogenix, Charles River Laboratories) to determine binding specificity. The antibody was screened for binding against fixed, transfected human HEK293 cells, individually expressing 6,105 full-length human plasma membrane proteins, secreted proteins, and cell surface-tethered secreted proteins plus a further 400 human heterodimers. The bispecific antibody was demonstrated to bind specifically to its primary target, EMR2, with strong intensity, and did not show any off-target interactions. Of note, TRBV19 was not in the library screen, and thus binding to TRBV19 could not be demonstrated for the bispecific antibody with this methodology. However, the bispecific antibody was shown herein to bind to TRBV19⁺ cells by flow cytometry. These results indicate a low risk for off-target mediated toxicity for the bispecific antibody.

### Example 8. Binding of the Bispecific Antibody to TRBV19-Positive T Cells

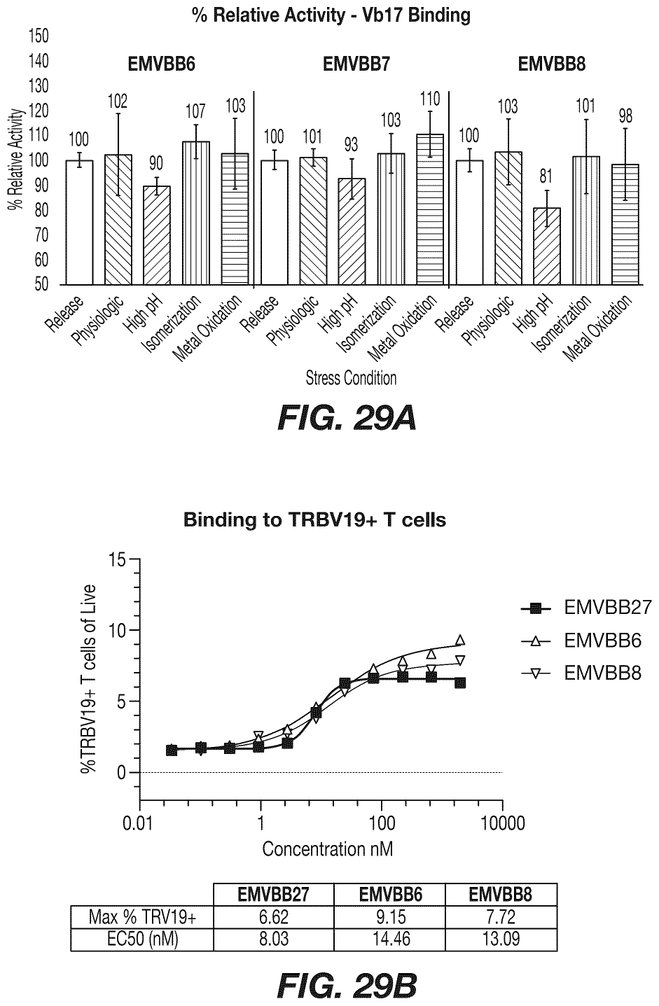

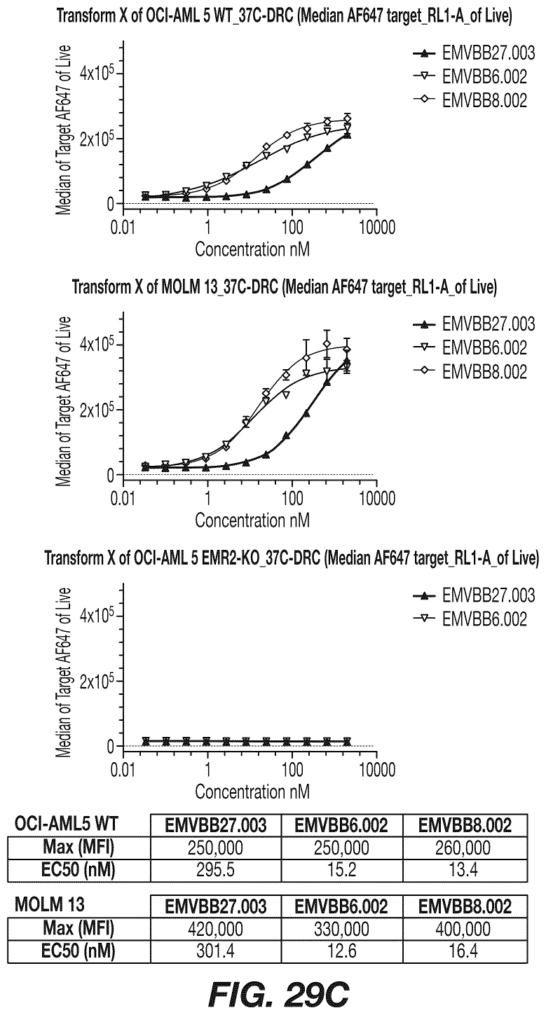

Flow cytometry was used to measure specific binding of the TRBV19 arm of bispecific EMR2xTRBV19 leads to TRBV19-positive T cells from 2 different healthy human pan-T cell donors. When pan T cells were administered increasing concentrations of bispecific antibody for 1 hour at 37° C., concentration-dependent binding was observed. The EC50 (nM) of detection of TRBV19-positive cells was between 8.03 to 14.46 nM. (FIG. 29B).

Flow cytometry was also used to measure EMR2 arm binding affinity of bispecific lead molecules to varying EMR2 expressing cell lines (OCI-AML5 and MOLM13). When these cell lines were administered increasing concentrations of bispecific antibodies at 1 hour, 37° C., concentration-dependent binding was observed. OCI-AML 5 EMR2-KO cells were used, which had the EMR2 gene genetically deleted, no detectable binding was observed. (FIG. 29C).

### Example 9. Chemical and Post-Translational Modification (PTM) in Degradation Conditions of EMVBB8

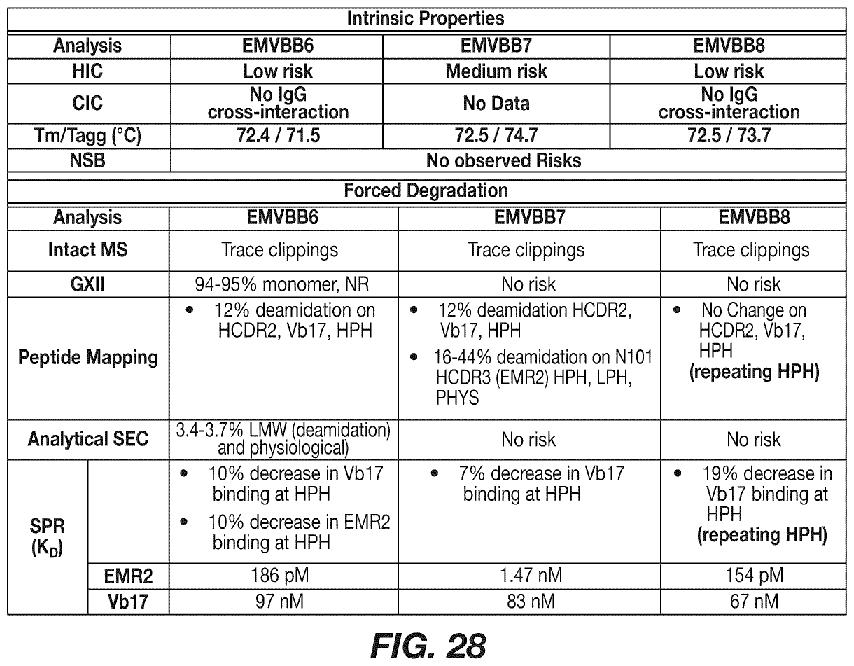

Experiments were performed to determine the binding of the bispecific antibody EMVBB8 in a high pH of 8.5 under forced degradation conditions. Initial binding experiments by SPR were performed using the listed conditions: CM4 sensorchip coupled with anti-human-Fab antibody. Antibodies were captured to the chip surface and a titration of recombinant TRBV19 concentration ranging from 140-17.5 nM, 3-fold dilution was flown over the chip surface to assess kinetic profiles. Results for the SPR binding experiments are shown in FIG. 28. As indicated in the first set of binding experiments, there was a 19% decrease in Vb17 binding at high pH (HPH) as shown in FIG. 28. However, experiments were repeated in the same assay format but using a recombinant TRBV19 concentration range of 350-43.75 nM, 2-fold dilution and show that the binding of the EMR2 arm and the TRBV19 arm bind with similar binding affinity at pH 8.5 and 5.0 using SPR (Table 6). The optimized antigen concentration range allowed for full saturation of the TRBV19 arm and data analysis software was able to accurately determine Rmax values used in % Activity calculations. This differed from the first set of conditions shown in FIG. 28 where software was approximating Rmax causing variation and error in reported Rmax and subsequently % Activity. Furthermore, the second set of data utilized a different sample batch, wherein the second batch of bispecific antibodies for Table 6 was prepared in 10 mM Acetate, pH 5.5 for enhanced stability. The first batch of bispecific antibodies was prepared in phosphate buffered saline (PBS).

### Embodiments

The disclosure provided herein also provides the following non-limiting embodiments:
1. An antibody or an antigen-binding fragment thereof, that specifically binds the G-protein-coupled receptor auto-proteolysis inducing (GAIN) domain and/or GPCR proteolytic site (GPS) motif of epidermal-growth-factor-like module-containing mucin-like hormone receptor 2 (EMR2).
2. The antibody or the antigen-binding fragment thereof, according to item 1, wherein the GAIN domain comprises amino acid residues D261-Q478 of SEQ ID NO: 205.
3. The antibody or the antigen-binding fragment thereof, according to item 1, wherein the GAIN domain comprises the amino acid sequence SEQ ID NO: 215.
4. The antibody or the antigen-binding fragment thereof, according to any one of items 1-3, wherein the GPS motif comprises the amino acid sequence SEQ ID NO: 216.
5. The antibody or the antigen-binding fragment thereof, according to any one of items 1-4, wherein the antibody, or the antigen-binding fragment thereof, binds to an epitope comprising the amino acid sequence of SEQ ID NO: 217 or SEQ ID NO: 218.
6. The antibody or the antigen-binding fragment thereof, according to any one of items 1-5, wherein the antibody, or the antigen-binding fragment thereof, binds to human EMR2 with a dissociation constant (KD) between about 0.01 nM to about 5 nM.
7. The antibody or the antigen-binding fragment thereof, according to any one of items 1-5, wherein the antibody, or the antigen-binding fragment thereof, binds to human EMR2 with a half maximal effective concentration EC50 between about 0.1 nM to about 15 nM.
8. The antibody or the antigen-binding fragment thereof, according to any one of items 1-7, wherein the antibody, or the antigen-binding fragment thereof, is or comprises a, a Fab fragment, a F(ab′)2 fragment, F(ab)′3 fragments, a scFv, a bis-scFv, a (scFv)2, a stapled scFv (spFv), a diabody, a minibody, a nanobody, a triabody, a tetrabody, a disulfide stabilized Fv protein (dsFv), a single-domain antibody (sdAb), an Immunoglobulin New Antigen Receptor (Ig NAR), a single heavy chain antibody, a camelid antibody, a shark antibody, or a chemically modified derivative thereof.
9. The antibody or the antigen-binding fragment thereof, according to any one of items 1-8, wherein the antibody, or the antigen-binding fragment thereof, comprises a Fab.
10. The antibody or the antigen-binding fragment thereof, according to any one of items 1-9, wherein the antibody, or the antigen-binding fragment thereof, further comprises a first constant Ig domain of the heavy chain (CH1 domain).
11. The antibody or the antigen-binding fragment thereof, according to any one of items 1-8, wherein the antibody, or the antigen-binding fragment thereof, does not comprise a CH1 domain.
12. The antibody or the antigen-binding fragment thereof, according to any one of items 1-8 and 11, wherein the antibody, or the antigen-binding fragment thereof, comprises a scFv or a spFv.
13. The antibody or the antigen-binding fragment thereof, according to item 12, wherein the scFv or spFv comprises a signal sequence, a heavy chain variable sequence, a GS-Linker, and a light chain variable sequence.
14. The antibody or the antigen-binding fragment thereof, according to any one of items 1-8 further comprising an Fc domain.
15. The antibody or the antigen-binding fragment thereof, according to item 14, wherein the Fc domain of the antibody or the antigen-binding fragment thereof, is an IgA, an IgG, an IgE, or an IgM.
16. The antibody or the antigen-binding fragment thereof, according to item 14 or item 15, wherein the Fc domain of the antibody or the antigen-binding fragment thereof, is an IgG.
17. The antibody or the antigen-binding fragment thereof, according to item 16, wherein the IgG is IgG1 or IgG4.
18. The antibody or the antigen-binding fragment thereof, according to any one of items 14-17, wherein the Fc domain comprises one or more different mutations which promote heterodimerization.
19. The antibody or the antigen-binding fragment thereof, according to any one of items 14-18, wherein the Fc domain comprises mutations T366S, L368A and Y407V (EU numbering) or mutation T366W (EU numbering).
20. The antibody or the antigen-binding fragment thereof, according to any one of items 14-19, wherein the Fe domains of HC1 and/or HC2 further comprise one or more mutations which reduce Fc binding to a Fc receptor.
21. The antibody or the antigen-binding fragment thereof, according to item 20, wherein the Fcγ receptor is FcyRI, FcyRIIA, FcyRIIB, FcyRIIIA, and/or FcyRIIIB.
22. The antibody or the antigen-binding fragment thereof, according to any one of items 14-21, wherein the Fc domain comprises one or more mutations selected from L234A, L235A, and D265S (EU numbering).
23. The antibody or the antigen-binding fragment thereof, according to any one of items 14-22, wherein the Fc domain comprises mutations L234A, L235A, and D265S (EU numbering).
24. The antibody or the antigen-binding fragment thereof, according to any one of claims 14-23, wherein the Fc domain further comprises one or more mutations which reduce Fc binding to protein A.
25. The antibody or the antigen-binding fragment thereof, according to any one of items 14-24, wherein the Fc domain comprises mutations H435R and/or Y436F (EU numbering).
26. The antibody or the antigen-binding fragment thereof, according to any one of items 14-25, wherein the Fc domain comprises mutations H435R and Y436F (EU numbering).
27. The antibody or the antigen-binding fragment thereof, according to any one of items 14-26, wherein the antibody, or the antigen-binding fragment thereof, comprises a humanized antibody or an antigen binding fragment thereof, a human antibody or an antigen binding fragment thereof, a murine antibody or an antigen binding fragment thereof, a chimeric antibody or an antigen binding fragment thereof, a monospecific antibody or a monospecific antigen binding fragment thereof, a bispecific antibody or a bispecific antigen binding fragment thereof, a multispecific antibody or a multispecific antigen binding fragment thereof.
28. The antibody or the antigen-binding fragment thereof, according to any one of items 1-27, wherein the antibody, or the antigen-binding fragment thereof, comprises: a) a heavy chain complementarity determining region (HCDR) 1, a HCDR2 and a HCDR3 of the heavy chain variable region (VH) of SEQ ID NO: 63, and a light chain complementarity determining region (LCDR) 1, a LCDR2 and a LCDR3 of the light chain variable region (VL) of SEQ ID NO: 64; b) a HCDR1, a HCDR2, and a HCDR3 of the VH of SEQ ID NO: 95, and a LCDR1, a LCDR2, and a LCDR3 of the VL of SEQ ID NO: 96; c) a HCDR1, a HCDR2, and a HCDR3 of the VH of SEQ ID NO: 127, and a LCDR1, a LCDR2, and a LCDR3 of the VL of SEQ ID NO: 128; or d) a HCDR1, a HCDR2, and a HCDR3 of the VH of SEQ ID NO: 191, and a LCDR1, a LCDR2, and a LCDR3 of the VL of SEQ ID NO: 192.
29. The antibody or the antigen-binding fragment thereof, according to any one of items 1-28, wherein the antibody, or the antigen-binding fragment thereof, comprises the HCDR1, the HCDR2, the HCDR3, the LCDR1, the LCDR2 and the LCDR3 of: a) SEQ ID NO: 33, 34, 35, 48, 49, and 50, respectively; b) SEQ ID NO: 36, 37, 38, 51, 52, and 53, respectively; c) SEQ ID NO: 39, 40, 41, 54, 55, and 56, respectively; d) SEQ ID NO: 42, 43, 44, 57, 58, and 59, respectively; e) SEQ ID NO: 45, 46, 47, 60, 61, and 62, respectively; f) SEQ ID NO: 65, 66, 67, 80, 81, and 82, respectively; g) SEQ ID NO: 68, 69, 70, 83, 84, and 85, respectively; h) SEQ ID NO: 71, 72, 73, 86, 87, and 88, respectively; i) SEQ ID NO: 74, 75, 76, 89, amino acid sequence AAI, and SEQ ID NO: 91, respectively; j) SEQ ID NO: 77, 78, 79, 92, 93, and 94, respectively; k) SEQ ID NO: 97, 98, 99, 112, 113, and 114, respectively; l) SEQ ID NO: 100, 101, 102, 115, 116, and 117, respectively; m) SEQ ID NO: 103, 104, 105, 118, 119, and 120, respectively; n) SEQ ID NO: 106, 107, 108, 121, amino acid sequence DNN, and SEQ ID NO: 123, respectively; o) SEQ ID NO: 109, 110, 111, 124, 125, and 126, respectively; p) SEQ ID NO: 161, 162, 163, 176, 177, and 178, respectively; q) SEQ ID NO: 164, 165, 166, 179, 180, and 181, respectively; r) SEQ ID NO: 167, 168, 169, 182, 18, and 184, respectively; s) SEQ ID NO: 170, 171, 172, 185, amino acid sequence EVS, and SEQ ID NO:187, respectively; or t) SEQ ID NO: 173, 174, 175, 188, 189, and 190, respectively.
30. The antibody or the antigen-binding fragment thereof, according to any one of items 1-29, wherein the antibody, or the antigen-binding fragment thereof, comprises a variable heavy chain region (VH) comprising an amino acid sequence that is at least 90%, at least 91%, at least 92%, at least 93%, at least 94%, at least 95%, at least 96%, at least 97%, at least 98%, at least 99% or 100% identical to: a) the VH of SEQ ID NO: 63; b) the VH of SEQ ID NO: 95; c) the VH of SEQ ID NO: 127; or d) the VH of SEQ ID NO: 191.
31. The antibody or the antigen-binding fragment thereof, according to any one of items 1-30, wherein the antibody, or the antigen-binding fragment thereof, comprises or further comprises a variable light chain region (VL) comprising an amino acid sequence that is at least 90%, at least 91%, at least 92%, at least 93%, at least 94%, at least 95%, at least 96%, at least 97%, at least 98%, at least 99% or 100% identical to: a) the VL of SEQ ID NO: 64; b) the VL of SEQ ID NO: 96; c) the VL of SEQ ID NO: 128; or d) the VL of SEQ ID NO: 192.
32. The antibody or the antigen-binding fragment thereof, according to any one of items 1-31, wherein the antibody, or the antigen-binding fragment thereof, comprises: a) the VH of SEQ ID NO: 63 and the VL of SEQ ID NO: 64; b) the VH of SEQ ID NO: 95 and the VL of SEQ ID NO: 96; c) the VH of SEQ ID NO: 127 and the VL of SEQ ID NO: 128; or d) the VH of SEQ ID NO: 191 and the VL of SEQ ID NO: 192.
33. The antibody or the antigen-binding fragment thereof, according to any one of items 1-32, wherein the antibody, or the antigen-binding fragment thereof, comprises a scFv or a spFV which comprises, from the N- to C-terminus, a VH, a linker (L) and a VL in the format VH-L-VL or a VL, a linker and a VH in the format VL-L-VH.
34. The antibody or the antigen-binding fragment thereof, according to item 33, wherein the VH comprises the amino acid sequence of SEQ ID NO: 191, the VL comprises the amino acid sequence of SEQ ID NO: 192, and the linker comprises SEQ ID NO: 221.
35. The antibody or the antigen-binding fragment thereof, according to item 33, wherein the linker comprises SEQ ID NO: 221.
36. The antibody or the antigen-binding fragment thereof, according to any one of items 1-35, comprising a heavy chain comprising the amino acid sequence of SEQ ID NO: 194, 196, 198, or 202.
37. The antibody or the -binding fragment thereof, according to any one of items 1-36, wherein the light chain comprises the amino acid sequence of SEQ ID NO: 195, 197 or 199.
38. The antibody or the antigen-binding fragment thereof, according to item 36 or item 37, wherein: a) the HC1 comprises the amino acid sequence of SEQ ID NO: 194 and the LC1 comprises the amino acid sequence of SEQ ID NO: 195; b) the HC1 comprises the amino acid sequence of SEQ ID NO: 196 and the LC1 comprises the amino acid sequence of SEQ ID NO: 197; or c) the HC1 comprises the amino acid sequence of SEQ ID NO: 198 and the LC1 comprises the amino acid sequence of SEQ ID NO: 199.
39. An antibody or an antigen-binding fragment thereof, that binds to the same epitope of EMR2 as the antibody or the antigen-binding fragment thereof, according to any one of items 27-37.
40. An antibody or an antigen-binding fragment thereof, that competes for binding to the same epitope of EMR2 with the antibody or the antigen-binding fragment thereof, according to any one of items 28-39.
41. The antibody or the antigen-binding fragment thereof, according to any one of items 1-40, wherein the antibody, or the antigen-binding fragment thereof, is a monospecific antibody.
42. The antibody or the antigen-binding fragment thereof, according to any one of items 1-40, wherein the antibody, or the antigen-binding fragment thereof, is a bispecific antibody which specifically binds to EMR2 and to a second antigen.
43. The antibody or the antigen-binding fragment thereof, according to item 42, wherein the second antigen is T-cell Receptor Variable 19 (TRBV19).
44. An isolated polynucleotide encoding the antibody or the antigen-binding fragment thereof, according to any one of items 1-43.
45. The isolated polynucleotide of item 44, wherein the polynucleotide comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: a) the VH of SEQ ID NO: 63; b) the VH of SEQ ID NO: 95; c) the VH of SEQ ID NO: 127; or d) the VH of SEQ ID NO: 191.
46. The isolated polynucleotide of item 44 or item 45, wherein the polynucleotide comprises or further comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: a) the VL of SEQ ID NO: 64; b) the VL of SEQ ID NO: 96; c) the VL of SEQ ID NO: 128; or d) the VL of SEQ ID NO: 192.
47. The isolated of any one of items 44-46, wherein the polynucleotide comprises a sequence comprising a nucleotide sequence encoding: a) the amino acid sequence of SEQ ID NO: 63 and/or SEQ ID NO: 64; b) the amino acid sequence of SEQ ID NO: 95 and/or SEQ ID NO: 96; c) the amino acid sequence of SEQ ID NO: 127 and/or SEQ ID NO: 128; or d) the amino acid sequence of SEQ ID NO: 191 and/or the SEQ ID NO: 192.
48. The isolated polynucleotide according to item 46 or item 47, wherein the polynucleotide comprises a sequence encoding a heavy chain comprising the amino acid sequence of SEQ ID NO: 194, 196, 198, or 202.
49. The isolated polynucleotide according to any one of items 44-48, wherein the polynucleotide comprises a sequence encoding a light chain comprising the amino acid sequence of SEQ ID NO: 195, 197, or 199.
50. The isolated polynucleotide of claim any one of items 44-49, comprising a nucleotide sequence encoding: a) the amino acid sequence of SEQ ID NO: 194 and/or SEQ ID NO: 195; b) the amino acid sequence of SEQ ID NO: 196 and/or SEQ ID NO: 197; c) the amino acid sequence of SEQ ID NO: 198 and/or the amino acid sequence of SEQ ID NO: 199; or d) the amino acid sequence of SEQ ID NO: 202.
51. A vector comprising the isolated polynucleotide according to any one of items 44-50.
52. The vector according to item 51, wherein the isolated polynucleotide is operably linked to an expression control sequence.
53. The vector according to item 51 or item 52, wherein the vector is a viral vector.
54. The vector according to item 53, wherein the viral vector is selected from an adenoviral vector, an adeno-associated viral vector, a retroviral vector, a lentiviral vector, a herpes simplex virus vector, and a poxvirus vector.
55. A pharmaceutical composition comprising (i) the antibody or the antigen-binding fragment thereof, according to any one of items 1-43, or the polynucleotide according to any one of items 44-50, or the vector according to any one of items 51-54, and (ii) a pharmaceutically acceptable carrier or excipient.
56. A host cell expressing the antibody or the antigen-binding fragment thereof, according to any one of items 1-43.
57. The host cell according to item 56, wherein the cell is a hybridoma.
58. The host cell according to item 56, wherein the antibody, or the antigen-binding fragment thereof, is recombinantly produced.
59. A host cell comprising the isolated polynucleotide according to any one of claims 44-50 or the vector according to any one of items 51-54.
60. A method for treating a cancer in a subject in need thereof, the method comprising administering to the subject a therapeutically effective amount of the antibody or the antigen-binding fragment thereof, according to any one of items 1-43, or the isolated polynucleotide according to any one of items 44-50, or the vector according to any one of items 51-54, or the pharmaceutical composition of claim 55, or the host cell according to any one of items 56-59.
61. A method for inducing cytotoxicity of a cancer cell or redirecting immune or T cells against cancer cells, the method comprising administering to the cell an effective amount of the antibody or the antigen-binding fragment thereof, according to any one of items 1-43, or the polynucleotide according to any one of items 44-50, or the vector according to any one of items 51-54, or the pharmaceutical composition of item 55, or the host cell according to any one of items 56-59, wherein the effective amount is sufficient to inhibit the growth or proliferation of the cancer cell.
62. The method according to item 61, wherein the cancer cell is in a subject and the antibody or the binding fragment, polynucleotide, vector, pharmaceutical composition, or host cell is administered to the subject.
63. The method according to item 61 or item 62, wherein the administration is conducted ex vivo.
64. The method according to any one of items 61-63, wherein the cancer is an EMR2-expressing cancer.
65. The method according to item 64, wherein the EMR2-expressing cancer is a hematological cancer.
66. The method according to item 65, wherein the hematological cancer is a myeloid malignancy.
67. The method according to item 66, wherein the hematological cancer is an acute myeloid leukemia (AML), a chronic myelogenous leukemia (CML), or a myelodysplastic neoplasm.
68. The method according to any one of items 60-67 further comprising administering a second therapeutic agent.
69. The method according to item 68, wherein the second therapeutic agent is a surgery, a chemotherapy, an androgen deprivation therapy, a radiation, or any combination thereof.
70. The antibody according to any one of items 1-43 for use in the method according to any one of items 60-69.
71. The polynucleotide according to any one of items 44-50 for use in the method according to any one of items 60-69.
72. The vector according to any one of items 51-54 for use in the method according to any one of items 60-69.
73. The pharmaceutical composition according to item 55 for use in the method according to any one of items 60-69.
74. The host cell according to any one of items 56-59 for use in the method according to any one of items 60-69.
75. A method for generating the antibody or the antigen-binding fragment thereof, according to any one of items 1-43, wherein the method comprises culturing the host cell according to any one of items 56-59 and isolating the antibody or the antigen-binding fragment thereof.
76. A kit comprising (i) the antibody or the antigen-binding fragment thereof, according to any one of items 1-43 and/or the isolated polynucleotide according to any one of items 44-50, and/or the vector according to any one of items 51-54, and/or the pharmaceutical composition of item 55, and/or the host cell according to any one of items 56-59, and (ii) packaging for the same and/or instructions for use.
77. A bispecific antibody or a bispecific antigen-binding fragment thereof, that specifically binds (i) TRBV19 with a first antigen-binding site and (ii) GAIN domain and/or GPS motif of EMR2 with a second antigen-binding site.
78. The bispecific antibody or the bispecific antigen-binding fragment thereof, according to item 77, comprising: a) a first heavy chain (HC1); b) optionally a first light chain (LC1); c) a second heavy chain (HC2); and d) optionally a second light chain (LC2), wherein (i) the HC1 and the LC1 form a first antigen-binding site that specifically binds a first antigen, (ii) the HC2 and the LC2 form a second antigen-binding site that specifically binds a second antigen, (iii) the HC1 and HC2 each comprise a Fc domain comprising a CH2-CH3 domain; and wherein the first antigen is TRBV19, and the second antigen is EMR2.
79. The bispecific antibody or the bispecific antigen-binding fragment thereof, according to item 77 or item 78, wherein the second antigen-binding site specifically binds the GAIN domain and/or GPS motif of EMR2.
80. The bispecific antibody or the bispecific antigen-binding fragment thereof, according to item 79, wherein the GAIN domain comprises amino acid residues D261-Q478 of SEQ ID NO: 205.
81. The bispecific antibody or the bispecific antigen-binding fragment thereof, according to item 79, wherein the GAIN domain comprises the amino acid sequence SEQ ID NO: 215.
82. The bispecific antibody or bispecific antigen-binding fragment thereof, according to any one of items 77-81, wherein the GPS motif comprises the amino acid sequence SEQ ID NO: 216.
83. The bispecific antibody or bispecific antigen-binding fragment thereof, according to any one of items 77-82, wherein the antibody, or the antigen-binding fragment thereof, binds to an epitope comprising the amino acid sequence of SEQ ID NO: 217 or SEQ ID NO: 218.
84. The bispecific antibody or the bispecific antigen-binding fragment thereof, according to any one of items 77-83, wherein the first antigen-binding site comprises a Fab or a scFv or a spFv.
85. The bispecific antibody or the bispecific antigen-binding fragment thereof, according to any one of items 77-84, wherein the Fc of the first antigen-binding site comprises a CH1 domain.
86. The bispecific antibody or the bispecific antigen-binding fragment thereof, according to any one of items 77-85, wherein the second antigen-binding site comprises a Fab or a scFv or a spFv.
87. The bispecific antibody or the bispecific antigen-binding fragment thereof, according to any one of items 77-86, wherein the Fc of the second antigen-binding site further comprises a CH1 domain.
88. The bispecific antibody or the bispecific antigen-binding fragment thereof, according to any one of items 77-87, wherein the first antigen-binding site comprises a Fab and the second antigen-binding site comprises a scFv or a spFv.
89. The bispecific antibody or the bispecific antigen-binding fragment thereof, according to any one of items 77-88, wherein the first antigen-binding site comprises a scFv or a spFv and the second antigen-binding site comprises a Fab.
90. The bispecific antibody or the bispecific antigen-binding fragment thereof, according to any one of items 77-89, wherein the Fc domain of the antibody or the antigen-binding fragment thereof, is an IgA, an IgG, an IgE, or an IgM.
91. The bispecific antibody or the bispecific antigen-binding fragment thereof, according to item 90, wherein the Fc domain of the antibody or the antigen-binding fragment thereof, is an IgG.
92. The bispecific antibody or the bispecific antigen-binding fragment thereof, according to item 91, wherein the IgG is IgG1 or IgG4.
93. The bispecific antibody or the bispecific antigen-binding fragment thereof, according to any one of items 77-92, wherein the Fc domains of HC1 and HC2 comprise one or more different mutations which promote heterodimerization.
94. The bispecific antibody or the bispecific antigen-binding fragment thereof, according to any one of items 77-93, wherein the Fc domain of the HC1 comprises mutations T366S, L368A and Y407V (EU numbering) and the Fc domain of the HC2 comprises mutation T366W (EU numbering).
95. The bispecific antibody or the bispecific antigen-binding fragment thereof, according to any one of items 77-94, wherein the Fc domain of the HC2 comprises mutations T366S, L368A and Y407V (EU numbering) and the Fc domain of the HC1 comprises mutation T366W (EU numbering).
96. The bispecific antibody or the bispecific antigen-binding fragment thereof, according to any one of items 77-95, wherein the Fc domains of HC1 and/or HC2 further comprise one or more mutations which reduce Fc binding to a Fc receptor.
97. The bispecific antibody or the bispecific antigen-binding fragment thereof, according to item 96, wherein the Fc receptor is FcγRI, FcγRIIA, FcγRIIB, FcyRIIIA, and/or FcγRIIIB.
98. The bispecific antibody or the bispecific antigen-binding fragment thereof, according to any one of items 77-97, wherein the Fc domains of HC1 and/or HC2 each comprise one or more mutations selected from L234A, L235A, and D265S (EU numbering).
99. The bispecific antibody or the bispecific antigen-binding fragment thereof, according to any one of items 77-98, wherein the Fe domains of HC1 and HC2 each comprise mutations L234A, L235A, and D265S (EU numbering).
100. The bispecific antibody or the bispecific antigen-binding fragment thereof, according to any one of items 77-99, wherein the Fc domains of HC1 or HC2 further comprises one or more mutations which reduce Fc binding to protein A.
101. The bispecific antibody or the bispecific antigen-binding fragment thereof, according to any one of items 77-100, wherein the Fc domains of HC1 or HC2 comprise mutations H435R and/or Y436F (EU numbering).
102. The bispecific antibody or the bispecific antigen-binding fragment thereof, according to any one of items 770101, wherein the Fc domain of HC1 comprises mutations H435R and Y436F (EU numbering).
103. The bispecific antibody or the bispecific antigen-binding fragment thereof, according to any one of items 77-102, wherein the Fc domain of HC2 comprises mutations H435R and Y436F (EU numbering).
104. The bispecific antibody or the bispecific antigen-binding fragment thereof, according to any one of items 77-103, wherein the HC1 or HC2 comprise mutation C220S (EU numbering).
105. The bispecific antibody or the bispecific antigen-binding fragment thereof, according to any one of items 77-104, wherein the HC1 comprises mutation C220S (EU numbering).
106. The bispecific antibody or the bispecific antigen-binding fragment thereof, according to any one of items 77-104, wherein the HC2 comprises mutation C220S (EU numbering).
107. The bispecific antibody or the bispecific antigen-binding fragment thereof, according to any one of items 77-106, wherein the antibody, or the antigen-binding fragment thereof, is a humanized antibody or an antigen binding fragment thereof, a human antibody or an antigen binding fragment thereof, a murine antibody or an antigen binding fragment thereof, a chimeric antibody or an antigen binding fragment thereof, or a chemically modified derivative thereof.
108. The bispecific antibody or the bispecific antigen-binding fragment thereof, according to any one of items 77-107, wherein the first antigen-binding site that specifically binds TRBV19 comprises: a) a HCDR1, a HCDR2 and a HCDR3 of the VH of SEQ ID NO: 31 and a LCDR1, a LCDR2 and a LCDR3 of the VL of SEQ ID NO: 32; or b) a HCDR1, a HCDR2, and a HCDR3 of the VH of SEQ ID NO: 159 and a LCDR1, LCDR2, and LCDR3 of the VL of SEQ ID NO: 160.
109. The bispecific antibody or the bispecific antigen-binding fragment thereof, according to item 108, wherein the first antigen-binding site that specifically binds TRBV19 comprises the HCDR1, the HCDR2, the HCDR3, the LCDR1, the LCDR2 and the LCDR3 of: a) SEQ ID NOs: 1, 2, 3, 16, 17, and 18, respectively; b) SEQ ID NOs: 4, 5, 6, 19, 20, and 21, respectively; c) SEQ ID NOs: 7, 8, 9, 22, 23, and 24, respectively; d) SEQ ID NOs: 10, 11, 12, 25, amino acid sequence KVS, and SEQ ID NO: 27, respectively; e) SEQ ID NOs: 13, 14, 15, 28, 29, and 30, respectively; f) SEQ ID NOs: 129, 130, 131, 144, 145, and 146, respectively; g) SEQ ID NOs: 132, 133, 134, 147, 148, and 149, respectively; h) SEQ ID NOs: 135, 136, 137, 150, 151, and 152, respectively; i) SEQ ID NOs: 138, 139, 140, 153, amino acid sequence KVS, and SEQ ID NO: 155, respectively; or j) SEQ ID NOs: 141, 142, 143, 156, 157, and 158, respectively.
110. The bispecific antibody or the bispecific antigen-binding fragment thereof, according to any one of items 77-109, wherein the first antigen-binding site that specifically binds TRBV19 comprises a VH comprising an amino acid sequence that is at least 90%, at least 91%, at least 92%, at least 93%, at least 94%, at least 95%, at least 96%, at least 97%, at least 98%, at least 99% or 100% identical to: the VH of SEQ ID NO: 31; or the VH of SEQ ID NO: 159.
111. The bispecific antibody or the bispecific antigen-binding fragment thereof, according to any one of items 77-110, wherein the first antigen-binding site that specifically binds TRBV19 comprises or further comprises a VL comprising an amino acid sequence that is at least 90%, at least 91%, at least 92%, at least 93%, at least 94%, at least 95%, at least 96%, at least 97%, at least 98%, at least 99% or 100% identical to: the VL of SEQ ID NO: 32; or the VL of SEQ ID NO: 160.
112. The bispecific antibody or the bispecific antigen-binding fragment thereof, according to any one of items 77-111, wherein the first antigen-binding site that specifically binds TRBV19 comprises: the VH of SEQ ID NO: 31 and the VL of SEQ ID NO: 32; or the VH of SEQ ID NO: 159 and the VL of SEQ ID NO: 160.
113. The bispecific antibody or the bispecific antigen-binding fragment thereof, according to any one of items 77-112, wherein the second antigen-binding site that specifically binds EMR2 comprises: a) a HCDR1, a HCDR2 and a HCDR3 of the VH of SEQ ID NO: 63, and a LCDR1, a LCDR2 and a LCDR3 of the VL of SEQ ID NO: 64; b) a HCDR1, a HCDR2, and a HCDR3 of the VH of SEQ ID NO: 95, and a LCDR1, a LCDR2, and a LCDR3 of the VL of SEQ ID NO: 96; c) a HCDR1, a HCDR2, and a HCDR3 of the VH of SEQ ID NO: 127, and a LCDR1, a LCDR2, and a LCDR3 of the VL of SEQ ID NO: 128; or d) a HCDR1, a HCDR2, and a HCDR3 of the VH of SEQ ID NO: 191, and a LCDR1, a LCDR2, and a LCDR3 of the VL of SEQ ID NO: 192.
114. The bispecific antibody or the bispecific antigen-binding fragment thereof, according to any one of items 77-113, wherein the second antigen-binding site that specifically binds EMR2 comprises the HCDR1, the HCDR2, the HCDR3, the LCDR1, the LCDR2 and the LCDR3 of: a) SEQ ID NO: 33, 34, 35, 48, 49, and 50, respectively; b) SEQ ID NO: 36, 37, 38, 51, 52, and 53, respectively; c) SEQ ID NO: 39, 40, 41, 54, 55, and 56, respectively; d) SEQ ID NO: 42, 43, 44, 57, amino acid sequence EVS, and SEQ ID NO: 59, respectively; e) SEQ ID NO: 45, 46, 47, 60, 61, and 62, respectively; f) SEQ ID NO: 65, 66, 67, 80, 81, and 82, respectively; g) SEQ ID NO: 68, 69, 70, 83, 84, and 85, respectively; h) SEQ ID NO: 71, 72, 73, 86, 87, and 88, respectively; i) SEQ ID NO: 74, 75, 76, 89, amino acid sequence AAI, and SEQ ID NO: 91, respectively; j) SEQ ID NO: 77, 78, 79, 92, 93, and 94, respectively; k) SEQ ID NO: 97, 98, 99, 112, 113, and 114, respectively; l) SEQ ID NO: 100, 101, 102, 115, 116, and 117, respectively; m) SEQ ID NO: 103, 104, 105, 118, 119, and 120, respectively; n) SEQ ID NO: 106, 107, 108, 121, amino acid sequence DNN, and SEQ ID NO: 123, respectively; o) SEQ ID NO: 109, 110, 111, 124, 125, and 126, respectively; p) SEQ ID NO: 161, 162, 163, 176, 177, and 178, respectively; q) SEQ ID NO: 164, 165, 166, 179, 180, and 181, respectively; r) SEQ ID NO: 167, 168, 169, 182, 18, and 184, respectively; s) SEQ ID NO: 170, 171, 172, 185, amino acid sequence EVS, and SEQ ID NO:187, respectively; or t) SEQ ID NO: 173, 174, 175, 188, 189, and 190, respectively.
115. The bispecific antibody or the bispecific antigen-binding fragment thereof, according to any one of items 77-114, wherein the bispecific antibody or the bispecific antigen-binding fragment thereof, comprises a VH comprising an amino acid sequence that is at least 90%, at least 91%, at least 92%, at least 93%, at least 94%, at least 95%, at least 96%, at least 97%, at least 98%, at least 99% or 100% identical to: a) the VH of SEQ ID NO: 63; b) the VH of SEQ ID NO: 95; c) the VH of SEQ ID NO: 127; or d) the VH of SEQ ID NO: 191.
116. The bispecific antibody or the bispecific antigen-binding fragment thereof, according to any one of items 77-115, wherein the bispecific antibody or the bispecific antigen-binding fragment thereof, comprises or further comprises a VL comprising an amino acid sequence that is at least 90%, at least 91%, at least 92%, at least 93%, at least 94%, at least 95%, at least 96%, at least 97%, at least 98%, at least 99% or 100% identical to: a) the VL of SEQ ID NO: 64; b) the VL of SEQ ID NO: 96; c) the VL of SEQ ID NO: 128; or d) the VL of SEQ ID NO: 192.
117. The bispecific antibody or the bispecific antigen-binding fragment thereof, according to any one of items 77-116, wherein the first antigen-binding site and/or the second antigen-binding site that specifically binds EMR2 comprises: a) the VH of SEQ ID NO: 63 and the VL of SEQ ID NO: 64; b) the VH of SEQ ID NO: 95 and the VL of SEQ ID NO: 96; c) the VH of SEQ ID NO: 127 and the VL of SEQ ID NO: 128; or d) the VH of SEQ ID NO: 191 and the VL of SEQ ID NO: 192.
118. The bispecific antibody or the bispecific antigen-binding fragment thereof, according to any one of items 77-117, wherein the bispecific antibody or the bispecific antigen-binding fragment thereof, comprises a scFv or a spFV which scFv or spFV comprises, from the N- to C-terminus, a VH, a linker (L) and a VL in the format VH-L-VL or a VL, a linker and a VH in the format VL-L-VH.
119. The bispecific antibody or the bispecific antigen-binding fragment thereof, according to item 118, wherein the VH comprises the amino acid sequence of SEQ ID NO: 159, the VL comprises the amino acid sequence of SEQ ID NO: 160, and the L comprises SEQ ID NO: 221.
120. The bispecific antibody or the bispecific antigen-binding fragment thereof, according to item 118, wherein the VH comprises the amino acid sequence of SEQ ID NO: 191, the VL comprises the amino acid sequence of SEQ ID NO: 192, and the L comprises SEQ ID NO: 221.
121. The bispecific antibody or the bispecific antigen-binding fragment thereof, according to item 118, wherein the L comprises SEQ ID NO: 221.
122. The bispecific antibody or the bispecific antigen-binding fragment thereof, according to any one of items 77-121, wherein the first antigen-binding site specifically binds to TRBV19 with a dissociation constant (KD) that is between about 15 nM to about 200 nM.
123. The bispecific antibody or the bispecific antigen-binding fragment thereof, according to any one of items 77-121, wherein the first antigen-binding site specifically binds to TRBV19 with an EC50 between about 1 nM to about 100 nM.
124. The bispecific antibody or the bispecific antigen-binding fragment thereof, according to any one of items 77-123, wherein the second antigen-binding site specifically binds to EMR2 with a dissociation constant (KD) that is between about 0.01 nM to about 5 nM.
125. The bispecific antibody or the bispecific antigen-binding fragment thereof, according to any one of items 77-123, wherein the second antigen-binding site specifically binds to EMR2 with an EC50 between about 0.1 nM to about 15 nM.
126. The bispecific antibody or the bispecific binding fragment thereof, according to any one of items 77-125, wherein the HC1 comprises the amino acid sequence of SEQ ID NO: 193 or 200.
127. The bispecific antibody or the bispecific binding fragment thereof, according to any one of items 77-126, wherein the LC1 comprises the amino acid sequence of SEQ ID NO: 201.
128. The bispecific antibody or the bispecific binding fragment thereof, according to any one of items 77-127, wherein a) the HC1 comprises the amino acid sequence of SEQ ID NO: 193; or b) the HC1 comprises the amino acid sequence of SEQ ID NO: 200 and the LC1 comprises the amino acid sequence of SEQ ID NO: 201.
129. The bispecific antibody or the bispecific binding fragment thereof, according to any one of items 77-128, wherein the HC2 comprises the amino acid sequence of SEQ ID NO: 194, 196, 198, or 202.
130. The bispecific antibody or the bispecific binding fragment thereof, according to any one of items 77-129, wherein the LC2 comprises the amino acid sequence of SEQ ID NO: 195, 197, or 199.
131. The bispecific antibody or the bispecific binding fragment thereof, according to item 130, wherein a) the HC2 comprises the amino acid sequence of SEQ ID NO: 194 and the LC2 comprises the amino acid sequence of SEQ ID NO: 195; b) the HC2 comprises the amino acid sequence of SEQ ID NO: 196 and the LC1 comprises the amino acid sequence of SEQ ID NO: 197; c) the HC2 comprises the amino acid sequence of SEQ ID NO: 198 and the LC1 comprises the amino acid sequence of SEQ ID NO: 199; or d) the HC2 comprises the amino acid sequence of SEQ ID NO: 202.
132. The bispecific antibody or the bispecific binding fragment thereof, according to item 130 or item 131, wherein a) the HC1 comprises the amino acid sequence of SEQ ID NO: 193, the HC2 comprises the amino acid sequence of SEQ ID NO: 194, and the LC2 comprises the amino acid sequence of SEQ ID NO 195; b) the HC1 comprises the amino acid sequence of SEQ ID NO: 193, the HC2 comprises the amino acid sequence of SEQ ID NO: 196, and the LC2 comprises the amino acid sequence of SEQ ID NO: 197; c) the HC1 comprises the amino acid sequence of SEQ ID NO: 193, the HC2 comprises the amino acid sequence of SEQ ID NO: 198, and the LC2 comprises the amino acid sequence of SEQ ID NO: 199; or d) the HC1 comprises the amino acid sequence of SEQ ID NO: 200, the LC1 comprises the amino acid sequence of SEQ ID NO: 201, and the HC2 comprises the amino acid sequence of SEQ ID NO: 202.
133. A bispecific antibody or a bispecific antigen-binding fragment thereof, that binds to the same epitope as the bispecific antibody or the bispecific antigen-binding fragment thereof, according to any one of items 77-132.
134. A bispecific antibody or a bispecific antigen-binding fragment thereof, that competes for binding to the same epitope with the bispecific antibody or the bispecific antigen-binding fragment thereof, according to any one of items 77-132.
135. A bispecific antibody, or a bispecific antigen-binding fragment thereof, wherein the bispecific antibody or the bispecific antigen-binding fragment thereof, comprises an HC1 comprising the amino acid sequence of SEQ ID NO: 193, an HC2 comprising the amino acid sequence of SEQ ID NO: 198, and an LC2 comprising the amino acid sequence of SEQ ID NO: 199.
136. An isolated polynucleotide encoding the bispecific antibody or the bispecific binding fragment of any one of items 77-135.
137. The polynucleotide according to item 136, wherein the polynucleotide comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: a) the VH of SEQ ID NO: 63; b) the VH of SEQ ID NO: 95; c) the VH of SEQ ID NO: 127; or d) the VH of SEQ ID NO: 191.
138. The polynucleotide according to item 136 or item 137, wherein the polynucleotide comprises or further comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: a) the VL of SEQ ID NO: 64; b) the VL of SEQ ID NO: 96; c) the VL of SEQ ID NO: 128; or d) the VL of SEQ ID NO: 192.
139. The polynucleotide according to any one of items 136-138, wherein the polynucleotide comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: a) the VH of SEQ ID NO: 31; or b) the VH of SEQ ID NO: 159.
140. The polynucleotide according to any one of items 136-138, wherein the polynucleotide comprises or further comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to: a) the VL of SEQ ID NO: 32; or b) the VL of SEQ ID NO: 160.
141. The polynucleotide according to any one of items 136-140, wherein the polynucleotide comprises a sequence encoding a first antigen-binding site that specifically binds TRBV19, the sequence comprising a nucleotide sequence encoding: a) the amino acid sequence of SEQ ID NO: 31 and/or the amino acid sequence of SEQ ID NO: 32; or b) the amino acid sequence of SEQ ID NO: 159 and/or the amino acid sequence of SEQ ID NO: 160.
142. The polynucleotide according to any one of items 136-141, wherein the polynucleotide comprises a sequence encoding a second antigen-binding site that specifically binds EMR2, the sequence comprising a nucleotide sequence encoding: a) the amino acid sequence of SEQ ID NO: 63 and/or the amino acid sequence of SEQ ID NO: 64; b) the amino acid sequence of SEQ ID NO: 95 and/or the amino acid sequence of SEQ ID NO: 96; c) the amino acid sequence of SEQ ID NO: 127 and/or the amino acid sequence of SEQ ID NO: 128; or d) the amino acid sequence of SEQ ID NO: 191 and/or the amino acid sequence of SEQ ID NO: 192.
143. The polynucleotide according to any one of items 136-142, wherein the polynucleotide comprises a sequence encoding an HC1 comprising the amino acid sequence of SEQ ID NO: 193 or 200.
144. The polynucleotide according to any one of items 136-143, wherein the polynucleotide comprises a sequence encoding a LC1 comprising the amino acid sequence of SEQ ID NO: 201.
145. The polynucleotide according to any one of items 136-144, comprising a nucleotide sequence encoding: a) the amino acid sequence of SEQ ID NO: 193; or b) the amino acid sequence of SEQ ID NO: 200 and the amino acid sequence of SEQ ID NO: 201.
146. The polynucleotide according to any one of items 136-145, wherein the polynucleotide comprises a sequence encoding an HC2 comprising the nucleotide sequence of SEQ ID NO: 194, 196, 198, or 202.
147. The polynucleotide according to any one of items 136-146, wherein the polynucleotide comprises a sequence encoding a LC2 comprising the nucleotide sequence of SEQ ID NO: 195, 197, or 199.
148. The polynucleotide according to any one of items 136-147, wherein the polynucleotide comprises a sequence encoding: a) the amino acid sequence of SEQ ID NO: 194 and/or the amino acid sequence of SEQ ID NO: 195; b) the amino acid sequence of SEQ ID NO: 196 and/or the amino acid sequence of SEQ ID NO: 197; c) the amino acid sequence of SEQ ID NO: 198 and/or the amino acid sequence of SEQ ID NO: 199; or d) the amino acid sequence of SEQ ID NO: 202.
149. The polynucleotide according to claim any one of items 136-148, wherein the polynucleotide comprises a sequence encoding: a) the amino acid sequence of SEQ ID NO: 193, the amino acid sequence of SEQ ID NO: 194 and the amino acid of SEQ ID NO: 195; b) the amino acid sequence of SEQ ID NO: 193, the amino acid sequence of SEQ ID NO: 196 and the amino acid sequence of SEQ ID NO: 197; c) the amino acid sequence of SEQ ID NO: 193, the amino acid sequence of SEQ ID NO: 198 and the amino acid sequence of SEQ ID NO: 199; or d) the amino acid sequence of SEQ ID NO: 200, the amino acid sequence of SEQ ID NO: 201, and the amino acid sequence of SEQ ID NO: 202.
150. A vector comprising the polynucleotide according to any one of items 136-149.
151. The vector according to item 150, wherein the polynucleotide is operably linked to an expression control sequence.
152. The vector according to item 150 or item 151, wherein the vector is a viral vector.
153. The vector according to item 152, wherein the viral vector is selected from an adenoviral vector, an adeno-associated viral vector, a retroviral vector, a lentiviral vector, a herpes simplex virus vector, and a poxvirus vector.
154. A pharmaceutical composition comprising (i) the bispecific antibody or the bispecific antigen-binding fragment thereof, according to any one of items 77-135, or the polynucleotide according to any one of items 136-149, or the vector according to any one of items 150-153, and (ii) a pharmaceutically acceptable carrier or excipient.
155. A host cell expressing the bispecific antibody or the bispecific antigen-binding fragment thereof, according to any one of items 77-135.
156. The host cell according to item 155, wherein the cell is a hybridoma.
157. The host cell according to item 155, wherein the bispecific antibody or the bispecific antigen-binding fragment thereof, is recombinantly produced.
158. A host cell comprising the polynucleotide according to any one of items 136-149 or the vector according to any one of items 150-153.
159. A method for treating cancer in a subject in need thereof, the method comprising administering to the subject a therapeutically effective amount of the bispecific antibody or the bispecific antigen-binding fragment thereof, according to any one of items 77-135, or the polynucleotide according to any one of items 136-149, or the vector according to any one of items 150-153, or the pharmaceutical composition of item 154.
160. A method for inducing cytotoxicity of a cancer cell or redirecting immune or T cells to cancer cells, the method comprising administering to the cell an effective amount of the bispecific antibody or the bispecific antigen-binding fragment thereof, according to any one of items 77-135, or the polynucleotide according to any one of items 136-149, or the vector according to any one of items 150-153, or the pharmaceutical composition of item 154, or the host cell according to any one of items 155-158, wherein the effective amount is sufficient to inhibit the growth or proliferation of the cancer cell.
161. The method according to item 160, wherein the cancer cell is in a subject and the bispecific antibody or the bispecific antigen-binding fragment thereof, the polynucleotide, the vector, the pharmaceutical composition, or the host cell is administered to the subject.
162. The method according to item 160 or item 161, wherein the administration is conducted ex vivo.
163. A method of redirecting a T cell to EMR2-expressing cancer cells in a subject in need thereof, the method comprising administering to the subject a therapeutically effective amount of the bispecific antibody or the bispecific antigen-binding fragment thereof, according to any one of items 77-135, or the polynucleotide according to any one of items 136-149, or the vector according to any one of items 150-153, or the pharmaceutical composition of item 154, or the host cell according to any one of items 155-158.
164. The method according to item 163, wherein the therapeutically effective amount is sufficient to direct the T cell response to the cancer cells.
165. The method according to any one of items 159-164, wherein the cancer is an EMR2-expressing cancer.
166. The method according to item 165, wherein the EMR2-expressing cancer is a hematological cancer.
167. The method according to item 166, wherein the hematological cancer is a myeloid malignancy.
168. The method according to item 167, wherein the cancer is acute myeloid leukemia (AML), chronic myelogenous leukemia (CML), or myelodysplastic neoplasms.
169. The method according to any one of items 161-168 further comprising administering a second therapeutic agent.
170. The method according to item 169, wherein the second therapeutic agent is a surgery, a chemotherapy, an androgen deprivation therapy, or a radiation, or any combination thereof.
171. The bispecific antibody or the bispecific antigen-binding fragment thereof, according to any one of items 77-135 for use in the method according to any one of claims 159-170.
172. The polynucleotide according to any one of items 133-146 for use in the method according to any one of items 159-170.
173. The vector according to any one of items 150-153 for use in the method according to any one of items 159-170.
174. The pharmaceutical composition according to item 154 for use in the method according to any one of items 159-170.
175. The host cell according to any one of items 155-158, for use in the method according to any one of items 159-170.
176. A method for generating the bispecific antibody or the bispecific antigen-binding fragment thereof, according to any one of items 77-135, wherein the method comprises culturing the host cell according to any one of items 155-158, and isolating the bispecific antibody or the bispecific binding fragment.
177. A kit comprising (i) the bispecific antibody or the bispecific antigen-binding fragment thereof, according to any one of items 77-135, or the polynucleotide according to any one of items 136-149, or the vector according to any one of items 150-153, or the pharmaceutical composition of item 154, or the host cell according to any one of items 155-158, and (ii) packaging for the same and/or instructions for use.

Any references in the description or in the claims to methods of treatment refer to the compounds, compositions, pharmaceutical compositions and medicaments for use in a method of treatment of the human (or animal) body by therapy (or for diagnosis).

### List of Sequences

VR000015623 HC AbM CDR1
SEQ ID NO: 1
GYSITSGYFWN
VR000015623 HC AbM CDR2
SEQ ID NO: 2
YISYDGSNN
VR000015623 HC AbM CDR3
SEQ ID NO: 3
PSPGTGYAVDY
VR000015623 HC KABAT CDR1
SEQ ID NO: 4
SGYFWN
VR000015623 HC KABAT CDR2
SEQ ID NO: 5
YISYDGSNNYNPSLKS
VR000015623 HC KABAT CDR3
SEQ ID NO: 6
PSPGTGYAVDY
VR000015623 HC CHOTHIA CDR1
SEQ ID NO: 7
GYSITSGY
VR000015623 HC CHOTHIA CDR2
SEQ ID NO: 8
SYDGS
VR000015623 HC CHOTHIA CDR3
SEQ ID NO: 9
PSPGTGYAVDY
VR000015623 HC IMGT CDR1
SEQ ID NO: 10
GYSITSGYF
VR000015623 HC IMGT CDR2
SEQ ID NO: 11
ISYDGSN
VR000015623 HC IMGT CDR3
SEQ ID NO: 12
ASPSPGTGYAVDY
VR000015623 HC CONTACT CDR1
SEQ ID NO: 13
TSGYFWN
VR000015623 HC CONTACT CDR2
SEQ ID NO: 14
WIGYISYDGSNN
VR000015623 HC CONTACT CDR3
SEQ ID NO: 15
ASPSPGTGYAVD
VR000015623 LC AbM CDR1
SEQ ID NO: 16
RSSQSLVHSSGNTYLH
VR000015623 LC AbM CDR2
SEQ ID NO: 17
KVSNRFS
VR000015623 LC AbM CDR3
SEQ ID NO: 18
SQSTHVPFT
VR000015623 LC KABAT CDR1
SEQ ID NO: 19
RSSQSLVHSSGNTYLH
VR000015623 LC KABAT CDR2
SEQ ID NO: 20
KVSNRFS
VR000015623 LC KABAT CDR3
SEQ ID NO: 21
SQSTHVPFT
VR000015623 LC CHOTHIA CDR1
SEQ ID NO: 22
RSSQSLVHSSGNTYLH
VR000015623 LC CHOTHIA CDR2
SEQ ID NO: 23
KVSNRFS
VR000015623 LC CHOTHIA CDR3
SEQ ID NO: 24
SQSTHVPFT
VR000015623 LC IMGT CDR1
SEQ ID NO: 25
QSLVHSSGNTY
VR000015623 LC IMGT CDR2
KVS
VR000015623 LC IMGT CDR3
SEQ ID NO: 27
SQSTHVPFT
VR000015623 LC CONTACT CDR1
SEQ ID NO: 28
VHSSGNTYLHWY
VR000015623 LC CONTACT CDR2
SEQ ID NO: 29
FLIYKVSNRF
VR000015623 LC CONTACT CDR3
SEQ ID NO: 30
SQSTHVPF
VR000015623 VH sequence
SEQ ID NO: 31
QVQLQESGPGLVKPSETLSLTCTVSGYSITSGYFWNWIRQPPGKGLEWIGYISYD
GSNNYNPSLKSRVTISRDTSKNQFSLKLSSVTAADTAVYYCASPSPGTGYAVDYWGQGT
LVTVSS
VR000015623 VL sequence
SEQ ID NO: 32
DIQMTQSPSSLSASVGDRVTITCRSSQSLVHSSGNTYLHWYQQKPGKAPKFLIYK
VSNRFSGVPSRFSGSGSGTDFTLTISSLQPEDFATYYCSQSTHVPFTFGQGTKLEIK
VR000049550 HC AbM CDR1
SEQ ID NO: 33
GFTFSSYWMH
VR000049550 HC AbM CDR2
SEQ ID NO: 34
RISSHGSSTS
VR000049550 HC AbM CDR3
SEQ ID NO: 35
GGNYYDSPYFDY
VR000049550 HC KABAT CDR1
SEQ ID NO: 36
SYWM
VR000049550 HC KABAT CDR2
SEQ ID NO: 37
RISSHGSSTSYADSVKG
VR000049550 HC KABAT CDR3
SEQ ID NO: 38
GGNYYDSPYFDY
VR000049550 HC CHOTHIA CDR1
SEQ ID NO: 39
GFTFSSY
VR000049550 HC CHOTHIA CDR2
SEQ ID NO: 40
SSHGSS
VR000049550 HC CHOTHIA CDR3
SEQ ID NO: 41
GGNYYDSPYFDY
VR000049550 HC IMGT CDR1
SEQ ID NO: 42
GFTFSSYW
VR000049550 HC IMGT CDR2
SEQ ID NO: 43
ISSHGSST
VR000049550 HC IMGT CDR3
SEQ ID NO: 44
ARGGNYYDSPYFDY
VR000049550 HC CONTACT CDR1
SEQ ID NO: 45
SSYWMH
VR000049550 HC CONTACT CDR2
SEQ ID NO: 46
WVSRISSHGSSTS
VR000049550 HC CONTACT CDR3
SEQ ID NO: 47
ARGGNYYDSPYFD
VR000049550 LC AbM CDR1
SEQ ID NO: 48
TGTSSDVGGYNYVS
VR000049550 LC AbM CDR2
SEQ ID NO: 49
EVSYRPS
VR000049550 LC AbM CDR3
SEQ ID NO: 50
SSYTSSSTYVV
VR000049550 LC KABAT CDR1
SEQ ID NO: 51
TGTSSDVGGYNYVS
VR000049550 LC KABAT CDR2
SEQ ID NO: 52
EVSYRPS
VR000049550 LC KABAT CDR3
SEQ ID NO: 53
SSYTSSSTYVV
VR000049550 LC CHOTHIA CDR1
SEQ ID NO: 54
TGTSSDVGGYNYVS
VR000049550 LC CHOTHIA CDR2
SEQ ID NO: 55
EVSYRPS
VR000049550 LC CHOTHIA CDR3
SEQ ID NO: 56
SSYTSSSTYVV
VR000049550 LC IMGT CDR1
SEQ ID NO: 57
SSDVGGYNY
VR000049550 LC IMGT CDR2
EVS
VR000049550 LC IMGT CDR3
SEQ ID NO: 59
SSYTSSSTYVV
VR000049550 LC CONTACT CDR1
SEQ ID NO: 60
VGGYNYVSWY
VR000049550 LC CONTACT CDR2
SEQ ID NO: 61
LMIYEVSYRP
VR000049550 LC CONTACT CDR3
SEQ ID NO: 62
SSYTSSSTYV
VR000049550 VH sequence
SEQ ID NO: 63
EVQLVESGGGLVQPGGSLRLSCAASGFTFSSYWMHWVRQVPGKGLVWVSRISS
HGSSTSYADSVKGRFTISRDNAKNALYLQMNSLRAEDTAVYYCARGGNYYDSPYFDY
WGQGTLVTVSS
VR000049550 VL sequence
SEQ ID NO: 64
QSALTQPASVSGSPGQSITISCTGTSSDVGGYNYVSWYQQHPGKAPKLMIYEVSY
RPSGISNRFSGSKSGDTASLTISGLQAEDEADYYCSSYTSSSTYVVFGGGTKVTVL
VR000049625 HC AbM CDR1
SEQ ID NO: 65
GFTFSRYSMN
VR000049625 HC AbM CDR2
SEQ ID NO: 66
YISSSSNTIY
VR000049625 HC AbM CDR3
SEQ ID NO: 67
DQGTPFDY
VR000049625 HC KABAT CDR1
SEQ ID NO: 68
RYSMN
VR000049625 HC KABAT CDR2
SEQ ID NO: 69
YISSSSNTIYYADSVKG
VR000049625 HC KABAT CDR3
SEQ ID NO: 70
DQGTPFDY
VR000049625 HC CHOTHIA CDR1
SEQ ID NO: 71
GFTFSRY
VR000049625 HC CHOTHIA CDR2
SEQ ID NO: 72
SSSSNT
VR000049625 HC CHOTHIA CDR3
SEQ ID NO: 73
DQGTPFDY
VR000049625 HC IMGT CDR1
SEQ ID NO: 74
GFTFSRYS
VR000049625 HC IMGT CDR2
SEQ ID NO: 75
ISSSSNTI
VR000049625 HC IMGT CDR3
SEQ ID NO: 76
ARDQGTPFDY
VR000049625 HC CONTACT CDR1
SEQ ID NO: 77
SRYSMN
VR000049625 HC CONTACT CDR2
SEQ ID NO: 78
WVSYISSSSNTIY
VR000049625 HC CONTACT CDR3
SEQ ID NO: 79
ARDQGTPFD
VR000049625 LC AbM CDR1
SEQ ID NO: 80
RASQDISSWLG
VR000049625 LC AbM CDR2
SEQ ID NO: 81
AAISLQS
VR000049625 LC AbM CDR3
SEQ ID NO: 82
HQANSFPLT
VR000049625 LC KABAT CDR1
SEQ ID NO: 83
RASQDISSWLG
VR000049625 LC KABAT CDR2
SEQ ID NO: 84
AAISLQS
VR000049625 LC KABAT CDR3
SEQ ID NO: 85
HQANSFPLT
VR000049625 LC CHOTHIA CDR1
SEQ ID NO: 86
RASQDISSWLG
VR000049625 LC CHOTHIA CDR2
SEQ ID NO: 87
AAISLQS
VR000049625 LC CHOTHIA CDR3
SEQ ID NO: 88
HQANSFPLT
VR000049625 LC IMGT CDR1
SEQ ID NO: 89
QDISSW
VR000049625 LC IMGT CDR2
AAI
VR000049625 LC IMGT CDR3
SEQ ID NO: 91
HQANSFPLT
VR000049625 LC CONTACT CDR1
SEQ ID NO: 92
SSWLGWY
VR000049625 LC CONTACT CDR2
SEQ ID NO: 93
LLIYAAISLQ
VR000049625 LC CONTACT CDR3
SEQ ID NO: 94
HQANSFPL
VR000049625 VH sequence
SEQ ID NO: 95
EVQLVESGGGLVQPGGSLRLSCTASGFTFSRYSMNWVRQAPGEGLEWVSYISSS
SNTIYYADSVKGRFTISRDNAKNSLYLQMNSLRDEDTAVYYCARDQGTPFDYWGQGTL
VTVSS
VR000049625 VL sequence
SEQ ID NO: 96
EIVLTQSPSSVSASIGDRVTITCRASQDISSWLGWYQQKPGKAPKLLIYAAISLQSG
VPSRFSGSESGTDFTLTISSLQPEDFATYYCHQANSFPLTFGGGTKVEIK
VR000049630 HC AbM CDR1
SEQ ID NO: 97
GFIFRSYSMN
VR000049630 HC AbM CDR2
SEQ ID NO: 98
SISSSSSYIY
VR000049630 HC AbM CDR3
SEQ ID NO: 99
REYGGPFDY
VR000049630 HC KABAT CDR1
SEQ ID NO: 100
SYSMN
VR000049630 HC KABAT CDR2
SEQ ID NO: 101
SISSSSSYIYYADSVKG
VR000049630 HC KABAT CDR3
SEQ ID NO: 102
REYGGPFDY
VR000049630 HC CHOTHIA CDR1
SEQ ID NO: 103
GFIFRSY
VR000049630 HC CHOTHIA CDR2
SEQ ID NO: 104
SSSSSY
VR000049630 HC CHOTHIA CDR3
SEQ ID NO: 105
REYGGPFDY
VR000049630 HC IMGT CDR1
SEQ ID NO: 106
GFIFRSYS
VR000049630 HC IMGT CDR2
SEQ ID NO: 107
ISSSSSYI
VR000049630 HC IMGT CDR3
SEQ ID NO: 108
ARREYGGPFDY
VR000049630 HC CONTACT CDR1
SEQ ID NO: 109
RSYSMN
VR000049630 HC CONTACT CDR2
SEQ ID NO: 110
WVSSISSSSSYIY
VR000049630 HC CONTACT CDR3
SEQ ID NO: 111
ARREYGGPFD
VR000049630 LC AbM CDR1
SEQ ID NO: 112
SGSSSNIGNDYVS
VR000049630LC AbM CDR2
SEQ ID NO: 113
DNNKRPS
VR000049630 LC AbM CDR3
SEQ ID NO: 114
GTWDSSLSAGYV
VR000049630 LC KABAT CDR1
SEQ ID NO: 115
SGSSSNIGNDYVS
VR000049630 LC KABAT CDR2
SEQ ID NO: 116
DNNKRPS
VR000049630 LC KABAT CDR3
SEQ ID NO: 117
GTWDSSLSAGYV
VR000049630 LC CHOTHIA CDR1
SEQ ID NO: 118
SGSSSNIGNDYVS
VR000049630 LC CHOTHIA CDR2
SEQ ID NO: 119
DNNKRPS
VR000049630 LC CHOTHIA CDR3
SEQ ID NO: 120
GTWDSSLSAGYV
VR000049630 LC IMGT CDR1
SEQ ID NO: 121
SSNIGNDY
VR000049630 LC IMGT CDR2
DNN
VR000049630 LC IMGT CDR3
SEQ ID NO: 123
GTWDSSLSAGYV
VR000049630 LC CONTACT CDR1
SEQ ID NO: 124
IGNDYVSWY
VR000049630 LC CONTACT CDR2
SEQ ID NO: 125
LLIYDNNKRP
VR000049630 LC CONTACT CDR3
SEQ ID NO: 126
GTWDSSLSAGY
VR000049630 VH sequence
SEQ ID NO: 127
EVQLVQSGGGLVKPGGSLRLSCVASGFIFRSYSMNWVRQAPGKGLEWVSSISSSS
SYIYYADSVKGRFTISRDNAKNSLYLQMDSLRAEDTAVYYCARREYGGPFDYWGQGTL
VTVSS
VR000049630 VL sequence
SEQ ID NO: 128
QSVLTQPPSVFAAPGEKVTISCSGSSSNIGNDYVSWYQQLPGTAPKLLIYDNNKR
PSGIPDRESGSKSGTSATLGITGLQTGDEADYYCGTWDSSLSAGYVFGTGTKVTVL
VR000071196 HC AbM CDR1
SEQ ID NO: 129
GYSITSGYFWN
VR000071196 HC AbM CDR2
SEQ ID NO: 130
YISYDGSNN
VR000071196 HC AbM CDR3
SEQ ID NO: 131
PSPGTGYAVDY
VR000071196 HC KABAT CDR1
SEQ ID NO: 132
SGYFWN
VR000071196 HC KABAT CDR2
SEQ ID NO: 133
YISYDGSNNYNPSLKS
VR000071196 HC KABAT CDR3
SEQ ID NO: 134
PSPGTGYAVDY
VR000071196 HC CHOTHIA CDR1
SEQ ID NO: 135
GYSITSGY
VR000071196 HC CHOTHIA CDR2
SEQ ID NO: 136
SYDGS
VR000071196 HC CHOTHIA CDR3
SEQ ID NO: 137
PSPGTGYAVDY
VR000071196 HC IMGT CDR1
SEQ ID NO: 138
GYSITSGYF
VR000071196 HC IMGT CDR2
SEQ ID NO: 139
ISYDGSN
VR000071196 HC IMGT CDR3
SEQ ID NO: 140
ASPSPGTGYAVDY
VR000071196 HC CONTACT CDR1
SEQ ID NO: 141
TSGYFWN
VR000071196 HC CONTACT CDR2
SEQ ID NO: 142
WMGYISYDGSNN
VR000071196 HC CONTACT CDR3
SEQ ID NO: 143
ASPSPGTGYAVD
VR000071196 LC AbM CDR1
SEQ ID NO: 144
RSSQSLVHSNRNTYLH
VR000071196 LC AbM CDR2
SEQ ID NO: 145
KVSNRFS
VR000071196 LC AbM CDR3
SEQ ID NO: 146
SQSTHVPFT
VR000071196 LC KABAT CDR1
SEQ ID NO: 147
RSSQSLVHSNRNTYLH
VR000071196 LC KABAT CDR2
SEQ ID NO: 148
KVSNRFS
VR000071196 LC KABAT CDR3
SEQ ID NO: 149
SQSTHVPFT
VR000071196 LC CHOTHIA CDR1
SEQ ID NO: 150
RSSQSLVHSNRNTYLH
VR000071196 LC CHOTHIA CDR2
SEQ ID NO: 151
KVSNRFS
VR000071196 LC CHOTHIA CDR3
SEQ ID NO: 152
SQSTHVPFT
VR000071196 LC IMGT CDR1
SEQ ID NO: 153
QSLVHSNRNTY
VR000071196 LC IMGT CDR2
KVS
VR000071196 LC IMGT CDR3
SEQ ID NO: 155
SQSTHVPFT
VR000071196 LC CONTACT CDR1
SEQ ID NO: 156
VHSNRNTYLHWY
VR000071196 LC CONTACT CDR2
SEQ ID NO: 157
LLIYKVSNRF
VR000071196 LC CONTACT CDR3
SEQ ID NO: 158
SQSTHVPF
VR000071196 VH sequence
SEQ ID NO: 159
QVQLQESGPGLVKPSETLSLTCTVSGYSITSGYFWNWIRQFPGKGLEWMGYISYD
GSNNYNPSLKSRITISRDTSKNQFSLKLSSVTAADTAVYYCASPSPGTGYAVDYWGCGT
TVTVSS
VR000071196 VL sequence
SEQ ID NO: 160
DIQMTQSPSSLSASVGDRVTITCRSSQSLVHSNRNTYLHWYQQKPGCAPKLLIYK
VSNRFSGVPSRFSGSGSGTDFTLTISSLQPEDFATYYCSQSTHVPFTFGQGTKLEIK
VR000079933 HC AbM CDR1
SEQ ID NO: 161
GFTFRSYWMH
VR000079933 HC AbM CDR2
SEQ ID NO: 162
RISSGGSTTN
VR000079933 HC AbM CDR3
SEQ ID NO: 163
GDNYYDSPYFDY
VR000079933 HC KABAT CDR1
SEQ ID NO: 164
SYWMH
VR000079933 HC KABAT CDR2
SEQ ID NO: 165
RISSGGSTTNYADSVKG
VR000079933 HC KABAT CDR3
SEQ ID NO: 166
GDNYYDSPYFDY
VR000079933 HC CHOTHIA CDR1
SEQ ID NO: 167
GFTFRSY
VR000079933 HC CHOTHIA CDR2
SEQ ID NO: 168
SSGGST
VR000079933 HC CHOTHIA CDR3
SEQ ID NO: 169
GDNYYDSPYFDY
VR000079933 HC IMGT CDR1
SEQ ID NO: 170
GFTFRSYW
VR000079933 HC IMGT CDR2
SEQ ID NO: 171
ISSGGSTT
VR000079933 HC IMGT CDR3
SEQ ID NO: 172
ARGDNYYDSPYFDY
VR000079933 HC CONTACT CDR1
SEQ ID NO: 173
RSYWMH
VR000079933 HC CONTACT CDR2
SEQ ID NO: 174
WVSRISSGGSTTN
VR000079933 HC CONTACT CDR3
SEQ ID NO: 175
ARGDNYYDSPYFD
VR000079933 LC AbM CDR1
SEQ ID NO: 176
TGTSSDVGGYNYVS
VR000079933 LC AbM CDR2
SEQ ID NO: 177
EVSYRPS
VR000079933 LC AbM CDR3
SEQ ID NO: 178
SSYTSSSTYVV
VR000079933 LC KABAT CDR1
SEQ ID NO: 179
TGTSSDVGGYNYVS
VR000079933 LC KABAT CDR2
SEQ ID NO: 180
EVSYRPS
VR000079933 LC KABAT CDR3
SEQ ID NO: 181
SSYTSSSTYVV
VR000079933 LC CHOTHIA CDR1
SEQ ID NO: 182
TGTSSDVGGYNYVS
VR000079933 LC CHOTHIA CDR2
SEQ ID NO: 183
EVSYRPS
VR000079933 LC CHOTHIA CDR3
SEQ ID NO: 184
SSYTSSSTYVV
VR000079933 LC IMGT CDR1
SEQ ID NO: 185
SSDVGGYNY
VR000079933 LC IMGT CDR2
EVS
VR000079933 LC IMGT CDR3
SEQ ID NO: 187
SSYTSSSTYVV
VR000079933 LC CONTACT CDR1
SEQ ID NO: 188
VGGYNYVSWY
VR000079933 LC CONTACT CDR2
SEQ ID NO: 189
LMIYEVSYRP
VR000079933 LC CONTACT CDR3
SEQ ID NO: 190
SSYTSSSTYV
VR000079933 VH sequence
SEQ ID NO: 191
EVQLVESGGGLVQPGGSLRLSCAASGFTFRSYWMHWVRQAPGKGLVWVSRISS
GGSTTNYADSVKGRFTISRDNAKNTLYLQMNSLRAEDTAIYYCARGDNYYDSPYFDYW
GCGTLVTVSS
VR000079933 VL sequence
SEQ ID NO: 192
QSALTQPASVSGSPGQSITISCTGTSSDVGGYNYVSWYQQHPGCAPKLMIYEVSY
RPSGVSNRFSGSKSGNTASLTISGLQAEDEADYYCSSYTSSSTYVVFGGGTKLTVL
EMVBB6 Heavy Chain 1
SEQ ID NO: 193
DIQMTQSPSSLSASVGDRVTITCRSSQSLVHSNRNTYLHWYQQKPGCAPKLLIYK
VSNRFSGVPSRFSGSGSGTDFTLTISSLQPEDFATYYCSQSTHVPFTFGQGTKLEIKGGGS
GGSGGCPPCGGSGGQVQLQESGPGLVKPSETLSLTCTVSGYSITSGYFWNWIRQFPGKG
LEWMGYISYDGSNNYNPSLKSRITISRDTSKNQFSLKLSSVTAADTAVYYCASPSPGTGY
AVDYWGCGTTVTVSSEPKSSDKTHTCPPCPAPEAAGGPSVFLFPPKPKDTLMISRTPEVT
CVVVSVSHEDPEVKFNWYVDGVEVHNAKTKPREEQYNSTYRVVSVLTVLHQDWLNG
KEYKCKVSNKALPAPIEKTISKAKGQPREPQVYTLPPSREEMTKNQVSLWCLVKGFYPS
DIAVEWESNGQPENNYKTTPPVLDSDGSFFLYSKLTVDKSRWQQGNVFSCSVMHEALH
NHYTQKSLSLSPGK
EMVBB6 Heavy Chain 2
SEQ ID NO: 194
EVQLVQSGGGLVKPGGSLRLSCVASGFIFRSYSMNWVRQAPGKGLEWVSSISSSS
SYIYYADSVKGRFTISRDNAKNSLYLQMDSLRAEDTAVYYCARREYGGPFDYWGQGTL
VTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAP
EAAGGPSVFLFPPKPKDTLMISRTPEVTCVVVSVSHEDPEVKFNWYVDGVEVHNAKTKP
REEQYNSTYRVVSVLTVLHQDWLNGKEYKCKVSNKALPAPIEKTISKAKGQPREPQVY
TLPPSREEMTKNQVSLSCAVKGFYPSDIAVEWESNGQPENNYKTTPPVLDSDGSFFLVSK
LTVDKSRWQQGNVFSCSVMHEALHNRFTQKSLSLSPGK
EMVBB6 Light Chain 2
SEQ ID NO: 195
QSVLTQPPSVFAAPGEKVTISCSGSSSNIGNDYVSWYQQLPGTAPKLLIYDNNKR
PSGIPDRESGSKSGTSATLGITGLQTGDEADYYCGTWDSSLSAGYVFGTGTKVTVLGQP
KAAPSVTLFPPSSEELQANKATLVCLISDFYPGAVTVAWKADSSPVKAGVETTTPSKQS
NNKYAASSYLSLTPEQWKSHRSYSCQVTHEGSTVEKTVAPTECS
EMVBB7 Heavy Chain 2
SEQ ID NO: 196
EVQLVESGGGLVQPGGSLRLSCAASGFTFSSYWMHWVRQVPGKGLVWVSRISS
HGSSTSYADSVKGRFTISRDNAKNALYLQMNSLRAEDTAVYYCARGGNYYDSPYFDY
WGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDKTH
TCPPCPAPEAAGGPSVFLFPPKPKDTLMISRTPEVTCVVVSVSHEDPEVKFNWYVDGVE
VHNAKTKPREEQYNSTYRVVSVLTVLHQDWLNGKEYKCKVSNKALPAPIEKTISKAKG
QPREPQVYTLPPSREEMTKNQVSLSCAVKGFYPSDIAVEWESNGQPENNYKTTPPVLDS
DGSFFLVSKLTVDKSRWQQGNVFSCSVMHEALHNRFTQKSLSLSPGK
EMVBB7 Light Chain 2
SEQ ID NO: 197
QSALTQPASVSGSPGQSITISCTGTSSDVGGYNYVSWYQQHPGKAPKLMIYEVSY
RPSGISNRFSGSKSGDTASLTISGLQAEDEADYYCSSYTSSSTYVVFGGGTKVTVLGQPK
AAPSVTLFPPSSEELQANKATLVCLISDFYPGAVTVAWKADSSPVKAGVETTTPSKQSN
NKYAASSYLSLTPEQWKSHRSYSCQVTHEGSTVEKTVAPTECS
EMVBB8 Heavy Chain 2
SEQ ID NO: 198
EVQLVESGGGLVQPGGSLRLSCTASGFTFSRYSMNWVRQAPGEGLEWVSYISSS
SNTIYYADSVKGRFTISRDNAKNSLYLQMNSLRDEDTAVYYCARDQGTPFDYWGQGTL
VTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAP
EAAGGPSVFLFPPKPKDTLMISRTPEVTCVVVSVSHEDPEVKFNWYVDGVEVHNAKTKP
REEQYNSTYRVVSVLTVLHQDWLNGKEYKCKVSNKALPAPIEKTISKAKGQPREPQVY
TLPPSREEMTKNQVSLSCAVKGFYPSDIAVEWESNGQPENNYKTTPPVLDSDGSFFLVSK
LTVDKSRWQQGNVFSCSVMHEALHNRFTQKSLSLSPGK
EMVBB8 Light Chain 2
SEQ ID NO: 199
EIVLTQSPSSVSASIGDRVTITCRASQDISSWLGWYQQKPGKAPKLLIYAAISLQSG
VPSRFSGSESGTDFTLTISSLQPEDFATYYCHQANSFPLTFGGGTKVEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC
EMVBB27 Heavy Chain 1
SEQ ID NO: 200
QVQLQESGPGLVKPSETLSLTCTVSGYSITSGYFWNWIRQPPGKGLEWIGYISYD
GSNNYNPSLKSRVTISRDTSKNQFSLKLSSVTAADTAVYYCASPSPGTGYAVDYWGQGT
LVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPCPA
PEAAGGPSVFLFPPKPKDTLMISRTPEVTCVVVSVSHEDPEVKFNWYVDGVEVHNAKTK
PREEQYNSTYRVVSVLTVLHQDWLNGKEYKCKVSNKALPAPIEKTISKAKGQPREPQV
YTLPPSREEMTKNQVSLWCLVKGFYPSDIAVEWESNGQPENNYKTTPPVLDSDGSFFLY
SKLTVDKSRWQQGNVFSCSVMHEALHNRFTQKSLSLSPGK
EMVBB27 Light Chain 1
SEQ ID NO: 201
DIQMTQSPSSLSASVGDRVTITCRSSQSLVHSSGNTYLHWYQQKPGKAPKFLIYK
VSNRFSGVPSRFSGSGSGTDFTLTISSLQPEDFATYYCSQSTHVPFTFGQGTKLEIKRTVA
APSVFIFPPSDEQLKSGTASVVCLLNNFYPREAKVQWKVDNALQSGNSQESVTEQDSKD
STYSLSSTLTLSKADYEKHKVYACEVTHQGLSSPVTKSFNRGEC
EMVBB27 Heavy Chain 2
SEQ ID NO: 202
QSALTQPASVSGSPGQSITISCTGTSSDVGGYNYVSWYQQHPGCAPKLMIYEVSY
RPSGVSNRFSGSKSGNTASLTISGLQAEDEADYYCSSYTSSSTYVVFGGGTKLTVLGGGS
GGSGGCPPCGGSGGEVQLVESGGGLVQPGGSLRLSCAASGFTFRSYWMHWVRQAPGK
GLVWVSRISSGGSTTNYADSVKGRFTISRDNAKNTLYLQMNSLRAEDTAIYYCARGDN
YYDSPYFDYWGCGTLVTVSSEPKSSDKTHTCPPCPAPEAAGGPSVFLFPPKPKDTLMISR
TPEVTCVVVSVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLSCAVKG
FYPSDIAVEWESNGQPENNYKTTPPVLDSDGSFFLVSKLTVDKSRWQQGNVFSCSVMHE
ALHNHYTQKSLSLSPGK
huEMR2 GAIN domain + GPS, D261-T540 UniprotKB Q9UHX3 with
C-terminal Avi/6x His tag (“6x His tag” disclosed as
SEQ ID NO: 226), EMR2W4
SEQ ID NO: 203
MARKSALLALALLLLGFPGAWGDMTFSTWTPPPGVHSQTLSRFFDKVQDLGRD
YKPGLANNTIQSILQALDELLEAPGDLETLPRLQQHCVASHLLDGLEDVLRGLSKNLSNG
LLNFSYPAGTELSLEVQKQVDRSVTLRQNQAVMQLDWNQAQKSGDPGPSVVGLVSIPG
MGKLLAEAPLVLEPEKQMLLHETHQGLLQDGSPILLSDVISAFLSNNDTQNLSSPVTFTF
SHRSVIPRQKVLCVFWEHGQNGCGHWATTGCSTIGTRDTSTICRCTHLSSFAVLMAHYD
VQEEDPVLTVITGGGGSGLNDIFEAQKIEWHEGGGGSHHHHHHG
huEMR2 GAIN domain, D261-Q478 UniprotKB Q9UHX3 with C-
terminal Avi/6x His tag (“6x His tag” disclosed as
SEQ ID NO: 226), EMR2W6
SEQ ID NO: 204
MARKSALLALALLLLGFPGAWGDMTFSTWTPPPGVHSQTLSRFFDKVQDLGRD
YKPGLANNTIQSILQALDELLEAPGDLETLPRLQQHCVASHLLDGLEDVLRGLSKNLSNG
LLNFSYPAGTELSLEVQKQVDRSVTLRQNQAVMQLDWNQAQKSGDPGPSVVGLVSIPG
MGKLLAEAPLVLEPEKQMLLHETHQGLLQDGSPILLSDVISAFLSNNDTQNLSSPVTFTF
SHRSVIPRQGGGGSGLNDIFEAQKIEWHEGGGGSHHHHHHG
huEMR2 full length, M1-N823 UniprotKB Q9UHX3-1, EMR2W12
SEQ ID NO: 205
MGGRVFLVFLAFCVWLTLPGAETQDSRGCARWCPQDSSCVNATACRCNPGFSS
FSEIITTPMETCDDINECATLSKVSCGKFSDCWNTEGSYDCVCSPGYEPVSGAKTFKNES
ENTCQDVDECQQNPRLCKSYGTCVNTLGSYTCQCLPGFKLKPEDPKLCTDVNECTSGQ
NPCHSSTHCLNNVGSYQCRCRPGWQPIPGSPNGPNNTVCEDVDECSSGQHQCDSSTVCF
NTVGSYSCRCRPGWKPRHGIPNNQKDTVCEDMTFSTWTPPPGVHSQTLSRFFDKVQDL
GRDYKPGLANNTIQSILQALDELLEAPGDLETLPRLQQHCVASHLLDGLEDVLRGLSKN
LSNGLLNFSYPAGTELSLEVQKQVDRSVTLRQNQAVMQLDWNQAQKSGDPGPSVVGL
VSIPGMGKLLAEAPLVLEPEKQMLLHETHQGLLQDGSPILLSDVISAFLSNNDTQNLSSP
VTFTFSHRSVIPRQKVLCVFWEHGQNGCGHWATTGCSTIGTRDTSTICRCTHLSSFAVL
MAHYDVQEEDPVLTVITYMGLSVSLLCLLLAALTFLLCKAIQNTSTSLHLQLSLCLFLAH
LLFLVAIDQTGHKVLCSIIAGTLHYLYLATLTWMLLEALYLFLTARNLTVVNYSSINRFM
KKLMFPVGYGVPAVTVAISAASRPHLYGTPSRCWLQPEKGFIWGFLGPVCAIFSVNLVL
FLVTLWILKNRLSSLNSEVSTLRNTRMLAFKATAQLFILGCTWCLGILQVGPAARVMAY
LFTIINSLQGVFIFLVYCLLSQQVREQYGKWSKGIRKLKTESEMHTLSSSAKADTSKPSTV
N
huEMR2 GAIN + GPCR 7TM, D261-N823 UniprotKB Q9UHX3, EMR2W13
SEQ ID NO: 206
MARKSALLALALLLLGFPGAWGDMTFSTWTPPPGVHSQTLSRFFDKVQDLGRD
YKPGLANNTIQSILQALDELLEAPGDLETLPRLQQHCVASHLLDGLEDVLRGLSKNLSNG
LLNFSYPAGTELSLEVQKQVDRSVTLRQNQAVMQLDWNQAQKSGDPGPSVVGLVSIPG
MGKLLAEAPLVLEPEKQMLLHETHQGLLQDGSPILLSDVISAFLSNNDTQNLSSPVTFTF
SHRSVIPRQKVLCVFWEHGQNGCGHWATTGCSTIGTRDTSTICRCTHLSSFAVLMAHYD
VQEEDPVLTVITYMGLSVSLLCLLLAALTFLLCKAIQNTSTSLHLQLSLCLFLAHLLFLVA
IDQTGHKVLCSIIAGTLHYLYLATLTWMLLEALYLFLTARNLTVVNYSSINRFMKKLMF
PVGYGVPAVTVAISAASRPHLYGTPSRCWLQPEKGFIWGFLGPVCAIFSVNLVLFLVTL
WILKNRLSSLNSEVSTLRNTRMLAFKATAQLFILGCTWCLGILQVGPAARVMAYLFTIIN
SLQGVFIFLVYCLLSQQVREQYGKWSKGIRKLKTESEMHTLSSSAKADTSKPSTVN
TCR Va10.2 x Vb17 -KiHFc (knob-in-hole Fc), B17W38 Va10.2 -
knob-in-hole Fc chain 1
SEQ ID NO: 211
MAWVWTLLFLMAAAQSIQAQLLEQSPQFLSIQEGENLTVYCNSSSVFSSLQWYR
QEPGEGPVLLVTVVTGGEVKKLKRLTFQFGDARKDSSLHITAAQPGDTGLYLCAGAGS
QGNLIFGKGTKLSVKPNIQNPDPAVYQLRDSKSSDKSVCLFTDFDSQTNVSQSKDSDVYI
TDKTVLDMRSMDFKSNSAVAWSNKSDFACANAFNNSIIPEDTFFPSEPKSCDKTHTCPPC
PAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQ
VYVYPPSREEMTKNQVSLTCLVKGFYPSDIAVEWESNGQPENNYKTTPPVLDSDGSFAL
VSKLTVDKSRWQQGNVFSCSVMHEALHNHYTQKSLSLSPG
Vb17 -knob-in-hole Fc chain 2
SEQ ID NO: 212
MAWVWTLLFLMAAAQSIQAVDGGITQSPKYLFRKEGQNVTLSCEQNLNHDAM
YWYRQDPGQGLRLIYYSQIVNDFQKGDIAEGYSVSREKKESFPLTVTSAQKNPTAFYLC
ASSSRSSYEQYFGPGTRLTVTEDLKNVFPPEVAVFEPSEAEISHTQKATLVCLATGFYPD
HVELSWWVNGKEVHSGVSTDPQPLKEQPALNDSRYSLSSRLRVSATFWQNPRNHFRCQ
VQFYGLSENDEWTQDRAKPVTQIVSAEAWGRADEPKSCDKTHTCPPCPAPELLGGPSVF
LFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYVLPPSREE
MTKNQVSLLCLVKGFYPSDIAVEWESNGQPENNYLTWPPVLDSDGSFFLYSKLTVDKSR
WQQGNVFSCSVMHEALHNHYTQKSLSLSPG
TCR Va10.2 x Vb17 -KiHFc (knob-in-hole Fc), B17W40 Va10.2-
knob-in-hole Fc Chain 1
SEQ ID NO: 213
MAWVWTLLFLMAAAQSIQATQLLEQSPQFLSIQEGENLTVYCNSSSVFSSLQWY
RQEPGEGPVLLVTVVTGGEVKKLKRLTFQFGDARKDSSLHITAAQPGDTGLYLCAGAGS
QGNLIFGKGTKLSVKPNIQNPDPAVYQLRDSKSSDKSVCLFTDFDSQTNVSQSKDSDVYI
TDKTVLDMRSMDFKSNSAVAWSNKSDFACANAFNNSIIPEDTFFPSEPKSCDKTHTCPPC
PAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQ
VYVYPPSREEMTKNQVSLTCLVKGFYPSDIAVEWESNGQPENNYKTTPPVLDSDGSFAL
VSKLTVDKSRWQQGNVFSCSVMHEALHNHYTQKSLSLSPG
Vb17-knob-in-hole-Fc Chain 2
SEQ ID NO: 214
MARKSALLALALLLLFGPAWADGGITQSPKYLFRKEGQNVTLSCEQNLNHDAM
YWYRQDPGQGLRLIYYSQIVNDFQKGDIAEGYSVSREKKESFPLTVTSAQKNPTAFYLC
ASSSRSSYEQYFGPGTRLTVTEDLKNVFPPEVAVFEPSEAEISHTQKATLVCLATGFYPD
HVELSWWVNGKEVHSGVSTDPQPLKEQPALNDSRYCLSSRLRVSATFWQNPRNHFRCQ
VQFYGLSENDEWTQDRAKPVTQIVSAEAWGRADEPKSCDKTHTCPPCPAPELLGGPSVF
LFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYVLPPSREE
MTKNQVSLLCLVKGFYPSDIAVEWESNGQPENNYLTWPPVLDSDGSFFLYSKLTVDKSR
WQQGNVFSCSVMHEALHNHYTQKSLSLSPG
GAIN domain residues D261-Q478 of human EMR2 (UniprotKB
Q9UHX3 numbering)
SEQ ID NO: 215
DMTFSTWTPPPGVHSQTLSRFFDKVQDLGRDYKPGLANNTIQSILQALDELLEAP
GDLETLPRLQQHCVASHLLDGLEDVLRGLSKNLSNGLLNFSYPAGTELSLEVQKQVDRS
VTLRQNQAVMQLDWNQAQKSGDPGPSVVGLVSIPGMGKLLAEAPLVLEPEKQMLLHE
THQGLLQDGSPILLSDVISAFLSNNDTQNLSSPVTFTFSHRSVIPRQ
GPCR proteolytic site (GPS) motif of epidermal-growth-factor-
like module-containing mucin-like hormone receptor 2 (EMR2)
SEQ ID NO: 216
KVLCVFWEHGQNGCGHWATTGCSTIGTRDTSTICRCTHLSSFAVLMAHYDV
Epitope for EMR2 encompassing Residues 120-128
SEQ ID NO: 217
NQAVMQLDW
Epitope for EMR2 encompassing Residues 227-250
SEQ ID NO: 218
HGQNGCGHWATTGCSTIGTRDTST
Epitope for TRBV19 encompassing Residues 53-56
SEQ ID NO: 219
IVND
Epitope for TRBV19 encompassing Residues 53-56
YSQ
Linker
SEQ ID NO: 221
GGGSGGSGGCPPCGGSGG
Kappa region
SEQ ID NO: 222
RTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
Lambda region
SEQ ID NO: 223
FPPSSEELQANKATLVCLISDFYPGAVTVAWKADSSPVKAGVETTTPSKQSNNKY
AASSYLSLTPEQWKSHRSYSCQVTHEGSTVEKTVAPTECS
hulgG1_G1m(17) CH region
SEQ ID NO: 224
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKV
hulgG1_G1m(17) CH1 region
SEQ ID NO: 225
EPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQKSLSLSP
GK

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. An antibody, or an antigen-binding fragment thereof, that specifically binds the G-protein-coupled receptor auto-proteolysis inducing (GAIN) domain and/or the GPCR proteolytic site (GPS) motif of epidermal-growth-factor-like module-containing mucin-like hormone receptor 2 (EMR2).

2. The antibody, or the antigen-binding fragment thereof, according to claim 1, wherein the GAIN domain comprises amino acid residues D261-Q478 of SEQ ID NO: 205.

3. The antibody, or the antigen-binding fragment thereof, according to claim 1, wherein the GAIN domain comprises the amino acid sequence of SEQ ID NO: 215.

4. The antibody, or the antigen-binding fragment thereof, according to claim 1, wherein the GPS motif comprises the amino acid sequence of SEQ ID NO: 216.

5. The antibody, or the antigen-binding fragment thereof, according to claim 1, wherein the antibody or the antigen-binding fragment thereof binds to an epitope comprising the amino acid sequence of SEQ ID NO: 217 or SEQ ID NO: 218.

6. The antibody, or the antigen-binding fragment thereof, according to claim 1, wherein the antibody or the antigen-binding fragment thereof binds to human EMR2 with a dissociation constant (K_{D}) between about 0.01 nM to about 5 nM.

7. The antibody, or the antigen-binding fragment thereof, according to claim 1, wherein the antibody, or the antigen-binding fragment thereof, binds to human EMR2 with an half maximal effective concentration (EC₅₀) between about 0.1 nM to about 15 nM.

8. The antibody, or the antigen-binding fragment thereof, according to claim 1, wherein the antibody, or the antigen-binding fragment thereof, is or comprises a fragment antigen-binding (Fab), a F(ab′)2 fragment, F(ab)′3 fragments, a single-chain variable fragment (scFv), a bis-scFv, a (scFv)2, a stapled scFv (spFv), a diabody, a minibody, a nanobody, a triabody, a tetrabody, a disulfide stabilized Fv protein (dsFv), a single-domain antibody (sdAb), an Immunoglobulin New Antigen Receptor (Ig NAR), a single heavy chain antibody, a camelid antibody, a shark antibody, or a chemically modified derivative thereof.

9. The antibody, or the antigen-binding fragment thereof, according to claim 8, wherein the antibody, or the antigen-binding fragment thereof, comprises a Fab.

10. The antibody, or the antigen-binding fragment thereof, according to claim 9, wherein the antibody or the antigen-binding fragment thereof further comprises a first constant Ig domain of the heavy chain (CH1 domain).

11. The antibody, or the antigen-binding fragment thereof, according to claim 8, wherein the antibody or the antigen-binding fragment thereof does not comprise a CH1 domain.

12. The antibody, or the antigen-binding fragment thereof, according to claim 8, wherein the antibody, or the antigen-binding fragment thereof, comprises a scFv or a spFv.

13. The antibody, or the antigen-binding fragment thereof, according to claim 12, wherein the scFv or the spFv comprises a signal sequence, a heavy chain variable sequence, a GS-Linker, and a light chain variable sequence.

14. The antibody, or the antigen-binding fragment thereof, according to claim 8, further comprising a fragment crystallizable (Fc) domain.

15. The antibody, or the antigen-binding fragment thereof according to claim 14, wherein the Fc domain of the antibody, or the antigen-binding fragment thereof, is an IgA, an IgG, an IgE, or an IgM.

16. The antibody, or the antigen-binding fragment thereof, according to claim 15, wherein the Fc domain of the antibody, or the antigen-binding fragment thereof, is an IgG.

17. The antibody, or the antigen-binding fragment thereof, according to claim 16, wherein the IgG is IgG1 or IgG4.

18. The antibody, or the antigen-binding fragment thereof, according to claim 17, wherein the Fc domain comprises one or more different mutations which promote heterodimerization.

19. The antibody, or the antigen-binding fragment thereof, according to claim 18, wherein the Fc domain comprises mutations T366S, L368A, and Y407V (EU numbering) or mutation T366W (EU numbering).

20. The antibody, or the antigen-binding fragment thereof, according to claim 17, wherein the Fc domains of the first heavy chain (HC1) and/or the second heavy chain (HC2) further comprise one or more mutations which reduce Fc binding to a Fcγ receptor.

21. The antibody, or the antigen-binding fragment thereof, according to claim 20, wherein the Fc receptor is FcγRI, FcyRIIA, FcγRIIB, FcyRIIIA, and/or FcγRIIIB.

22. The antibody, or the antigen-binding fragment thereof, according to claim 21, wherein the Fc domain comprises one or more mutations selected from L234A, L235A, and D265S (EU numbering).

23. The antibody, or the antigen-binding fragment thereof, according to claim 21, wherein the Fc domain comprises mutations L234A, L235A, and D265S (EU numbering).

24. The antibody, or the antigen-binding fragment thereof, according to claim 14, wherein the Fc domain further comprises one or more mutations which reduce Fc binding to protein A.

25. The antibody, or the antigen-binding fragment thereof, according to claim 24, wherein the Fc domain comprises mutations H435R and/or Y436F (EU numbering).

26. The antibody, or the antigen-binding fragment thereof, according to claim 24, wherein the Fc domain comprises mutations H435R and Y436F (EU numbering).

27. The antibody, or the antigen-binding fragment thereof, according to claim 1, wherein the antibody, or the antigen-binding fragment thereof, comprises a humanized antibody, or an antigen binding fragment thereof, a human antibody, or an antigen binding fragment thereof, a murine antibody or an antigen binding fragment thereof, a chimeric antibody, or an antigen binding fragment thereof, a monospecific antibody, or a monospecific antigen binding fragment thereof, a bispecific antibody, or a bispecific antigen binding fragment thereof, a multispecific antibody, or a multispecific antigen binding fragment thereof.

28. The antibody, or the antigen-binding fragment thereof according to claim 1, wherein the antibody or the antigen-binding fragment thereof comprises:
a) a heavy chain complementarity determining region (HCDR) 1, a HCDR2 and a HCDR3 of the heavy chain variable region (VH) of SEQ ID NO: 63, and a light chain complementarity determining region (LCDR) 1, a LCDR2 and a LCDR3 of the light chain variable region (VL) of SEQ ID NO: 64;
b) a HCDR1, a HCDR2, and a HCDR3 of the VH of SEQ ID NO: 95, and a LCDR1, a LCDR2, and a LCDR3 of the VL of SEQ ID NO: 96;
c) a HCDR1, a HCDR2, and a HCDR3 of the VH of SEQ ID NO: 127, and a LCDR1, a LCDR2, and a LCDR3 of the VL of SEQ ID NO: 128; or
d) a HCDR1, a HCDR2, and a HCDR3 of the VH of SEQ ID NO: 191, and a LCDR1, a LCDR2, and a LCDR3 of the VL of SEQ ID NO: 192.

29. The antibody, or the antigen-binding fragment thereof, according to claim 28, wherein the antibody, or the antigen-binding fragment thereof, comprises the HCDR1, the HCDR2, the HCDR3, the LCDR1, the LCDR2 and the LCDR3 of:
a) SEQ ID NO: 33, 34, 35, 48, 49, and 50, respectively b) SEQ ID NO: 36, 37, 38, 51, 52, and 53, respectively;
c) SEQ ID NO: 39, 40, 41, 54, 55, and 56, respectively;
d) SEQ ID NO: 42, 43, 44, 57, amino acid sequence EVS, and SEQ ID NO: 59, respectively;
e) SEQ ID NO: 45, 46, 47, 60, 61, and 62, respectively;
f) SEQ ID NO: 65, 66, 67, 80, 81, and 82, respectively;
g) SEQ ID NO: 68, 69, 70, 83, 84, and 85, respectively;
h) SEQ ID NO: 71, 72, 73, 86, 87, and 88, respectively;
i) SEQ ID NO: 74, 75, 76, 89, amino acid sequence AAI, and SEQ ID NO: 91, respectively;
j) SEQ ID NO: 77, 78, 79, 92, 93, and 94, respectively;
k) SEQ ID NO: 97, 98, 99, 112, 113, and 114, respectively;
l) SEQ ID NO: 100, 101, 102, 115, 116, and 117, respectively;
m) SEQ ID NO: 103, 104, 105, 118, 119, and 120, respectively;
n) SEQ ID NO: 106, 107, 108, 121, amino acid sequence DNN, and SEQ ID NO: 123, respectively;
o) SEQ ID NO: 109, 110, 111, 124, 125, and 126, respectively;
p) SEQ ID NO: 161, 162, 163, 176, 177, and 178, respectively;
q) SEQ ID NO: 164, 165, 166, 179, 180, and 181, respectively;
r) SEQ ID NO: 167, 168, 169, 182, 18, and 184, respectively;
s) SEQ ID NO: 170, 171, 172, 185, amino acid sequence EVS, and SEQ ID NO:187, respectively; or
t) SEQ ID NO: 173, 174, 175, 188, 189, and 190, respectively.

30. The antibody, or the antigen-binding fragment thereof, according to claim 29, wherein the antibody or the antigen-binding fragment thereof, comprises a variable heavy chain region (VH) comprising an amino acid sequence that is at least 90%, at least 91%, at least 92%, at least 93%, at least 94%, at least 95%, at least 96%, at least 97%, at least 98%, at least 99% or 100% identical to:
a) the VH of SEQ ID NO: 63;
b) the VH of SEQ ID NO: 95;
c) the VH of SEQ ID NO: 127; or
d) the VH of SEQ ID NO: 191.

31. The antibody, or the antigen-binding fragment thereof, according to claim 29, wherein the antibody, or the antigen-binding fragment thereof, comprises or further comprises a variable light chain region (VL) comprising an amino acid sequence that is at least 90%, at least 91%, at least 92%, at least 93%, at least 94%, at least 95%, at least 96%, at least 97%, at least 98%, at least 99% or 100% identical to:
a) the VL of SEQ ID NO: 64;
b) the VL of SEQ ID NO: 96;
c) the VL of SEQ ID NO: 128; or
d) the VL of SEQ ID NO: 192.

32. The antibody, or the antigen-binding fragment thereof, according to claim 29, wherein the antibody, or the antigen-binding fragment thereof, comprises:
a) the VH of SEQ ID NO: 63 and the VL of SEQ ID NO: 64;
b) the VH of SEQ ID NO: 95 and the VL of SEQ ID NO: 96;
c) the VH of SEQ ID NO: 127 and the VL of SEQ ID NO: 128; or
d) the VH of SEQ ID NO: 191 and the VL of SEQ ID NO: 192.

33. The antibody, or the antigen-binding fragment thereof, according to claim 1, wherein the antibody, or the antigen-binding fragment thereof, comprises a scFv or a spFV which comprises, from the N- to C-terminus, a VH, a linker and a VL in the format VH-L-VL or the VL, a linker and a VH in the format VL-L-VH.

34. The antibody, or the antigen-binding fragment thereof, according to claim 33, wherein the VH comprises the amino acid sequence of SEQ ID NO: 191, the VL comprises the amino acid sequence of SEQ ID NO: 192, and the linker comprises the amino acid sequence of SEQ ID NO: 221.

35. The antibody, or the antigen-binding fragment thereof according to claim 33, wherein the linker comprises the amino acid sequence of SEQ ID NO: 221.

36. The antibody, or the antigen-binding fragment thereof, according to claim 1, comprising a heavy chain comprising the amino acid sequence of SEQ ID NO: 194, 196, 198, or 202.

37. The antibody, or the antigen-binding fragment thereof, according to claim 36, comprising a light chain comprising the amino acid sequence of SEQ ID NO: 195, 197 or 199.

38. The antibody or the antigen-binding fragment thereof according to claim 37, wherein the antibody or the antigen-binding fragment thereof comprises:
a) an HC1 comprising the amino acid sequence of SEQ ID NO: 194 and a first light chain (LC1) comprising the amino acid sequence of SEQ ID NO: 195;
b) an HC1 comprising the amino acid sequence of SEQ ID NO: 196 and a LC1 comprising the amino acid sequence of SEQ ID NO: 197; or
c) an HC1 comprising the amino acid sequence of SEQ ID NO: 198 and a LC1 comprising the amino acid sequence of SEQ ID NO: 199.

39. An antibody, or an antigen-binding fragment thereof, that binds to the same epitope of EMR2 as the antibody, or the antigen-binding fragment thereof, according to claim 28.

40. An antibody, or an antigen-binding fragment thereof, that competes for binding to the same epitope of EMR2 with the antibody, or the antigen-binding fragment thereof according to claim 28.

41. The antibody, or the antigen-binding fragment thereof, according to claim 1, wherein the antibody, or the antigen-binding fragment thereof, is a monospecific antibody.

42. The antibody, or the antigen-binding fragment thereof, according to claim 1, wherein the antibody, or the antigen-binding fragment thereof, is a bispecific antibody which specifically binds to EMR2 and to a second antigen.

43. The antibody, or the antigen-binding fragment thereof, according to claim 42, wherein the second antigen is T-cell Receptor Variable 19 (TRBV19).

44. An isolated polynucleotide encoding the antibody, or the antigen-binding fragment thereof according to claim 1.

45. The isolated polynucleotide of claim 44, wherein the polynucleotide comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to:
a) the VH of SEQ ID NO: 63;
b) the VH of SEQ ID NO: 95
c) the VH of SEQ ID NO: 127; or
d) the VH of SEQ ID NO: 191.

46. The isolated polynucleotide of claim 45, wherein the polynucleotide comprises or further comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to:
a) the VL of SEQ ID NO: 64;
b) the VL of SEQ ID NO: 96;
c) the VL of SEQ ID NO: 128; or
d) the VL of SEQ ID NO: 192.

47. The isolated polynucleotide of claim 46, wherein the polynucleotide comprises a sequence comprising a nucleotide sequence encoding:
a) the amino acid sequence of SEQ ID NO: 63 and/or SEQ ID NO: 64;
b) the amino acid sequence of SEQ ID NO: 95 and/or SEQ ID NO: 96;
c) the amino acid sequence of SEQ ID NO: 127 and/or SEQ ID NO: 128; or
d) the amino acid sequence of SEQ ID NO: 191 and/or the SEQ ID NO: 192.

48. The isolated polynucleotide according to claim 47, wherein the polynucleotide comprises a sequence encoding a heavy chain comprising the amino acid sequence of SEQ ID NO: 194, 196, 198, or 202.

49. The isolated polynucleotide according to claim 48, wherein the polynucleotide comprises a sequence encoding a light chain comprising the amino acid sequence of SEQ ID NO: 195, 197, or 199.

50. The isolated polynucleotide of claim 49, comprising a nucleotide sequence encoding:
a) the amino acid sequence of SEQ ID NO: 194 and/or SEQ ID NO: 195;
b) the amino acid sequence of SEQ ID NO: 196 and/or SEQ ID NO: 197;
c) the amino acid sequence of SEQ ID NO: 198 and/or the amino acid sequence of SEQ ID NO: 199; or
d) the amino acid sequence of SEQ ID NO: 202.

51. A vector comprising the isolated polynucleotide according to claim 44.

52. The vector according to claim 51, wherein the isolated polynucleotide is operably linked to an expression control sequence.

53. The vector according to claim 52, wherein the vector is a viral vector.

54. The vector according to claim 53, wherein the viral vector is selected from an adenoviral vector, an adeno-associated viral vector, a retroviral vector, a lentiviral vector, a herpes simplex virus vector, and a poxvirus vector.

55. A pharmaceutical composition comprising (i) the antibody, or the antigen-binding fragment thereof, according to claim 141, or an isolated polynucleotide encoding the antibody, or the antigen-binding fragment thereof, or a vector comprising the isolated polynucleotide, and (ii) a pharmaceutically acceptable carrier or excipient.

56. A host cell expressing the antibody, or the antigen-binding fragment thereof, according to claim 1.

57. The host cell, according to claim 56, wherein the cell is a hybridoma.

58. The host cell, according to claim 56, wherein the antibody, or the antigen-binding fragment thereof, is recombinantly produced.

59. A host cell comprising the isolated polynucleotide according to claim 44, or a vector comprising the isolated polynucleotide.

60. A method for treating a cancer in a subject in need thereof, the method comprising administering to the subject a therapeutically effective amount of the antibody, or the antigen-binding fragment thereof, according to claim 1, or an isolated polynucleotide encoding the antibody, or the antigen-binding fragment thereof, or a vector comprising the isolated polynucleotide, or a pharmaceutical composition comprising the antibody, or the antigen-binding fragment thereof, or a host cell comprising the antibody, or the antigen-binding fragment thereof.

61. A method for inducing cytotoxicity of a cancer cell or redirecting immune or T cells to cancer cells, the method comprising administering to the cancer cell an effective amount of the antibody or, the antigen-binding fragment thereof, according to claim 1, or an isolated polynucleotide encoding the antibody, or the antigen-binding fragment thereof, or a vector comprising the isolated polynucleotide, or a pharmaceutical composition comprising the antibody, or the antigen-binding fragment thereof, or a host cell comprising the antibody, or the antigen-binding fragment thereof, wherein the effective amount is sufficient to inhibit the growth or proliferation of the cancer cell.

62. The method according to claim 61, wherein the cancer cell is in a subject and the antibody, or the antigen-binding fragment thereof, the polynucleotide, the vector, the pharmaceutical composition, or the host cell is administered to the subject.

63. The method according to claim 62, wherein the administration is conducted ex vivo.

64. The method according to 63, wherein the cancer is an EMR2-expressing cancer.

65. The method according to claim 64, wherein the EMR2-expressing cancer is a hematological cancer.

66. The method according to claim 65, wherein the hematological cancer is a myeloid malignancy.

67. The method according to claim 66, wherein the hematological cancer is acute myeloid leukemia (AML), chronic myelogenous leukemia (CML), or myelodysplastic neoplasms.

68. The method according claim 60, further comprising administering a second therapeutic agent.

69. The method according to claim 68, wherein the second therapeutic agent is a surgery, a chemotherapy, an androgen deprivation therapy, a radiation, or any combination thereof.

70. The antibody, or the antigen binding fragment thereof, according to claim 1, for use in a method for treating cancer or a method for inducing cytotoxicity of a cancer cell or redirecting immune or T cells to cancer cells.

71. The isolated polynucleotide, according to claim 44, for use in a method for treating cancer or a method for inducing cytotoxicity of a cancer cell or redirecting immune or T cells to cancer cells.

72. The vector, according to claim 51, for use in a method for treating cancer or a method for inducing cytotoxicity of a cancer cell or redirecting immune or T cells to cancer cells.

73. The pharmaceutical composition, according to claim 55, for use in a method for treating cancer or a method for inducing cytotoxicity of a cancer cell or redirecting immune or T cells to cancer cells.

74. The host cell according to claim 56, for use in a method for treating cancer or a method for inducing cytotoxicity of a cancer cell or redirecting immune or T cells to cancer cells.

75. A method for generating the antibody, or the antigen-binding fragment thereof, according to claim 1, wherein the method comprises culturing a host cell comprising the antibody, or the antigen-binding fragment thereof, and isolating the antibody, or the antigen-binding fragment thereof.

76. A kit comprising (i) the antibody, or the antigen-binding fragment thereof according to claim 1, and/or an isolated polynucleotide encoding the antibody, or the antigen-binding fragment thereof, and/or a vector comprising the isolated polynucleotide, and/or a pharmaceutical composition comprising the antibody, or the antigen-binding fragment thereof, and/or a host cell comprising the antibody, or the antigen-binding fragment thereof, and (ii) packaging for the same and/or instructions for use.

77. A bispecific antibody, or a bispecific antigen-binding fragment thereof, that specifically binds (i) TRBV19 with a first antigen-binding site and (ii) the GAIN domain and/or the GPS motif of EMR2 with a second antigen-binding site.

78. The bispecific antibody, or the bispecific antigen-binding fragment thereof, according to claim 77, comprising:
a) a first heavy chain (HC1);
b) optionally a first light chain (LC1);
c) a second heavy chain (HC2); and
d) optionally a second light chain (LC2),
wherein
(i) the HC1 and the LC1 form a first antigen-binding site that specifically binds a first antigen,
(ii) the HC2 and the LC2 form a second antigen-binding site that specifically binds a second antigen,
(iii) the HC1 and HC2 each comprise a Fc domain comprising a CH2-CH3 domain; and
wherein the first antigen is TRBV19, and the second antigen is EMR2.

79. The bispecific antibody, or the bispecific antigen-binding fragment thereof, according to claim 77, wherein the second antigen-binding site specifically binds the GAIN domain and/or the GPS motif of EMR2.

80. The bispecific antibody, or the bispecific antigen-binding fragment thereof, according to claim 79, wherein the GAIN domain comprises amino acid residues of SEQ ID NO: 205.

81. The bispecific antibody, or the bispecific antigen-binding fragment thereof, according to claim 79, wherein the GAIN domain comprises the amino acid sequence of SEQ ID NO: 215.

82. The bispecific antibody, or the bispecific antigen-binding fragment thereof, according to claim 77, wherein the GPS motif comprises the amino acid sequence of SEQ ID NO: 216.

83. The bispecific antibody, or the bispecific antigen-binding fragment thereof, according to claim 77, wherein the antibody or antigen-binding fragment thereof binds to an epitope comprising the amino acid sequence of SEQ ID NO: 217 or SEQ ID NO: 218.

84. The bispecific antibody, or the bispecific antigen-binding fragment thereof, according to claim 77, wherein the first antigen-binding site comprises a Fab or a scFv or a spFv.

85. The bispecific antibody, or the bispecific antigen-binding fragment thereof, according to claim 77, wherein the Fc of the first antigen-binding site comprises a CH1 domain.

86. The bispecific antibody, or the bispecific antigen-binding fragment thereof, according to claim 77, wherein the second antigen-binding site comprises a Fab, a scFv or a spFv.

87. The bispecific antibody, or the bispecific antigen-binding fragment thereof, according to claim 77, wherein the Fc of the second antigen-binding site further comprises a CH1 domain.

88. The bispecific antibody, or the bispecific antigen-binding fragment thereof, according to claim 77, wherein the first antigen-binding site comprises a Fab and the second antigen-binding site comprises an scFv or a spFv.

89. The bispecific antibody, or the bispecific antigen-binding fragment thereof, according to claim 77, wherein the first antigen-binding site comprises an scFv or a spFv and the second antigen-binding site comprises a Fab.

90. The bispecific antibody, or the bispecific antigen-binding fragment thereof, according to claim 77, wherein the Fc domain of the antibody or antigen-binding fragment thereof is an IgA, an IgG, an IgE, or an IgM.

91. The bispecific antibody, or the bispecific antigen-binding fragment thereof, according to claim 90, wherein the Fc domain of the antibody, or the antigen-binding fragment thereof, is an IgG.

92. The bispecific antibody, or the bispecific antigen-binding fragment thereof, according to claim 91, wherein the IgG is IgG1 or IgG4.

93. The bispecific antibody, or the bispecific antigen-binding fragment thereof, according to claim 92, wherein the Fc domains of HC1 and HC2 comprise one or more different mutations which promote heterodimerization.

94. The bispecific antibody, or the bispecific antigen-binding fragment thereof, according to claim 93, wherein the Fc domain of the HC1 comprises mutations T366S, L368A and Y407V (EU numbering) and the Fc domain of the HC2 comprises mutation T366W (EU numbering).

95. The bispecific antibody, or the bispecific antigen-binding fragment thereof, according to claim 93, wherein the Fc domain of the HC2 comprises mutations T366S, L368A and Y407V (EU numbering) and the Fc domain of the HC1 comprises mutation T366W (EU numbering).

96. The bispecific antibody, or the bispecific antigen-binding fragment thereof, according to claim 90, wherein the Fc domains of HC1 and/or HC2 further comprise one or more mutations which reduce Fc binding to a Fcγ receptor.

97. The bispecific antibody, or the bispecific antigen-binding fragment thereof, according to claim 96, wherein the Fc receptor is FcγRI, FcγRIIA, FcγRIIB, FcγRIIIA, and/or FcγRIIIB.

98. The bispecific antibody, or the bispecific antigen-binding fragment thereof, according to claim 97, wherein the Fc domains of HC1 and/or HC2 each comprise one or more mutations selected from L234A, L235A, and D265S (EU numbering).

99. The bispecific antibody, or the bispecific antigen-binding fragment thereof, according to claim 97, wherein the Fc domains of HC1 and HC2 each comprise mutations L234A, L235A, and D265S (EU numbering).

100. The bispecific antibody, or the bispecific antigen-binding fragment thereof, according to claim 92, wherein the Fc domains of HC1 or HC2 further comprises one or more mutations which reduce Fc binding to protein A.

101. The bispecific antibody, or the bispecific antigen-binding fragment thereof according to claim 100, wherein the Fc domains of HC1 or HC2 comprise mutations H435R and/or Y436F (EU numbering).

102. The bispecific antibody, or the bispecific antigen-binding fragment thereof, according to claim 101, wherein the Fc domain of HC1 comprises mutations H435R and Y436F (EU numbering).

103. The bispecific antibody, or the bispecific antigen-binding fragment thereof, according to claim 101, wherein the Fc domain of HC2 comprises mutations H435R and Y436F (EU numbering).

104. The bispecific antibody, or the bispecific antigen-binding fragment thereof, according to claim 78, wherein the HC1 or HC2 comprise mutation C220S (EU numbering).

105. The bispecific antibody, or the bispecific antigen-binding fragment thereof, according to claim 78, wherein the HC1 comprises mutation C220S (EU numbering).

106. The bispecific antibody, or the bispecific antigen-binding fragment thereof, according to claim 78, wherein the HC2 comprises mutation C220S (EU numbering).

107. The bispecific antibody, or the bispecific antigen-binding fragment thereof, according to claim 77, wherein the bispecific antibody, or the bispecific antigen-binding fragment thereof comprises a humanized antibody, or an antigen binding fragment thereof, a human antibody, or an antigen binding fragment thereof, a murine antibody, or an antigen binding fragment thereof, a chimeric antibody, or an antigen binding fragment thereof, or a chemically modified derivative thereof.

108. The bispecific antibody, or the bispecific antigen-binding fragment thereof, according to claim 77, wherein the first antigen-binding site that specifically binds TRBV19 comprises:
a) a HCDR1, a HCDR2 and a HCDR3 of the VH of SEQ ID NO: 31 and a LCDR) 1, a LCDR2 and a LCDR3 of the VL of SEQ ID NO: 32; or
b) a HCDR1, a HCDR2, and a HCDR3 of the VH of SEQ ID NO: 159 and a LCDR1, LCDR2, and LCDR3 of the VL of SEQ ID NO: 160.

109. The bispecific antibody, or the bispecific antigen-binding fragment thereof, according to claim 108, wherein the first antigen-binding site that specifically binds TRBV19 comprises the HCDR1, the HCDR2, the HCDR3, the LCDR1, the LCDR2 and the LCDR3 of:
a) SEQ ID NOs: 1, 2, 3, 16, 17, and 18, respectively;
b) SEQ ID NOs: 4, 5, 6, 19, 20, and 21, respectively;
c) SEQ ID NOs: 7, 8, 9, 22, 23, and 24, respectively;
d) SEQ ID NOs: 10, 11, 12, 25, 26, and 27, respectively;
e) SEQ ID NOs: 13, 14, 15, 28, 29, and 30, respectively;
f) SEQ ID NOs: 129, 130, 131, 144, 145, and 146, respectively;
g) SEQ ID NOs: 132, 133, 134, 147, 148, and 149, respectively;
h) SEQ ID NOs: 135, 136, 137, 150, 151, and 152, respectively;
i) SEQ ID NOs: 138, 139, 140, 153, amino acid sequence KVS, and SEQ ID NO: 155, respectively; or
j) SEQ ID NOs: 141, 142, 143, 156, 157, and 158, respectively.

110. The bispecific antibody, or the bispecific antigen-binding fragment thereof, according to claim 109, wherein the first antigen-binding site that specifically binds TRBV19 comprises a VH comprising an amino acid sequence that is at least 90%, at least 91%, at least 92%, at least 93%, at least 94%, at least 95%, at least 96%, at least 97%, at least 98%, at least 99% or 100% identical to:
a) the VH of SEQ ID NO: 31; or
b) the VH of SEQ ID NO: 159.

111. The bispecific antibody, or the bispecific antigen-binding fragment thereof, according to claim 109, wherein the first antigen-binding site that specifically binds TRBV19 comprises or further comprises a VL comprising an amino acid sequence that is at least 90%, at least 91%, at least 92%, at least 93%, at least 94%, at least 95%, at least 96%, at least 97%, at least 98%, at least 99% or 100% identical to:
a) the VL of SEQ ID NO: 32; or
b) the VL of SEQ ID NO: 160.

112. The bispecific antibody, or the bispecific antigen-binding fragment thereof, according to claim 109, wherein the first antigen-binding site that specifically binds TRBV19 comprises:
a) the VH of SEQ ID NO: 31 and the VL of SEQ ID NO: 32; or
b) the VH of SEQ ID NO: 159 and the VL of SEQ ID NO: 160.

113. The bispecific antibody, or the bispecific antigen-binding fragment thereof, according to claim 78, wherein the second antigen-binding site that specifically binds EMR2 comprises:
a) a HCDR1, a HCDR2 and a HCDR3 of the VH of SEQ ID NO: 63, and a LCDR 1, a LCDR2 and a LCDR3 of the VL of SEQ ID NO: 64;
b) a HCDR1, a HCDR2, and a HCDR3 of the VH of SEQ ID NO: 95, and a LCDR1, a LCDR2, and a LCDR3 of the VL of SEQ ID NO: 96;
c) a HCDR1, a HCDR2, and a HCDR3 of the VH of SEQ ID NO: 127, and a LCDR1, a LCDR2, and a LCDR3 of the VL of SEQ ID NO: 128; or
d) a HCDR1, a HCDR2, and a HCDR3 of the VH of SEQ ID NO: 191, and a LCDR1, a LCDR2, and a LCDR3 of the VL of SEQ ID NO: 192.

114. The bispecific antibody, or the bispecific antigen-binding fragment thereof, according to claim 113, wherein the second antigen-binding site that specifically binds EMR2 comprises the HCDR1, the HCDR2, the HCDR3, the LCDR1, the LCDR2 and the LCDR3 of:
a) SEQ ID NO: 33, 34, 35, 48, 49, and 50, respectively;
b) SEQ ID NO: 36, 37, 38, 51, 52, and 53, respectively;
c) SEQ ID NO: 39, 40, 41, 54, 55, and 56, respectively;
d) SEQ ID NO: 42, 43, 44, 57, 58, and 59, respectively;
e) SEQ ID NO: 45, 46, 47, 60, 61, and 62, respectively;
f) SEQ ID NO: 65, 66, 67, 80, 81, and 82, respectively;
g) SEQ ID NO: 68, 69, 70, 83, 84, and 85, respectively;
h) SEQ ID NO: 71, 72, 73, 86, 87, and 88, respectively;
i) SEQ ID NO: 74, 75, 76, 89, amino acid sequence AAI, and SEQ ID NO: 91, respectively;
j) SEQ ID NO: 77, 78, 79, 92, 93, and 94, respectively;
k) SEQ ID NO: 97, 98, 99, 112, 113, and 114, respectively;
l) SEQ ID NO: 100, 101, 102, 115, 116, and 117, respectively;
m) SEQ ID NO: 103, 104, 105, 118, 119, and 120, respectively;
n) SEQ ID NO: 106, 107, 108, 121, amino acid sequence DNN, and SEQ ID NO: 123, respectively;
o) SEQ ID NO: 109, 110, 111, 124, 125, and 126, respectively;
p) SEQ ID NO: 161, 162, 163, 176, 177, and 178, respectively;
q) SEQ ID NO: 164, 165, 166, 179, 180, and 181, respectively;
r) SEQ ID NO: 167, 168, 169, 182, 18, and 184, respectively;
s) SEQ ID NO: 170, 171, 172, 185, amino acid sequence EVS, and SEQ ID NO:187, respectively; or
t) SEQ ID NO: 173, 174, 175, 188, 189, and 190, respectively.

115. The bispecific antibody, or the bispecific antigen-binding fragment thereof, according to claim 114 wherein the bispecific antibody or bispecific antigen-binding fragment thereof comprises a VH comprising an amino acid sequence that is at least 90%, at least 91%, at least 92%, at least 93%, at least 94%, at least 95%, at least 96%, at least 97%, at least 98%, at least 99% or 100% identical to:
a) the VH of SEQ ID NO: 63;
b) the VH of SEQ ID NO: 95;
c) the VH of SEQ ID NO: 127; or
d) the VH of SEQ ID NO: 191.

116. The bispecific antibody, or the bispecific antigen-binding fragment thereof, according to claim 114, wherein the bispecific antibody or bispecific antigen-binding fragment thereof comprises or further comprises a VL comprising an amino acid sequence that is at least 90%, at least 91%, at least 92%, at least 93%, at least 94%, at least 95%, at least 96%, at least 97%, at least 98%, at least 99% or 100% identical to:
a) the VL of SEQ ID NO: 64;
b) the VL of SEQ ID NO: 96;
c) the VL of SEQ ID NO: 128; or
d) the VL of SEQ ID NO: 192.

117. The bispecific antibody, or the bispecific antigen-binding fragment thereof, according to claim 114, wherein the first antigen-binding site and/or the second antigen-binding site that specifically binds EMR2 comprises:
a) the VH of SEQ ID NO: 63 and the VL of SEQ ID NO: 64;
b) the VH of SEQ ID NO: 95 and the VL of SEQ ID NO: 96;
c) the VH of SEQ ID NO: 127 and the VL of SEQ ID NO: 128; or
d) the VH of SEQ ID NO: 191 and the VL of SEQ ID NO: 192.

118. The bispecific antibody, or the bispecific antigen-binding fragment thereof, according to claim 77, wherein the bispecific antibody or bispecific antigen-binding fragment thereof comprises an scFv or a spFV, wherein the scFv or the spFV comprises, from the N- to C-terminus, a VH, a linker (L) and a VL in the format VH-L-VL or the VL, the linker and the VH in the format VL-L-VH.

119. The bispecific antibody, or the bispecific antigen-binding fragment thereof, according to claim 118, wherein the VH comprises the amino acid sequence of SEQ ID NO: 159, the VL comprises the amino acid sequence of SEQ ID NO: 160, and the linker comprises the amino acid sequence of SEQ ID NO: 221.

120. The bispecific antibody, or the bispecific antigen-binding fragment thereof, according to claim 118, wherein the VH comprises the amino acid sequence of SEQ ID NO: 191, the VL comprises the amino acid sequence of SEQ ID NO: 192, and the linker comprises the amino acid sequence of SEQ ID NO: 221.

121. The bispecific antibody, or the bispecific antigen-binding fragment thereof, according to claim 118, wherein the linker comprises the amino acid sequence of SEQ ID NO: 221.

122. The bispecific antibody, or the bispecific antigen-binding fragment thereof, according to claim 77, wherein the first antigen-binding site specifically binds to TRBV19 with a K_{D} that is between about 15 nM to about 200 nM.

123. The bispecific antibody, or the bispecific antigen-binding fragment thereof, according to claim 77, wherein the first antigen-binding site specifically binds to TRBV19 with an EC₅₀ between about 1 nM to about 100 nM.

124. The bispecific antibody, or the bispecific antigen-binding fragment thereof, according to claim 77, wherein the second antigen-binding site specifically binds to EMR2 with a K_{D} that is between about 0.01 nM to about 5 nM.

125. The bispecific antibody, or the bispecific antigen-binding fragment thereof, according to claim 77, wherein the second antigen-binding site specifically binds to EMR2 with an EC₅₀ between about 0.1 nM to 15 about nM.

126. The bispecific antibody, or the bispecific binding fragment thereof, according to claim 77, wherein the HC1 comprises the amino acid sequence of SEQ ID NO: 193 or 200.

127. The bispecific antibody, or the bispecific binding fragment thereof, according to claim 77, wherein the LC1 comprises the amino acid sequence of SEQ ID NO: 201.

128. The bispecific antibody, or the bispecific binding fragment thereof, according to claim 77, wherein
a) the HC1 comprises the amino acid sequence of SEQ ID NO: 193; or
b) the HC1 comprises the amino acid sequence of SEQ ID NO: 200 and the LC1 comprises the amino acid sequence of SEQ ID NO: 201.

129. The bispecific antibody, or the bispecific binding fragment thereof, according to claim 77, wherein the HC2 comprises the amino acid sequence of SEQ ID NO: 194, 196, 198, or 202.

130. The bispecific antibody, or the bispecific binding fragment thereof, according to claim 77, wherein the LC2 comprises the amino acid sequence of SEQ ID NO: 195, 197, or 199.

131. The bispecific antibody, or the bispecific binding fragment thereof, according to claim 77, wherein
a) the HC2 comprises the amino acid sequence of SEQ ID NO: 194 and the LC2 comprises the amino acid sequence of SEQ ID NO: 195;
b) the HC2 comprises the amino acid sequence of SEQ ID NO: 196 and the LC1 comprises the amino acid sequence of SEQ ID NO: 197;
c) the HC2 comprises the amino acid sequence of SEQ ID NO: 198 and the LC1 comprises the amino acid sequence of SEQ ID NO: 199; or
d) the HC2 comprises the amino acid sequence of SEQ ID NO: 202.

132. The bispecific antibody, or the bispecific binding fragment thereof, according to claim 77, wherein
a) the HC1 comprises the amino acid sequence of SEQ ID NO: 193, the HC2 comprises the amino acid sequence of SEQ ID NO: 194, and the LC2 comprises the amino acid sequence of SEQ ID NO 195;
b) the HC1 comprises the amino acid sequence of SEQ ID NO: 193, the HC2 comprises the amino acid sequence of SEQ ID NO: 196, and the LC2 comprises the amino acid sequence of SEQ ID NO: 197;
c) the HC1 comprises the amino acid sequence of SEQ ID NO: 193, the HC2 comprises the amino acid sequence of SEQ ID NO: 198, and the LC2 comprises the amino acid sequence of SEQ ID NO: 199; or
d) the HC1 comprises the amino acid sequence of SEQ ID NO: 200, the LC1 comprises the amino acid sequence of SEQ ID NO: 201, and the HC2 comprises the amino acid sequence of SEQ ID NO: 202.

133. A bispecific antibody, or a bispecific antigen-binding fragment thereof, that binds to the same epitope as the bispecific antibody, or the bispecific antigen-binding fragment thereof, according to claim 77.

134. A bispecific antibody, or a bispecific antigen-binding fragment thereof, that competes for binding to the same epitope with the bispecific antibody, or the bispecific antigen-binding fragment thereof, according to claim 77.

135. A bispecific antibody, or a bispecific antigen-binding fragment thereof, wherein the bispecific antibody, or the bispecific antigen-binding fragment thereof comprises an HC1 comprising the amino acid sequence of SEQ ID NO: 193, an HC2 comprising the amino acid sequence of SEQ ID NO: 198, and an LC2 comprising the amino acid sequence of SEQ ID NO: 199.

136. An isolated polynucleotide encoding the bispecific antibody, or the bispecific binding fragment thereof, of claim 77.

137. The isolated polynucleotide according to claim 136, wherein the polynucleotide comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to:
a) the VH of SEQ ID NO: 63;
b) the VH of SEQ ID NO: 95;
c) the VH of SEQ ID NO: 127; or
d) the VH of SEQ ID NO: 191.

138. The isolated polynucleotide according to claim 137, wherein the polynucleotide comprises or further comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to:
a) the VL of SEQ ID NO: 64;
b) the VL of SEQ ID NO: 96;
c) the VL of SEQ ID NO: 128; or
d) the VL of SEQ ID NO: 192.

139. The isolated polynucleotide according to claim 138, wherein the polynucleotide comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to:
a) the VH of SEQ ID NO: 31; or
b) the VH of SEQ ID NO: 159.

140. The isolated polynucleotide according to claim 139, wherein the polynucleotide comprises or further comprises a sequence comprising a nucleotide sequence coding for an amino acid sequence that is at least 90%, at least 91%, at least 92%, at least 93%, at least 94%, at least 95%, at least 96%, at least 97%, at least 98%, at least 99% or 100% identical to:
a) the VL of SEQ ID NO: 32; or
b) the VL of SEQ ID NO: 160.

141. The isolated polynucleotide according to claim 140, wherein the polynucleotide comprises a sequence encoding a first antigen-binding site that specifically binds TRBV19, the sequence comprising a nucleotide sequence encoding:
a) the amino acid sequence of SEQ ID NO: 31 and/or the amino acid sequence of SEQ ID NO: 32; or
b) the amino acid sequence of SEQ ID NO: 159 and/or the amino acid sequence of SEQ ID NO: 160.

142. The isolated polynucleotide according to claim 141, wherein the polynucleotide comprises a sequence encoding a second antigen-binding site that specifically binds EMR2, the sequence comprising a nucleotide sequence encoding:
a) the amino acid sequence of SEQ ID NO: 63 and/or the amino acid sequence of SEQ ID NO: 64;
b) the amino acid sequence of SEQ ID NO: 95 and/or the amino acid sequence of SEQ ID NO: 96;
c) the amino acid sequence of SEQ ID NO: 127 and/or the amino acid sequence of SEQ ID NO: 128; or
d) the amino acid sequence of SEQ ID NO: 191 and/or the amino acid sequence of SEQ ID NO: 192.

143. The isolated polynucleotide according to claim 142, wherein the polynucleotide comprises a sequence encoding an HC1 comprising the amino acid sequence of SEQ ID NO: 193 or 200.

144. The isolated polynucleotide according to claim 143, wherein the polynucleotide comprises a sequence encoding a LC1 comprising the amino acid sequence of SEQ ID NO: 201.

145. The isolated polynucleotide according to claim 144, comprising a nucleotide sequence encoding:
a) the amino acid sequence of SEQ ID NO: 193; or
b) the amino acid sequence of SEQ ID NO: 200 and the amino acid sequence of SEQ ID NO: 201.

146. The isolated polynucleotide according to claim 145, wherein the polynucleotide comprises a sequence encoding an HC2 comprising the nucleotide sequence of SEQ ID NO: 194, 196, 198, or 202.

147. The isolated polynucleotide according to claim 146, wherein the polynucleotide comprises a sequence encoding a LC2 comprising the nucleotide sequence of SEQ ID NO: 195, 197, or 199.

148. The isolated polynucleotide according to claim 147, wherein the polynucleotide comprises a sequence encoding:
a) the amino acid sequence of SEQ ID NO: 194 and/or the amino acid sequence of SEQ ID NO: 195;
b) the amino acid sequence of SEQ ID NO: 196 and/or the amino acid sequence of SEQ ID NO: 197;
c) the amino acid sequence of SEQ ID NO: 198 and/or the amino acid sequence of SEQ ID NO: 199; or
d) the amino acid sequence of SEQ ID NO: 202.

149. The polynucleotide according to claim 148, wherein the polynucleotide comprises a sequence encoding:
a) the amino acid sequence of SEQ ID NO: 193, the amino acid sequence of SEQ ID NO: 194 and the amino acid of SEQ ID NO: 195;
b) the amino acid sequence of SEQ ID NO: 193, the amino acid sequence of SEQ ID NO: 196 and the amino acid sequence of SEQ ID NO: 197;
c) the amino acid sequence of SEQ ID NO: 193, the amino acid sequence of SEQ ID NO: 198 and the amino acid sequence of SEQ ID NO: 199; or
d) the amino acid sequence of SEQ ID NO: 200, the amino acid sequence of SEQ ID NO: 201, and the amino acid sequence of SEQ ID NO: 202.

150. A vector comprising the isolated polynucleotide, according to claim 136.

151. The vector according to claim 150, wherein the polynucleotide is operably linked to an expression control sequence.

152. The vector, according to claim 151, wherein the vector is a viral vector.

153. The vector, according to claim 152, wherein the viral vector is selected from an adenoviral vector, an adeno-associated viral vector, a retroviral vector, a lentiviral vector, a herpes simplex virus vector, and a poxvirus vector.

154. A pharmaceutical composition comprising (i) the bispecific antibody, or the bispecific antigen-binding fragment thereof, according to claim 77, or an isolated polynucleotide encoding the bispecific antibody, or the bispecific antigen-binding fragment thereof, or a vector comprising the isolated polynucleotide, and (ii) a pharmaceutically acceptable carrier or excipient.

155. An isolated host cell expressing the bispecific antibody, or the bispecific antigen-binding fragment thereof, according to claim 77.

156. The host cell according to claim 155, wherein the cell is a hybridoma.

157. The host cell according to claim 155, wherein the bispecific antibody, or the bispecific antigen-binding fragment thereof, is recombinantly produced.

158. An isolated host cell comprising the isolated polynucleotide according to claim 136 or a vector comprising the isolated polynucleotide.

159. A method for treating a cancer in a subject in need thereof, the method comprising administering to the subject a therapeutically effective amount of the bispecific antibody, or the bispecific antigen-binding fragment thereof, according claim 77, or an isolated polynucleotide encoding the bispecific antibody, or the bispecific antigen-binding fragment thereof, or a vector comprising the isolated polynucleotide, or a pharmaceutical composition comprising the bispecific antibody, or the bispecific antigen-binding fragment thereof.

160. A method for inducing cytotoxicity of a cancer cell or redirecting immune or T cells against cancer cells, the method comprising administering to the cancer cells an effective amount of the bispecific antibody, or the bispecific antigen-binding fragment thereof, according to claim 77, or an isolated polynucleotide encoding the bispecific antibody, or the bispecific antigen-binding fragment thereof, or a vector comprising the isolated polynucleotide, or a pharmaceutical composition comprising the bispecific antibody, or the bispecific antigen-binding fragment thereof, or a host cell the bispecific antibody, or the bispecific antigen-binding fragment thereof, wherein the effective amount is sufficient to inhibit the growth or proliferation of the cancer cells.

161. The method, according to claim 160, wherein the cancer cells are in a subject and the bispecific antibody, or the bispecific antigen-binding fragment thereof, the polynucleotide, the vector, the pharmaceutical composition, or the host cell is administered to the subject.

162. The method, according to claim 161, wherein the administration is conducted ex vivo.

163. A method of redirecting a T cell to EMR2-expressing cancer cells in a subject in need thereof, the method comprising administering to the subject a therapeutically effective amount of the bispecific antibody, or the bispecific antigen-binding fragment thereof, according to claim 77, or a polynucleotide encoding the bispecific antibody, or the bispecific antigen-binding fragment thereof, or a vector comprising the isolated polynucleotide, or a pharmaceutical composition comprising the bispecific antibody, or the bispecific antigen-binding fragment thereof, or a host cell comprising the bispecific antibody, or the bispecific antigen-binding fragment thereof.

164. The method, according to claim 163, wherein the therapeutically effective amount administered is sufficient to redirect the T cell response to the cancer cells.

165. The method, according to claim 164, wherein the cancer is an EMR2-expressing cancer.

166. The method, according to claim 165, wherein the EMR2-expressing cancer is a hematological cancer.

167. The method, according to claim 166, wherein the hematological cancer is a myeloid malignancy.

168. The method, according to claim 167, wherein the cancer is acute myeloid leukemia (AML), chronic myelogenous leukemia (CML), or myelodysplastic neoplasms.

169. The method, according to claim 159, further comprising administering a second therapeutic agent.

170. The method, according to claim 169, wherein the second therapeutic agent is a surgery, a chemotherapy, an androgen deprivation therapy, or a radiation, or any combination thereof.

171. The bispecific antibody, or the bispecific antigen-binding fragment thereof, according to claim 77, for use in a method for treating a cancer in a subject in need thereof or for use in a method for inducing cytotoxicity of a cancer cell or redirecting immune or T cells against cancer cells.

172. The polynucleotide, according to claim 136, for use in a method for treating a cancer in a subject in need thereof or for use in a method for inducing cytotoxicity of a cancer cell or redirecting immune or T cells against cancer cells.

173. The vector, according to claim 150, for use in a method for treating a cancer in a subject in need thereof or for use in a method for inducing cytotoxicity of a cancer cell or redirecting immune or T cells against cancer cells.

174. The pharmaceutical composition, according to claim 154, for use in a method for treating a cancer in a subject in need thereof or for use in a method for inducing cytotoxicity of a cancer cell or redirecting immune or T cells against cancer cells.

175. The host cell according to claim 155, for use in a method for treating a cancer in a subject in need thereof or for use in a method for inducing cytotoxicity of a cancer cell or redirecting immune or T cells against cancer cells.

176. A method for generating the bispecific antibody, or the bispecific antigen-binding fragment thereof, according to claim 77, wherein the method comprises culturing a host cell comprising the bispecific antibody, or the bispecific antigen-binding fragment thereof, and isolating the bispecific antibody, or the bispecific antigen-binding fragment thereof.

177. A kit comprising (i) the bispecific antibody, or the bispecific antigen-binding fragment thereof, according to claim 77, or an isolated polynucleotide encoding the bispecific antibody, or the bispecific antigen-binding fragment thereof, or a vector comprising the isolated polynucleotide, or a pharmaceutical composition comprising the bispecific antigen-binding fragment thereof, or a host cell comprising the bispecific antigen-binding fragment thereof, and (ii) packaging for the same and/or instructions for use.
